(19) United States
(12) Patent Application Publication
(10) Pub. No.: US 2010/0081693 A1
(43) Pub. Date: Apr. 1, 2010

(54) THIADIAZOLE COMPOUND AND USE THEREOF

(75) Inventors: Hayato Takyo, Osaka (JP); Hideki Ihara, Osaka (JP)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: SUMITOMO CHEMICAL COMPANY, LIMITED, Chuo-ku, Tokyo (JP)

(21) Appl. No.: 12/441,052

(22) PCT Filed: Sep. 12, 2007
(86) PCT No.: PCT/JP2007/068216

§ 371 Date: Mar. 12, 2009

(30) Foreign Application Priority Data
Sep. 13, 2006 (JP) 2006-247806
Jun. 29, 2007 (JP) 2007-171885

(51) Int. Cl. A61K 31/454; C07D 285/08; A01N 43/836; C07D 417/12

(52) U.S. Cl. 514/326; 548/129; 514/361; 546/209

(57) ABSTRACT

A thiadiazole compound represented by the formula (I):

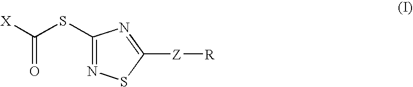

wherein R is a hydrogen atom, an optionally substituted C1-C7 chain hydrocarbon group etc., Z is an oxygen atom or a sulfur atom, X is a —NR²R³ group etc., R² and R³ each independently are a hydrogen atom, a C1-C4 alkyl group, a C3-C4 alkenyl group, a C1-C4 alkoxy group, or a phenyl group, or R² and R³ bind to each other at the ends thereof to form a C2-C7 alkanediyl group, has excellent controlling effect on a noxious arthropod.

### TECHNICAL FIELD

The present invention relates to a thiadiazole compound and use thereof for controlling a noxious arthropod.

### BACKGROUND ART

Previously, many compounds have been developed as an active ingredient of a pesticide, and have been put into practical use. In addition, a thiadiazole compound having a dimethylcarbamoyloxy group at a 3-position is described in J. Heterocyclic Chem., 16,961-971(1979).

### DISCLOSURE OF THE INVENTION

An object of the present invention is to provide a compound having excellent noxious arthropod controlling efficacy.

In order to find out a compound having excellent noxious arthropod controlling efficacy, the present inventors have intensively studied and, as a result, have found out that a thiadiazole compound represented by the following formula (I) has excellent noxious arthropod controlling efficacy, resulting in completion of the present invention.

Thus, the present invention is as follows:

[1] A thiadiazole compound represented by the formula (I):

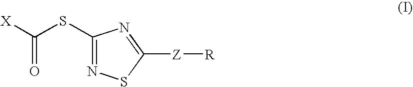

wherein

R is a hydrogen atom, (1) a C1-C7 chain hydrocarbon group optionally substituted with one or more substituents selected from the following A group, (2) a C3-C6 alkanoyl group, (3) a -Q group, (4) a -T-Q group, (5) a -T-O-Q group, or (6) a -T-O-T-Q group;

X is a —NR²R³ group or a group represented by the formula:

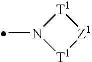

wherein Z is an oxygen atom or a sulfur atom;

Q is (1) a 3- to 10-membered carbocyclic group optionally substituted with one or more substituents selected from the following B group, or optionally substituted with one or more substituents selected from the following C group at the same position or adjacent positions, or (2) a 3- to 10-membered heterocyclic group optionally substituted with one or more substituents selected from the following B group, or optionally substituted with one or more substituents selected from the following C group at the same position or adjacent positions;

T is a C1-C4 alkanediyl group;

R² and R³ each independently are a hydrogen atom, a C1-C4 alkyl group, a C3-C4 alkenyl group, a C1-C4 alkoxy group, a benzyl group, or a phenyl group, or R² and R³ bind to each other at the ends thereof to form a C2-C7 alkanediyl group;

T¹ is a C2-C7 alkanediyl group; and

Z¹ is an oxygen atom, a sulfur atom, a —NH— group, or a —N(C1-C6 alkyl)- group;

A group: a monovalent substituent selected from the group consisting of a halogen atom, a cyano group, a nitro group, a —Z²-(T-Z²)r-R¹⁰ group, a —(Z²)p-C(═O)-(Z³)q-R¹⁰ group, and a —C(═NO—R¹⁰)—R¹¹ group;

B group: a monovalent substituent selected from the group consisting of a halogen atom, a cyano group, a nitro group, a —R¹² group, a —Z²-(T-Z²)r-R¹⁰ group, a -(T-Z²)s-R¹⁰ group, a —(Z²)p-C(═O)—(Z³)q-R¹⁰ group, a —C(═NO—R¹⁰)—R¹¹ group, a -Q group, a —Z²-Q¹ group, a -T-Q¹ group, a —Z²-T-Q¹ group, and a -T-Z²-Q¹ group;

C group: a divalent substituent selected from the group consisting of an oxygen atom, a sulfur atom, a -T- group, a —Z⁴-T-Z⁵- group, and a -T-Z⁴-T- group;

wherein r is 0, 1 or 2, p and q each independently are 0 or 1, s is 1 or 2,

Z² and Z³ each independently are an oxygen atom, a sulfur atom, a —NH— group, or —N(C1-C6 alkyl)- group,

Z⁴ and Z⁵ each independently are an oxygen atom or a sulfur atom,

R¹⁰ and R¹¹ each independently are (1) a C1-C7 chain hydrocarbon group optionally substituted with a halogen atom, or (2) a hydrogen atom,

R¹² is a C1-C7 chain hydrocarbon group optionally substituted with a halogen atom, and

Q¹ is (1) a 3- to 10-membered carbocyclic group optionally substituted with one or more substituents selected from the above A group, or optionally substituted with one or more substituents selected from the above C group at the same position or adjacent positions, or (2) a 3- to 10-membered heterocyclic group optionally substituted with one or more substituents selected from the above A group, or optionally substituted with one or more substituents selected from the above C group at the same position or adjacent positions (hereinafter referred to as the present compound 1).

[2] The thiadiazole compound according to the above [1], wherein X is a —NR²R³ group or a morpholino group, and R² and R³ each independently are a hydrogen atom, a C1-C4 alkyl group, a C3-C4 alkenyl group, a C1-C4 alkoxy group, a benzyl group, or a phenyl group, or a R² and R³ bind to each other at the ends thereof to form a C2-C7 alkanediyl group in the formula (I).

[3] The thiadiazole compound according to the above [1], wherein X is a —NR²R³ group or a morpholino group, and R² and R³ each independently are a C1-C4 alkyl group or a phenyl group, or R² and R³ bind to each other at the ends thereof to form a C2-C7 alkanediyl group in the formula (I).

[4] The thiadiazole compound according to any one of the above [1] to [3], wherein R¹ is a C1-C7 chain hydrocarbon group optionally substituted with one or more substituents selected from the above A group, a -Q group, a -T-Q group, a -T-O-Q group, or a -T-O-T-Q group,

Q is (1) a 3- to 10-membered carbocyclic group optionally substituted with one or more substituents selected from the above B group, or optionally substituted with one or more substituents selected from the above C group at the same position or adjacent positions, or (2) a 3- to 10-membered heterocyclic group optionally substituted with one or more substituents selected from the above B group, or optionally substituted with one or more substituents selected from the above C group at the same position or adjacent positions, and

T is a C1-C4 alkanediyl group in the formula (I).

[5] The thiadiazole compound according to any one of the above [1] to [3], wherein R is a C1-C7 chain hydrocarbon group optionally substituted with one or more substituents selected from the following D group, a -Q² group, a -T-Q² group, a -T-O-Q² group, or a -T-O-T-Q² group,

wherein Q² is (1) a 3- to 10-membered carbocyclic group optionally substituted with one or more substituents selected from the following E group, or optionally substituted with one or more substituents selected from the following F group at the same position or adjacent positions, or (2) a 3- to 10-membered heterocyclic group optionally substituted with one or more substituents selected from the following E group, or optionally substituted with one or more substituents selected from the above F group at the same position or adjacent positions, and

T is a C1-C4 alkanediyl group in the formula (I);

D group: a monovalent substituent selected from the group consisting of a halogen atom, a —Z²-(T-Z²)r-R¹⁰ group, and a —(Z²)p-C(═O)—(Z³)q-R¹⁰ group;

E group: a monovalent substituent selected from the group consisting of a halogen atom, a —R¹² group, a —Z² -(T-Z²)r-R¹⁰ group, a -(T-Z²)s-R¹⁰ group, a —(Z²)p-C(═O)—(Z³)q-R¹⁰ group, a -Q³ group, a -Z²-Q³ group, a -T-Q³ group, a —Z²-T-Q³ group, and a -T-Z²-Q¹ group;

F group: a divalent substituent selected from the group consisting of an oxygen atom, a -T- group, and a —Z⁴-T-Z⁵ - group;

wherein Q³ is a 3- to 10-membered carbocyclic group or a 3- to 10-membered heterocyclic group and r, p, q, s, Z², Z³, Z⁴, Z⁵,R¹⁰ and R¹² are as defined above.

[6] The thiadiazole compound according to any one of the above [1] to [3], wherein R is (1) a C1-C7 chain hydrocarbon group optionally substituted with one or more substituents selected from the above D group, (2) a -Q⁴ group, (3) a -T-Q⁴ group, (4) a -T-O-Q⁴ group, or (5) a -T-O-T-Q⁴ group,

Q⁴ is (1) a 3- to 6-membered carbocyclic group optionally substituted with one or more substituents selected from the above B group, or optionally substituted with one or more substituents selected from the above C group at the same position or adjacent positions, or (2) a 3- to 6-membered saturated heterocyclic group optionally substituted with one or more substituents selected from the above B group, or optionally substituted with one or more substituents selected from the above C group at the same position or adjacent positions in the formula (I).

[7] The thiadiazole compound according to any one of the above [1] to [3], wherein R is (1) a C1-C7 chain hydrocarbon group optionally substituted with one or more substituents selected from the above D group, (2) a -Q⁶ group, (3) -T-Q⁶ group, (4) a -T-O-Q⁶ group, or (5) a -T-O-T-Q⁶ group,

Q⁶ is a 3- to 6-membered carbocyclic group optionally substituted with one or more substituents selected from the above E group, or optionally substituted with one or more substituents selected from the above F group at the same position or adjacent positions, or (2) a 3- to 6-membered saturated heterocyclic group optionally substituted with one or more substituents selected from the above E group, or optionally substituted with one or more substituents selected from the above F group at the same position or adjacent positions, and

T is a C1-C4 alkanediyl group in the formula (I).

[8] The thiadiazole compound according to any one of the above [1] to [3], wherein R is (1) a C1-C7 chain hydrocarbon group optionally substituted with one or more substituents selected from the above D group, (2) a -Q⁷ group or (3) a -T-Q⁷ group,

Q⁷ is (1) a C3-C8 cycloalkyl group optionally substituted with one or more substituents selected from the above E group, or optionally substituted with one or more substituents selected from the above F group at the same position or adjacent positions, or (2) a group represented by the formula:

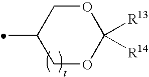

wherein t is 0 or 1, and

R¹³ and R¹⁴ each independently are a hydrogen atom, a C1-C4 alkyl group, a C2-C7 alkenyl group, a C2-C4 alkynyl group, a C1-C4 alkoxyalkyl group, or a -Q⁸ group, or R¹³ and R¹⁴ bind to each other at the ends thereof to form a C2-C7 alkanediyl group, or a —Z⁴-T-Z⁵— group,

Q⁸ is (1) a 3- to 10-membered carbocyclic group optionally substituted with one or more substituents selected from the above D group, or optionally substituted with one or more substituents selected from the above F group at the same position or adjacent positions, or (2) a 3- to 10-membered heterocyclic group optionally substituted with one or more substituents selected from the above D group, or optionally substituted with one or more substituents selected from the above F group at the same position or adjacent positions,

Z⁴ and Z⁵ each independently are an oxygen atom or a sulfur atom, and

T is a C1-C4 alkanediyl group in the formula [I].

[9] A thiadiazole compound represented by the formula (I′):

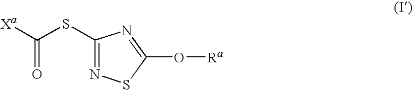

wherein

R^{a} is (1) a hydrogen atom, (2) a C1-C7 alkyl group, (3) a C1-C6 haloalkyl group, (4) a C3-C6 alkenyl group, (5) a C3-C6 haloalkenyl group, (6) a C3-C6 alkynyl group, (7) a C3-C6 haloalkynyl group, (8) a C2-C7 alkoxyalkyl group, (9) a C2-C6 alkylthioalkyl group, (10) a C3-C8 cycloalkyl group optionally substituted with one or more substituents selected from the following H group, (11) a C1-C4 alkyl group substituted with a C3-C8 cycloalkyl group optionally substituted with one or more substituents selected from the following H group, (12) a C5-C8 cycloalkenyl group optionally substituted with one or more substituents selected from the following H group, (13) a C1-C4 alkyl group substituted with a C5-C8 cycloalkenyl group optionally substituted with one or more substituents selected from the following H group, (14) a heterocyclic group optionally substituted with one or more substituents selected from the I group, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-membered heterocyclic group containing only one sulfur atom as the heteroatom thereof, (d) a 6-membered heterocyclic group containing only one or two sulfur atoms as the heteroatoms thereof, (e) a 5-membered heterocyclic group containing only one or two nitrogen atoms as the heteroatoms thereof, (f) a 5-membered heterocyclic group containing only a sulfur atom and a nitrogen atom as the heteroatoms thereof, (g) a 5-member heterocyclic group containing only a oxygen atom and a nitrogen atom as the heteroatoms thereof, and (h) a 6-membered heterocyclic group containing only one or two nitrogen atoms as the heteroatoms thereof, (15) a C1-C4 alkyl group substituted with a heterocyclic group optionally substituted with one or more substituents selected from the following I group, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-membered heterocyclic group containing only one sulfur atom as the heteroatom thereof, (d) a 6-membered heterocyclic group containing only one or two sulfur atoms as the heteroatoms thereof, (e) a 5-membered heterocyclic group containing only one or two nitrogen atoms as the heteroatoms thereof, (f) a 5-membered heterocyclic group containing only a sulfur atom and a nitrogen atom as the heteroatoms thereof, (g) a 5-member heterocyclic group containing only an oxygen atom and a nitrogen atom as the heteroatoms thereof, and (h) a 6-membered heterocyclic group containing only one or two nitrogen atoms as the heteroatoms thereof, (16) a phenyl group optionally substituted with one or more substituents selected from the following I group, (17) a C1-C4 alkyl group substituted with a phenyl group optionally substituted with one or more substituents selected from the following I group, (18) a C2-C6 formylalkyl group, (19) a C2-C6 cyanoalkyl group, (20) a C2-C6 hydroxyiminoalkyl group, (21) a C3-C7 alkoxyiminoalkyl group, (22) a C2-C8 alkylaminoalkyl group, (23) a C2-C6 alkoxycarbonylalkyl group, (24) a C2-C6 hydroxyalkyl group, or (25) a C3-C6 alkanoyl group; and

X^{a} is a morpholino group, or a —NR²R³ group, wherein R² and R³ each independently represent a hydrogen atom, a C1-C4 alkyl group, a C3-C4 alkenyl group, a C1-C4 alkoxy group, or a phenyl group, or R² and R³ bind to each other at the ends thereof to form a C2-C7 alkanediyl group;

H group: a monovalent substituent selected from the group consisting of a C1-C4 alkyl group optionally substituted with a halogen atom, a C2-C4 alkenyl group optionally substituted with a halogen atom, a C2-C4 alkynyl group optionally substituted with a halogen atom, and a halogen atom;

I group: a monovalent substituent selected from the group consisting of a C1-C4 alkyl group optionally substituted with a halogen atom, a C1-C4 alkoxy group optionally substituted with a halogen atom, a C1-C4 alkylthio group, a halogen atom, a cyano group, a nitro group, and a formyl group (hereinafter referred to as the present compound 2, further hereinafter both present compounds 1 and 2 together are referred to as the present compounds).

[10] The thiadiazole compound according to the above [9], wherein R^{a} is (1) a C1-C7 alkyl group, (2) a C1-C6 haloalkyl group, (3) a C3-C6 alkenyl group, (4) a C3-C6 haloalkenyl group, (5) a C3-C6 alkynyl group, (6) a C2-C7 alkoxyalkyl group, (7) a C2-C6 alkylthioalkyl group, (8) a C3-C8 cycloalkyl group optionally substituted with one or more substituents selected from the following J group, (9) a C1-C4 alkyl group substituted with a C3-C8 cycloalkyl group optionally substituted with one or more substituents selected from the following J group, (10) a C1-C4 alkyl group substituted with a C5-C8 cycloalkenyl group optionally substituted with one or more substituents selected from the following J group, (11) a heterocyclic group optionally substituted with one or more substituents selected from the following K group, said heterocyclic group being selected from the group consisting of (a) a 5-membered heterocyclic group containing only one or two oxygen atoms as the heteroatoms thereof, and (b) a 6-membered heterocyclic group containing only one or two oxygen atoms as the heteroatoms thereof, (12) a C1-C4 alkyl group substituted with a heterocyclic group optionally substituted with one or more substituents selected from the following K group, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-membered heterocyclic group containing only one or two nitrogen atoms as the heteroatoms thereof, (d) a 5-membered heterocyclic group containing only a sulfur atom and a nitrogen atom as the heteroatoms thereof, and (e) a 6-membered heterocyclic group containing only one or two nitrogen atoms as the heteroatom thereof, or (13) a C1-C4 alkyl group substituted with a phenyl group optionally substituted with one or more substituent selected from the following L group in the formula (I′);

J group: a monovalent substituent selected from the group consisting of a C1-C4 alkyl group optionally substituted with a halogen atom, a C2-C4 alkynyl group, and a halogen atom;

K group: a monovalent substituent selected from the group consisting of a C1-C4 alkyl group, and a halogen atom;

L group: a monovalent substituent selected from the group consisting of a C1-C4 alkyl group optionally substituted with a halogen atom, a C1-C4 alkoxy group optionally substituted with a halogen atom, an alkylthio group, and a halogen atom.

[11] The thiadiazole compound according to the above [9], wherein R^{a} is (1) a C1-C7 alkyl group, (2) a C1-C6 haloalkyl group, (3) a C3-C6 alkenyl group, (4) a C3-C6 haloalkenyl group, (5) a C3-C6 alkynyl group, (6) a C2-C7 alkoxyalkyl group, (7) a heterocyclic group optionally substituted with one or more C1-C4 alkyl groups, said heterocyclic group being selected from the group consisting of (a) a 5-membered heterocyclic group containing only one or two oxygen atoms as the heteroatom thereof, and (b) a 6-membered heterocyclic group containing only one or two oxygen atoms as the heteroatoms thereof, or (8) a C1-C4 alkyl group substituted with a heterocyclic group optionally substituted with one or more C1-C4 alkyl groups, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-membered heterocyclic group containing only one or two nitrogen atoms as the heteroatoms thereof, and (d) and a 6-membered heterocyclic group containing one or two nitrogen atoms as the heteroatoms thereof in the formula (I′).

[12] The thiadiazole compound according to any one of the above [9] to [11], wherein X^{a} is a morpholino group, or a —NR²R³ group, wherein R² and R³ each independently are a C1-C4 alkyl group or a phenyl group, or R² and R³ bind to each other at the ends thereof to form a C2-C7 alkanediyl group in the formula (I′).

[13] A thiadiazole compound represented by the formula (II):

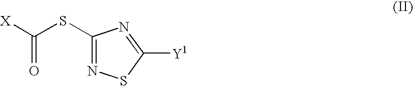

wherein

Y¹ is a halogen atom,

X is a —NR²R³ group or a group represented by the formula:

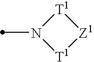

R² and R³ each independently are a hydrogen atom, a C1-C4 alkyl group, a C3-C4 alkenyl group, a C1-C4 alkoxy group, a benzyl group or a phenyl group, or R² and R³ bind to each other at the ends thereof to form a C2-C7 alkanediyl group,

T¹ is a C2-C7 alkanediyl group, and

Z¹ is an oxygen atom, a sulfur atom, a —NH— group or a —N(C1-C6 alkyl)- group (hereinafter referred to as the present intermediate).

[14] The thiadiazole compound according to the above [13], wherein X is a —NR²R³ group or a morpholino group, and R² and R³ each independently are a C1-C4 alkyl group, or a phenyl group, or R² and R³ bind at an end to be C2-C7 alkanediyl group in the formula (II).

[15] An agent for controlling a noxious arthropod comprising a compound according to any one of the above [1] to [14] as an active ingredient.

[16] Use of a compound according to any one of the above [1] to [14] for controlling a noxious arthropod.

[17] A method for controlling a noxious arthropod comprising applying a compound according to any one of the above [1] to [14] to a noxious arthropod or a place where a noxious arthropod inhabits.

### BEST MODE FOR CARRYING OUT THE INVENTION

Hereinafter, various substituents used herein will be explained by referring to examples thereof. In the present invention, for example, the term the “C2-C6” in the expression of “C2-C6 alkoxyalkyl group” means that a total number of carbons constituting the alkoxyalkyl group is 2 to 6. The similar expression is also used with respect to the other substituents.

In addition to the A group, the B group and the C group, substituents selected from the following groups are defined as particular substituents used herein. D group: a monovalent substituent selected from the group consisting of a halogen atom, a —Z²-(T-Z²)r-R¹⁰ group, and a —(Z²)p-C(═O)—(Z³)q-R¹⁰ group;
E group: a monovalent substituent selected from the group consisting of a halogen atom, a —R¹² group, a —Z²-(T-Z2)r-R¹⁰ group, a -(T-Z²)s-R¹⁰ group, a —(Z²)p-C(═O)—(Z³)q-R¹⁰ group, a -Q³ group, a —Z²-Q³ group, a -T-Q³ group, a —Z²-T-Q³ group, and a-T-Z²-Q³ group; and
F group: a divalent substituent selected from the group consisting of an oxygen atom, a -T- group, and a —Z⁴ -T-Z⁵— group;
wherein Q³ represents a 3- to 10-membered carbocyclic group or a 3- to 10-membered heterocyclic group, and r, p, q, S, Z², Z³, Z⁴, Z⁵, R¹⁰ and R¹² are as defined above.
H group: a monovalent substituent selected from the group consisting of a C1-C4 alkyl group optionally substituted with a halogen atom, a C2-C4 alkenyl group optionally substituted with a halogen atom, a C2-C4 alkynyl group optionally substituted with a halogen atom, and a halogen atom.
I group: a monovalent substituent selected from the group consisting of a C1-C4 alkyl group optionally substituted with a halogen atom, a C1-C4 alkoxy group optionally substituted with a halogen atom, a C1-C4 alkylthio group, a halogen atom, a cyano group, a nitro group, and a formyl group.
J group: a monovalent substituent selected from the group consisting of a C1-C4 alkyl group optionally substituted with a halogen atom, a C2-C4 alkynyl group, and a halogen atom.
K group: a monovalent substituent selected from the group consisting of a C1-C4 alkyl group, and a halogen atom.
L group: a monovalent substituent selected from the group consisting of a C1-C4 alkyl group optionally substituted with a halogen atom, a C1-C4 alkoxy group optionally having a halogen atom, an alkylthio group, and a halogen atom.

Examples of the “—NR²R³ group” of X include the group wherein R² is a hydrogen atom and R³ is a hydrogen atom (i.e., amino group); the group wherein R² is a hydrogen atom and R³ is a C1-C4 alkyl group (e.g., methylamino group, ethylamino group, etc.); the group wherein R² is a C1-C4 alkyl group and R³ is a C1-C4 alkyl group (e.g., dimethylamino group, diethylamino group, dipropylamino group, etc.); the group wherein R² is a hydrogen atom and R³ is a C3-C4 alkenyl group (e.g., allylamino group. etc.); the group wherein R² is a C3-C4 alkenyl group and R³ is a C3-C4 alkenyl group (e.g., diallylamino group, etc.); the group wherein R² is a hydrogen atom and R³ is a benzyl group (i.e., benzylamino group); the group wherein R² is a C1-C4 alkyl group and R³ is a benzyl group (e.g., N-methyl-N-benzylamino group, N-ethyl-N-benzylamino group, etc.); the group wherein R² is a benzyl group and R³ is a benzyl group (dibenzylamino group); the group wherein R is a hydrogen atom and R³ is a phenyl group (phenylamino group); the group wherein R² is a C1-C4 alkyl group and R³ is a phenyl group (e.g., N-methyl-N-phenylamino group, N-ethyl-N-phenylamino group, etc.); the group wherein R² is a phenyl group and R³ is a phenyl group (diphenylamino group); the group wherein R² and R³ bind to each other at the ends thereof to form a C2-C7 alkanediyl group (e.g., 1-pyrrolidinyl group, 2,5-dimethyl-1-pyrrolidinyl group, piperidino group, etc.).

Examples of the group represented by the formula:

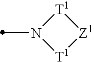

of X include the group wherein Z¹ is an oxygen atom and T¹ is a C2-C7 alkanediyl group (e.g., morpholino group, 2,6-dimethylmorpholino group, etc.); the group wherein Z¹ is a sulfur atom and T¹ is a C2-C7 alkanediyl group (e.g., thiomorpholino group, etc.); the group wherein Z¹ is a —N(C1-C4 alkyl group)- and T¹ is a C2-C7 alkanediyl group (e.g., 4-methyl-1-piperazinyl group, etc.).

Examples of the “C1-C7 chain hydrocarbon group optionally substituted with one or more substituents selected from the A group” of R include a C1-C7 alkyl group, a C3-C7 alkenyl group, a C3-C7 alkynyl group, a C1-C7 haloalkyl group, a C3-C7 haloalkenyl group, a C3-C7 haloalkynyl group, a (C1-C7 alkoxy)C1-C7 alkyl group, a {(C1-C7 alkoxy)C1-C4 alkoxy}C1-C7 alkyl group, a [{(C1-C7 alkoxy)C1-C4 alkoxy}C1-C4 alkoxy]C1-C7 alkyl group, a (C1-C7 haloalkoxy)C1-C7 alkyl group, a (C3-C7 alkenyloxy)C1-C7 alkyl group, a C3-C7 alkynyloxy)C1-C7 alkyl group, a (C3-C7 haloalkenyloxy)C1-C7 alkyl group, a (C3-C7 haloalkynyloxy)C1-C7 alkyl group, a (C1-C7 alkylthio)alkyl group, a C1-C7 hydroxyiminoalkyl group, a (C1-C7 alkoxyimino)C1-C7 alkyl group, a (C1-C7 alkyoamino)C1-C7 alkyl group, a C2-C8 cyanoalkyl group, a C2-C8 formylalkyl group, a (C2-C8 alkanoyl)C1-C7 alkyl group, a (C2-C8 alkoxycarbonyl)C1-C7 alkyl group, a C1-C7 hydroxyalkyl group, and a (C2-C8 alkylcarbonyloxy)C1-C7 alkyl group; preferably a C1-C6 alkyl group, a C3-C6 alkenyl group, a C3-C6 alkynyl group, a C1-C6 haloalkyl group, a C3-C6 haloalkenyl group, a C3-C6 haloalkynyl group, a C2-C7 alkoxyalkyl group, a C2-C6 alkylthioalkyl group, a C2-C6 formylalkyl group, a C2-C6 cyanoalkyl group, a C2-C6 hydroxyiminoalkyl group, a C3-C7 alkoxyiminoalkyl group, a C3-C10 alkylaminoalkyl group, a C2-C6 alkoxycarbonylalkyl group, and a C2-C6 hydroxyalkyl group.

The “-Q group” of R is a 3- to 10-membered carbocyclic group optionally substituted with one or more substituents selected from the B group, or optionally substituted with one or more substituents selected from the C group at the same position or adjacent positions; or a 3- to 10-membered heterocyclic group optionally substituted with one or more substituents selected from B group, or optionally substituted with one or more substituents selected from the C group at the same position or adjacent positions.

Examples of the “3- to 10-membered carbocyclic group optionally substituted with one or more substituents selected from the B group, or optionally substituted with one or more substituents selected from the C group at the same position or adjacent positions” include a C3-C8 cycloalkyl group optionally substituted with one or more substituents selected from the B group and the C group, a C5-C8 cycloalkenyl group optionally substituted with one or more substituents selected from the B group and the C group, and a phenyl group optionally substituted with one or more substituents selected from the B group and the C group; preferably a C3-C8 cycloalkyl group optionally substituted with one or more substituents selected from the H group, a C5-C8 cycloalkenyl group optionally substituted with one or more substituents selected from the H group, and a phenyl group optionally substituted with one or more substituents selected from the I group.

Examples of the “3- to 10-membered heterocyclic group optionally substituted with one or more substituents selected from the B group, or optionally substituted with one or more substituents selected from the C group at the same position or adjacent positions” include a 3- to 6-membered saturated heterocyclic group optionally substituted with one or more substituents selected from the B group and the C group whose heteroatom(s) are only oxygen atom(s) or sulfur atom(s), a 3- to 6-membered saturated heterocyclic group optionally substituted with one or more substituents selected from the B group and the C group whose heteroatom(s) are only nitrogen atom(s), a 5- to 6-membered unsaturated heterocyclic group optionally substituted with one or more substituents selected from the B group and the C group whose heteroatom(s) are only oxygen atom(s) or sulfur atom(s), a 5- to 6-membered unsaturated heterocyclic group optionally substituted with one or more substituents selected from the B group and the C group whose heteroatom(s) are only nitrogen atom(s), and a 5- to 6-membered unsaturated heterocyclic group optionally substituted with one or more substituents selected from the B group and the C group whose heteroatom(s) are sulfur(s) atom and nitrogen atom(s), or only oxygen atom(s) and a nitrogen atom(s); preferably a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 6-membered heterocyclic group containing only one or two sulfur atoms as the heteroatoms, a 5-membered heterocyclic group containing only one or two nitrogen atoms as the heteroatoms, a 5-membered heterocyclic group containing only a sulfur atom and a nitrogen atom as the heteroatoms, a 5-membered heterocyclic group containing only an oxygen atom and a nitrogen atom as the heteroatoms, and a 6-membered heterocyclic group containing only one or two nitrogen atoms as the heteroatoms, said heterocyclic group being optionally substituted with one or more substituents selected from the B group and the C group; further preferably a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 6-membered heterocyclic group containing only one or two sulfur atoms as the heteroatoms, a 5-membered heterocyclic group containing only one or two nitrogen atoms as the heteroatoms, a 5-membered heterocyclic group containing only a sulfur atom and a nitrogen atom as the heteroatoms, a 5-membered heterocyclic group containing only an oxygen atom and a nitrogen atom as the heteroatoms, and a 6-membered heterocyclic group containing only one or two nitrogen atoms as the heteroatoms, said heterocyclic group being optionally substituted with one or more substituents selected from the I group.

The “-T-Q group” of R is a C1-C4 alkyl group substituted with a 3- to 10-membered carboxylic group optionally substituted with one or more substituents selected from the B group, or optionally substituted with one or more substituents selected from the C group at the same position or adjacent positions; or a C1-C4 alkyl group substituted with a 3- to 10-membered heterocyclic group optionally substituted with one or more substituents selected from the B group, or optionally substituted with one or more substituents selected from the C group at the same position or adjacent positions.

Examples of the “C1-C4 alkyl group substituted with 3- to 10-membered carbocyclic group optionally substituted with one or more substituents selected from the B group, or optionally substituted with one or more substituents selected from the C group at the same position or adjacent positions” include a C1-C4 alkyl group substituted with a C3-C8 cycloalkyl group optionally substituted with one or more monovalent groups selected from the B group and the C group, a C1-C4 alkyl group substituted with a C5-C8 cycloalkenyl group optionally substituted with one or more monovalent groups selected from the B group and the C group, and a C1-C4 alkyl group substituted with a phenyl group optionally substituted with one or more monovalent groups selected from the B group and the C group; preferably a C1-C4 alkyl group substituted with a C3-C8 cycloalkyl group optionally substituted with one or more monovalent groups selected from the H group, a C1-C4 alkyl group substituted with a C5-C8 cycloalkenyl group optionally substituted with one or more groups selected from the H group, and a C1-C4 alkyl group substituted with a phenyl group optionally substituted with one or more monovalent groups selected from the I group.

Examples of the “C1-C4 alkyl group substituted with a 3- to 10-membered heterocyclic group optionally substituted with one or more substituents selected from the B group, or optionally substituted with one or more substituents selected from the C group at the same position or adjacent positions” include a C1-C4 alkyl group substituted with a 3- to 6-membered saturated heterocyclic group optionally substituted with one or more substituents selected from the B group and the C group whose heteroatom(s) are only oxygen atom(s) or sulfur atom(s), a C1-C4 alkyl group substituted with a 3- to 6-membered saturated heterocyclic group optionally substituted with one or more substituents selected from the B group and the C group whose heteroatom(s) are only nitrogen atom(s), a C1-C4 alkyl group substituted with a 5- to 6-membered unsaturated heterocyclic group optionally substituted with one or more substituents selected from the B group and the C group whose heteroatom(s) are only oxygen atom(s) or sulfur atom(s), a C1-C4 alkyl group substituted with a 5- to 6-membered unsaturated heterocyclic group optionally substituted with one or more substituents selected from the B group and the C group whose heteroatom(s) are only nitrogen atom(s), and a C1-C4 alkyl group substituted with a 5- to 6-membered unsaturated heterocyclic group optionally substituted with one or more substituents selected from the B group and the C group whose heteroatom(s) are sulfur atom(s) and nitrogen atom(s), or only oxygen atom(s) and nitrogen atom(s); preferably a C1-C4 alkyl group substituted with a 5-membered heterocyclic group containing only one or two oxygen atoms as the heteroatoms, a C1-C4 alkyl group substituted with a 6-membered heterocyclic group containing only one or two oxygen atoms as the heteroatoms, a C1-C4 alkyl group substituted with a 5-membered heterocyclic group containing only one sulfur atom as the heteroatom, a C1-C4 alkyl group substituted with a 6-membered heterocyclic group containing only one or two sulfur atoms as the heteroatoms, a C1-C4 alkyl group substituted with a 5-membered heterocyclic group containing only one or two nitrogen atoms as the heteroatoms, a C1-C4 alkyl group substituted with a 5-membered heterocyclic group containing only a sulfur atom and a nitrogen atom as the heteroatoms, a C1-C4 alkyl group substituted with a 5-membered heterocyclic group containing only an oxygen atom and a nitrogen atom as the heteroatoms, and a C1-C4 alkyl group substituted with a 6-membered heterocyclic group containing only one or two nitrogen atoms as the heteroatoms, said heterocyclic group being optionally substituted with one or more substituents selected from the B group and the C group; further preferably a C1-C4 alkyl group substituted with a 5-membered heterocyclic group containing only one or two oxygen atoms as the heteroatoms, a C1-C4 alkyl group substituted with a 6-membered heterocyclic group containing only one or two oxygen atoms as the heteroatoms, a C1-C4 alkyl group substituted with a 5-membered heterocyclic group containing only one sulfur atom as the heteroatom, a C1-C4 alkyl group substituted with a 6-membered heterocyclic group containing only one or two sulfur atoms as the heteroatoms, a C1-C4 alkyl group substituted with a 5-membered heterocyclic group containing only one or two nitrogen atoms as the heteroatoms, a C1-C4 alkyl group substituted with a 5-membered heterocyclic group containing only a sulfur atom and a nitrogen atom as the heteroatoms, a C1-C4 alkyl group substituted with a 5-membered heterocyclic group containing only an oxygen atom and a nitrogen atom as the heteroatoms, and a C1-C4 alkyl group substituted with a 6-membered heterocyclic group containing only one or two nitrogen atoms as the heteroatoms said heterocyclic group being optionally substituted with one or more substituents selected from the I group.

The “-T-O-Q group” of R is a C1-C4 alkyl group substituted with a 3- to 10-membered carbocyclic group optionally substituted with one or more substituents selected from the B group, or optionally substituted with one or more substituents selected from the C group at the same position or adjacent positions via an oxygen atom; or a C1-C4 alkyl group is substituted with a 3- to 10-membered heterocyclic group optionally substituted with one or more substituents selected from the B group, or optionally substituted with one or more substituents selected from the C group at the same position or adjacent positions via an oxygen atom. Examples thereof include a C1-C4 alkyl group substituted with a phenyloxy group optionally substituted with one or more substituents selected from the B group and the C group, and a C1-C4 alkyl group substituted with a 3- to 10-membered heterocyclic group optionally substituted with one or more substituents selected from the B group and the C group via an oxygen atom.

The “-T-O-T-Q group” of R is a C1-C4 alkoxy group substituted with a 3- to 10-membered carbocyclic group optionally substituted with one or more substituents selected from the B group, or optionally substituted with one or more substituents selected from the C group at the same position or adjacent positions; or a C1-C4 alkoxy group substituted with a 3- to 10-membered heterocyclic group optionally substituted with one or more substituents selected from the B group, or optionally substituted with one or more substituents selected from the C group at the same position or adjacent positions. Examples thereof include a C1-C4 alkyl group substituted with a benzyloxy group optionally substituted with one or more substituents selected from the B group and the C group.

Examples of the “3- to 10-membered carbocyclic group” include a C3-C8 cycloalkyl group, a C5-C8 cycloalkenyl group, a phenyl group, and a naphthyl group.

Examples of the “3- to 10-membered heterocyclic group” include a 3- to 8-membered heterocyclic group having, as the ring constituting atom(s), at least one kind of atom selected from the group consisting of an oxygen atom, a sulfur atom and a nitrogen atom, for example, a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 6-membered heterocyclic group containing only one or two sulfur atoms as the heteroatoms, a 5-membered heterocyclic group containing only one or two nitrogen atoms as the heteroatoms, a 5-membered heterocyclic group containing only a sulfur atom and a nitrogen atom as the heteroatoms, a 5-membered heterocyclic group containing only an oxygen atom and a nitrogen atom as the heteroatoms, and a 6-membered heterocyclic group containing only one or two nitrogen atoms as the heteroatoms.

Examples of the “—Z²-(T-Z²)r-R¹⁰ group” as a substituent of the A group and the B group include the group wherein r is 0, Z² is an oxygen atom and R¹⁰ is a hydrogen atom (i.e., hydroxyl group); the group wherein r is 0, Z² is an oxygen atom and R¹⁰ is a C1-C7 chain hydrocarbon group optionally substituted with a halogen atom (e.g., methoxy group, ethoxy group, propoxy group, isopropoxy group, 2-propenyloxy group, 2,2,2-trifluoroethoxy group, 3,3-dichloro-2-propenyloxy group, etc.); the group wherein r is 0, Z² is a sulfur atom and R¹⁰ is a C1-C7 chain hydrocarbon group optionally substituted with a halogen atom (e.g., methylthio group, ethylthio group etc.); and the group in which r is 1, Z² is an oxygen atom, T is a C1-C4 alkanediyl group and R¹⁰ is a C1-C7 chain hydrocarbon group optionally substituted with a halogen atom (e.g., methoxymethoxy group, ethoxymethoxy group, 2-methoxyethoxy group, 2-ethoxyethoxy group, etc.).

Examples of the “—(Z²)p-C(═O)—(Z³)q-R¹⁰ group” as a substituent of the A group and the B group include the group wherein p is 0, q is 0 and R¹⁰ is a hydrogen atom (i.e., formyl group); the group wherein p is 0, q is 0 and R¹⁰ is a C1-C7 chain hydrocarbon group optionally substituted with a halogen atom (e.g., acetyl group, propanoyl group, etc.); the group wherein p is 1, q is 0, Z² is an oxygen atom and R¹⁰ is a hydrogen atom (i.e., formyloxy group); the group wherein p is 1, q is 0, Z² is an oxygen atom and R¹⁰ is a C1-C7 chain hydrocarbon group optionally substituted with halogen atom (e.g., acetyloxy group, propanoyloxy group, etc.); the group wherein p is 0, q is 1, Z³ is an oxygen atom and R¹⁰ is a hydrogen atom (i.e., carboxyl group); the group wherein p is 0, q is 1, Z³ is an oxygen atom and R¹⁰ is a C1-C7 chain hydrocarbon group optionally substituted with a halogen atom (e.g., methoxycarbonyl group, ethoxycarbonyl group, tert-butoxycarbonyl group, etc.); and the group wherein p is 1, q is 1, Z² is a sulfur atom, Z³ is a —N(C1-C6 alkyl group)- group and R¹⁰ is a C1-C7 chain hydrocarbon group optionally substituted with a halogen atom (e.g., —SC(═O)NMe₂, —SC(═O)NEt₂, etc.).

Examples of the “—C(═NO—R¹⁰)—R¹¹ group” as a substituent of the A group and the B group include the group wherein R¹⁰ is a hydrogen atom and R¹¹ is a hydrogen atom (i.e., hydroxyiminomethyl group); the group wherein R¹⁰ is a C1-C7 chain hydrocarbon group optionally substituted with a halogen atom and R¹¹ is a hydrogen atom (e.g., methoxyiminomethyl group, ethoxyiminomethyl group, etc.); and the group wherein R¹⁰ is a C1-C7 chain hydrocarbon group optionally substituted with a halogen atom and R¹¹ is a C1-C7 chain hydrocarbon group optionally substituted with a halogen atom (e.g., 2-(methoxyimino)ethyl group, 2-(ethoxyimino)ethyl group, etc.).

Examples of the “-(T-Z²)s-R¹⁰ group” as a substituent of the B group include the group wherein s is 1, Z² is an oxygen atom, T is a C1-C4 alkanediyl group and R¹⁰ is a hydroxy group (e.g., hydroxymethyl group, 2-hydroxyethyl group, etc.); the group wherein s is 1, Z² is an oxygen atom, T is a C1-C4 alkanediyl group and R¹⁰ is a C1-C7 chain hydrocarbon group optionally substituted with a halogen atom (e.g., methoxymethyl group, ethoxymethyl group, 2-methoxyethyl group, 2-ethoxyethyl group, etc.).

Examples of the “—Z²-Q¹ group” as a substituent of the B group include the group wherein a 3- to 10-membered carbocyclic group binds thereto via an oxygen atom (e.g., phenoxy group, cyclohexyloxy group, etc.); the group wherein a 3- to 10-membered heterocyclic group binds thereto via an oxygen atom (e.g., 4-pyridyloxy group, etc.), and an amino group substituted with a 3- to 10-membered carbocyclic group (e.g., phenylamino group, etc.).

Examples of the “-T-Q¹ group” as a substituent of the B group include a C1-C4 alkyl group substituted with a 3- to 10-membered carbocyclic group (e.g., benzyl group, cyclohexylmethyl group, etc.); and a C1-C4 alkyl group substituted with a 3- to 10-membered heterocyclic group (e.g., 4-pyridylmethyl group, etc.).

Examples of the “—Z²-T-Q¹ group” as a substituent of the B group include a C1-C4 alkoxy group substituted with a 3- to 10-membered carbocyclic group (e.g., benzyloxy group, etc.).

Examples of the “-T-Z²-Q¹ group” as a substituent of the B group include the group wherein a 3- to 10-membered carbocyclic group binds to a C1-C4 alkoxy group via an oxygen atom (e.g., phenoxymethyl group, 1-phenoxyethyl group, etc.).

Examples of the “—Z⁴-T-Z⁵-group” as a divalent group of the C group include the group wherein Z is an oxygen atom and Z⁵ is an oxygen atom (e.g., —OCH₂CH₂O—, —OC(CH₃)₂O—, etc.).

Examples of the “-T-Z⁴-T-group” as a divalent group of the C group include the group wherein Z⁴ is an oxygen atom (e.g., —CH₂OCH₂—, —CH₂CH₂OCH₂CH₂—, etc).

The state where a 3- to 10-membered carbocyclic group or a 3- to 10-membered heterocyclic group is replaced by a substituent selected from the C group at the same position will be shown below, by way of an example, in case of a cyclohexyl group.

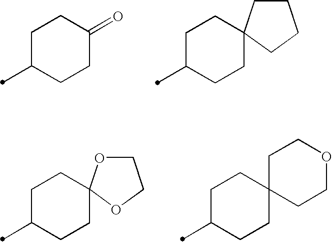

In addition, the state where a 3- to 10-membered carbocyclic group or a 3- to 10-membered heterocyclic group is replaced by a substituent selected from the C group at adjacent positions will be shown below, by way of an example, in case of a cyclohexyl group.

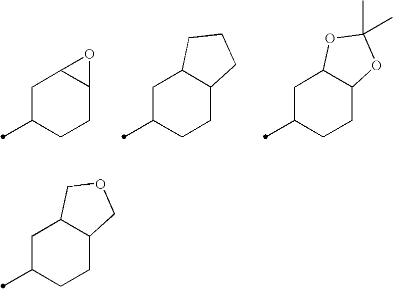

As a substituent of the H group, examples of the “C1-C4 alkyl group optionally substituted with a halogen atom” include a methyl group, an ethyl group, a propyl group, an isopropyl group, a tert-butyl group, a trifluoromethyl group, a difluoromethyl group, and a pentafluoroethyl group; examples of the “C2-C4 alkenyl group optionally substituted with a halogen atom” include a vinyl group, and an allyl group; examples of the “C2-C4 alkynyl group optionally substituted with a halogen atom” include an ethynyl group, and example of the “halogen atom” include a fluorine atom, and a chlorine atom.

As a substituent of the I group, examples of the “C1-C4 alkyl group optionally substituted with a halogen atom” include a methyl group, an ethyl group, a propyl group, an isopropyl group, a tert-butyl group, a trifluoromethyl group, a difluoromethyl group, and an a pentafluoromethyl group; examples of the “C1-C4 alkoxy group optionally substituted with a halogen atom” include a methoxy group, an ethoxy group, a propoxy group, an isopropoxy group, a trifluoromethoxy group, and a difluoromethoxy group; and examples of the “C1-C4 alkylthio group” include a methylthio group, and an ethylthio group.

Examples of the “halogen atom” includes a fluorine atom, a chlorine atom, a bromine atom, and an iodine atom.

The “C1-C7 alkyl group” means a monovalent group of a C1-C7 straight or branched saturated hydrocarbon, and examples thereof includes a methyl group, an ethyl group, a propyl group, an isopropyl group, a butyl group, isobutyl group, a sec-butyl group, a tert-butyl, a pentyl group, an isopentyl group, a neopentyl group, a sec-pentyl group, a tert-pentyl group, a 2-methylbutyl group, a 1,2-dimethylpropyl group, 1-ethylpropyl group, a hexyl group, a 3,3-dimethylbutyl group, a 1,2-dimethylbutyl group, a 2-methylpentyl group, a 3-methylpentyl group, a 4-methylpentyl group, a 2,2-dimethylbutyl group, a 2,3-dimethylbutyl group, a 2-ethylbutyl group, a 1-methylpentyl group, a 1,2,2-trimethylpropyl group, a 1,3-dimethylbutyl group, a 1-ethylbutyl group, a 1-ethyl-2-methylpropyl group, a heptyl group, a 1-ethyl-2,2-dimethylpropyl group, a 1-methylhexyl group, a 2-methylhexyl group, a 3-methylhexyl group, a 4-methylhexyl group, a 5-methylhexyl group, a 1,2-dimethylpentyl group, a 1,3-dimethylpentyl group, a 1,4-dimethylpentyl group, a 2,2-dimethylpentyl group, a 2,3-dimethylpentyl group, a 2,4-dimethylpentyl group, a 3,3-dimethylpentyl group, a 3,4-dimethylpentyl group, a 4,4-dimethylpentyl group, a 1-ethylpentyl group, a 2-ethylpentyl group, a 3-ethylpentyl group, a 1-propylbutyl group, a 2-ethyl-1-methylbutyl group, a 1-ethyl-2-methylbutyl-group, a 1-ethyl-3-metylbutyl group, a 1-tert-butylpropyl group, and a 3-ethyl-4-methylbutyl group.

The “C3-C7 alkenyl group” means a monovalent group of a C3-C6 straight or branched hydrocarbon having at least one double bond, and examples thereof include a 2-propenyl group, a 2-butenyl group, a 3-butenyl group, a 1-methyl-2-butenyl group, a 2-methyl-2-propenyl group, 2-pentenyl group, a 3-pentenyl group, a 4-pentenyl group, a 2-methyl-2-butenyl group, a 2-methyl-2-butenyl group, a 2-methyl-3-butenyl group, a 3-methyl-2-butenyl group, a 3-methyl-3-butenyl group, a 1-methyl-1-butenyl group, a 1-methyl-3-butenyl group, a 1,2-dimethyl-2-propenyl group, a 1-ethyl-2-propenyl group, a 2-hexenyl group, a 3-hexenyl group, a 4-hexenyl group, a 5-hexenyl group, a 1-methyl-3-pentenyl group, a 1-methyl-4-pentenyl group, a 2-methyl-2-pentenyl group, a 3-methyl-3-pentenyl group, a 3-methyl-4-pentenyl group, a 4-methyl-3-pentenyl group, a 4-methyl-4-pentenyl group, a 2-propyl-2-propenyl group, a 1-propyl-2-propenyl group, a 1,2-dimethyl-2-butenyl group, a 1,2-dimethyl-3-butenyl group, a 1,3-dimethyl-2-butenyl group, a 1,3-dimethyl-3-butenyl group, a 1-ethyl-2-methyl-2-propenyl group, a 1-(1-methylethyl)-27-propenyl group, a 1-ethyl-2-butenyl group, and a 1-ethyl-3-butenyl group.

The “C3-C7 alkylnyl group” means a monovalent group of a C3-C6 straight or branched hydrocarbon having at least one triple bond, and examples thereof include a 2-propynyl group, a 1-methyl-2-propynyl group, a 1,1-dimethyl-2-propynyl group, a 1-ethyl-2-propynyl group, a 1-propyl-2-propynyl group, a 1-(1-methylethyl)-2-propnyl group, a 2-butynyl group, a 1-methyl-2-butynyl group, a 1-ethyl-2-butynyl group, a 2-pentynyl group, a 1-methyl-2-pentynyl group, a 2-hexynyl group, a 3-butynyl group, a 1-methyl-3-butynyl group, a 1-ethyl-3-butynyl group, a 3-pentynyl group, a 1-methyl-3-pentynyl group, a 3-hexynyl group, a 4-pentynyl group, and a 5-hexynyl group.

The “C1-C7 haloalkyl group” means C1-C7 alkyl group substituted with one or more halogen-atoms, and examples thereof include a 2-fluoroethyl group, a 2,2-difluoroethyl group, a 2,2,2-trifluoroethyl group, a 3-fluoropropyl group, a 3,3-difluoropropyl group, a 3,3,3-trifluoropropyl group, a 2,2,3,3-tetrafluoropropyl group, a 2,2,3,3,3-pentafluoropropyl group, a 1-methyl-2-fluoroethyl group, a 1-methyl-2,2,2-trifluoroethyl group, a 2-fluoro-1-(fluoromethyl)ethyl group, a 2,2,2-trifluoro-1-(trifluoromethyl)ethyl group, a 4-fluorobutyl group, a 4,4-difluorobutyl group, a 4,4,4-trifluorobutyl group, a 3,3,4,4,4-pentafluorobutyl group, a 2,2,3,3,4,4-hexafluorobutyl group, a 2,2,3,3,4,4,4-heptafluorobutyl group, a 1-trifluoromethyl-propyl group, a 3,3,3-trifluoro-1-methylpropyl group, a 2,2,3,3-tetrafluoro-1-methylpropyl group, a 2,2,3,3,3-pentafluoro-1-methylpropyl group, a 2,2,3,3,3-pentafluoro-1-trifluoromethyl-propyl group, a 5-fluoropentyl group, a 5,5,5-trifluoropentyl group, a 6-fluorohexyl group, a 6,6,6-trifluorohexyl group, a 2,2,3,4,4-pentafluoro-3-butenyl group, a 2,2,3,3,3-pentafluoro-1-methylpropyl group, a 2,2,3,3,4,4,4-heptafluorobutyl group, a 2-chloroethyl group, a 2,2-dichloroethyl group, a 2,2,2-trichloroethyl group, a 3-chloropropyl group, a 2-chloropropyl group, a 3-chloro-2,2-dimethylpropyl group, a 3,3-dichloropropyl group, a 2,3-dichloropropyl group, a 2-chloro-1-methylethyl group, a 2-chloro-1-(chloromethyl)ethyl group, a 1-methyl-2,2,2-trichloroethyl group, a 4-chlorobutyl group, a 1-chlorobutyl group, a 3-chloro-1-(chloromethyl)propyl group, a 2-chloro-2-methylpropyl group, a 5-chloropentyl group, 6-chlorohexyl group, a 2-bromoethyl group, a 2,2,2-tribromoethyl group, a 3-bromopropyl group, a 2,3-dibromopropyl group, a 2-bromo-1-methylethyl group, a 2-bromo-1-(bromomethyl)ethyl group, a 4-bromobutyl group, a 3-bromo-1-(bromomethyl)propyl group, a 2-(bromomethyl)propyl group, a 3-bromo-2-(bromomethyl)propyl group, a 2-iodoethyl group, and a 3-iodopropyl group.

The “C3-C7 haloalkenyl group” means a C3-C7 alkenyl group substituted with one or more halogen atoms, and examples thereof include a 3-fluoro-2-propenyl group, a 2-fluoro-2-propenyl group, a 3,3-difluoro-2-propenyl group, a 2,3-difluoro-2-propenyl group, a 2,3,3-trifluoro-2-propenyl group, a 4,4-difluoro-3-butenyl group, a 3,4,4-trifluoro-3-butenyl group, a 2,3-difluoro-2-butenyl group, a 2-fluoro-3-methyl-2-butenyl group, a 5,5-difluoro-4-pentenyl group, 4,5,5-trifluoro-4-pentenyl group, a 4,4,4-trifluoro-3-(trifluoromethyl)-2-butenyl group, a 2,4,4,4-tetrafluoro-2-butenyl group, a 4,4,4-trifluoro-3-methyl-2-butenyl group, a 4,4,4-trifluoro-3-(trifluoromethyl)-2-butenyl group, a 3-chloro-2-propenyl group, a 2-chloro-2-propenyl group, a 3,3-dichloro-2-propenyl group, a 2,3-dichloro-2-propenyl group, a 2,3,3-trichloro-2-propenyl group, a 4-chloro-3-butenyl group, a 4,4-dichloro-3-butenyl group, a 3,4-dichloro-3-butenyl group, a 3-chloro-2-butenyl group, a 2-chloro-2-butenyl group, a 2,3-dichloro-2-butenyl group, a 2-chloro-3-methyl-2-butenyl group, a 5-chloro-4-pentenyl group, a 4-chloro-4-pentenyl group, a 4,5-dichloro-4-pentenyl group, a 3-bromo-2-propenyl group, a 2-bromo-2-propenyl group, a 3,3-dibromo-2-propenyl group, a 2,3-dibromo-2-propenyl group, a 4-bromo-3-butenyl group, a 4.4-dibromo-3-butenyl group, a 3,4-dibromo-3-butenyl group, a 3,4,4-tribromo-3-butenyl group, a 3-bromo-2-butenyl group, a 2-bromo-2-butenyl group, a 2,3-dibromo-2-butenyl group, a 2-bromo-3-methyl-2-butenyl group, a 4-bromo-4-pentenyl group, a 4,5-dibromo-4-pentenyl group, and a 4,5,5-tribromo-4-pentenyl group.

The “C3-C7 haloalkynyl group” means a C3-C7 alkynyl group substituted with one or more halogen atoms, and examples thereof include a 3-chloro-propynyl group, a 3-chloro-1-methyl-2-propynyl group, a 3-chloro-1,1-dimethyl-2-propynyl group, a 3-chloro-1-ethyl-2-propynyl group, a 3-chloro-1-propyl-2-propynyl group, a 3-chloro-1-(1-methylethyl)-2-propynyl group, a 4-chloro-3-butynyl group, 4-chloro-1-methyl-3-butynyl group, a 4-chloro-1-ethyl-3-butynyl group, a 5-chloro-4-pentynyl group, a 6-chloro-5-hexynyl group, a 3-bromopropynyl group, a 3-bromo-1-methyl-2-propynyl group, a 3-bromo-l,l-dimethyl-2-propynyl group, a 3-bromo-1-ethyl-2-propynyl group, a 3-bromo-1-propyl-2-propynyl group, a 3-bromo-1-isopropyl-2-propyl group, a 4-bromo-3-butynyl group, a 4-bromo-1-methyl-3-butynyl group, a 4-bromo-1-ethyl-3-butynyl group, a 5-bromo-4-pentynyl group, and a 6-bromo-5-hexynyl group,.

The “(C1-C7 alkoxy)C1-C7 alkyl group” means a C1-C7 alkyl group substituted with one or more C1-C7 alkoxy groups, and examples thereof include a methoxymethyl group, a 2-methoxyethyl group, a 2-methoxy-1-methylethyl group, a 2-methoxy-2-methylethyl group, a 2-ethyl-2-methoxyethyl group, a 2-ethoxyethyl group, a 2-propoxyethyl group, a 2-(1-methylethyl)oxyethyl group, a 2-butoxyethyl group, a 2-isobutoxyethyl group, a 2-(sec-butoxy)ethyl group, a 2-(tert-butoxy)ethyl group, a 3-methoxypropyl group, a 3-methoxy-3-methylpropyl group, a 3-methoxy-3,3-dimethylpropyl group, a 3-ethoxypropyl group, a 3-propoxypropyl group, a 3-(1-methylethyl)oxypropyl group, a 3-buthoxypropyl group, a 3-isobutoxypropyl group, a 3-(sec-butoxy)propyl group, a 3-(tert-butoxy)propyl group, a 3,3-diethoxypropyl group, a 2,2-diethoxyethyl group, and groups represented by the formulas:

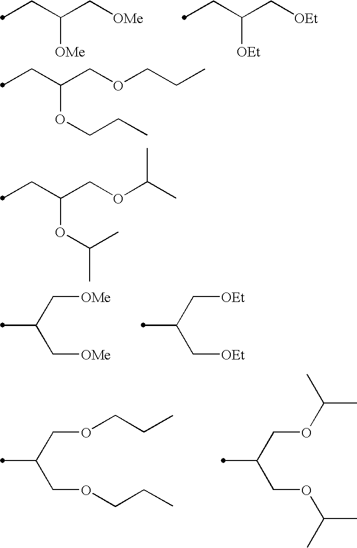

Examples of the “{(C1-C7 alkoxy)C1-C4 alkoxy}C1-C7 alkyl group” include a 2-(methoxymethoxy)ethyl group, and groups represented by the formulas:

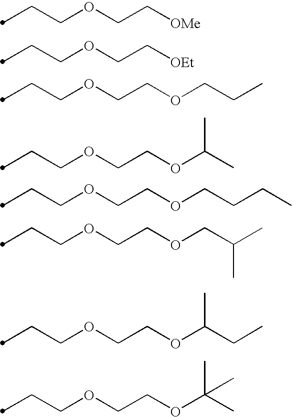

Examples of the “[{(C1-C7 alkoxy)C1-C4 alkoxy}C1-C4 alkoxy]C1-C7 alkyl group” include groups represented by the formulas:

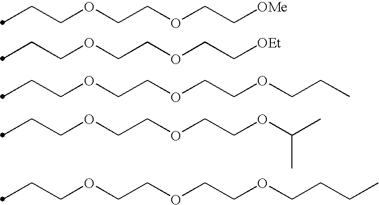

The “(C1-C7 haloalkoxy)C1-C7 alkyl group” means a C1-C7 alkyl group substituted with one or more C1-C7 haloalkoxy groups, and examples thereof include a 3-(2,2,2-ethoxy)propyl group, a 2-(2-fluoroethoxy)ethyl group, a 2-(2-chloroethoxy)ethyl group, a 2-(2-bromoethoxy)ethyl group, a 2-(2-iodoethoxy)ethyl group, a 2-(2,2,2-trifluoroethoxy)ethyl group, a 3-(2-chloroethoxy)propyl group, a 3-(2-bromoethoxy)propyl group, a 3-(2-iodoethoxy)propyl group, and a 3-(2,2,2-trifluoroethoxy)propyl group.

The “(C3-C7 alkenyloxy)C1-C7 alkyl group” means a C1-C7 alkyl group substituted with one or more C3-C7 alkenyloxy groups, and examples thereof include groups represented by the formulas:

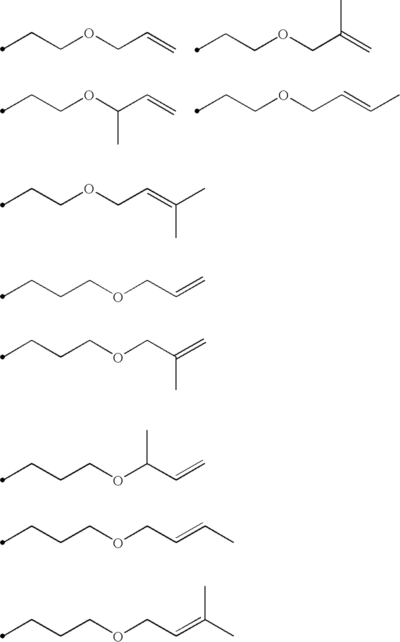

The “(C3-C7 alkynyloxy)C1-C7 alkyl group” means a C1-C7 alkyl group substituted with one or more C3-C7 alkynyloxy groups, and examples thereof include groups represented by the formulas:

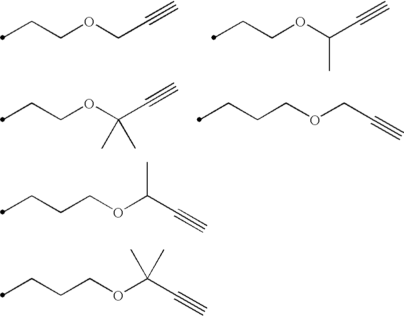

The “(C3-C7 haloalkenyloxy)C1-C7 alkyl group” means a C1-C7 alkyl group substituted with one or more C1-C7 haloalkenyloxy groups, and examples thereof include groups represented by the formulas:

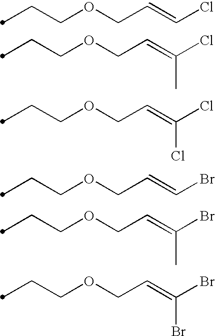

The “(C1-C7 alkylthio)alkyl group” means a C1-C7 alkyl group substituted with one or more C1-C7 alkylthio groups, and examples thereof include a methylthiomethyl group, a 2-methylthioethyl group, a 2-ethylthioethyl group, a 2-propylthioethyl group, a 2-isopropylthioethyl group, a 2-butylthioethyl group, a 2-isobutylthioethyl group, a 2-(sec-butylthio)ethyl group, a 2-(tert-butylthio)ethyl group, a 3-methylthiopropyl group, a 3-ethylthiopropyl group, a 3-propylthiopropyl group, a 3-butylthiobutyl group, and a 3-(tert-butylthio)propyl group.

The “C1-C7 hydroxyiminoalkyl group” means a C1-C7 alkyl group substituted with one or more hydroxyimino groups, and examples thereof include a 1-hydroxyiminoethyl group, a 2-hydroxyiminoethyl group, a 3-hydroxyiminopropyl group, a 4-hydroxyiminobutyl group, a 5-(hydroxyimino)pentyl group and a 6-(hydroxyimino)hexyl group.

The “(C1-C7 alkoxyimino)C1-C7 alkyl group” means a C1-C7 alkyl group substituted with one or more C1-C7 alkoxyimino groups, and examples thereof include a 2-(methoxyimino)ethyl group, a 2-(ethoxyimino)ethyl group, a 2-(propoxyimino)ethyl group, a 2-(isopropoxyimino)ethyl group, a 3-(methoxyimino)propyl group, a 3-(ethoxyimino)propyl group, a 3-(propoxyimino)propyl group, a 3-(isopropoxyimino)propyl group, a 4-(methoxyimino)butyl group, a 4-(ethoxyimino)butyl group, a 4-(propoxyimino)butyl group, and a 4-(isopropoxyimino)butyl group.

The “(C1-C7 alkylamino)C1-C7 alkyl group” means a C1-C7 alkyl group substituted with one or more C1-C7 alkylamino groups, and examples thereof include a 2-(methylamino)ethyl group, a 3-(methylamino)propyl group, a 4-(methylamino)butyl group, a 5-(methylamino)pentyl group, a 6-(methylamino)hexyl group, a 2-(dimethylamino)ethyl group, a 3-(dimethylamino)propyl group, a 4-(dimethylamino)butyl group, a 5-(dimethylamino)pentyl group, and a 6-(dimethylamino)hexyl group.

The “C2-C8 cyanoalkyl group” means a C1-C7 alkyl group substituted with one or more cyano groups, and examples thereof include a cyanomethyl group, a 1-cyanoethyl group, a 2-cyanoethyl group, a 3-cyanopropyl group, a 4-cyanobutyl group, and a 5-cyanopentyl group.

The “C2-C8 formylalkyl group” means a C1-C7 alkyl group substituted with one or more formyl groups, and examples thereof include a formylmethyl group, a 1-formylethyl group, a 2-formylethyl group, a 3-formylpropyl group, a 4-formylbutyl group, and a 5-formylpentyl group.

The “(C2-C8 alkanoyl)C1-C7 alkyl group” means a C1-C7 alkyl group substituted with one or more C2-C8 alkanoyl groups, and examples thereof include an acetylmethyl group, a propionylmethyl group, a butyrylmethyl group, a valerylmethyl group, a 2-acetylethyl group, a 2-propionylethyl group, a 2-butyrylethyl group, a 3-acetylpropyl group, a 3-propionylpropyl group, and a 4-acetylbutyl group.

The “(C2-C8 alkoxycarbonyl)C1-C7 alkyl group” means a C1-C7 alkyl group substituted with one or more C2-C8 alkoxycarbonyl groups, and examples thereof include a 2-(methoxycarbonyl)ethyl group, a 2-(ethoxycarbonyl)ethyl group, a 3-(methoxycarbonyl)propyl group, a 3-(ethoxycarbonyl)propyl group, a 4-(methoxycarbonyl)butyl group, a 4-(ethoxycarbonyl)butyl group, a 5-(methoxycarbonyl)pentyl group, and a 5-(ethoxycarbonyl)pentyl group.

The “C1-C7 hydroxyalkyl group” means a C1-C7 alkyl group substituted with one or more hydroxy groups, and examples thereof include a 1-hydroxyethyl group, a 2-hydroxyethyl group, a 3-hydroxypropyl group, a 4-hydoxybutyl group, a 5-hydoxypentyl group, a 6-hydroxyhexyl group, and groups represented by the formulas:

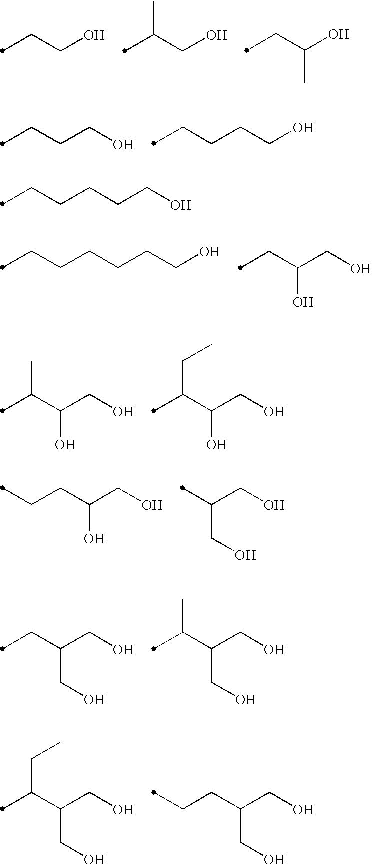

The “(C2-C8 alkylcarbonyloxy)C1-C7 alkyl group” means a C1-C7 alkyl group substituted with one or more C2-C8 alkylcarbonyloxy groups, and examples thereof include groups represented by the formulas:

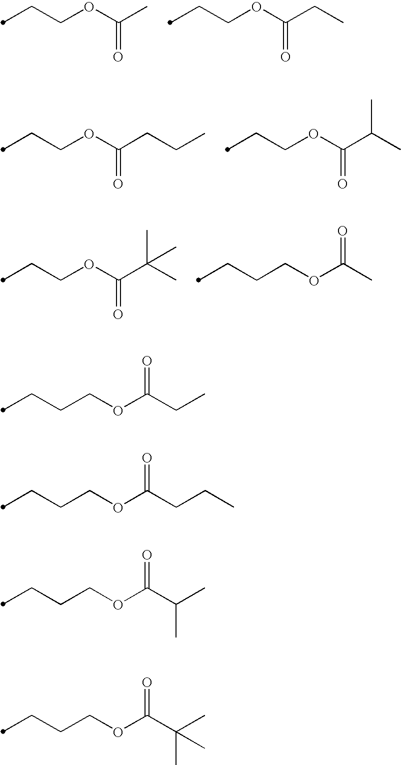

Examples of the “C3-C6 alkanoyl group” include a propionyl group, a butyryl group, an isobutyryl group, a valeryl group, an isovaleryl group, and a pivaloyl group.

Examples of the “C3-C8 cycloalkyl group optionally substituted with one or more substituents selected from the B group and the C group” include a C3-C8 cycloalkyl group optionally substituted with one or more monovalent groups selected from the group consisting of a methyl group, an ethyl group, a propyl group, an isopropyl group, a tert-butyl group, trifluoromethyl group, a difluoromethyl group, a pentafluoroethyl group, a vinyl group, an allyl group, an ethynyl group, a fluorine atom, a chlorine atom, a bromine group, and more specific examples thereof include a cyclopropyl group, a cyclobutyl group, a cyclopentyl group, a cyclohexyl group, a cycloheptyl group, cyclooctyl group, a 2-methylcyclohexyl group, a 3-methylcyclohexyl group, a 4-methylcyclohexyl group, a 1-vinylcyclohexyl group, a 1-allylcyclohexyl group, a 1-ethynylcyclohexyl group, a 2-chlorocyclohexyl group, a 4-chlorocyclohexyl group, a 2-fluorocyclohexyl group, a 2-methoxycyclobutyl group, a 2-methoxycyclopentyl group, a 3-methoxycyclopentyl group, a 2-methoxycyclohexyl group, a 3-methoxycyclohexyl group, a 4-methoxycyclohexyl group, a 2-methoxycycloheptyl group, and a 2-methoxycyclooctyl group.

Example of the “C5-C8 cycloalkenyl group optionally substituted with one or more substituents selected from the B group and the C group” include a C5-C8 cycloalkenyl optionally substituted with one or more monovalent groups selected from the group consisting of a methyl group, an ethyl group, a propyl group, an isopropyl group, a tert-butyl group, a trifluoromethyl group, a difluoromethyl group, a pentafluoroethyl group, a vinyl group, an allyl group, an ethynyl group, a fluorine atom, a chlorine atom and a bromine atom, more specifically, groups represented by the formulas:

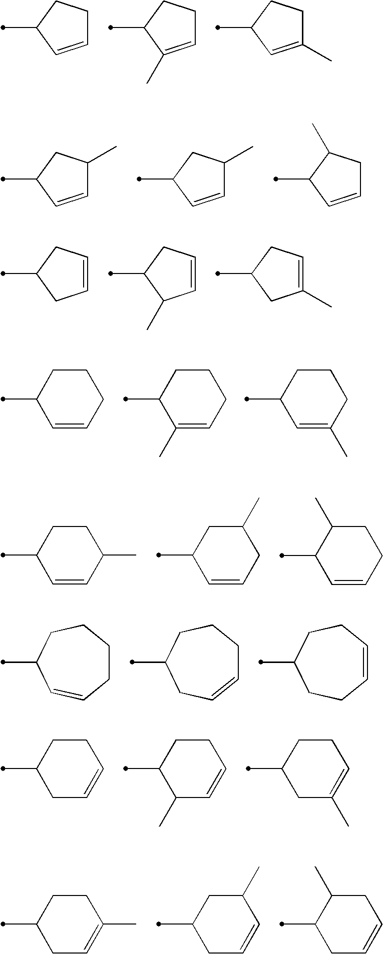

Examples of the “phenyl group optionally substituted with one or more substituents selected from the B group and the C group” include a phenyl group optionally substituted with one or more monovalent groups selected from the group consisting of a methyl group, an ethyl group, a propyl group, an isopropyl group, a tert-butyl group, a trifluoromethyl group, a difluoromethyl group, a pentafluoroethyl group, a methoxy group, an ethoxy group, a propoxy group, an isopropoxy group, a trifluoromethoxy group, a difluoromethoxy group, a methylthio group, an ethylthio group, a fluorine atom, a chlorine atom, a bromine atom, a cyano group, a nitro group, and a formyl group, more specifically include a phenyl group, a 2-fluorophenyl group, a 3-fluorophenyl group, a 4-fluorophenyl group, a 2,3-difluorophenyl group, a 2,4-difluorophenyl group, a 2,5-difluorophenyl group, a 2,6-difluorophenyl group, a 3,4-difluorophenyl group, a 3,5-difluorophenyl group, a 2-chlorophenyl group, a 3-chlorophenyl group, a 4-chlorophenyl group, a 2,3-dichlorophenyl group, a 2,4-dichlorophenyl group, a 2,5-dichlorophenyl group, a 2,6-dichlorophenyl group, a 3,4-dichlorophenyl group, a 3,5-dichlorophenyl group, a 2-bromophenyl group, a 3-bromophenyl group, a 4-bromophenyl group, a 2,3-dibromophenyl group, a 2,4-dibromophenyl group, a 2,5-dibromophenyl group, a 2,6-dibromophenyl group, a 3,4-dibromophenyl group, a 3,5-dibromophenyl group, a 2-iodophenyl group, a 3-iodophenyl group, a 4-iodophenyl group, a 2-methylphenyl group, a 3-methylphenyl group, a 4-methylphenyl group, a 2,3-dimethylphenyl group, a 2,4-dimethylphenyl group, a 2,5-dimethylphenyl group, a 2,6-dimethylphenyl group, a 3,4-dimethylphenyl group, a 3,5-dimethylphenyl group, a 2-methoxyphenyl group, a 3-methoxyphenyl group, a 4-methoxyphenyl group, a 2,3-dimethoxyphenyl group, a 2,4-dimethoxyphenyl group, a 2,5-dimethoxyphenyl group, a 2,6-dimethoxyphenyl group, a 3,4-dimethoxyphenyl group, a 3,5-dimethoxyphenyl group, a 2-ethylphenyl group, a 3-ethylphenyl group, a 4-ethylphenyl group, a 2-(trifluoromethyl)phenyl group, a 3-(trifluoromethyl)phenyl group, a 4-(trifluoromethyl)phenyl group, a 2-methylthiophenyl group, a 3-methylthiophenyl group, a 4-methylthiophenyl group, a 2-(trifluoromethoxy)phenyl group, a 3-(trifluoromethoxy)phenyl group, a 4-(trifluoromethoxy)phenyl group, a 2-nitrophenyl group, a 3-nitrophenyl group, a 4-nitrophenyl group, a 2-cyanophenyl group, a 3-cyanophenyl group, a 4-cyanophenyl group, and groups represented by the formulas:

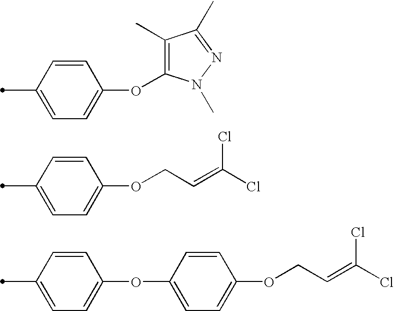

Examples of the “3- to 10-membered heterocyclic group optionally substituted with one or more substituents selected from the B group, or optionally substituted with one or more substituents selected from the C group at the same position or adjacent positions” include a heterocyclic group such as a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 6-membered heterocyclic group containing only one or two sulfur atoms as the heteroatoms, a 5-membered heterocyclic group containing only one or two nitrogen atoms as the heteroatoms, a 5-membered heterocyclic group containing only a sulfur atom and a nitrogen atom as the heteroatoms, a 5-membered heterocyclic group containing only an oxygen atom and a nitrogen atom as the heteroatoms, or a 6-membered heterocyclic group containing only one or two nitrogen atoms as the heteroatoms, optionally substituted with one or more substituents selected from the group consisting of a methyl group, an ethyl group, a propyl group, an isopropyl group, a tert-butyl group, a trifluoromethyl group, a difluoromethyl group, a pentafluoroethyl group, a methoxy group, an ethoxy group, a propoxy group, an isopropoxy group, a trifluoromethoxy group, a difluoromethoxy group, a methylthio group, an ethylthio group, a fluorine atom, a chlorine atom, a bromine atom, a cyano group, a nitro group, and a formyl group.

Examples of the “3- to 6-membered saturated heterocyclic group optionally substituted with one or more substituents selected from the B group and the C group whose heteroatom(s) are only oxygen atom(s) or sulfur atom(s)” include an oxacycloalkyl group optionally substituted with one or more substituents selected from the B group and the C group {specifically, groups represented by the formulas:

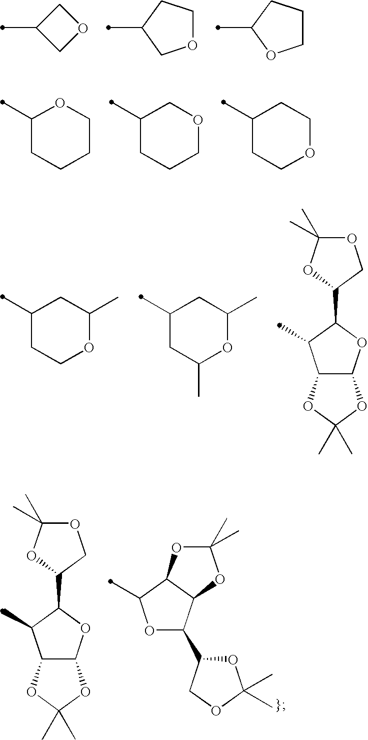

a dioxolanyl group optionally substituted with one or more substituents selected from the B group and the C group {specifically, groups represented by the formulas:

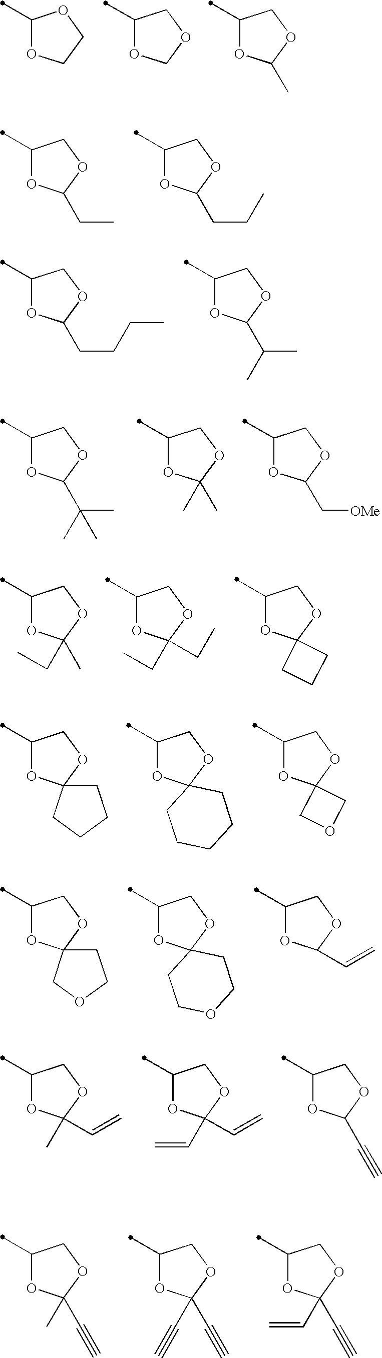

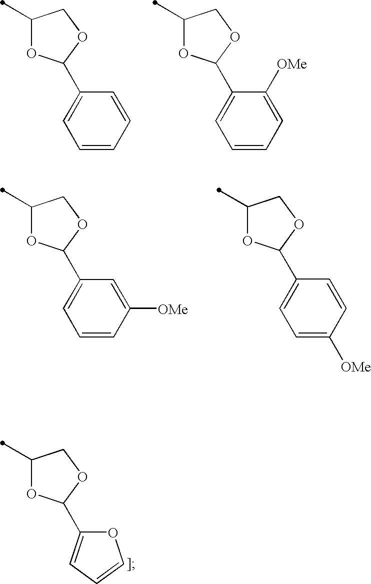

a dioxanyl group optionally substituted with one or more substituents selected from the B group and the C group {specifically, groups represented by the formulas:

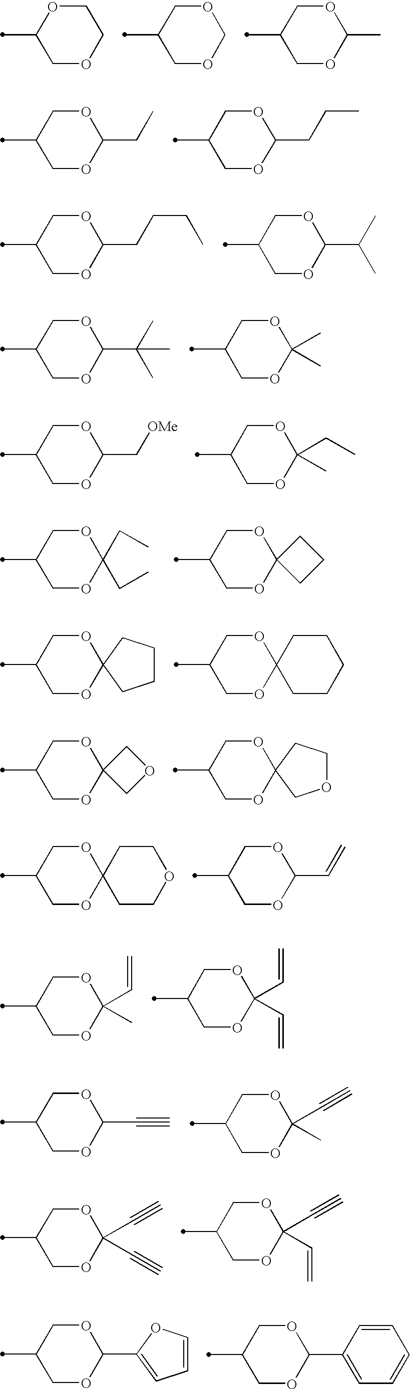

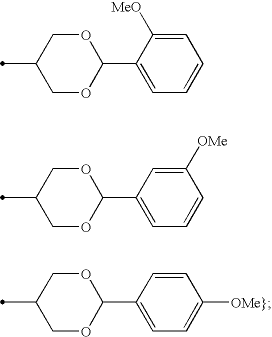

a thiacyloalkyl group optionally substituted with one or more substituents selected from the B group and the C group {specifically, groups represented by the formulas:

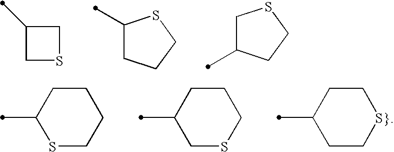

Examples of the “3- to 6-membered saturated heterocyclic group optionally substituted with one or more substituents selected from the B group and the C group whose heteroatom is only a nitrogen atom” include a pyrrolidinyl group optionally substituted with one or more substituents selected from the B group and the C group {specifically, groups represented by the formulas:

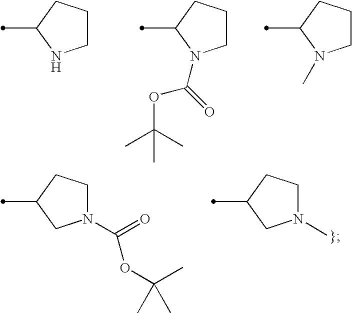

a piperidyl group optionally substituted with one or more substituents selected from the B group and the C group {specifically, groups represented by the formulas:

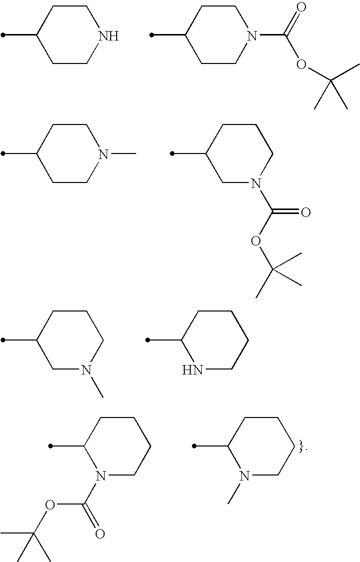

Examples of the “5- to 6-membered unsaturated heterocyclic group optionally substituted with one or more substituents selected from the B group and the C group whose heteroatom is only an oxygen atom or a sulfur atom” include a furyl group optionally substituted with one or more substituents selected from the B group and the C group {specifically, groups represented by the formulas:

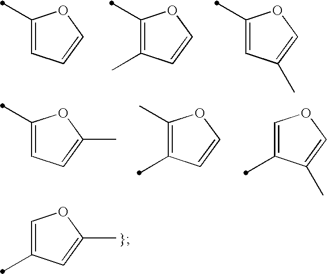

a pyranyl group optionally substituted with one or more substituents selected from the B group and the C group {specifically, groups represented by the formulas:

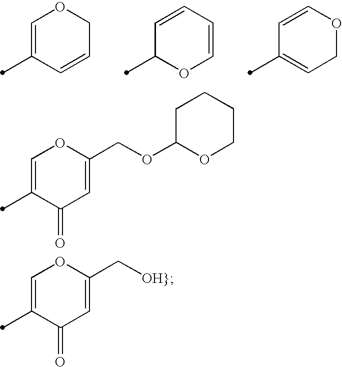

a thienyl group optionally substituted with one or more substituents selected from the B group and the C group {specifically 2-thienyl group, 3-thienyl group}.

Examples of the “5- to 6-membered unsaturated heterocyclic group optionally substituted with one or more substituents selected from the B group and the C group whose heteroatom is only a nitrogen atom” include a pyrrolyl group optionally substituted with one or more substituents selected from the B group and the C group {specifically, groups represented by the formulas:

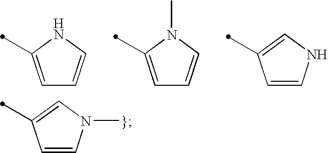

a pyridyl group optionally substituted with one or more substituents selected from the B group and the C group {specifically 2-pyridyl group, 3-pyridyl group, 4-pyridyl group}; a pyrimidinyl group optionally substituted with one or more substituents selected from the B group and the C group {specifically 2-pyrimidinyl group, 4-pyrimidinyl group, 5-pyrimidinyl group}; a pyrazinyl group optionally substituted with one or more substituents selected from the B group and the C group {specifically 2-pyrazinyl group}: a pyridazinyl group optionally substituted with one or more substituents selected from the B group and the C group {specifically 3-pyridazinyl group, 4-pyridazinyl group}; an imidazolyl group optionally substituted with one or more substituents selected from the B group and the C group {specifically, groups represented by the formulas:

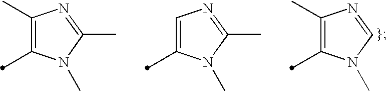

a pyrazolyl group optionally substituted with one or more substituents selected from the B group and the C group {specifically, groups represented by the formulas:

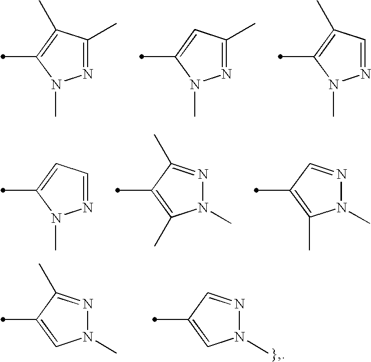

Examples of the “5- to 6-membered unsaturated heterocyclic group optionally substituted with one or more substituents selected from the B group and the C group whose heteroatoms are only a sulfur atom and a nitrogen atom, or only an oxygen atom and a nitrogen atom” include a thiazolyl group optionally substituted with one or more substituents selected from the B group and the C group {specifically, groups represented by the formulas:

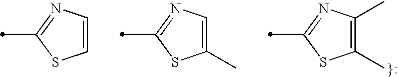

an isothiazolyl group optionally substituted with one or more substituents selected from the B group and the C group {specifically, groups represented by the formulas:

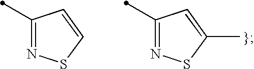

an isoxazolyl group optionally substituted with one or more substituents selected from the B group and the C group {specifically, groups represented by the formulas:

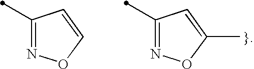

Examples of the “C1-C4 alkyl group substituted with a C3-C8 cycloalkyl group optionally substituted with one or more substituents selected from the B group and the C group” include a C1-C4 alkyl group substituted with a C3-C8 cycloalkyl group optionally substituted with one or more substituents selected from the group consisting of a methyl group, an ethyl group, a propyl group, an isopropyl group, a tert-butyl group, a trifluoromethyl group, a difluoromethyl group, a pentafluoroethyl group, a vinyl group, an allyl group, an ethynyl group, a fluorine atom, a chlorine atom and a bromine atom, more specifically, groups represented by the formulas:

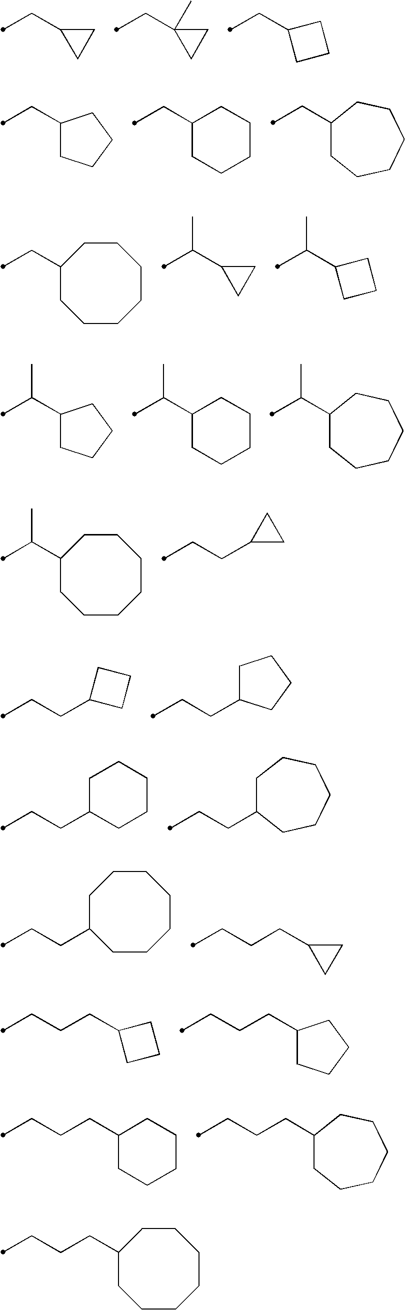

Examples of the “C1-C4 alkyl group substituted with a C5-C8 cycloalkenyl group optionally substituted with one or more substituents selected from the B group and the C group” include a C1-C4 alkyl group substituted with a C5-C8 cycloalkenyl group optionally substituted with one or more substituents selected from the group consisting of a methyl group, an ethyl group, a propyl group, an isopropyl group, a tert-butyl group, a trifluoromethyl group, a difluoromethyl group, a pentafluoroethyl group, a vinyl group, an allyl group, an ethynyl group, a fluorine atom, a chlorine atom and a bromine atom, more specifically, groups represented by the formulas:

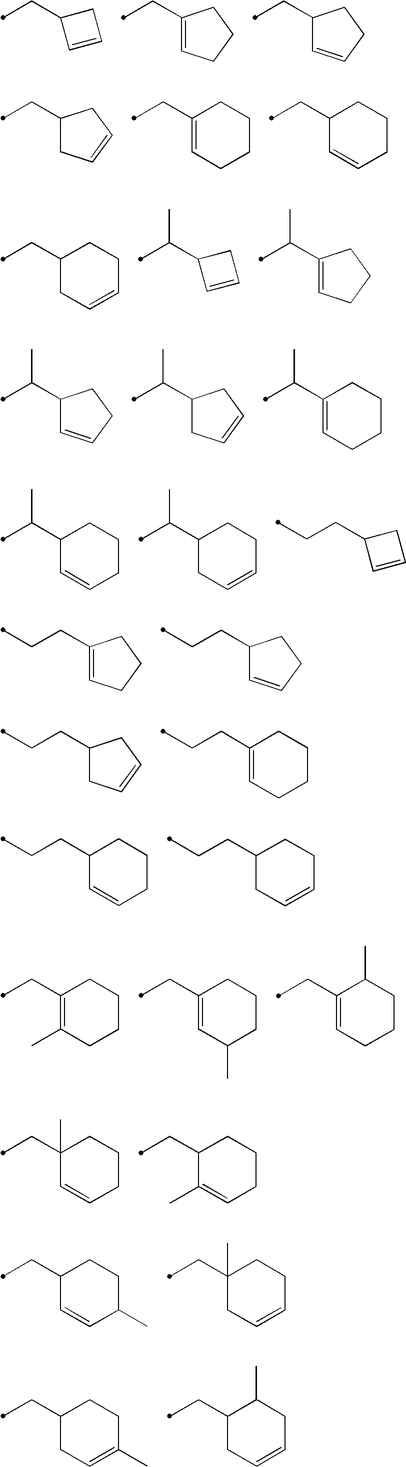

Examples of the “C1-C4 alkyl group substituted with a phenyl group optionally substituted with one or more substituents selected from the B group and the C group” include a C1-C4 alkyl group substituted with a phenyl group optionally substituted with one or more substituents selected from the group consisting of a methyl group, an ethyl group, a propyl group, an isopropyl group, a tert-butyl group, a trifluoromethyl group, a difluoromethyl group, a pentafluoroethyl group, a methoxy group, an ethoxy group, a propoxy group, an isopropoxy group, a trifluoromethoxy group, a difluoromethoxy group, a methylthio group, an ethylthio group, a fluorine atom, a chlorine atom, a bromine atom, a cyano group, a nitro group, and a formyl group, more specifically include a benzyl group, a 2-fluorobenzyl group, a 3-fluorobenzyl group, a 4-fluorobenzyl group, a 2,3-difluorobenzyl group, a 2,4-difluorobenzyl group, a 2,5-difluorobenzyl group, a 2,6-difluorobenzyl group, a 3,4-difluorobenzyl group, a 3,5-difluorobenzyl group, a 2-chlorobenzyl group, a 3-chlorobenzyl group, a 4-chlorobenzyl group, a 2,3-dichlorobenzyl group, a 2,4-dichlorobenzyl group, a 2,5-dichlorobenzyl group, a 2,6-dichlorobenzyl group, a 3,4-dichlorobenzyl group, a 3,5-dichlorobenzyl group, a 2-bromobenzyl group, a 3-bromobenzyl group, a 4-bromobenzyl group, a 2,3-dibromobenzyl group, a 2,4-dibromobenzyl group, a 2,5-dibromobenzyl group, a 3,4-dibromobenzyl group, a 2,5-dibromobenzyl group, a 3,5-dibromobenzyl group, a 2-iodobenzyl group, a 3-iodobenzyl group, a 4-iodobenzyl group, a 2-methylbenzyl group, a 3-methylbenzyl group, a 4-methylbenzyl group, a 2-(trifluoromethyl)benzyl group, a 3-(trifluoromethyl)benzyl group, a 4-(trifluoromethyl)benzyl group, a 2-methoxybenzyl group, a 3-methoxybenzyl group, a 4-methoxybenzyl group, a 2,5-dimethoxybenzyl group, a 3,5-dimethoxybenzyl group, a 2-methylthiobenzyl group, a 3-methylthiobenzyl group, a 4-methylthiobenzyl group, a 2-(trifluoromethoxy)benzyl group, a 3-(trifluoromethoxy)benzyl group, a 4-(trifluoromethoxy)benzyl group, a 2-nitrobenzyl group, a 3-nitrobenzyl group, a 4-nitrobenzyl group, a 2-cyanobenzyl group, a 3-cyanobenzyl group, a 4-cyanobenzyl group, a 2-ethoxybenzyl group, a 3-ethoxybenzyl group, a 4-ethoxybenzyl group, a 4-isopropylbenzyl group, a 4-tert-butylbenzyl group, a 2-fluoro-4-(trifluoromethyl)benzyl group, a 2-fluoro-5-(trifluoromethyl)benzyl group, a 4-fluoro-3-(trifluoromethyl)benzyl group, a 2,4-bis(trifluoromethyl)benzyl group, a 5-fluoro-2-methylbenzyl group, a pentafluorobenzyl group, and a phenethyl group.

Examples of the “C1-C4 alkyl group substituted with a 3- to 10-membered heterocyclic group optionally substituted with one or more substituents selected from the B group, or optionally substituted with one or more substituents selected from the C group at the same position or adjacent positions” include a C1-C4 alkyl group substituted with a heterocyclic group optionally substituted with one or more substituents selected from the group consisting of a methyl group, an ethyl group, a propyl group, an isopropyl group, a tert-butyl group, a trifluoromethyl group, a difluoromethyl group, a pentafluoroethyl group, a methoxy group, an ethoxy group, a propoxy group, an isopropoxy group, a trifluoromethoxy group, a difluoromethoxy group, a methylthio group, an ethylthio group, a fluorine atom, a chlorine atom, a bromine atom, a cyano group, a nitro group, and a formyl group, said heterocyclic group being a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 6-membered heterocyclic group containing only one or two sulfur atoms as the heteroatoms, a 5-membered heterocyclic group containing only one or two nitrogen atoms as the heteroatoms, a 5-membered heterocyclic group containing only a sulfur atom and a nitrogen atom as the heteroatoms, a 5-membered heterocyclic group containing only an oxygen atom and a nitrogen atom as the heteroatoms, or a 6-membered heterocyclic group containing only one or two nitrogen atoms as the heteroatoms.

Examples of the “C1-C4 alkyl group substituted with a 3- to 6-membered saturated heterocyclic group optionally substituted with one or more substituents selected from the B group and the C group whose heteroatom(s) are only oxygen atom(s) or sulfur atom(s)” include a C1-C4 alkyl group substituted with an oxacycloalkyl group optionally substituted with one or more substituents selected from the B group and the C group {specifically, groups represented by the formula:

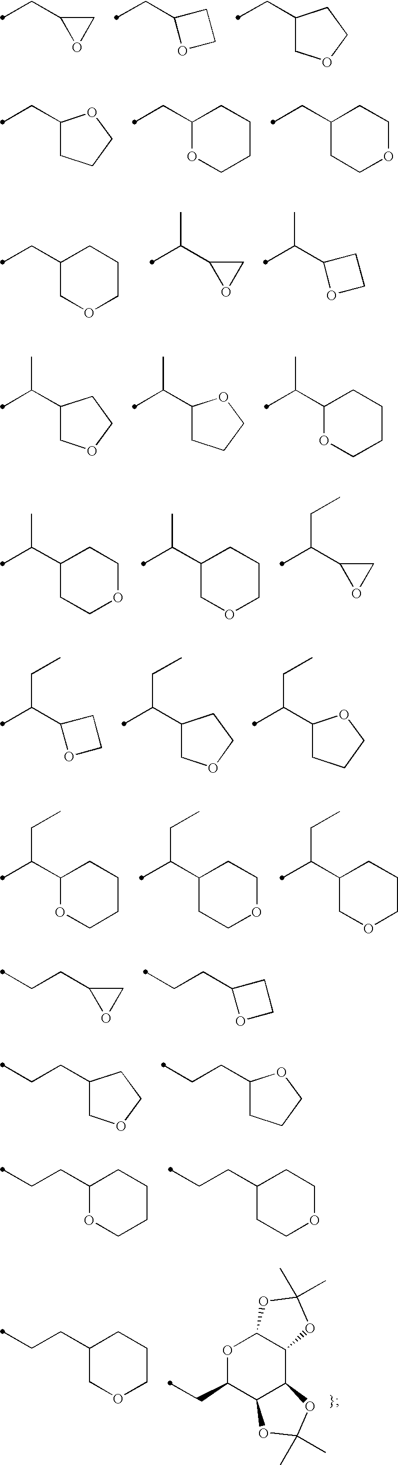

a C1-C4 alkyl group substituted with a dioxolanyl group optionally substituted with one or more substituents selected from the B group and the C group {specifically, groups represented by the formulas:

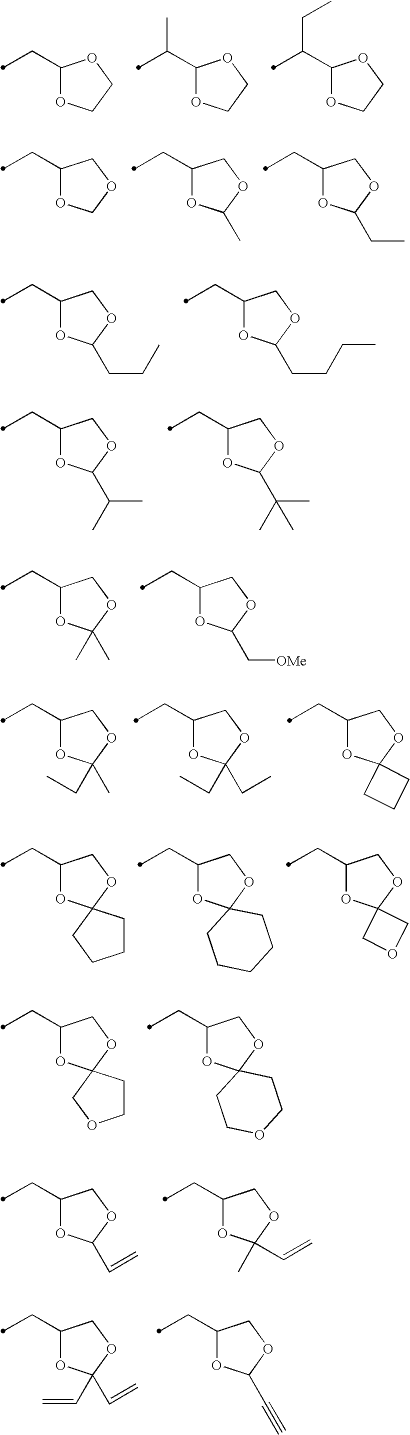

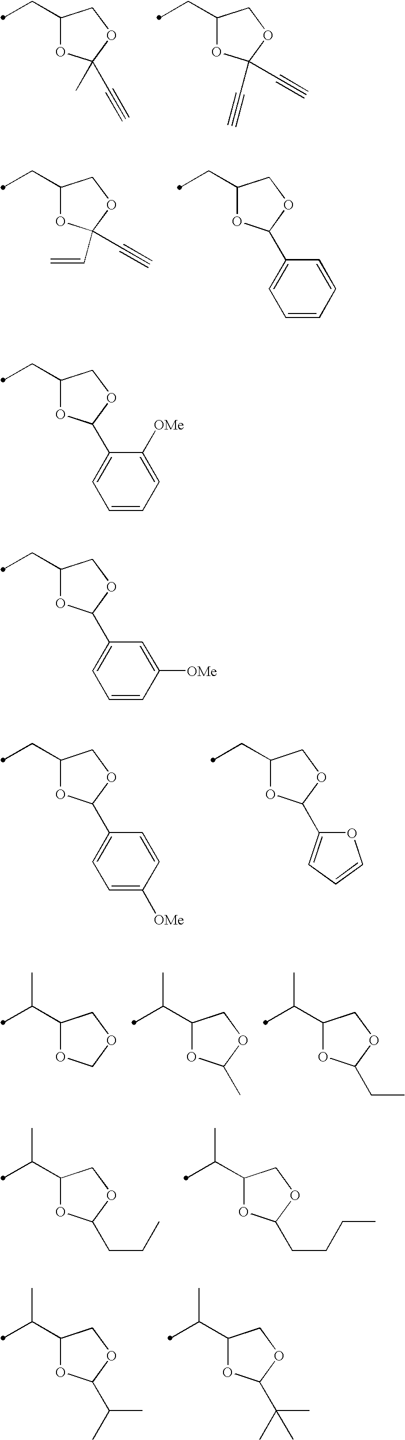

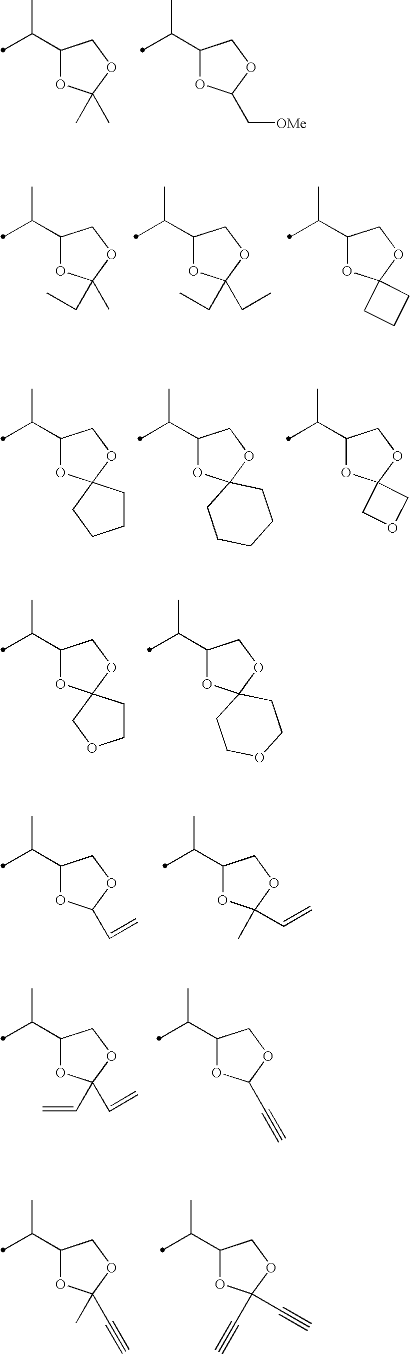

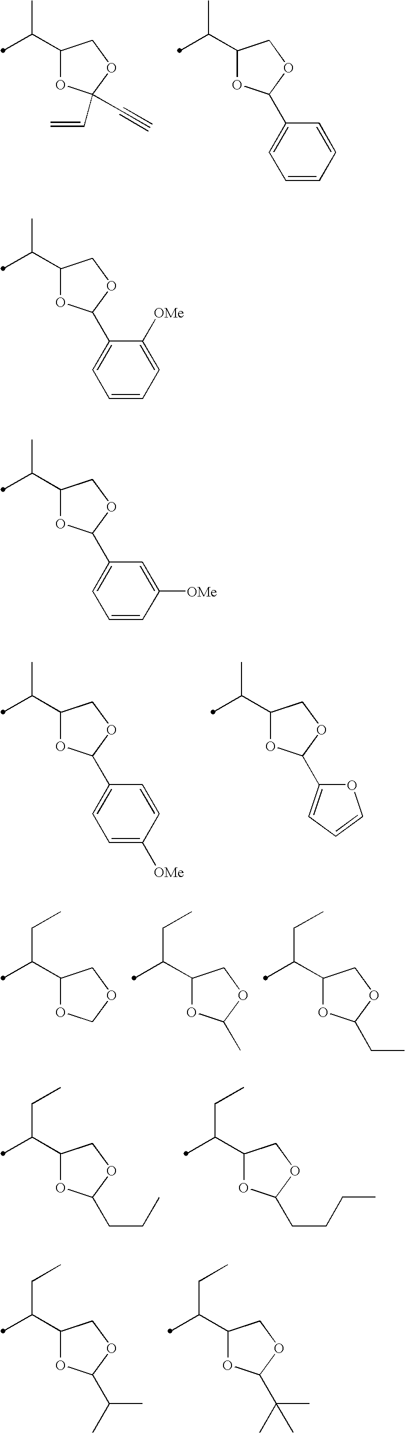

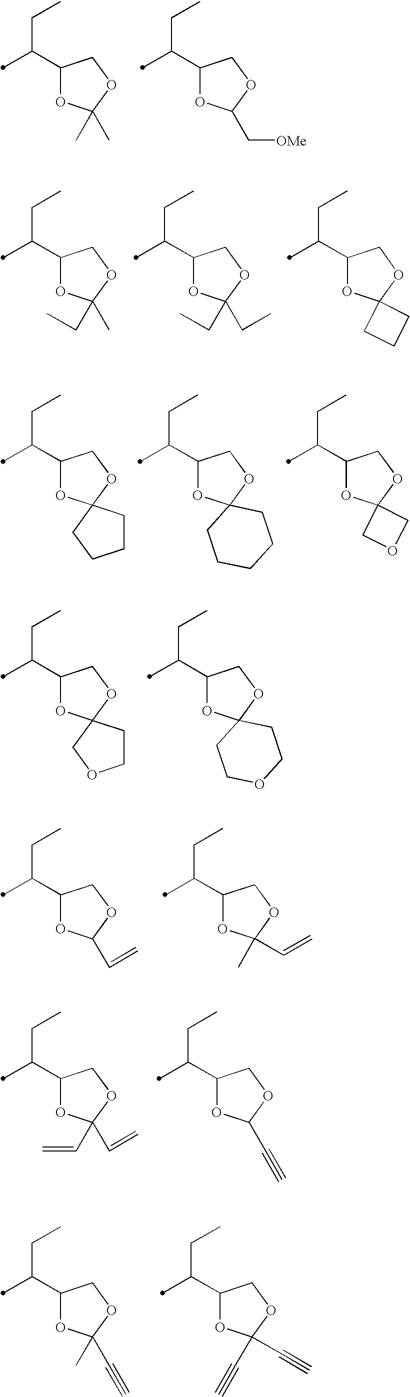

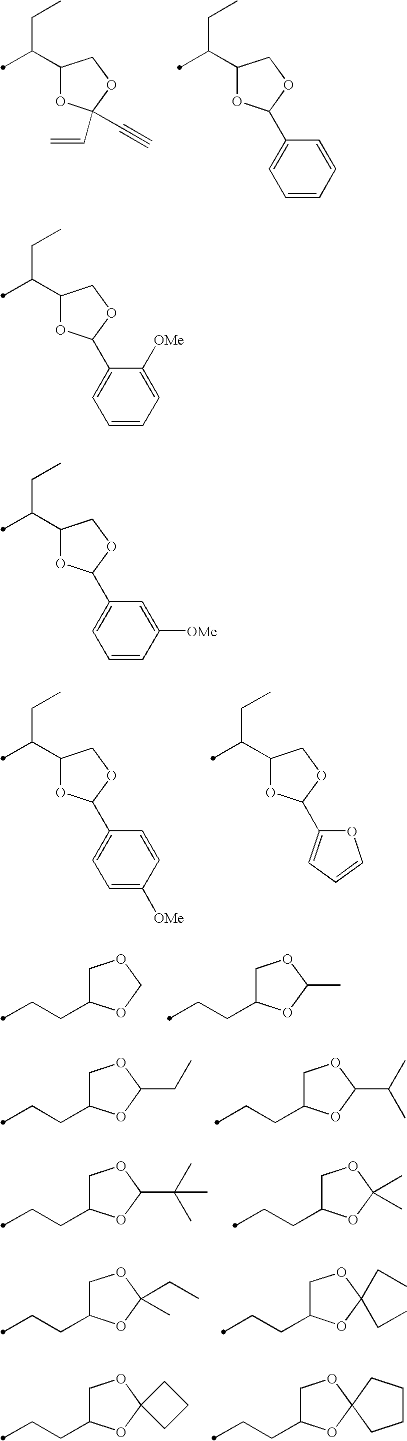

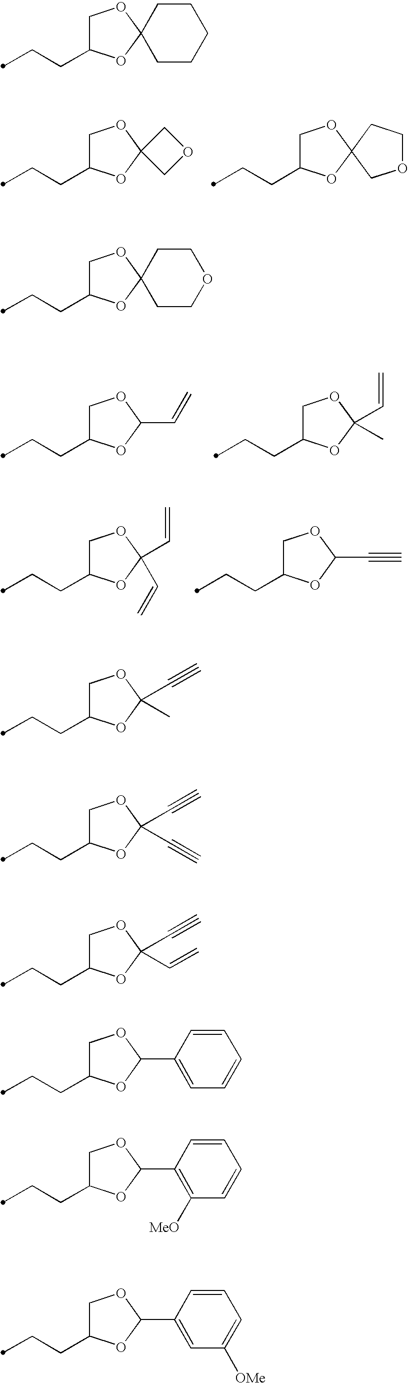

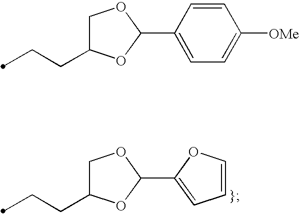

a C1-C5 alkyl group substituted with a dioxanyl group optionally substituted with one or more substituents selected from the B group and the C group {specifically groups represented by the formulas:

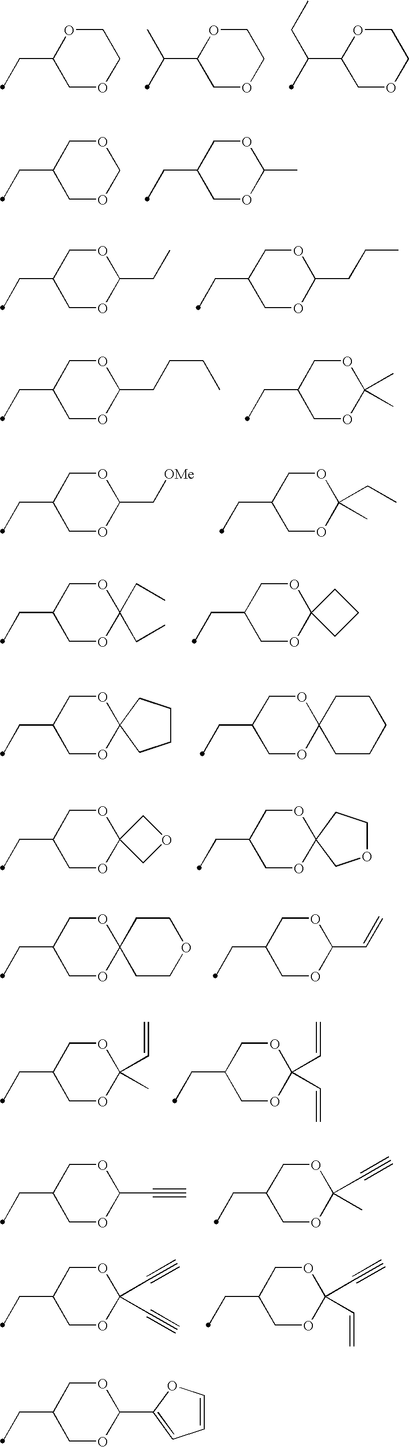

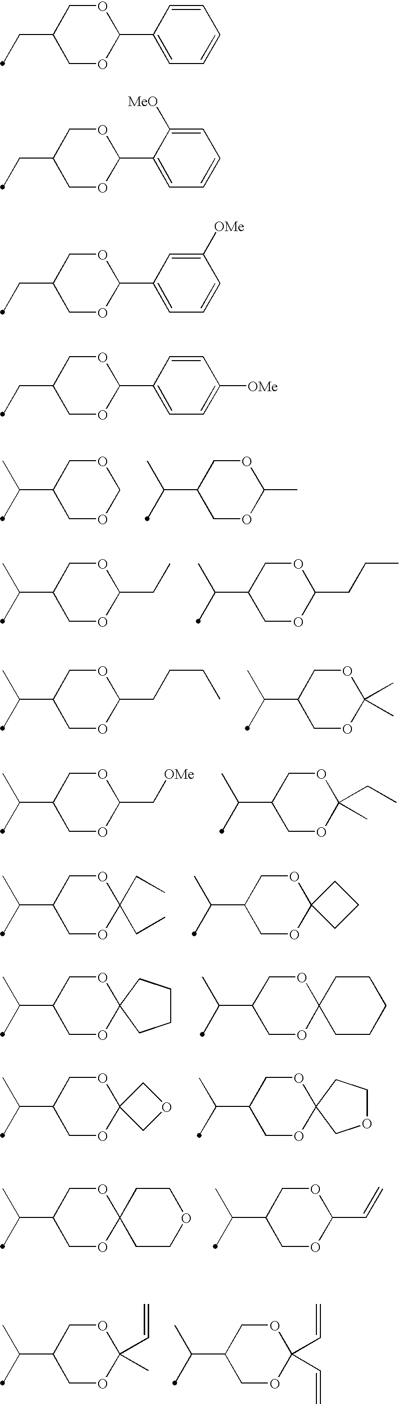

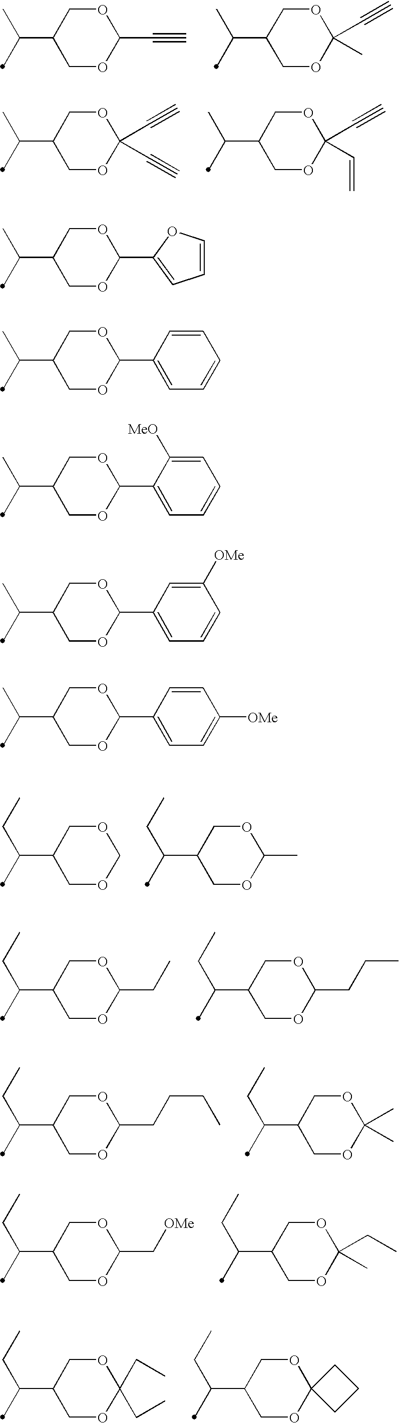

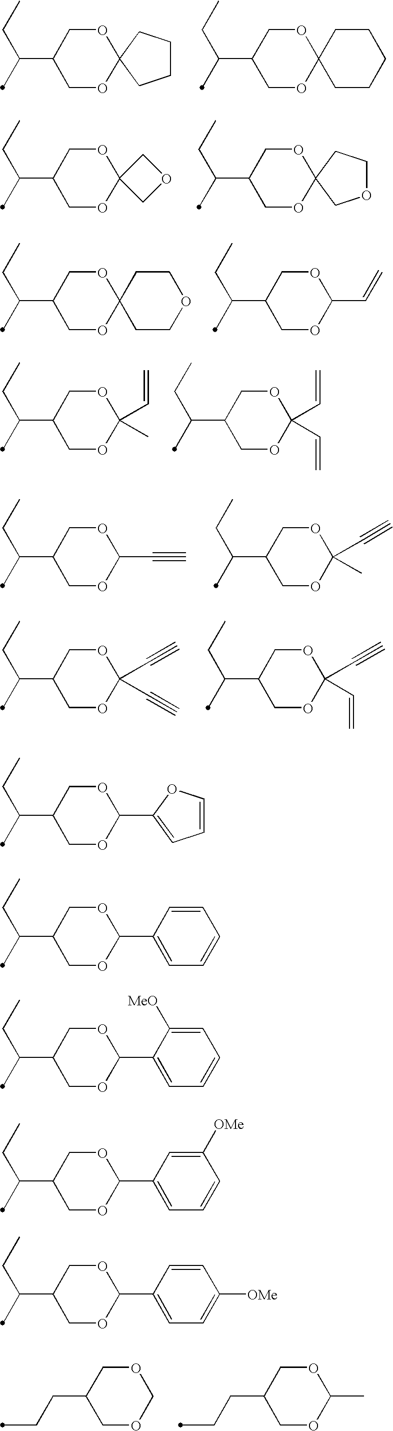

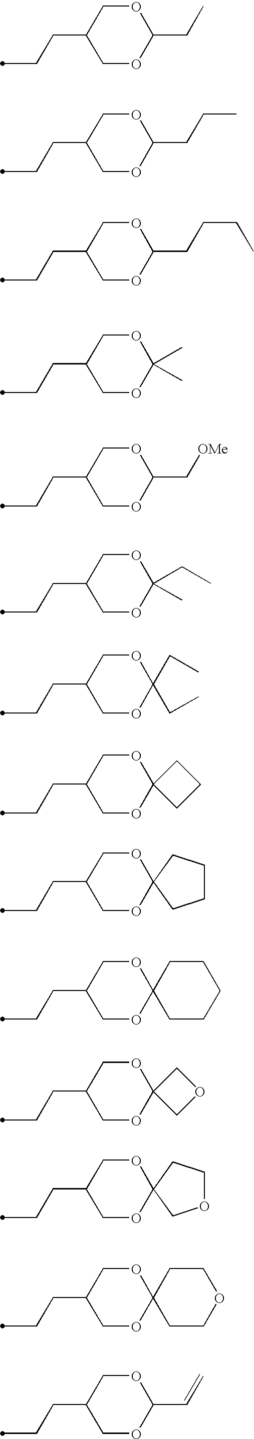

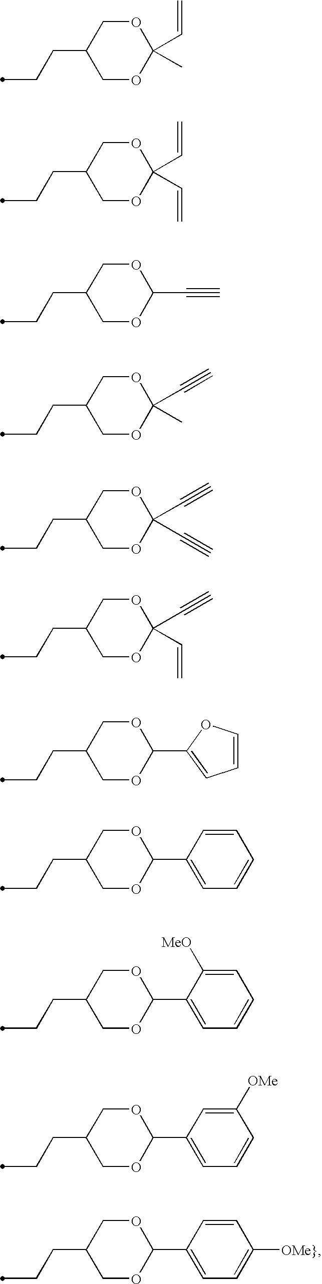

a C1-C4 alkyl group substituted with a thiacycloalkyl group optionally substituted with one or more substituents selected from the B group and the C group {specifically, groups represented by the formulas:

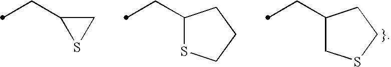

Examples of the “C1-C4 alkyl group substituted with a 3- to 6-membered saturated heterocyclic group optionally substituted with one or more substituents selected from the B group and the C group whose heteroatom(s) are only nitrogen atom(s)” include a C1-C4 alkyl group substituted with a pyrrolidinyl group optionally substituted with one or more substituents selected from the B group and the C group {specifically, groups represented by the formulas:

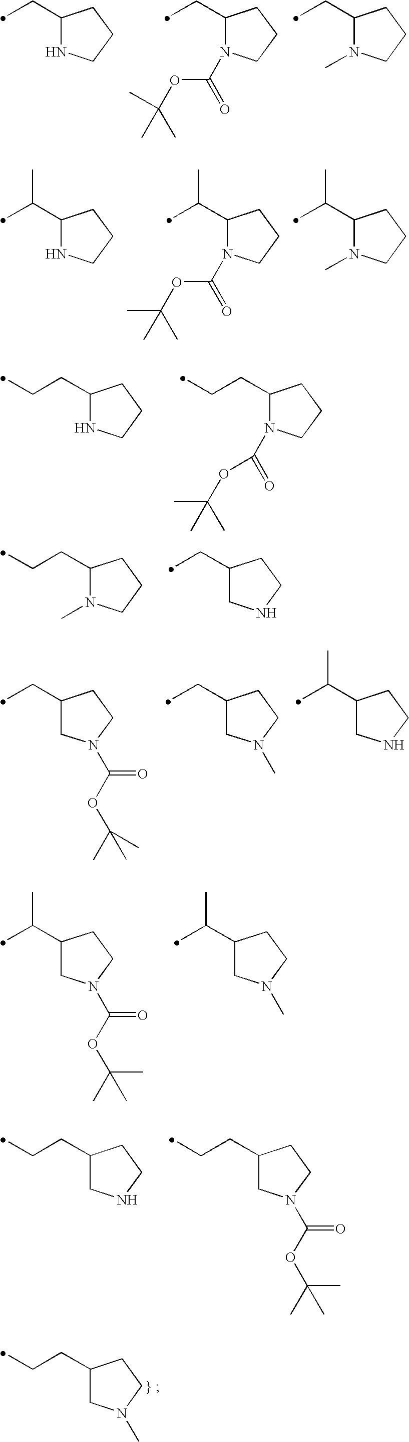

a C1-C4 alkyl group substituted with a piperidyl group optionally substituted with one or more substituents selected from the B group and the C group {specifically, groups represented by the formulas:

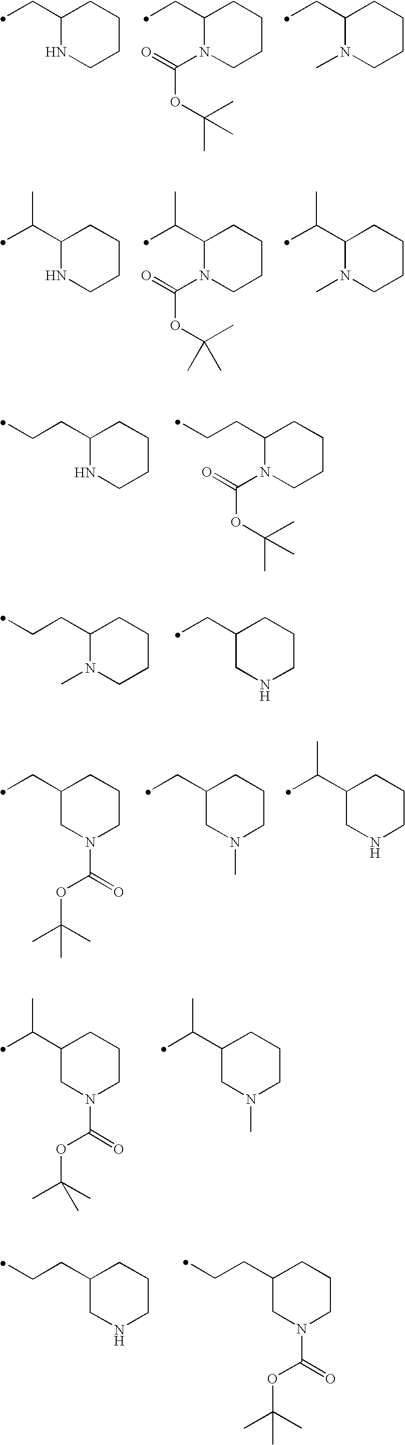

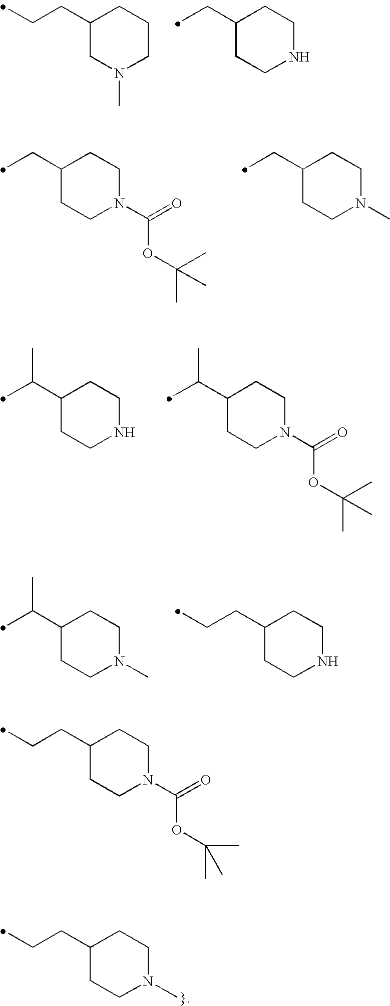

Examples of the “C1-C4 alkyl group substituted with a 5- to 6-membered unsaturated heterocyclic group optionally substituted with one or more substituents selected from the B group and the C group whose heteroatom(s) are only oxygen atom(s) or sulfur atom(s)” include a C1-C4 alkyl group substituted with a furyl group optionally substituted with one or more substituents selected from the B group and the C group {specifically, groups represented by the formulas:

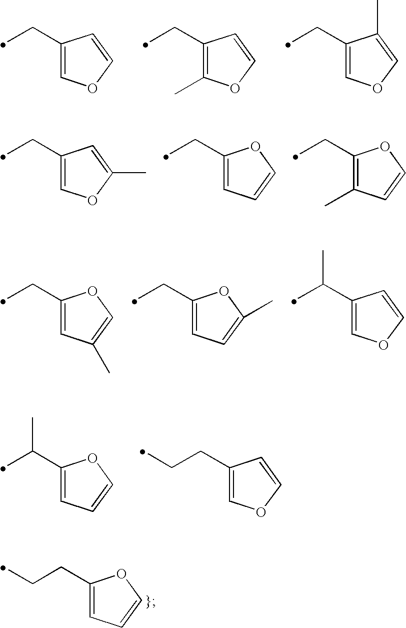

a C1-C4 alkyl group substituted with a thienyl group optionally substituted with one or more substituents selected from the B group and the C group {specifically, groups represented by the formulas:

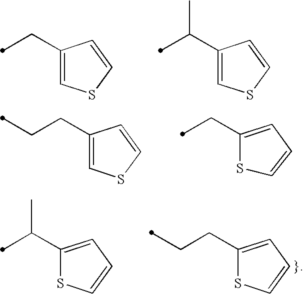

Examples of the “C1-C4 alkyl group substituted with a pyrrolidinyl group optionally substituted with one or more substituents selected from the B group and the C group whose heteroatom(s) are only nitrogen atom(s)” include a C1-C4 alkyl group substituted with a pyrrolyl group optionally substituted with one or more substituents selected from the B group and the C group {specifically, groups represented by the formulas:

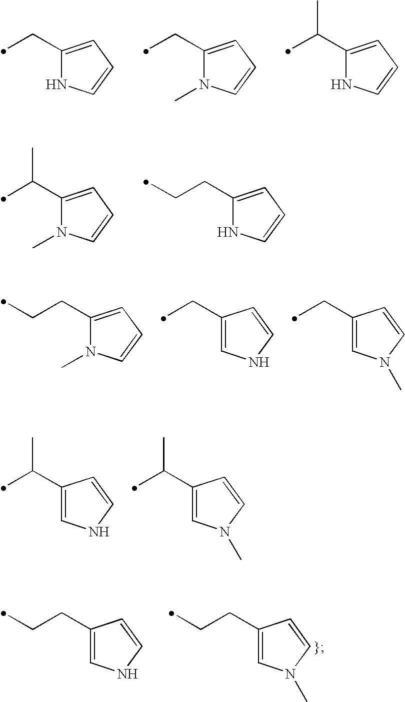

a C1-C4 alkyl group substituted with a pyridyl group optionally substituted with one or more substituents selected from the B group and the C group {specifically. groups represented by the formulas:

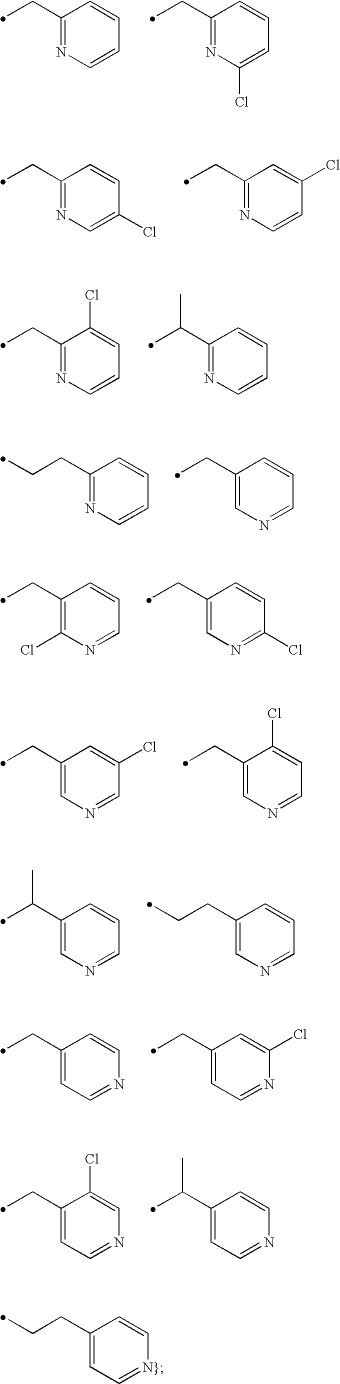

a C1-C4 alkyl group substituted with a pyrimidinyl group optionally substituted with one or more substituents selected from the B group and the C group {specifically, groups represented by the formulas:

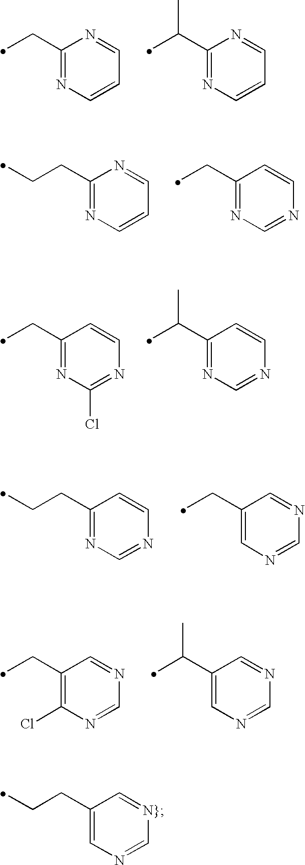

a C1-C4 alkyl group substituted with a pyrazinyl group optionally substituted with one or more substituents selected from the B group and the C group {specifically, groups represented by the formulas:

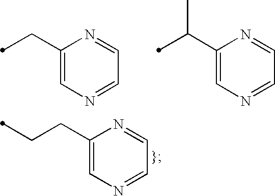

a C1-C4 alkyl group substituted with a pyridazinyl group optionally substituted with one or more substituents selected from the B group and the C group {specifically, groups represented by the formulas:

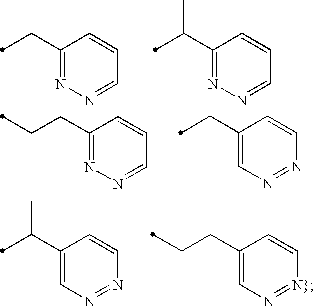

a C1-C4 alkyl group substituted with an imidazolyl group optionally substituted with one or more substituents selected from the B group and the C group {specifically, groups represented by the formulas:

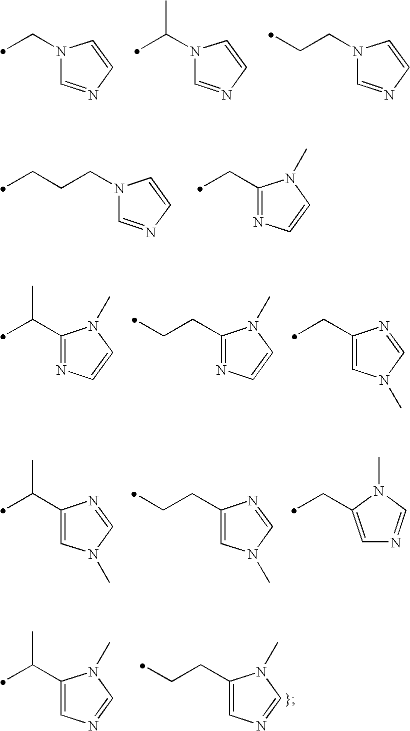

a C1-C4 alkyl group substituted with a pyrazolyl group optionally substituted with one or more substituents selected from the B group and the C group {specifically, groups represented by the formulas:

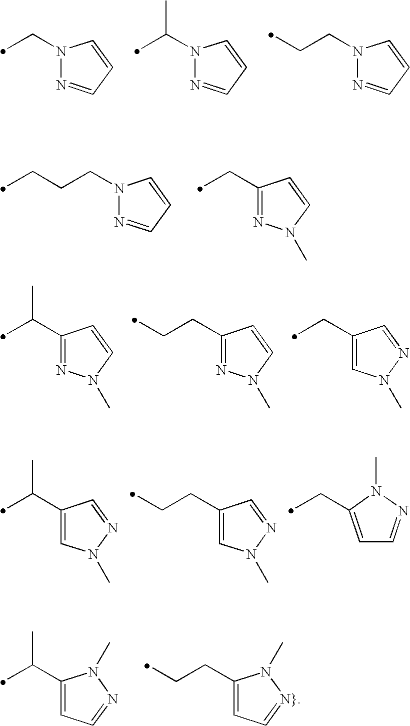

Examples of the “C1-C4 alkyl group substituted with a 5- to 6-membered unsaturated heterocyclic group optionally substituted with one or more substituents selected from the B group and the C group whose heteroatoms are only a sulfur atom and a nitrogen atom, or an oxygen atom and a nitrogen atom” include a C1-C4 alkyl group substituted with a thiazolyl group optionally substituted with one or more substituents selected from the B group and the C group {specifically, groups represented by the formulas:

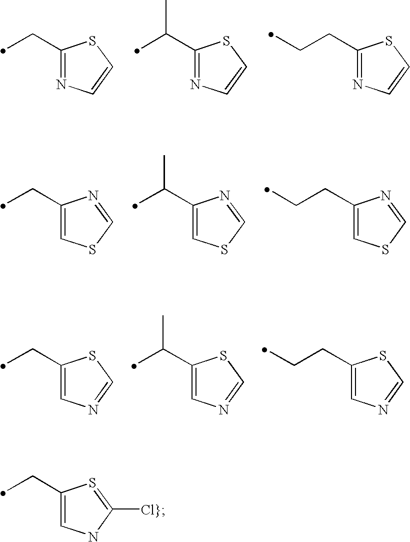

a C1-C4 alkyl group substituted with an isothiazolyl group optionally substituted with one or more substituents selected from the B group and the C group {specifically, groups represented by the formulas:

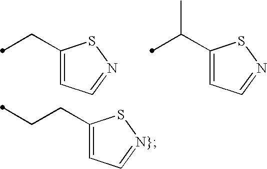

a C1-C4 alkyl group substituted with an isoxazolyl group optionally substituted with one or more substituents selected from the B group and the C group {specifically, groups represented by the formulas:

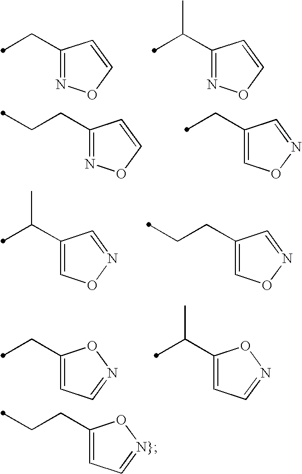

a C1-C4 alkyl group substituted with an oxazolyl group optionally substituted with one or more substituents selected from the B group and the C group {specifically, groups represented by the formulas:

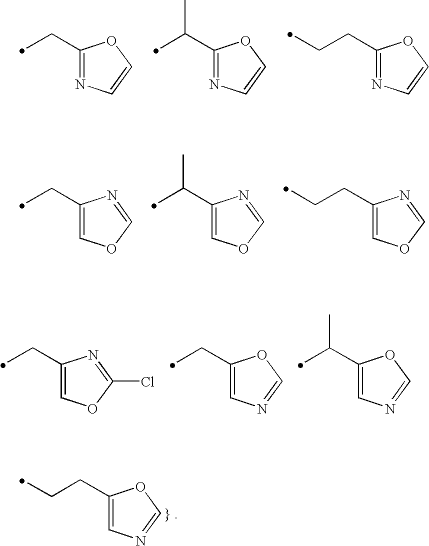

Examples of the “C1-C4 alkyl group substituted with a phenyloxy group optionally substituted with one or more substituents selected from the B group and the C group” include a 2-phenyloxyethyl group, a 2-(2-fluorophenyloxy)ethyl group, a 2-(3-fluorophenyloxy)ethyl group, a 2-(4-fluorophenyloxy)ethyl group, a 2-(2,3-difluorophenyloxy)ethyl group, a 2-(2,4-difluorophenyloxy)ethyl group, a 2-(2,5-difluorophenyloxy)ethyl group, a 2-(2,6-difluorophenyloxy)ethyl group, a 2-(3,4-difluorophenyloxy)ethyl group, a 2-(3,5-difluorophenyloxy)ethyl group, a 2-(2-chlorophenyloxy)ethyl group, a 2-(3-chlorophenyloxy)ethyl group, a 2-(4-chlorophenyloxy)ethyl group, a 2-(2,3-dichlorophenyloxy)ethyl group, a 2-(2,4-dichlorophenyloxy)ethyl group, a 2-(2,5-dichlorophenyloxy)ethyl group, a 2-(2,6-dichlorophenyloxy)ethyl group, a 2-(3,4-dichlorophenyloxy)ethyl group, a 2-(3,5-dichlorophenyloxy)ethyl group, a 2-(2-bromophenyloxy)ethyl group, a 2-(3-bromophenyloxy)ethyl group, a 2-(4-bromophenyloxy)ethyl group, a 2-(2,3-dibromophenyloxy)ethyl group, a 2-(2,4-dibromophenyloxy)ethyl group, a 2-(2,5-dibromophenyloxy)ethyl group, a 2-(2,6-dibromophenyloxy)ethyl group, a 2-(3,4-dibromophenyloxy)ethyl group, a 2-(3,5-dibromophenyloxy)ethyl group, a 2-(2-iodophenyloxy)ethyl group, a 2-(3-iodophenyloxy)ethyl group, a 2-(4-iodophenyloxy)ethyl group, a 2-(2-methylphenyloxy)ethyl group, a 2-(3-methylphenyloxy)ethyl group, a 2-(4-methylphenyloxy)ethyl group, a 2-(2,3-dimethylphenyloxy)ethyl group, a 2-(2,4-dimethylphenyloxy)ethyl group, a 2-(2,5-dimethylphenyloxy)ethyl group, a 2-(2,6-dimethylphenyloxy)ethyl group, a 2-(3,4-dimethylphenyloxy)ethyl group, a 2-(3,5-dimethylphenyloxy)ethyl group, a 2-(2-methoxyphenyloxy)ethyl group, a 2-(3-methoxyphenyloxy)ethyl group, a 2-(4-methoxyphenyloxy)ethyl group, a 2-(2,3-dimethoxyphenyloxy)ethyl group, a 2-(2,4-dimethoxyphenyloxy)ethyl group, a 2-(2,5-dimethoxyphenyloxy)ethyl group, a 2-(2,6-dimethoxyphenyloxy)ethyl group, a 2-(3,4-dimethoxyphenyloxy)ethyl group, a 2-(3,5-dimethoxyphenyloxy)ethyl group, a 2-(2-ethylphenyloxy)ethyl group, a 2-(3-ethylphenyloxy)ethyl group, a 2-(4-ethylphenyloxy)ethyl group, a 2-(2-(trifluoromethyl)phenyloxy)ethyl group, a 2-(3-(trifluoromethyl)phenyloxy)ethyl group, a 2-(4-(trifluoromethyl)phenyloxy)ethyl group, a 2-(2-methylthiophenyloxy)ethyl group, a 2-(3-methylthiophenyloxy)ethyl group, a 2-(4-methylthiophenyloxy)ethyl group, a 2-(2-(trifluoromethoxy)phenyloxy)ethyl group, a 2-(3-(trifluoromethoxy)phenyloxy)ethyl group, a 2-(4-(trifluoromethoxy)phenyloxy)ethyl group, a 2-(2-nitrophenyloxy)ethyl group, a 2-(3-nitrophenyloxy)ethyl group, a 2-(4-nitrophenyloxy)ethyl group, a 2-(2-cyanophenyloxy)ethyl group, a 2-(3-cyanophenyloxy)ethyl group, a 2-(4-cyanophenyloxy)ethyl group, and a 3-phenyloxypropyl group.

Examples of the “group in which a C1-C4 alkyl group is substituted with a 3- to 10-membered heterocyclic group optionally substituted with one or more substituents selected from the B group and the C group via an oxygen atom” include a C1-C4 alkyl group substituted with a thiadiazolyloxy group optionally substituted with one or more substituents selected from the B group and the C group {specifically, groups represented by the formulas:

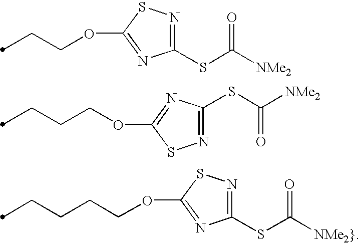

Examples of the “C1-C4 alkyl group substituted with a benzyloxy group optionally substituted with one or more substituents selected from the B group and the C group” include a 2-benzylokyethyl group, a 2-(2-fluorobenzyloxy)ethyl group, a 2-(3-fluorobenzyloxy)ethyl group, a 2-(4-fluorobenzyloxy)ethyl group, a 2-(2,3-difluorobenzyloxy)ethyl group, a 2-(2,4-difluorobenzyloxy)ethyl group, a 2-(2,5-difluorobenzyloxy)ethyl group, a 2-(2,6-difluorobenzyloxy)ethyl group, a 2-(3,4-difluorobenzyloxy)ethyl group, a 2-(3,5-difluorobenzyloxy)ethyl group, a 2-(2-chlorobenzyloxy)ethyl group, a 2-(3-chlorobenzyloxy)ethyl group, a 2-(4-chlorobenzyloxy)ethyl group, a 2-(2,3-dichlorobenzyloxy)ethyl group, a 2-(2,4-dichlorobenzyloxy)ethyl group, a 2-(2,5-dichlorobenzyloxy)ethyl group, a 2-(2,6-dichlorobenzyloxy)ethyl group, a 2-(3,4-dichlorobenzyloxy)ethyl group, a 2-(3,5-dichlorobenzyloxy)ethyl group, a 2-(2-bromobenzyloxy)ethyl group, a 2-(3-bromobenzyloxy)ethyl group, a 2-(4-bromobenzyloxy)ethyl group, a 2-(2,3-dibromobenzyloxy)ethyl group, a 2-(2,4-dibromobenzyloxy)ethyl group, a 2-(2,5-dibromobenzyloxy)ethyl group, a 2-(2,6-dibromobenzyloxy)ethyl group, a 2-(3,4-dibromobenzyloxy)ethyl group, a 2-(3,5-dibromobenzyloxy)ethyl group, a 2-(2-iodobenzyloxy)ethyl group, a 2-(3-iodobenzyloxy)ethyl group, a 2-(4-iodobenzyloxy)ethyl group, a 2-(2-methylbenzyloxy)ethyl group, a 2-(3-methylbenzyloxy)ethyl group, a 2-(4-methylbenzyoxy)ethyl group, a 2-(2-(trifluoromethyl)benzyloxy)ethyl group, a 2-(3-(trifluoromethyl)benzyloxy)ethyl group, a 2-(4-(trifluoromethyl)benzyloxy)ethyl group, a 2-(2-methoxybenzyloxy)ethyl group, a 2-(3-methoxybenzyloxy)ethyl group, a 2-(4-methoxybenzyloxy)ethyl group, a 2-(2,5-dimethoxybenzyloxy)ethyl group, a 2-(3,5-dimethoxybenzyloxy)ethyl group, a 2-(2-methylthiobenzyloxy)ethyl group, a 2-(3-methylthiobenzyloxy)ethyl group, a 2-(4-methylthiobenzyloxy)ethyl group, a 2-(2-(trifluoromethoxy)benzyloxy)ethyl group, a 2-(3-(trifluoromethoxy)benzyloxy)ethyl group, a 2-(4-(trifluoromethoxy)benzyloxy)ethyl group, a 2-(2-nitrobenzyloxy)ethyl group, a 2-(3-nitrobenzyloxy)ethyl group, a 2-(4-nitrobenzyloxy)ethyl group, a 2-(2-cyanobenzyloxy)ethyl group, a 2-(3-cyanobenzyloxy)ethyl group, a 2-(4-cyanobenzyloxy)ethyl group, a 2-(2-ethoxybenzyloxy)ethyl group, a 2-(3-ethoxybenzyloxy)ethyl group, a 2-(4-ethoxybenzyloxy)ethyl group, a 2-(4-isopropylbenzyloxy)ethyl group, a 2-(4-tert-butylbenzyloxy)ethyl group, a 2-(2-fluoro-4-(trifluoromethyl)benzyloxy)ethyl group, a 2-(2-fluoro-5-(trifluoromethyl)benzyloxy)ethyl group, a 2-(4-fluoro-3-(trifluoromethyl)benzyloxy)ethyl group, a 2-(2,4-bis(trifluoromethyl)benzyloxy)ethyl group, a 2-(5-fluoro-2-methylbenzyloxy)ethyl group, a 2-(pentafluorobenzyloxy)ethyl group, and a 3-benzyloxypropyl group.

Examples of the “C1-C4 alkyl group” include a methyl group, an ethyl group, a propyl group, an isopropyl group, a butyl group, an isobutyl group, a sec-butyl group, and a tert-butyl group.

Examples of the “C3-C4 alkenyl group” include a 2-propenyl group, a 2-butenyl group, a 3-butenyl group, and a 2-methyl-2-propenyl group.

Examples of the “C1-C4 alkoxy group” include a methoxy group, an ethoxy group, a propoxy group, an isopropoxy group, a butoxy group, an isobutoxy group, a sec-butoxy group, and a tert-butoxy group.

Examples of the “C2-C7 alkanediyl group” include an ethylene group, a propylene group, a butane-1,4-diyl group, a pentane-1,5-diyl group, a hexane-2,5-diyl group, and a heptane-2,6-diyl group.

Examples of the “C1-C4 alkanediyl group” include a methylene group, an ethylene group, a propylene group, a propane-1,3-diyl group, and a butane-1,4-diyl group.

Examples of the “morpholino group” include a morpholino group, and a 2,6-dimethylmorpholino group.

Aspects of the present compounds are exemplified as follows:

### “Aspect 1”

The thiadiazole compound of the formula (I), wherein X is a —NR²R³ group or a morpholino group, and
R² and R³ each independently are a hydrogen atom, a C1-C4 alkyl group, a C3-C4 alkenyl group, a C1-C4 alkoxy group, a benzyl group, or a phenyl group, or R² and R³ bind to each other at the ends thereof to form a C2-C7 alkanediyl group.

### “Aspect 2”

The thiadiazole compound of the formula (I), wherein X is a —NR²R³ group or a morpholino group, and R² and R³ each independently are a C1-C4 alkyl group, or a phenyl group, or R² and R³ bind to each other at the ends thereof to form a C2-C7 alkanediyl group.

### “Aspect 3”

The thiadiazole compound of the formula (I), wherein R is a C1-C7 chain hydrocarbon group optionally substituted with one or more substituents selected from the A group, a -Q group, a -T-Q group, a -T-O-Q group, or a -T-O-T-Q group, Q is (1) a 3- to 10-membered carbocyclic group optionally substituted with one or more substituents selected from the B group, or optionally substituted with one or more substituents selected from the C group at the same position or adjacent positions, or (2) a 3- to 10-membered heterocyclic group optionally substituted with one or more substituents selected from the B group, or optionally substituted with one or more substituents selected from the C group at the same position or adjacent positions, and
T is a C1-C4 alkanediyl group.

### “Aspect 4”

The thiadiazole compound of the formula (I), wherein R is a C1-C7 chain hydrocarbon group optionally substituted with one or more substituents selected from the D group, a -Q² group, a -T-Q² group, a -T-O-Q² group, or a -T-O-T-Q² group,
Q² is (1) a 3- to 10-membered carbocyclic group optionally substituted with one or more substituents selected from the E group, or optionally substituted with one or more substituents selected from the F group at the same position or adjacent positions, or (2) a 3- to 10-membered heterocyclic group optionally substituted with one or more substituents selected from the E group, or optionally substituted with one or more substituents selected from the F group at the same position or adjacent positions, and
T is a C1-C4 alkanediyl group.

### “Aspect 5”

The thiadiazole compound of the formula (I), wherein R is a C1-C7 chain hydrocarbon group optionally substituted with one or more substituents selected from the D group, a -Q⁴ group, a -T-Q⁴ group, a -T-O-Q⁴ group, or a -T-O-T-Q⁴ group,
Q⁴ is (1) a 3- to 6-membered carbocyclic group optionally substituted with one or more substituents selected from the B group, or optionally substituted with one or more substituents selected from the C group at the same position or adjacent positions, or (2) a 3- to 6-membered saturated heterocyclic group optionally substituted with one or more substituents selected from the B group, or optionally substituted with one or more substituents selected from the C group at the same position or adjacent positions, and
T is a C1-C4 alkanediyl group.

### “Aspect 6”

The thiadiazole compound of the formula (I), wherein R is a C1-C7 chain hydrocarbon group optionally substituted with one or more substituents selected from the D group, a -Q⁶ group, a -T-Q⁶ group, a -T-O-Q⁶ group, or a -T-O-T-Q⁶ group,
Q⁶ is (1) a 3- to 6-membered carbocyclic group optionally substituted with one or more substituents selected from the E group, or optionally substituted with one or more substituents selected from the F group at the same position or adjacent positions, or (2) a 3- to 6-membered saturated heterocyclic group optionally substituted with one or more substituents selected from the E group, or optionally substituted with one or more substituents selected from the F group at the same position or adjacent positions, and
T is a C1-C4 alkanediyl group.

### “Aspect 7”

The thiadiazole compound of the formula (I), wherein R is a C1-C7 chain hydrocarbon group optionally substituted with one or more substituents selected from the D group, a -Q⁷ group, or a -T-Q⁷ group,
Q⁷ is (1) a C3-C8 cycloalkyl group optionally substituted with one ore more substituents selected from the E group, or optionally substituted with one ore more substituents selected from the F group at the same position or adjacent positions, or (2) a group represented by

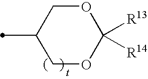

wherein
t is 0 or 1,
R¹³ and R¹⁴ each independently are a hydrogen atom, a C1-C4 alkyl group, a C2-C7 alkenyl group, a C2-C4 alkynyl group, a C1-C4 alkoxyalkyl group, or a -Q⁸ group, or R¹³ and R¹⁴ bind to each other at the ends thereof to form a C2-C7 alkanediyl group, or a —Z⁴-T-Z⁵-group,
Q⁸ is (1) a 3- to 10-membered carbocyclic group optionally substituted with one or more substituents selected from the D group, or optionally substituted with one or more substituents selected from the F group at the same position or adjacent positions, or (2) a 3- to 10-membered heterocyclic group optionally substituted with one or more substituents selected from the D group, or optionally substituted with one or more substituents selected from the F group at the same position or adjacent positions,
Z⁴ and Z⁵ each independently are an oxygen atom or a sulfur atom, and
T is a C1-C4 alkanediyl group.

### “Aspect 8”

The thiadiazole compound of the formula (I), wherein R is a C1-C7 chain hydrocarbon group optionally substituted with one or more substituents selected from the D group, a -Q⁷ group or a -T-Q⁷ group,
Q⁷ is (1) a C3-C8 cycloalkyl group optionally substituted with one ore more substituents selected from the E group, or optionally substituted with one ore more substituents selected from the F group at the same position or adjacent positions, or (2) a group represented by

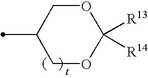

wherein
t is 0 or 1,
R¹³ and R¹⁴ each independently are a hydrogen atom, a C1-C4 alkyl group, a C2-C7 alkenyl group, a C2-C4 alkynyl group, a C1-C4 alkoxyalkyl group, or a -Q group, or R¹³ and R¹⁴ bind to each other at the ends thereof to form a C2-C7 alkanediyl group, or a —Z⁴-T-Z⁵— group,
Q⁸ is (1) a 3- to 10-membered carbocyclic group optionally substituted with one or more substituents selected from the D group, or optionally substituted with one or more substituents selected from the F group at the same position or adjacent positions, or (2) a 3- to 10-membered heterocyclic group optionally substituted with one or more substituents selected from the D group, or optionally substituted with one or more substituents selected from the F group at the same position or adjacent positions,
Z⁴ and Z⁵ each independently are an oxygen atom or a sulfur atom, and
T is a C1-C4 alkanediyl group.

### “Aspect 9”

The thiadiazole compound of the formula (I), wherein R is a C1-C7 chain hydrocarbon group optionally substituted with one ore more substituents selected from the D group, a -Q⁹ group or a -T-Q⁹ group,
Q⁹ is (1) a phenyl group optionally substituted with one or more substituents selected from the E group, or (2) a 5- to 6-membered unsaturated heterocyclic group optionally substituted with one ore more substituents selected from the E group, and
T is a C1-C4 alkanediyl group. “Aspect 10”
The thiadiazole compound represented by the formula (I′):

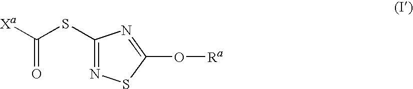

wherein R^{a} is (i) a C1-C7 alkyl group, (ii) a C1-C6 haloalkyl group, (iii) a C3-C6 alkenyl group, (iv) a C3-C6 haloalkenyl group, (v) a C3-C6 alkynyl group, (vi) a C3-C6 haloalkynyl group, (vii) a C2-C7 alkoxyalkyl group, (viii) a C2-(ix) C6 alkylthioalkyl group, (x) a C3-C8 cycloalkyl group optionally substituted with one or more substitutents selected from the H group, (xi) a C1-C4 alkyl group substituted with a C3-C8 cycloalkyl group optionally substituted with one ore more substituents selected from the H group, (xii) a C5-C8 cycloalkenyl group optionally substituted with one or more substitutents selected from the H group, (xiii) a C1-C4 alkyl group substituted with a C5-C8 cycloalkenyl group optionally substituted with one or more substitutents selected from the H group, (xiv) a heterocyclic group optionally substituted with one or more substituents selected from the I group, said heterocyclic group representing a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 6-membered heterocyclic group containing only one or two sulfur atoms as the heteroatoms, a 5-membered heterocyclic group containing only one or two nitrogen atoms as the heteroatoms, a 5-membered heterocyclic group containing only a sulfur atom and a nitrogen atom as the heteroatoms, a 5-membered heterocyclic group containing only an oxygen atom and a nitrogen atom as the heteroatoms, or a 6-membered heterocyclic group containing only one or two nitrogen atoms as heteroatoms, (xv) a C1-C4 alkyl group substituted with a heterocyclic group optionally substituted with one or more substituents selected from the I group, said heterocyclic group representing a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 6-membered heterocyclic group containing only one or two sulfur atoms as the heteroatoms, a 5-membered heterocyclic group containing only one or two nitrogen atoms as the heteroatoms, a 5-membered heterocyclic group containing only a sulfur atom and a nitrogen atom as the heteroatoms, a 5-membered heterocyclic group containing only an oxygen atom and a nitrogen atom as the heteroatoms, or a 6-membered heterocyclic group containing only one or two nitrogen atoms as the heteroatom, (xvi) a phenyl group optionally substituted with one or more substituents selected from the I group, (xvii) a C1-C4 alkyl group substituted with a phenyl group optionally substituted with one or more substituents selected from the I group, (xviii) a C2-C6 formylalkyl group, (xix) a C2-C6 cyanoalkyl group, (xx) a C2-C6 hydroxyiminoalkyl group, (xxi) a C3-C7 alkoxyiminoalkyl group, (xxii) a C2-C8 alkylaminoalkyl group, (xxiii) a C2-C6 alkoxycarbonylalkyl group, (xxiv) a C2-C6 hydroxyalkyl group, or (xxv) a C3-C6 alkanoyl group, and
X^{a} represents a morpholino group, or a —NR²R³ group (wherein R² and R³ each independently represent a hydrogen atom, a C1-C4 alkyl group, a C3-C4 alkenyl group, a C1-C4 alkoxy group, or a phenyl group, or R² and R³ bind to each other at the ends thereof to form a C2-C7 alkanediyl group).

### “Aspect 11”

The thiadiazoyl compound of the formula (I′), wherein R^{a} is (i) a C1-C7 alkyl group, (ii) a C1-C6 haloalkyl group, (iii) a C3-C6 alkenyl group, (iv) a C3-C6 haloalkenyl group, (v) a C3-C6 alkynyl group, (vi) a C2-C6 alkoxyalkyl group, (vii) a C2-C6 alkylthioalkyl group, (viii) a C3-C8 cycloalkyl group optionally substituted with one or more substituents selected from the J group, (ix) a C1-C4 alkyl group substituted with a C3-C8 cycloalkyl group optionally substituted with one or more substituents selected from the J group, (x) a C1-C4 alkyl group substituted with a C5-C8 cycloalkenyl group optionally substituted with one or more substituents selected from the J group, (xi) a heterocyclic group optionally substituted with one or more substituents selected from the K group, said heterocyclic group being a 5-membered heterocyclic group containing only one or two oxygen atoms as the heteroatoms, or a 6-membered heterocyclic group containing only one or two oxygen atoms as the heteroatoms, (xii) a C1-C4 alkyl group substituted with a heterocyclic group optionally substituted with one or more substituents selected from the K group, said heterocyclic group being a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 5-membered heterocyclic group containing only one or two nitrogen atoms as the heteroatoms, a 5-membered heterocyclic group containing only a sulfur atom and a nitrogen atom as the heteroatoms, or a 6-membered heterocyclic group containing only one or two nitrogen atoms as the heteroatoms, or (xiii) a C1-C4 alkyl group substituted with a phenyl group optionally substituted with one or more substituents selected from the L group and,
X^{a} is a morpholino group, or a NR²R³ group (wherein R² and R³ each independently are a lower alkyl group, or a phenyl group, or R² and R³ bind to each other at the ends thereof to form a C2-C7 alkanediyl group).

### “Aspect 12”

The thiadiazole compound of the formula (I′), wherein R^{a} is (i) a C1-C7 alkyl group, (ii) a C1-C6 haloalkyl group, (iii) a C3-C6 alkenyl group, (iv) a C3-C6 haloalkenyl group, (v) a C3-C6 alkynyl group, (vi) a C2-C6 alkoxyalkyl group, (vii) a C3-C8 cycloalkyl group optionally substituted with one or more substituents selected from the J group, (viii) a C1-C4 alkyl group substituted with a C3-C8 cycloalkyl group optionally substituted with one or more substituents selected from the J group, (ix) a C1-C4 alkyl group substituted with a C5-C8 cycloalkenyl group optionally substituted with one or more substituents selected from the J group, (x) a heterocyclic group optionally substituted with one or more substituents selected from the K group, said heterocyclic group being a 5-membered heterocyclic group containing only one or two oxygen atoms as the heteroatoms, or a 6-membered heterocyclic group containing one or two oxygen atoms as the heteroatoms, (xi) a C1-C4 alkyl group substituted with a heterocyclic group optionally substituted with one or more substituents selected from the K group, said heterocyclic group being a 5-membered heterocyclic group containing only one or two oxygen atoms as the heteroatoms, a 6-membered heterocyclic group containing only one or two oxygen atoms as the heteroatoms, a 5-membered heterocyclic group containing only one sulfur atom as the heteroatom, a 5-membered heterocyclic group containing only one or two nitrogen atoms as the heteroatoms, or a 6-membered heterocyclic group containing only one or two nitrogen atoms as the heteroatoms, or (xii) a C1-C4 alkyl group substituted with a phenyl group optionally substituted with one or more substituents selected from the L group, and
X^{a} is a morpholino group, or a group represented by NR²R³ group (wherein R² and R³ each independently are a C1-C4 alkyl group, or a phenyl group, or R² and R³ bind to each other at the ends thereof to form a C2-C7 alkanediyl group).

### “Aspect 13”

The thiadiazole compound of the formula (I′), wherein R^{a} is (i) a C1-C7 alkyl group, (ii) a C1-C6 haloalkyl group, (iii) a C3-C6 alkenyl group, (iv) a C3-C6 haloalkenyl group, (vi) a C3-C6 alkynyl group, (vii) a C2-C7 alkoxyalkyl group, (viii) a heterocyclic group optionally substituted with one or more C1-C4 alkyl groups, said heterocyclic group being a 5-membered heterocyclic group containing only one or two oxygen atoms as the heteroatoms, or a 6-membered heterocyclic group containing only one or two oxygen atom as the heteroatoms, or (ix) a C1-C4 alkyl group substituted with a heterocyclic group optionally substituted with one or more C1-C4 alkyl groups, said heterocyclic group being a 5-membered heterocyclic group containing only one or two oxygen atoms as the heteroatoms, a 6-membered heterocyclic group containing one or two oxygen atoms as the heteroatoms, or a 5-membered heterocyclic group containing only one or two nitrogen atoms as the heteroatoms, and
X^{a} is a morpholino group, or NR²R³ (wherein R² and R³ each independently are a C1-C4 alkyl group, or a phenyl group, or R² and R³ are taken together with the nitrogen atom to which they bind to form a 3- to 8-membered ring).

### “Aspect 14”

The thiadiazole compound of the formula (I′), wherein R^{a} is a methyl group, an ethyl group, a propyl group, an isopropyl group, a butyl group, a 1,2-dimethylbutyl group, a 2-methylbutyl group, a 1-ethylpropyl group, a 1,2-dimethylpropyl group, a neopentyl group, a heptyl group, a 3,3-dimethylbutyl group, a 1-tert-butylpropyl group, a 2,2,2-trifluoroethyl group, a 3-chloropropyl group, a 4-chlorobutyl group, a 6-chlorohexyl group, a 3-chloro-2,2-dimethylpropyl group, a 2,2-dichloroethyl group, a 2,3-dichloropropyl group, a 2-fluoroethyl group, a 2,2-difluoroethyl group, a 2-fluoro-1-(fluoromethyl)ethyl group, a 2,2,2-trifluoro-1-(trifluoromethyl)ethyl group, a 2,2,3,3,3-pentafluoropropyl group, a 2,2,3,3,3-pentafluoro-1-methylpropyl group, a 2,2,3,3,4,4,4-heptafluorobutyl group, a 2-chloroethyl group, a 2-chloro-1-methylethyl group, a 3-butenyl group, a 4-pentenyl group, a 1-methyl-2-propenyl group, a 2-methyl-2-propenyl group, a 2,2,3,4,4-pentafluoro-3-butenyl group, a 2-propynyl group, a 2-butynyl group, a 2-pentynyl group, a 3-butynyl group, a 1-ethyl-2-propynyl group, a 1-methyl-3-butenyl group, a 1-methyl-2-propynyl group, a 1,1-dimethyl-2-propynyl group, a 2-methoxyethyl group, a 3-methoxypropyl group, a 2-methylthioethyl group, a cyclopentyl group, a cyclohexyl group, a 2-chlorocyclohexyl group, a 1-ethynylcylohexyl group, a cycloheptyl group, a cyclooctyl group, a cyclopropylmethyl group, a cyclopropyl(methyl)methyl group, a cyclobutylmethyl group, a 1-cyclopentylethyl group, a 1-cyclohexylethyl group, a cyclohexylpropyl group, a cyclopentylethyl group, a cyclohexylmethyl group, a (3-cyclohexan-1-yl)methyl group, a 1,3-dioxan-5-yl group, a tetrahydro-4-pyranyl group, a tetrahydro-3-furyl group, a tetrahydro-2-furylmethyl group, a tetrahydro-3-furylmethyl group, a tetrahydro-2-pyranylmethyl group, a (2,2-dimethyl-1,3-dioxolan-4-yl)methyl group, a (1,3-dioxolan-4-yl)methyl group, an oxylanylmethyl group, a 6-chloro-2-pyridylmethyl group, a 3-(1H-pyrazol-1-yl)propyl group, a (2-chlorothiazol-5-yl)methyl group, a 2-(5,5-dimethyl-1,3-dioxan-2-yl)ethyl group, a 3-furylmethyl group, a 2-furylmethyl group, a 2-thienylmethyl group, a 3-thienylethyl group, a 2-fluorobenzyl group, a 3-fluorobenzyl group, a 4-fluorobenzyl group, a 2-chlorobenzyl group, a 3-chlorobenzyl group, a 4-chlorobenzyl group, a 2-bromobenzyl group, a 3-bromobenzyl group, a 4-bromobenzyl group, a 2-iodobenzyl group, a 4-ethylbenzyl group, a 3-methylbenzyl group, a 4-methylbenzyl group, a 2-methoxybenzyl group, a 3-methoxybenzyl group, a 4-methoxybenzyl group, a 2-ethoxy-benzyl group, a 4-ethoxybenzyl group, a 4-isopropylbenzyl group, a 4-methylthiobenzyl group, a 4-tert-butylbenzyl group, a 2,3-dichlorobenzyl group, a 2,4-dichlorobenzyl group, a 2,5-dichlorobenzyl group, a 2,6-dichlorobenzyl group, a 2,3-dichlorobenzyl group, a 3,5-dichlorobenzyl group, a 2,5-difluorobenzyl group, a 2,6-difluorobenzyl group, a 3,4-difluorobenzyl group, a 3,5-difluorobenzyl group, a 2-fluoro-4-(trifluoromethyl)benzyl group, a 2-fluoro-5-(trifluoromethyl)benzyl group, a 4-fluoro-3-(trifluoromethyl)benzyl group, a 2,4-bis(trifluoromethyl)benzyl group, a 2,4-dimethylbenzyl group, a 3,4-dimethylbenzyl group, a 2,5-dimethoxybenzyl group, a 3,5-dimethoxybenzyl group, a 5-fluoro-2-methylbenzyl group, or a pentafluorobenzyl group, and
X^{a} is a morpholino group, a pyrrolidino group, a pyperidino group, a dimethylamino group, a diethylemino group, a diphenylamino group, or a methylphenylamino group.

### “Aspect 15”

The thiadiazole compound of the formula (I′), wherein R^{a} is a methyl group, an ethyl group, a propyl group, an isopropyl group, a butyl group, a 1,2-dimethylbutyl group, a 2-methylbutyl group, a 1-ethylpropyl group, a 1,2-dimethylpropyl group, a neopentyl group, a 3,3-dimethylbutyl group, a 1-tert-butylpropyl group, a 2,2,2-trifluoroethyl group, a 3-chloropropyl group, a 4-chlorobutyl group, a 6-chlorohexyl group, a 3-chloro-2,2-dimethylpropyl group, a 2,2-dichloroethyl group, a 2,3-dichloropropyl group, a 2-fluoroethyl group, a 2,2-difluoroethyl group, a 2-fluoro-1-(fluoromethyl)ethyl group, a 2,2,3,3,3-pentafluoropropyl group, a 2,2,3,3,3-pentafluoro-1-methylpropyl group, a 2,2,3,3,4,4,4-heptafluorobutyl group, a 2-chloroethyl group, a 2-chloro-1-methylethyl group, a 3-butenyl group, a 4-pentenyl group, a 2-methyl-2-propenyl group, a 2-propynyl group, a 2-butynyl group, a 2-pentynyl group, a 3-butynyl group, a 1-ethyl-2-propynyl group, a 1-methyl-3-butynyl group, a 1-methyl-2-propynyl group, a 2-methoxyethyl group, a 3-methoxypropyl group, a cyclopentyl group, a cyclohexyl group, a 2-chlorocyclohexyl group, a cyclooctyl group, a cyclopropylmethyl group, a cyclopropyl(methyl)methyl group, a cyclobutylmethyl group, a (3-cyclohexen-1-yl)methyl group, a 1,3-dioxan-5-yl group, a tetrahydro-4-pyranyl group, a tetrahydro-3-furyl group, a tetrahydro-2-furylmethyl group, a tetrahydro-3-furylmethyl group, a tetrahydro-2-pyranylmethyl group, a (2,2-dimethyl-1,3-dioxolan-4-yl)methyl group, a (1,3-dioxolan-4-yl)methyl group, a 6-chloro-2-pyridylmethyl group, a 3-(1H-pyrazol-1-yl)propyl group, a 2-(5,5-dimethyl-1,3-dioxan-2-yl)ethyl group, a 3-thienylmethyl group, a 2-fluorobenzyl group, a 3-fluorobenzyl group, a 4-fluorobenzyl group, a 2-chlorobenzyl group, a 3-chlorobenzyl group, a 4-chlorobenzyl group, a 3-bromobenzyl group, a 4-bromobenzyl group, a 2-iodobenzyl group, a 4-ethylbenzyl group, a 3-methylbenzyl group, a 2-methoxybenzyl group, a 3-methoxybenzyl group, a 4-methoxybenzyl group, a 4-ethoxybenzyl group, a 4-isopropylbenzyl group, a 4-methylthiobenzyl group, a 2,3-dichlorobenzyl group, a 2,5-dichlorobenzyl group, a 2,6-dichlorobenzyl group, a 2,3-dichlorobenzyl group, a 2,5-difluorobenzyl group, a 2,6-difluorobenzyl group, a 3,4-difluorobenzyl group, a 3,5-difluorobenzyl group, a 2-fluoro-4-(trifluoromethyl)benzyl group, a 4-fluoro-3-(trifluoromethyl)benzyl group, a 2,4-bis(trifluoromethyl)benzyl group, a 5-fluoro-2-methylbenzyl group, or a pentafluorobenzyl group, and
X^{a} is a morpholino group, a pyrrolidino group, a piperidino group, a dimethylamino group, a diethylamino group, or a methylphenylamino group.

### “Aspect 16”

The thiadiazole compound of the formula (I′), wherein R^{a} is a methyl group, an ethyl group, a propyl group, an isopropyl group, a butyl group, a 2-methylbutyl group, a 1-ethylpropyl group, a 1,2-dimethylpropyl group, a neopentyl group, a 2,2,2-trifluoroethyl group, a 3-chloropropyl group, a 4-chlorobutyl group, a 3-chloro-2,2-dimethylpropyl group, a 2,3-dichloropropyl group, a 2-fluoroethyl group, a 2,2-difluoroethyl group, a 2-fluoro-1-(fluoromethyl)ethyl group, a 2,2,3,3,3-pentafluoropropyl group, a 2,2,3,3,3-pentafluoro-1-methylpropyl group, a 2-chloroethyl group, a 3-butenyl group, a 4-pentenyl group, a 2-methyl-2-propenyl group, a 2-propynyl group, a 2-butynyl group, a 2-pentynyl group, a 3-butynyl group, a 1-ethyl-2-propynyl group, a 1-methyl-3-butynyl group, a 1-methyl-2-propynyl group, a 2-methoxyethyl group, a 3-methoxypropyl group, a 1,3-dioxan-5-yl group, a tetrahydro-4-pyranyl group, a tetrahydro-3-furyl group, a tetrahydro-3-furylmethyl group, a tetrahydro-2-pyranylmethyl group, a (2,2-dimethyl-1,3-dioxolan-4-yl)methyl group, a (1,3-dioxolan-4-yl)methyl group, a 3-(1H-pyrazol-1-yl)propyl group, or a 2-(5,5-dimethyl-1,3-dioxan-2-yl)ethyl group, and
X^{a} is a morpholino group, a pyrrolidino group, a piperidino group, a dimethylamino group, a diethylamino group, or a methylphenylamino group.

### “Aspect 17”

The thiadiazole compound according to any one of “Aspect 3” to “Aspect 9”, wherein X is a di(C1-C4 alkyl)amino group in the formula (I).

### “Aspect 18”

The thiadiazole compound according to any one of “Aspect 3” to “Aspect 9”, wherein X is a dimethylamino group in the formula (I).

### “Aspect 19”

The thiadiazole compound according to any one of “Aspect 3” to “Aspect 9”, wherein X is a morpholino group in the formula (I).

### “Aspect 20”

The thiadiazole compound according to any one of “Aspect 10” to “Aspect 16”, wherein X^{a} is a di(C1-C4 alkyl)amino group in the formula (I′).

### “Aspect 21”

The thiadiazole compound according to any one of “Aspect 10” to “Aspect 16”, wherein X^{a} is a dimethylamino group in the formula (I′).

### “Aspect 22”

The thiadiazole compound according to any one of “Aspect 10” to “Aspect 16”, wherein X^{a} is a morpholino group in the formula (I′).

Aspects of the present intermediate are exemplified as follows:

### “Aspect 1 of the Present Intermediate”

The compound of the formula (II), wherein
Y is a chlorine atom,
X is a morpholino group or a group represented by NR²R³ (wherein R² and R³ each independently are a lower alkyl group, a benzyl group, or a phenyl group, or R² and R³ are taken together with the nitrogen atom to which they bind to form a 3- to 8-membered ring).

### “Aspect 2 of the Present Intermediate”

The compound of the formula (II), wherein
Y is a chlorine atom, and
X is a morpholino group, a pyrrolidino group, a piperidino group, a dimethylamino group, a diethylamino group, or a methylphenylamino group.

Hereinafter, a process for producing the present compound will be explained.

The present compound can be produced, for example, by the following (Process 1) to (Process 9).

### (Process 1)

Among the present compounds, a compound represented by the formula (Ia):

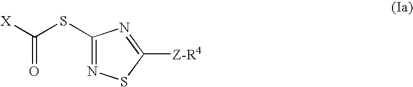

wherein R⁴ is a C1-C7 chain hydrocarbon group optionally substituted with one or more monovalent groups selected from the A group, a -Q group, a -T-Q group, a -T-O-Q group, or a -O-T-Q group,
X, Z, Q and T are as defined above,
can be produced by reacting a compound represented by the formula (II):

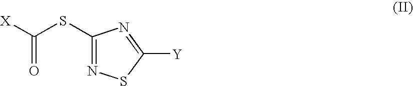

wherein X and Y are as defined above,
and a compound represented by the formula (III):

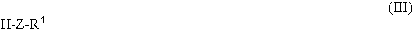

wherein Z and R⁴ are as defined above.

The reaction is performed usually in the presence of a base usually in a solvent.

Examples of the solvent used in the reaction include aliphatic hydrocarbons such as hexane, heptane and the like, aromatic hydrocarbons such as toluene, xylene and the like, ethers such as diethyl ether, tert-butyl methyl ether, tetrahydrofuran, 1,4-dioxane, 1,2-dimethoxyethane and the like, halogenated hydrocarbons such as methylene chloride, chloroform and the like, aprotic polar solvents such as N,N-dimethylformamide, N-methylpyrrolidone and the like, and a mixture thereof.

Examples of the base used in the reaction include hydroxides of alkali metals or alkaline earth metals such as sodium hydroxide, potassium hydroxide, calcium hydroxide and the like, hydrides of alkali metals or alkaline earth metals such as sodium hydride, potassium hydride, calcium hydride and the like, inorganic bases such as sodium carbonate, potassium carbonate and the like, and organic bases such as triethylamine.

The amount of the compound represented by the formula (III) is usually 1 to 2 mole, and the amount of the base is usually 1 to 1.5 mole relative to 1 mole of the compound represented by the formula (II).

The reaction temperature is usually in a range of −78° C. to 100° C., and the reaction time is usually in a range of 0.1 to 24 hours.

After completion of the reaction, a compound represented by the formula (Ia) can be isolated by performing a post-treatment procedure such as by pouring the reaction mixture into water, followed by extracting with an organic solvent, drying and then concentrating the organic layer. The isolated compound represented by the formula (Ia) can be further purified by recrystallization, column chromatography or the like.

### (Process 2)

Among the present compounds, a compound represented by the formula (Ia) can be produced by reacting a compound represented by the formula (IV):

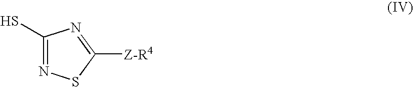

wherein Z and R⁴ are as defined above,
and a carbamoyl chloride compound represented by the formula (V):

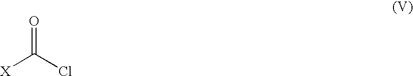

wherein X is as defined above.

The reaction is performed usually in the presence of a base usually in a solvent.

Examples of the solvent used in the reaction include ketones such as acetone, methyl ethyl ketone and the like, aromatic hydrocarbons such as toluene, xylene and the like, ethers such as diethyl ether, tert-butyl methyl ether, tetrahydrofuran, 1,4-dioxane, 1,2-dimethoxyethane and the like, aprotic polar solvents such as N,N-dimethylformamide, N-methylpyrrolidone and the like, and a mixture thereof; as well as a mixture of these solvents and water.

Examples of the base used in the reaction include hydroxides of alkali metals or alkaline earth metals such as sodium hydroxide and the like, hydrides of alkali metals or alkaline earth metals such as sodium hydride and the like, inorganic bases such as sodium carbonate, potassium carbonate, and the like, and organic bases such as pyridine, triethylamine and the like.

The amount of the compound represented by the formula (V) is usually 1 to 1.5 mole, and the amount of the base is usually 1 to 1.5 mole relative to 1 mole of the compound represented by the formula (IV). When a base to be used is a liquid under reaction conditions such as pyridine, it can be used in an excessive amount as the solvent.

The reaction temperature is usually in a range of 0° C. to 100° C., and the reaction time is usually in a range of 0.1 to 24 hours.

After completion of the reaction, the compound represented by the formula (Ia) can be isolated by performing a post-treatment procedure such as by pouring the reaction mixture into water, followed by extracting with an organic solvent, drying and concentrating the organic layer. The isolated compound represented by the formula (Ia) can be further purified by recrystallization, column chromatography or the like.

### (Process 3)

Among the present compounds, a compound represented by the formula (Ib):

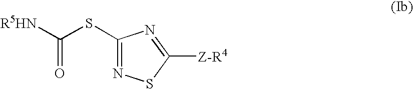

wherein R⁵ is a hydrogen atom, a C1-C4 alkyl group, a C3-C4 alkenyl group, a C1-C4 alkoxy group, or a phenyl group, and Z and R⁴ are as defined above,
can be produced by reacting the above compound represented by the formula (IV) and an isocyanate compound represented by the formula (VI):

R⁵—N═C═O (VI)
wherein R⁵ is as defined above.

The reaction is usually performed in a solvent.

Examples of the solvent used in the reaction include alcohols such as methanol, ethanol and the like, aromatic hydrocarbons such as toluene, xylene and the like, halogenated hydrocarbons such as methylene chloride, chloroform and the like, ethers such as diethyl ether, tert-butyl methyl ether, tetrahydrofuran, 1,4-dioxane, 1,2-dimethoxyethane and the like, aprotic polar solvents such as N,N-dimethylformamide, N-methylpyrrolidone and the like, and a mixture of these solvents.

The amount of the isocyanate compound represented by the formula (VI) is usually 1 to 1.5 mole relative to 1 mole of the compound represented by the formula (IV).

The reaction is performed, if necessary, in the presence of a base. Examples of the base which can be used include organic bases such as pyridine, triethylamine and the like, inorganic bases such as potassium carbonate and the like, and organic alkali metal compounds such as potassium tert-butoxide and the like.

When the reaction is performed in the presence of the base, the amount of the base is usually 1 to 1.5 mole relative to 1 mole of the compound represented by the formula (IV) and, when a base to be used is liquid under reaction conditions such as pyridine, the base can be used in an excessive amount as the solvent.

The reaction temperature is usually in a range of −20° C. to 100° C., and the reaction time is usually in a range of 0.1 to 24 hours.

After completion of the reaction, the compound represented by the formula (Ib) can be isolated by performing a post-treatment procedure such as by pouring the reaction mixture into water, followed by extracting with an organic solvent, and drying and concentrating the organic layer. The isolated compound represented by the formula (Ib) can be further purified by recrystallization, column chromatography or the like.

### (Process 4)

Among the present compounds, a compound represented by the formula (Ib) can be produced by reacting a compound represented by the formula (VII):

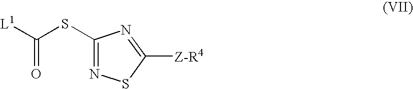

wherein L¹ is a leaving group such as a chlorine atom, a trichloromethyl group, a p-nitrophenoxy group and the like, and R⁴ is as defined above,
and an amine compound represented by the formula (VIII):

R⁵—NH₂ (VIII)
wherein R⁵ is as defined above.

The reaction is usually performed in a solvent.

Examples of the solvent used in the reaction include alcohols such as methanol, ethanol and the like, halogenated hydrocarbons such as methylene chloride, chloroform and the like, ethers such as diethyl ether, tert-butyl methyl ether, tetrahydrofuran, 1,4-dioxane, 1,2-dimethoxyethane and the like, aprotic polar solvents such as N,N-dimethylformamide, N-methylpyrrolidone and the like, and a mixture of these solvents; as well as a mixture of these solvents and water.

The amount of the compound represented by the formula (VIII) is usually 1 to 1.5 mole relative to 1 mole of the compound represented by the formula (VII).

The reaction can be performed, if necessary, in the presence of a base other than the amine compound represented by the formula (VIII). Examples of the base which can be used include organic bases such as pyridine, triethylamine and the like, hydroxides of alkali metals or alkaline earth metals such as sodium hydroxide and the like, inorganic bases such as sodium carbonate, sodium bicarbonate and the like, and organic alkali metal compounds such as sodium methoxide and the like.

When the reaction is performed in the presence of the base other than the amine compound represented by the formula (VIII), the amount of the base is usually 1 to 1.5 mole relative to 1 mole of the compound represented by the formula (VII).

The reaction temperature is usually in a range of 0° C. to 100° C., and the reaction time is usually in a range of 0.1 to 24 hours.

After completion of the reaction, the compound represented by the formula (Ib) can be isolated by performing a post-treatment procedure such as by pouring the reaction mixture into water, followed by extracting with an organic solvent, drying and concentrating the organic layer. The isolated compound represented by the formula (Ib) can be further purified by recrystallization, column chromatography or the like.

### (Process 5)

Among the present compounds, a compound represented by the formula (Ic):

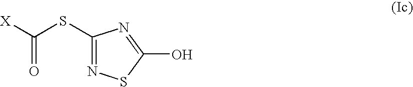

wherein X is as defined above,
can be produced by reacting a compound represented by the formula (IX):

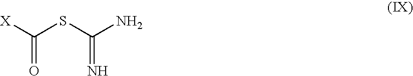

wherein X is as defined above,
or a salt thereof (hydrochloride, acetate, sulfate etc.) and chlorocarbonylsulfenyl chloride (Cl(C═O)SCl).

The reaction is performed usually in the presence of a base usually in a solvent.

Examples of the solvent used in the reaction include organic solvents such as esters such as ethyl acetate and the like, halogenated hydrocarbons such as methylene chloride, chloroform and the like, aromatic hydrocarbons such as toluene, xylene and the like, and ethers such as diethyl ether, tert-butyl methyl ether, tetrahydrofuran, 1,4-dioxane, 1,2-dimethoxyethane and the like, a mixture of these organic solvents, as well as a mixture of these organic solvents and water.

Examples of the base used in the reaction include hydroxides of alkali metals or alkaline earth metals such as sodium hydroxide and the like, and inorganic bases such as sodium bicarbonate, sodium carbonate, potassium carbonate and the like.

The amount of chlorocarbonylsulfenyl chloride is usually 1 to 1.5 mole, and the amount of the base is usually 2 to 4 mole relative to 1 mole of the compound represented by the formula (IX).

The reaction temperature is usually in a range of 0° C. to 100° C., and the reaction time is usually in a range of 0.1 to 48 hours.

After completion of the reaction, the compound represented by the formula (Ic) can be isolated by performing a post-treatment procedure such as by pouring the reaction mixture into water, followed by extracting this with an organic solvent, drying and concentrating the organic layer. The isolated compound represented by the formula (Ic) can be further purified by recrystallization, column chromatography or the like.

### (Process 6)

Among the present compounds, a compound represented by the formula (Ig):

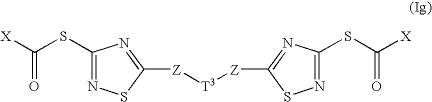

wherein T³ is a C2-C7 alkanediyl group, and X and Z are as defined above,
can be produced by reacting a compound represented by the formula (II), and a compound represented by the formula (IIIg):

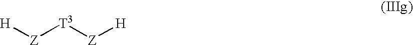

wherein T³ is a C2-C7 alkanediyl group, and Z is as defined above.

The reaction is performed usually in the presence of a base usually in a solvent.

Examples of the solvent used in the reaction include aliphatic hydrocarbons such as hexane, heptane and the like, aromatic hydrocarbons such as toluene, xylene and the like, ethers such as diethyl ether, tert-butyl methyl ether, tetrahydrofuran, 1,4-dioxane, 1,2-dimethoxyethane and the like, halogenated hydrocarbons such as methylene chloride, chloroform and the like, aprotic polar solvents such as N,N-dimethylformamide, N-methylpyrrolidone and the like and a mixture thereof.

Examples of the base used in the reaction include hydroxides of alkali metals or alkaline earth metals such as sodium hydroxide, potassium hydroxide, calcium hydroxide and the like, hydrides of alkali metals or alkaline earth metals such as sodium hydride, potassium hydride, calcium hydride and the like, inorganic bases such as sodium carbonate, potassium carbonate and the like and organic bases such as triethylamine and the like.

The amount of the compound represented by the formula (IIIg) is usually 0.3 to 0.6 mole, and the amount of the base is usually 1 to 1.5 mole relative to 1 mole of the compound represented by the formula (II).

The reaction temperature is usually in a range of −78° C. to 100° C., and the reaction time is usually in a range of 0.1 to 24 hours.

After completion of the reaction, the compound represented by the formula (Ig) can be isolated by performing a post-treatment procedure such as by pouring the reaction mixture into water, followed by extracting with an organic solvent, drying and concentrating the organic layer. The isolated compound represented by the formula (Ig) can be further purified by recrystallization, column chromatography or the like.

### (Process 7)

Among the present compounds, a compound represented by the formula (Ih):

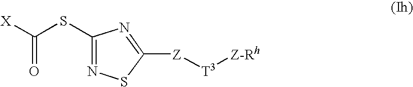

wherein R^{h} is a -(T-Z²)r-R¹⁰ group, a —C(═O)—(Z³)q-R¹⁰ group or a -Q group, and T, Z², R, R¹⁰, Z³, q, Q, T³, X and Z are as defined above,
can be produced by reacting a compound represented by the formula (Ij):

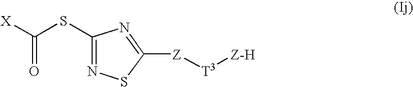

wherein T³, X and Z are as defined above,
and a compound represented by the formula (L):

L¹-R^{h} (L)
wherein L¹ represents a leaving group such as a chlorine atom, a bromine atom, and —SO₂Me, and R^{h} is as defined above.

The reaction is performed usually in the presence of a base usually in a solvent.

Examples of the solvent used in the reaction include aliphatic hydrocarbons such as hexane, heptane and the like, aromatic hydrocarbons such as toluene, xylene and the like, ethers such as diethyl ether, tert-butyl methyl ether, tetrahydrofuran, 1,4-dioxane, 1,2-dimethoxyethane and the like, halogenated hydrocarbons such as methylene chloride, chloroform and the like, aprotic polar solvents such as N,N-dimethylformamide, N-methylpyrrolidone and the like, and a mixture thereof.

Examples of the base used in the reaction include hydroxides of alkali metals or alkaline metals such as sodium hydroxide, potassium hydroxide, calcium hydroxide and the like, hydrides of alkali metals or alkaline earth metals such as sodium hydride, potassium hydride, calcium hydride and the like, inorganic bases such as sodium carbonate, potassium carbonate and the like, and organic bases such as triethylamine, diisopropylethylamine and the like.

The amount of the compound represented by the formula (L) is usually 1 to 3 mole, and the amount of the base is usually 1 to 1.5 mole relative to 1 mole of the compound represented by the formula (Ij).

The reaction temperature is usually in a range of −78° C. to 100° C., and the reaction time is usually in a range of 0.1 to 24 hours.

After completion of the reaction, the compound represented by the formula (Ih) can be isolated by performing a post-treatment procedure such as by pouring the reaction mixture into water, followed by extracting this with an organic solvent, drying and concentrating the organic layer. The isolated compound represented by the formula (Ih) can be further purified by recrystallization, column chromatography or the like.

### (Process 8)

Among the present compounds, a compound represented by the formula (Ik):

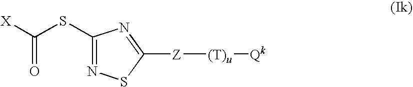

wherein u is 0 or 1, Q^{k} is a group represented by the formula:

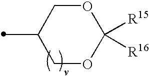

wherein v is 0 or 1, R¹⁵ and R¹⁶ each independently are a hydrogen atom, a C2-C4 alkyl group, a C2-C7 alkenyl group, a C2-C4 alkynyl group, a C1-C4 alkoxyalkyl group, or a -Q⁸ group, or R¹³ and R¹⁴ bind to each other at the ends thereof to form a C2-C7 alkanediyl group, or a -Z⁴-T-Z⁵— group, Q⁸ is a 3- to 10-membered carbocyclic group optionally substituted with one or more substituents selected from the above D group, or optionally substituted with one or more substituents selected from the above F group at the same position or adjacent positions, or a 3- to 10-membered heterocyclic group optionally substituted with one or more substituents selected from the above D group, or optionally substituted with one or more substituents selected from the above F group at the same position or adjacent positions, and R¹³, R¹⁴, Z⁴ and Z⁵ are as defined above, and
T, X, Z and Q are as defined above,
can be produced by reacting a compound represented by the formula (Im):

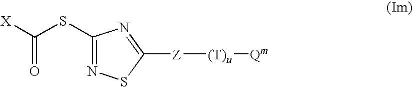

wherein Qm is a group represented by the following formula:

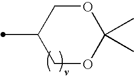

wherein v is as defined above, and
u, T, X and Z are as defined above,
and a carbonyl compound represented by the formula (LI):

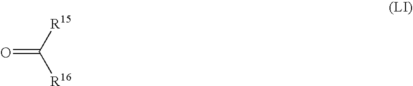

wherein R¹⁵ and R¹⁶ are as defined above,
or an equivalent thereof, i.e., a corresponding acetal compound.

The reaction is performed usually in the presence of an acid usually in a solvent.

Examples of the solvent used in the reaction include aliphatic hydrocarbons such as hexane, heptane and the like, aromatic hydrocarbons such as toluene, xylene and the like, ethers such as diethyl ether, tert-butyl methyl ether, tetrahydrofuran, 1,4-dioxane, 1,2-dimethoxyethane and the like, halogenated hydrocarbons such as methylene chloride, chloroform and the like, aprotic polar solvents such as N,N-dimethylformamide, N-methylpyrrolidone and the like, and a mixture of these solvents; as well as a mixture of these solvents and water.

Examples of the acid used in the reaction include mineral acids such as hydrochloric acid, sulfuric acid and the like, and organic acids such as acetic acid, trifluoroacetic acid, trichloroacetic acid, p-toluenesulfonic acid and the like.

The amount of the carbonyl compound represented by the formula (LI) or an equivalent thereof, i.e., a corresponding acetal compound is usually 1 to mole to excessive amount, and the amount of the acid catalyst is usually 0.1 to 1 mole relative to 1 mole of the compound represented by the formula (Im). When the carbonyl compound represented by the formula (LI) or an equivalent thereof, i.e., a corresponding acetal compound is used excessively, the reaction can be performed without using the aforementioned solvent.

The reaction temperature is usually in a range of −78° C. to 100° C., and the reaction time is usually in a range of 0.1 to 24 hours.

After completion of the reaction, the compound represented by the formula (Ik) can be isolated by performing a post-treatment procedure such as by pouring the reaction mixture into water, followed by extracting with an organic solvent, drying and concentrating the organic layer,. The isolated compound represented by the formula (Ik) can be further purified by recrystallization, column chromatography or the like.

### (Process 9)

Among the present compounds, a compound represented by the formula (In):

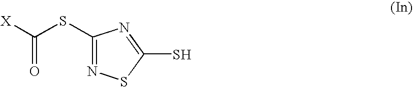

wherein X is as defined above,
can be produced by reacting the above compound represented by the formula (II) and thiourea.

The reaction is usually performed in a solvent.

Examples of the solvent used in a reaction include alcohols such as methanol, ethanol and the like, aromatic hydrocarbons such as toluene, xylene and the like, halogenated hydrocarbons such as methylene chloride, chloroform and the like, ethers such as diethyl ether, tert-butyl methyl ether, tetrahydrofuran, 1,4-dioxane, 1,2-dimethoxyethane and the like, aprotic polar solvents such as N,N-dimethylformamide, N-methylpyrrolidone and the like, a mixture thereof, and a mixture of them and water.

The amount of thiourea is usually 1 to 1.5 mole relative to 1 mole of the compound represented by the formula (II).

The reaction temperature is usually in a range of −78° C. to 100° C., and the reaction time is usually in a range of 0.1 to 24 hours.

After completion of the reaction, the compound represented by the formula (In) can be isolated by performing a post-treatment procedure such as by pouring the reaction mixture into water, followed by extracting with an organic solvent, drying and concentrating the organic layer. The isolated compound represented by the formula (In) can be further purified by recrystallization, column chromatography or the like.

Hereinafter, a process for producing the present intermediate compound will be explained.

### (Reference Process 1)

Among the present intermediates, a compound represented by the formula (IIa):

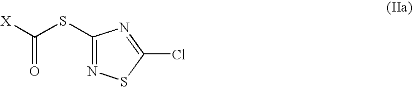

wherein X is as defined above,
can be produced by reacting a compound represented by the formula (IX):

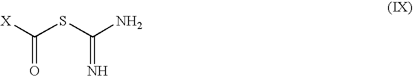

wherein X is as defined above,
or a salt thereof (hydrochloride, acetate, sulfate, etc.), and perchloromethylmercaptan (trichloromethylsulphenyl chloride).

The reaction is performed by usually in the presence of a base usually in a solvent.

Examples of the solvent used in the reaction include esters such as ethyl acetate, halogenated hydrocarbons such as methylene chloride, chloroform and the like, aromatic hydrocarbons such as toluene, xylene and the like, ethers such as diethyl ether, tert-butyl methyl ether, tetrahydrofuran, 1,4-dioxane, 1,2-dimethoxyethane and the like, a mixture of these organic solvents, as well as a mixture of these organic solvents and water.

Examples of the base used in the reaction include hydroxides of alkali metals or alkaline earth metals such as sodium hydroxide, and inorganic bases such as sodium bicarbonate, sodium carbonate, potassium carbonate and the like.

The amount of perchloromethylmercaptan is usually 1 to 1.5 mole, and the amount of the base is usually 4 to 6 mole relative to 1 mole of the compound represented by the formula (IX).

The reaction temperature is usually in a range of 0° C. to 100° C., and the reaction time is usually in a range of 0.1 to 48 hours.

After completion of the reaction, the compound represented by the formula (IIa) can be isolated by performing a post-treatment procedure such as by pouring the reaction mixture into water, followed by extracting with an organic solvent, drying and concentrating the organic layer. The isolated compound represented by the formula (IIa) can be further purified by recrystallization, column chromatography or the like.

### (Reference Process 2)

A compound represented by the formula (IV) can be produced by reacting a compound represented by the formula (LII):

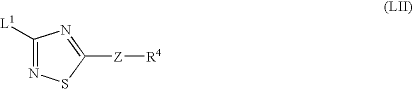

wherein L¹, Z, and R⁴ are as defined above,
and thiourea.

The reaction is performed usually in the presence of a base usually in a solvent.

Examples of the solvent used in the reaction include alcohols such as methanol, ethanol and the like, aromatic hydrocarbons such as toluene, xylene and the like, halogenated hydrocarbons such as methylene chloride, chloroform and the like, ethers such as diethyl ether, tert-butyl methyl ether, tetrahydrofuran, 1,4-dioxane, 1,2-dimethoxyethane and the like, aprotic organic solvents such as N,N-dimethylformamide, N-methylpyrrolidone and the like, a mixture thereof, and a mixture of them and water.

Examples of the base used in the reaction include hydroxides of alkali metals or alkaline earth metals such as sodium hydroxide, potassium hydroxide, calcium hydroxide and the like, inorganic bases such as sodium carbonate, potassium carbonate and the like, and organic bases such as triethylamine and the like.

The amount of thiourea is usually 1 to 2 mole, and the amount of the base is usually 1 to 1.5 mole relative to 1 mole of the compound represented by the formula (LII).

The reaction temperature is usually in a range of −78° C. to 100° C., and the reaction time is usually in a range of 0.1 to 24 hours.

After completion of the reaction, the compound represented by the formula (IV) can be isolated by performing a post-treatment procedure such as by pouring the reaction mixture into water, followed by extracting with an organic solvent, drying and concentrating the organic layer. The isolated compound represented by the formula (IV) can be further purified by recrystallization, column chromatography or the like.

### (Reference Process 3)

A compound represented by the formula (VII) can be produced by reacting the compound represented by the formula (IV) and a compound represented by the formula (X):

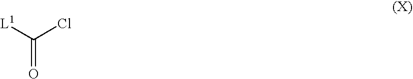

wherein L¹ is as defined above.

The reaction is usually performed in a solvent.

Examples of the solvent used in the reaction include halogenated hydrocarbons such as methylene chloride, chloroform and the like, aromatic hydrocarbons such as toluene, xylene and the like, and ethers such as diethyl ether, tert-butyl methyl ether, tetrahydrofuran, 1,4-dioxane, 1,2-dimethoxyethane and the like, and a mixture of these solvents.

The amount of the compound represented by the formula (X) is usually 1 to 1.5 mole relative to 1 mole of the compound represented by the formula (IV).

The reaction is performed, if necessary, in the presence of a base. Examples of the base which can be used include organic bases such as pyridine, triethylamine and the like, and inorganic bases such as potassium carbonate and the like.

When the reaction is performed in the presence of the base, the amount of the base is usually 1 to 1.5 mole relative to 1 mole of the compound represented by the formula (IV). When a base to be used is liquid under reaction conditions such as pyridine the base can be used in an excessive amount as a solvent.

The reaction temperature is usually in a range of 0° C. to 100° C., and the reaction time is usually in a range of 0.1 to 24 hours.

After completion of the reaction, the compound represented by the formula (VII) can be isolated by performing a post-treatment procedure such as by pouring the reaction mixture into water, followed by extracting with an organic solvent, drying and concentrating the organic layer,. The isolated compound represented by the formula (VII) can be further purified by recrystallization or the like.

### (Reference Process 4)

A compound represented by the formula (IX) or hydrochloride thereof can be produced by reacting a carbamoyl chloride compound represented by the formula (V) and thiourea.

The reaction is performed usually in the presence of a base usually in a solvent.

Examples of the solvent used in the reaction include alcohols such as methanol, ethanol and the like, ethers such as diethyl ether, tert-butyl methyl ether, tetrahydrofuran, 1,4-dioxane, 1,2-dimethoxyethane and the like, aromatic hydrocarbons such as toluene, xylene and the like, and halogenated hydrocarbons such as methylene chloride, chloroform and the like.

The amount of the carbamoyl chloride compound represented by the formula (V) is usually 1 to 1.5 mole relative to 1 mole of thiourea.

The reaction temperature is usually in a range of 0° C. to 100° C., and the reaction time is usually in a range of 0.1 to 24 hours.

After completion of the reaction, the compound represented by the formula (IX) can be isolated by performing a post-treatment procedure such as by pouring the reaction mixture into water, followed by extracting with an organic solvent, drying and concentrating the organic layer,. The isolated compound represented by the formula (IX) can be further purified by recrystallization or the like. Alternatively, the hydrochloride of the compound represented by the formula (IX) can also be isolated by filtering crystals formed by a procedure such as concentration of the reaction mixture under reduced pressure. The isolated hydrochloride of the compound represented by the formula (IX) can be further purified by recrystallization or the like.

### (Reference Process 5)

A compound represented by the formula (LII) can be produced by reacting a thiadiazole compound represented by the formula (LIII):

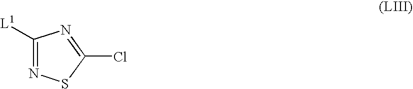

wherein L¹ is as defined above,
and the compound represented by the formula (III).

The reaction is performed usually in the presence of a base usually in a solvent.

Examples of the solvent used in the reaction include aliphatic hydrocarbons such as hexane, heptane and the like, aromatic hydrocarbons such as toluene, xylene and the like, ethers such as diethyl ether, tert-butyl methyl ether, tetrahydrofuran, 1,4-dioxane, 1,2-dimethoxyethane and the like, halogenated hydrocarbons such as methylene chloride, chloroform and the like, aprotic polar solvents such as N,N-dimethylformamide, N-methylpyrrolidone and the like, and a mixture thereof.

Examples of the base used in the reaction include hydroxides of alkali metals or alkaline earth metals such as sodium hydroxide, potassium hydroxide, calcium hydroxide and the like, hydrides of alkali metals or alkaline earth metals such as sodium hydride, potassium hydride, calcium hydride and the like, inorganic bases such as sodium carbonate, potassium carbonate and the like, and organic bases such as triethylamine and the like.

The amount of the compound represented by the formula (III) is usually 1 to 2 mole, and the amount of the base is usually 1 to 1.5 mole relative to 1 mole of the compound represented by the formula (LIII).

The reaction temperature is usually in a range of −78° C. to 100° C., and the reaction time is usually in a range of 0.1 to 24 hours.

After completion of the reaction, the compound represented by the formula (LII) can be isolated by performing a post-treatment procedure such as by pouring the reaction mixture into water, followed by extracting with an organic solvent, drying and concentrating the organic layer,. The isolated compound represented by the formula (LII) can be further purified by recrystallization, column chromatography or the like.

The compound represented by the formula (III), the compound represented by the formula (IIIg), the carbamoyl chloride compound represented by the formula (V), the isocyanate compound represented by the formula (VI), the amine compound represented by the formula (VIII), the thiadiazole compound represented by the formula (LIII), and the compound represented by the formula (X) are known compounds, or can be produced from known compounds according to known methods (see Journal of the American Chemical Society (1950),72(5),1888-1891, and Journal of Organic Chemistry (2003),68(19),7289-7297).

The compound represented by the formula (IV), the compound represented by the formula (VII), and the compound represented by the formula (IX) are known compounds, or can be produced from known compounds under the same reaction conditions as those of the production processes of analogous compounds shown in Examples herein.

In the present compounds, there are isomers such as geometrical isomers, steric isomers and the like and, all isomers including active isomers alone or a mixture thereof are included in the present compounds.

A noxious arthropod on which the present compound has efficacy includes noxious insects and noxious mites, and specific examples thereof are as follows:

Hemiptera: Delphacidae such as Laodelphax striatellus, Nilaparvata lugens, and Sogatella furcifera, Deltocephalidae such as Nephotettix cincticeps, Nephotettix virescens, and Empoasca onukii, Aphididae such as Aphis gossypii, Myzus persicae, Brevicoryne brassicae, Aphis spiraecola, Macrosiphum euphorbiae, Aulacorthum solani, Rhopalosiphum padi, Toxoptera citricidus, and Hyalopterus pruni, Pentatomidae such as Nezara antennata, Riptortus clavetus, Leptocorisa chinensis, Eysarcoris parvus, and Halyomorpha mista, Aleyrodidae such as Trialeurodes vaporariorum, Bemisia tabaci, Bemisia argentifolii, Dialeurodes citri, and Aleurocanthus spiniferus, Coccidae such as Aonidiella aurantii, Comstockaspis perniciosa, Unaspis citri, Ceroplastes rubens, Icerya purchasi, Planococcus kraunhiae, Pseudococcus longispinis, and Pseudaulacaspis pentagona, Tingidae , Psyliidae, and the like.

Lepidoptera: Pyralidae such as Chilo suppressalis, Tryporyza incertulas, Cnaphalocrocis medinalis, Notarcha derogata, Plodia interpunctella, Ostrinia furnacalis, Hellula undalis, and Pediasia teterrellus, Noctuidae such as Spodoptera litura, Tortricidae such as Spodoptera exigua, Pseudaletia separata, Mamestra brassicae, Agrotis ipsilon, Plusia nigrisigna, Thoricoplusia spp., Heliothis spp., and Helicoverpa spp., Pieridae such as Pieris rapae, Adoxophyes spp., Grapholita molesta, Leguminivora glycinivorella, Matsumuraeses azukivora, Adoxophyes orana fasciata, Adoxophyes sp., Homona magnanima, Archips fuscocupreanus, and Cydia pomonella, Gracillariidae such as Caloptilia theivora, and Phyllonorycter ringoneella, Carposinidae such as Carposina niponensis, Lyonetiidae such as Lyonetia spp., Lymantriidae such as Lymantria spp., and Euproctis spp., Yponomeutidae such as Plutella xylostella, Gelechiidae such as Pectinophora gossypiella, and Phthorimaea operculella, Arctiidae such as Hyphantria cunea, Tineidae such as Tinea translucens, Tineola bisselliella, and the like.

Thysanoptera: Thysanoptera such as Frankliniella occidentalis, Thrips parmi, Scirtothrips dorsalis, Thrips tabaci, and Frankliniella intonsa.

Diptera: Musca domestica, Culex popiens pallens, Tabanus trigonus, Hylemya antiqua, Hylemya platura, Anopheles sinensis, Agromyza oryzae, Hydrellia griseola, Chlorops oryzae, Dacus cucurbitae, Ceratitis capitata, Liriomyza trifolii, Liriomyza sativae, Chromatomyia horticola, and the like.

Coleoptera: Epilachna vigintioctopunctata, Aulacophora femoralis, Phyllotreta striolata, Oulema oryzae, Echinocnemus squameus, Lissorhoptrus oryzophilus, Anthonomus grandis, Callosobruchus chinensis, Sphenophorus venatus, Popillia japonica, Anomala cuprea, Diabrotica spp., Leptinotarsa decemlineata, Agriotes spp., Lasioderma serricorne, Anthrenus verbasci, Tribolium castaneum, Lyctus brunneus, Anoplophora malasiaca, Tomicus piniperda, and the like.

Orthoptera: Locusta migratoria, Gryllotalpa africana, Oxya yezoensis, Oxya japonica, and the like.

Hymenoptera: Athalia rosae, Acromyrmex spp., Solenopsis spp., and the like.

Blattodea: Blattella germanica, Periplaneta fuliginosa, Periplaneta americana, Periplaneta brunnea, Blatta orientalis, and the like.

Aphaniptera: Ctenocephalides felis, Ctenocephalides canis, Pulex irritans, Xenopsylla cheopis, and the like.

Anoplura: Pediculus humanus corporis, Phthirus pubis, Haematopinus eurysternus, Dalmalinia ovis, and the like.

Isoptera: Reticulitermes speratus, Coptotermes formosanus, and the like.

Acarina: Tetranychus urticae, Tetranychus kanzawai, Panonychus citri, Panonychus ulmi, Tetranychidae such as Oligonychus spp., Aculops pelekassi, Eriophyidae such as Phyllocoptruta citri, Aculops lycopersici, Calacarus carinatus, Acaphylla theavagrans, and Eriophyes chibaensis, Tarsonemidae such as Polyphagotarsonemus latus, Tenuipalpidae such as Brevipalpus phoenicis, Metastigmata such as Tuckerellidae, Haemaphysalis longicornis, Haemaphysalis flava, Dermacentor taiwanicus, Ixodes ovatus, Ixodes persulcatus, Boophilus microplus, and Rhipicephalus sanguineus, Acaridae such as Tyrophagus putrescentiae, and Tyrophagus similis, Pyroglyphidae such as Dermatophagoides farinae, and Dermatophagoides ptrenyssnus, Cheyletidae such as Cheyletus eruditus, Cheyletus malaccensis, and Cheyletus moorei, Dermanyssidae, and the like.

The noxious arthropod controlling agent of the present invention may be the present compound itself. However, usually, the present compound is mixed with an inert carrier such as a solid carrier, a liquid carrier, a gaseous carrier or the like and, if necessary, a surfactant and other additives for preparations are added, and can be formulated into emulsions, oil solutions, dusts, powders, granules, wettable powders, flowables, microcapsules, aerosols, fumigants, poison bait, resin preparations or the like. Such a preparation usually contains the present compound in an amount of 0.01 to 95% by weight based on the whole preparation.

Examples of the solid carrier to be used for the production of a preparation include finely-divided powders, particulates and the like of clays (kaolin clay, diatomaceous earth, bentonite, fubasami cray, acid clay, etc.), synthetic hydrated silicon dioxide, talc, ceramic, other inorganic minerals (sericite, quartz, sulfur, active carbon, calcium carbonate, hydrated silica etc.), chemical fertilizer (ammonium sulfate, ammonium phosphate, ammonium nitrate, urea, ammonium chloride etc.) and the like.

Examples of the liquid carrier include water, alcohols (methano, ethanol, isopropyl alcohol, butanol, hexanol, benzyl alcohol, ethylene glycol, propylene glycol, phenoxyethanol etc.), ketones (acetone, methyl ethyl ketone, cyclohexanone etc.), aromatic hydrocarbons (toluene, xylene, ethylbenzene, dodecylbenzene, phenylxylylethane, methylnaphthalene etc.), aliphatic hydrocarbons (hexane, cyclohexane, kerosene, gas oil etc.), esters (ethyl acetate, butyl acetate, isopropyl myristate, ethyl oleate, diisopropyl adipate, diisobutyl adipate, propylene glycol monomethyl ether acetate etc.), nitriles (acetonitrile, isobutylonitrile etc.), ethers (diisopropyl ether, 1,4-dioxane, ethylene glycol dimethyl ether, diethylene glycol dimethyl ether, diethylene glycol monomethyl ether, propylene glycol monomethyl ether, dipropylene glycol monomethyl ether, 3-methoxy-3-methyl-1-butanol etc.), acid amides (N,N-dimethylformamide, N,N-dimethylactamide etc.), halogenated hydrocarbons (dichloromethane, trichloroethane, carbon tetrachloride etc.), sulfoxides (dimethyl sulfoxide etc.), propylene carbonate, vegetable oils (soybean oil, cottonseed oil etc.) and the like.

Examples of the gaseous carrier include fluorocarbon, butane gas, LPG (liquefied petroleum gas), dimethyl ether, carbonic acid gas and the like.

Examples of the surfactant include nonionic surfactants such as polyoxyethylene alkyl ether, polyoxyethylene alkyl aryl ether, polyethylene glycol fatty acid ester and the like, and anionic surfactants such as alkylsulfonate, alkylbenzenesulfonate, alkyl sulfate and the like.

Examples of other additives for preparations include binders, dispersants, colorants, and stabilizers, specifically casein, gelatin, sugars (starch, gum arabic, cellulose derivatives, alginic acid, etc.), lignin derivatives, bentonite, synthetic water-soluble polymers (polyvinyl alcohol, polyvinyl pyrrolidone, polyacrylic acids etc.), PAP (acidic isopropyl phosphate), BHT (2,6-di-tert-butyl-4-methylphenol), BHA (mixture of 2-tert-butyl-4-methoxyphenol and 3-tert-butyl-4-methoxyphenol), and the like.

A method of controlling a noxious arthropod of the present invention can be usually carried out by applying the noxious arthropod controlling agent of the present invention to a noxious arthropod, or a place where a noxious arthropod inhabits (plant body, soil, in house, animal body, etc.).

When the noxious arthropod controlling agent of the present invention is used in controlling a noxious arthropod in an agricultural field, the amount to be applied is usually 1 to 10000 g per 10000 m² in terms of the amount of the present compound. When the noxious arthropod controlling agent of the present invention is formulated into emulsions, wettables, flowables or the like, such preparations are applied by diluting with water so that the active ingredient concentration becomes 0.01 to 10000 ppm. Usually, granules, powders or dusts can be applied as such.

These preparations and water-diluted preparations can be directly sprayed to a noxious arthropod, plants such as crops, etc. to be protected from a noxious arthropod. Alternatively, they can be applied to a soil of a cultivated land in order to control a noxious arthropod which inhabits on the soil.

Further, the active ingredient can be applied by winding a crop with a sheet-like or string-like resin preparation, by stretching a preparation in the vicinity of a crop, or spreading a preparation on a soil near a plant foot.

When the noxious arthropod controlling agent of the present invention is used for controlling a noxious arthropod inhabiting in houses (e.g. fly, mosquito, cockroach, etc.), the application amount is usually 0.01 to 1000 mg per 1 m² of a treatment area in terms of the amount of the present compound in case of planar treatment, and is usually 0.01 to 500 mg per 1 m³ of a treatment space in terms of the amount of the present compound in case of space treatment. When the noxious arthropod controlling agent of the present invention is formulated into preparations such as emulsions, wettables, flowables or the like, they are applied by diluting with water so that an active ingredient concentration becomes 0.1 to 1000 ppm, and oily agents, aerosols, fumigants, poison baits or the like can be applied as such.

The noxious arthropod controlling agent of the present invention may contain one or more other noxious arthropod controlling agents, nematocides, fungicide, herbicide, plant growth regulators, synergists, fertilizers, soil improving agents, animal feeds and the like.

Active ingredients of the aforementioned other noxious arthropod controlling agents, acaricides, and nematocides are, for example, as follows:

### (1) Organic Phosphorous Compounds

Aacephate, aluminum phosphide, butathiofos, cadusafos, chlorethoxyfos, chlorfenvinphos, chlorpyrifos, chlorpyrifos-methyl, cyanophos: CYAP, diazinon, dichlorodiisopropyl ether, dichlofenthion: ECP, dichlorvos: DDVP, dimethoate, dimethylvinphos, disulfoton, EPN, ethion, ethoprophos, etrimfos, fenthion: MPP, fenitrothion: MEP, fosthiazate, formothion, hydrogen phosphide, isofenphos, isoxathion, malathion, mesulfenfos, methidathion: DMTP, monocrotophos, naled: BRP, oxydeprofos: ESP, parathion, phosalone, phosmet: PMP, pirimiphos-methyl, pyridafenthion, quinalphos, phenthoate: PAP, profenofos, propaphos, prothiofos, pyraclorfos, salithion, sulprofos, tebupirimfos, temephos, tetrachlorvinphos, terbufos, thiometon, trichlorphon: DEP, vamidothion, and the like.

### (2) Carbamate Compounds

Alanycarb, bendiocarb, benfuracarb, BPMC, carbaryl, carbofuran, carbosulfan, cloethocarb, ethiofencarb, fenobucarb, fenothiocarb, fenoxycarb, furathiocarb, isoprocarb: MIPC, metolcarb, methomyl, methiocarb, NAC, oxamyl, pirimicarb, propoxur: PHC, XMC, thiodicarb, xylylcarb, and the like.

### (3) Synthetic Pyrethroid Compounds

Acrinathrin, allethrin, benfluthrin, beta-cyfluthrin, bifenthrin, cycloprothrin, cyfluthrin, cyhalothrin, cypermethrin, deltamethrin, esfenvalerate, ethofenprox, fenpropathrin, fenvalerate, flucythrinate, flufenoprox, flumethrin, fluvalinate, halfenprox, imiprothrin, permethrin, prallethrin, pyrethrins, resmethrin, sigma-cypermethrin, silafluofen, tefluthrin, tralomethrin, transfluthrin, 2,3,5,6-tetrafluoro-4-(methoxymethyl)benzil(EZ)-(1RS,3RS;1RS,3RS)-2,2-dimethyl-3-prop-1-enylcyclopropane carboxylate, 2,3,5,6-tetrafluoro-4-methylbenzil(EZ)-(1RS,3RS;1RS,3SR)-2,2-dimethyl-3-prop-1-enylcyclopropane carboxylate, 2,3,5,6-tetrafluoro-4-(methyoxymethyl)benzil(1RS,3RS;1RS,3SR)-2,2-dimethyl-3-(2-methyl-1-propenyl)cyclopropane carboxylate, and the like.

### (4) Nereistoxin Compounds

Cartap, bensultap, thiocyclam, monosultap, bisultap, and the like.

### (5) Neonicotinoid Compounds

Imidacloprid, nitenpyram, acetamiprid, thiamethoxam, thiacloprid, dinotefuran, clothianidin, and the like.

### (6) Benzoylurea Compounds

Chlorfluazuron, bistrifluron, diafenthiuron, diflubenzuron, fluazuron, flucycloxuron, flufenoxuron, hexaflumuron, lufenuron, novaluron, noviflumuron, teflubenzuron, triflumuron, and the like.

### (7) Phenylpyrazole Compounds

Acetoprole, ethiprole, fipronil, vaniliprole, pyriprole, pyrafluprole, and the like.

### (8) Bt Toxin Insecticides

Live spores derived from Bacillus thuringiensis and crystalline toxin produced, as well as a mixture thereof.

### (9) Hydrazine Compounds

Chromafenozide, halofenozide, methoxyfenozide, tebufenozide, and the like.

### (10) Organic Chlorine Compounds

Aldrin, dieldrin, dienochlor, endosulfan, methoxychlor, and the like.

### (11) Natural Insecticides

Machine oil, nicotine-sulfate, and the like.

### (12) Other Insectides

Avermectin-B, bromopropylate, buprofezin, chlorphenapyr, cyromazine, 1,3-Dichloropropene, emamectin-benzoate, fenazaquin, flupyrazofos, hydroprene, indoxacarb, metoxadiazone, milbemycin-A, pymetrozine, pyridalyl, pyriproxyfen, spinosad, sulfluramid, chlorantraniliprole, tolfenpyrad, triazamate, flubendiamide, SI-0009, cyflumetofen, acid, benclothiaz, Calcium cyanamide, Calcium polysulfide, chlordane, DDT, DSP, flufenerim, flonicamid, flurimfen, formetanate, lepimectin, metam-ammonium, metam-sodium, Methyl bromide, nidinotefuran, Potassium oleate, protrifenbute, spiromesifen, Sulfur, metaflumizone, spirotetramat, and the like.

### Insecticides for Ticks

Acequinocyl, amitraz, benzoximate, bromopropylate, chinomethionat, chlorobenzilate, chlorfenson, clofentezine, Kelthane(dicofol), etoxazole, fenbutatin oxide, fenothiocarb, fenpyroximate, fluacrypyrim, fluproxyfen, hexythiazox, propargite: BPPS, polynactins complex, pyridaben, Pyrimidifen, tebufenpyrad, tetradifon, spirodiclofen, amidoflumet, Bifenazate, Cyflumetofen, and the like.

### Active Ingredients of Insecticides for Nematodes

DCIP, fosthiazate, levamisol, methyisothiocyanate, morantel tartarate, and the like.

The present invention will be illustrated by the following Examples, Production Examples, Preparation Examples and Test Examples in more detail, but is not limited to these Examples.

Embodiments of the present compound will be shown.

Abbreviations used herein have the following meanings. Me: methyl group, Et: ethyl group, ⁿPr: propyl group, ⁱPr: isopropyl group, ⁿBu: butyl group, ⁱBu: isobutyl group, ^{s}Bu: sec-butyl group, ^{t}Bu: tert-butyl group, Bn: benzyl group, Ph: phenyl group.

The thiadiazole compounds represented by the formula (I-1) to formula (I-95):

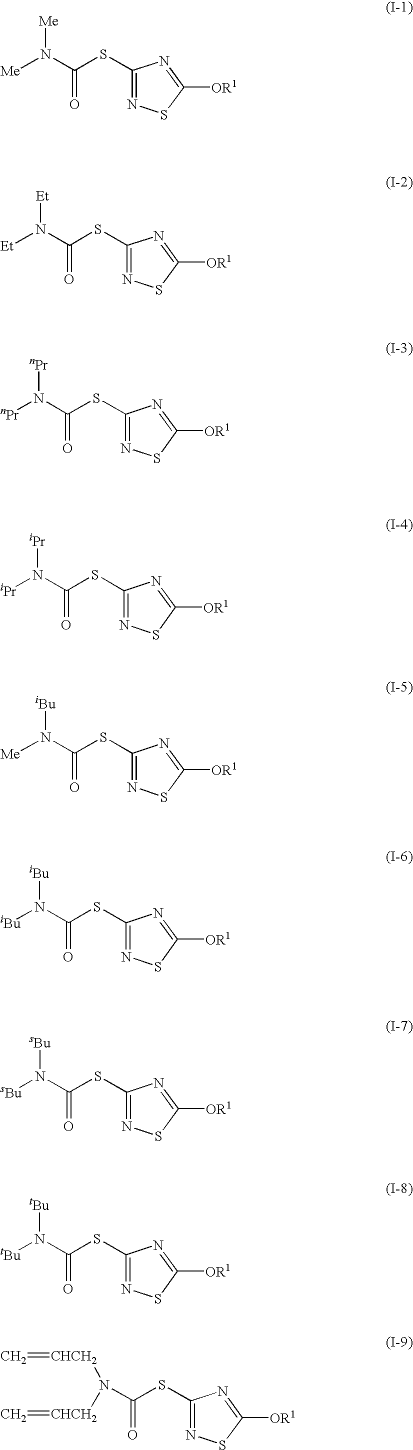

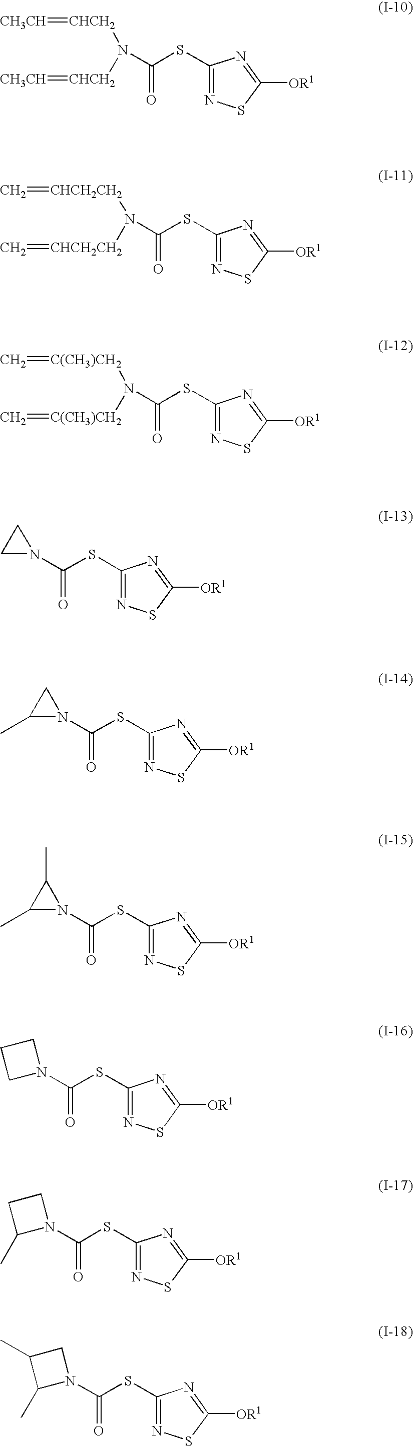

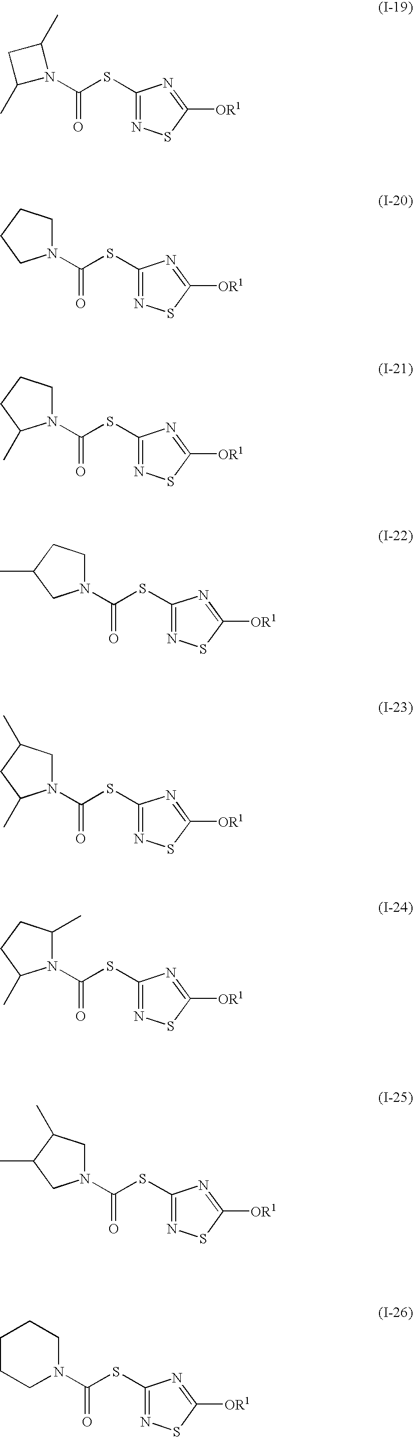

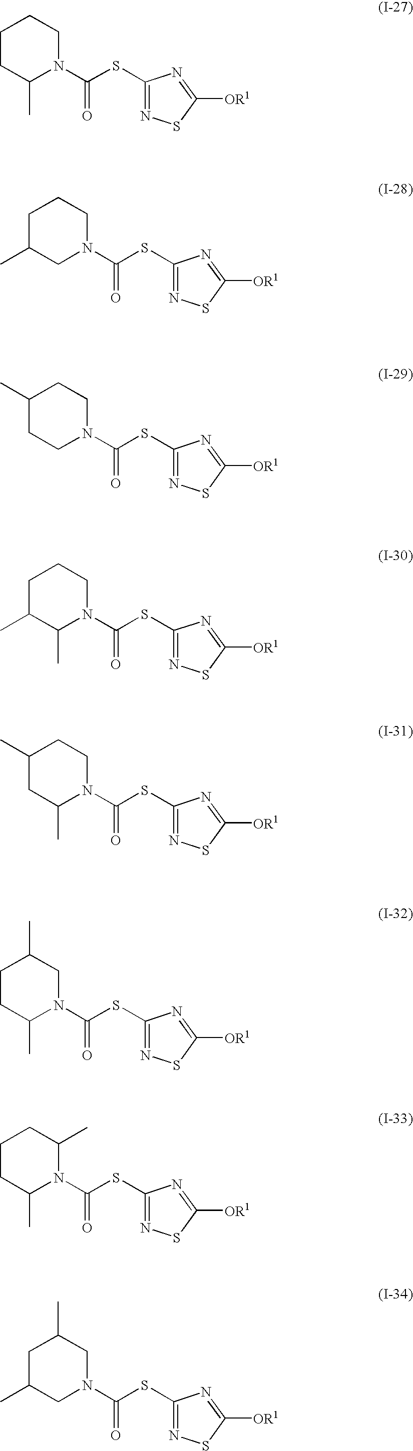

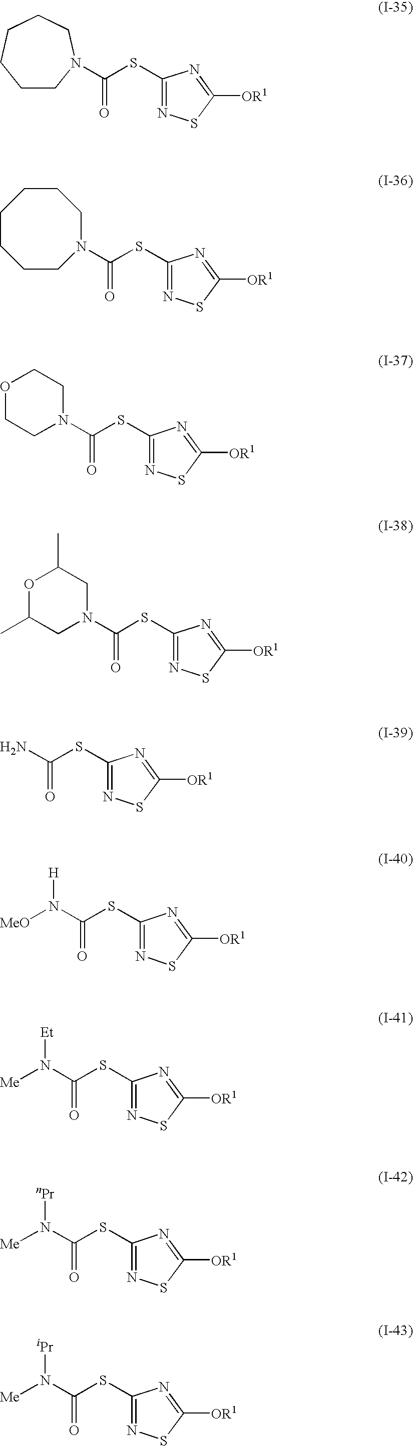

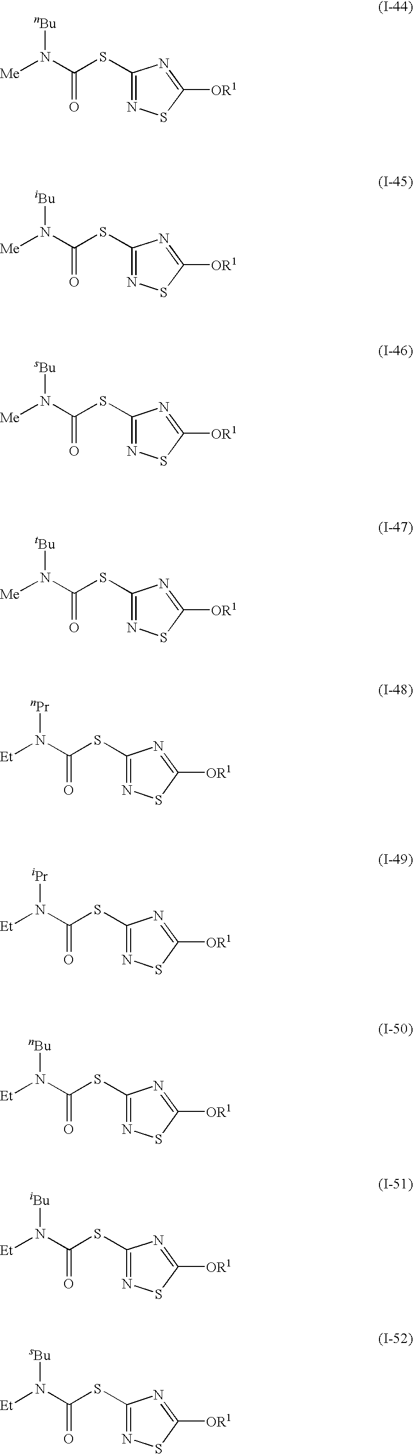

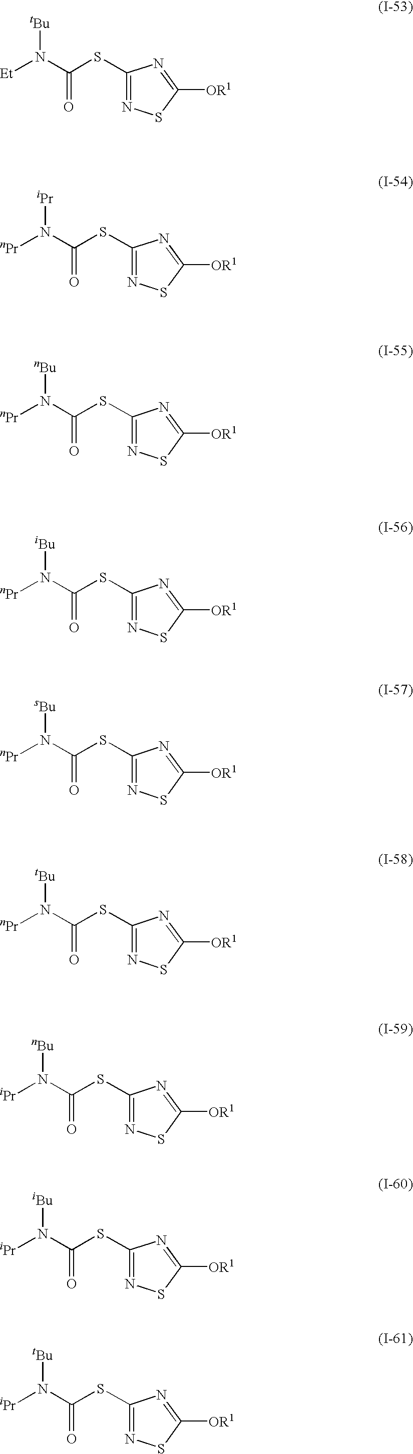

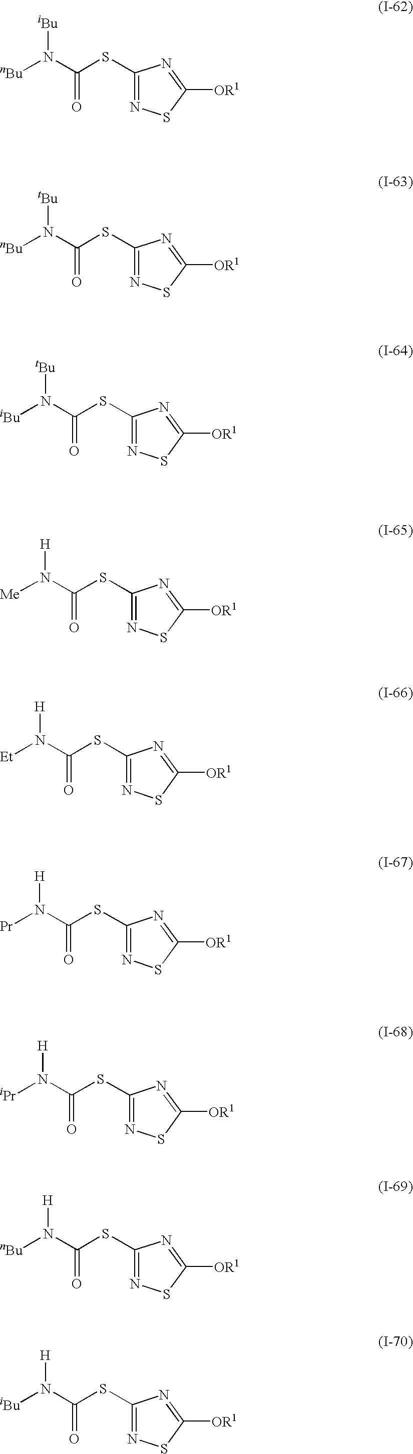

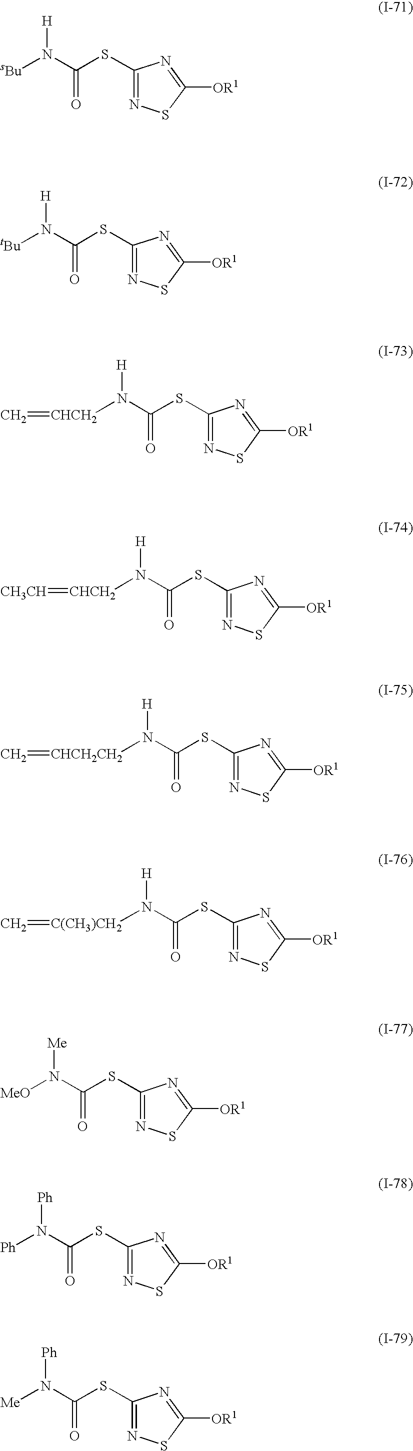

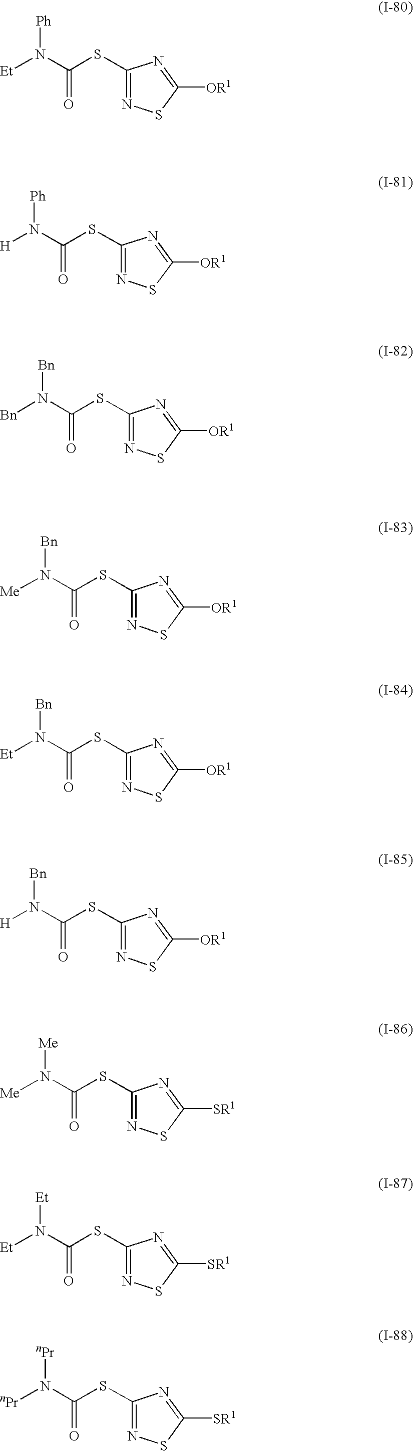

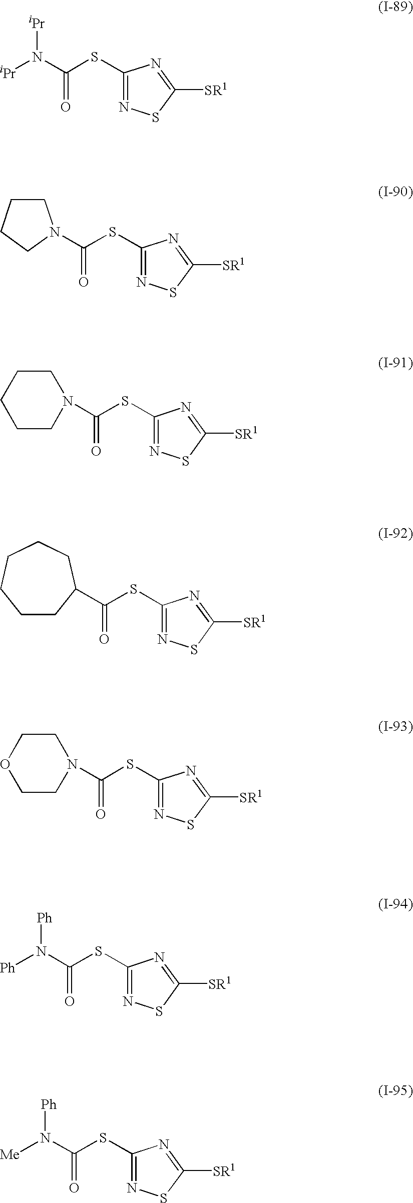

In the formula (I-1) to the formula (I-95), R¹ is a methyl group, an ethyl group, a propyl group, an isopropyl group, a butyl group, an isobutyl group, a sec-butyl group, a tert-butyl group, a pentyl group, an isopentyl group, a neopentyl group, a sec-pentyl group, a tert-pentyl group, a 2-methylbutyl group, a 1,2-dimethylpropyl group, a 1-ethylpropyl group, a hexyl group, a 3,3-dimethylbutyl group, a 1,2-dimethylbutyl group, a 2-methylpentyl group, a 3-methylpentyl group, a 4-methylpentyl group, a 2,2-dimethylbutyl group, a 2,3-dimethylbutyl group, a 2-ethylbutyl group, a 1-methylpentyl group, a 1,2,2-trimethylpropyl group, a 1,3-dimethylbutyl group, a 1-ethylbutyl group, a 1-ethyl-2-methylpropyl group, a heptyl group, a 1-ethyl-2,2-dimethylpropyl group, a 1-methylhexyl group, a 2-methylhexyl group, a 3-methylhexyl group, a 4-methylhexyl group, a 5-methylhexyl group, a 1,2-dimethylpentyl group, a 1,3-dimethylpentyl group, a 1,4-dimethylpentyl group, a 2,2-dimethylpentyl group, a 2,3-dimethylpentyl group, a 2,4-dimethylpentyl group, a 3,3-dimethylpentyl group, a 3,4-dimethylpentyl group, a 4,4-dimethylpentyl group, a 1-ethylpentyl group, a 2-ethylpentyl group, a 3-ethylpentyl group, a 1-propylbutyl group, a 2-ethyl-1-methylbutyl group, a 1-ethyl-2-methylbutyl group, a 1-ethyl-3-methylbutyl group, a 1-tert-butylpropyl group, a 3-ethyl-4-methylbutyl group, a 2-propenyl group, a 2-butenyl group, a 3-butenyl group, a 1-methyl-2-butenyl group, a 2-methyl-2-propenyl group, a 2-pentenyl group, a 3-pentenyl group, a 4-pentenyl group, a 2-methyl-2-butenyl group, a 2-methyl-2-butenyl group, a 2-methyl-3-butenyl group, a 3-methyl-2-butenyl group, a 3-methyl-3-butenyl group, a 1-methyl-1-butenyl group, a 1-methyl-3-butenyl group, a 1,2-dimethyl-2-propenyl group, a 1-ethyl-2-propenyl group, a 2-hexenyl group, a 3-hexenyl group, a 4-hexenyl group, a 5-hexenyl group, a 1-methyl-3-pentenyl group, a 1-methyl-4-pentenyl group, a 2-methyl-2-pentenyl group, a 3-methyl-3-pentenyl group, a 3-methyl-4-pentenyl group, a 4-methyl-3-pentenyl group, a 4-methyl-4-pentenyl group, a 2-propyl-2-propenyl group, a 1-propyl-2-propenyl group, a 1,2-dimethyl-2-butenyl group, a 1,2-dimethyl-3-butenyl group, a 1,3-dimethyl-2-butenyl group, a 1,3-dimethyl-3-butenyl group, a 1-ethyl-2-methyl-2-propenyl group, a 1-(1-methylethyl)-2-propenyl group, a 1-ethyl-2-butenyl group, a 1-ethyl-3-butenyl group, a 2-propynyl group, a 1-methyl-2-propynyl group, a 1,1-dimethyl-2-propynyl group, a 1-ethyl-2-propynyl group, a 1-propyl-2-propynyl group, a 1-(1-methylethyl)-2-propynyl group, a 2-butynyl group, a 1-methyl-2-butynyl group, a 1-ethyl-2-butynyl group, a 2-pentynyl group, a 1-methyl-2-pentynyl group, a 2-hexynyl group, a 3-butynyl group, a 1-methyl-3-butynyl group, a 1-ethyl-3-butynyl group, a 3-pentynyl group, a 1-methyl-3-pentynyl group, a 3-hexynyl group, a 4-pentynyl group, a 5-hexynyl group, a 2-fluoroethyl group, a 2,2-difluoroethyl group, a 2,2,2-trifluoroethyl group, a 3-fluoropropyl group, a 3,3-difluoropropyl group, a 3,3,3-trifluoropropyl group, a 2,2,3,3-tetrafluoropropyl group, a 2,2,3,3,3-pentafluoropropyl group, a 1-methyl-2-fluoroethyl group, a 1-methyl-2,2,2-trifluoroethyl group, a 2-fluoro-1-(fluoromethyl)ethyl group, a 2,2,2-trifluoro-1-(trifluoromethyl)ethyl group, a 4-fluorobutyl group, a 4,4-difluorobutyl group, a 4,4,4-trifluorobutyl group, a 3,3,4,4,4-pentafluorobutyl group, a 2,2,3,3,4,4-hexafluorobutyl group, a 2,2,3,3,4,4,4-heptafluorobutyl group, a 1-trifluoromethyl-propyl group, a 3,3,3-trifluoro-1-methylpropyl group, a 2,2,3,3-tetrafluoro-1-methylpropyl group, a 2,2,3,3,3-pentafluoro-1-methylpropyl group, a 2,2,3,3,3-pentafluoro-1-trifluoromethyl-propyl group, a 5-fluoropentyl group, a 5,5,5-trifluoropentyl group, a 6-fluorohexyl group, a 6,6,6-trifluorohexyl group, a 2,2,3,4,4-pentafluoro-3-butenyl group, a 2,2,3,3,3-pentafluoro-1-methylpropyl group, a 2,2,3,3,4,4,4-heptafluorobutyl group, a 2-chloroethyl group, a 2,2-dichloroethyl group, a 2,2,2-trichloroethyl group, a 3-chloropropyl group, a 2-chloropropyl group, a 3-chloro-2,2-dimethylpropyl group, a 3,3-dichloropropyl group, a 2,3-dichloropropyl group, a 2-chloro-1-methylethyl group, a 2-chloro-1-(chloromethyl)ethyl group, a 1-methyl-2,2,2-trichloroethyl group, a 4-chlorobutyl group, a 1-chlorobutyl group, a 3-chloro-1-(chloromethyl)propyl group, a 2-chloro-2-methylpropyl group, a 5-chloropentyl group, a 6-chlorohexyl group, a 2-bromoethyl group, a 2,2,2-tribromoethyl group, a 3-bromopropyl group, a 2,3-dibromopropyl group, a 2-bromo-1-methylethyl group, a 2-bromo-1-(bromomethyl)ethyl group, a 4-bromobutyl group, a 3-bromo-1-(bromomethyl)propyl group, a 2-(bromomethyl)propyl group, a 3-bromo-2-(bromomethyl)propyl group, a 2-iodoethyl group, a 3-iodopropyl group, a 3-fluoro-2-propenyl group, a 2-fluoro-2-propenyl group, a 3,3-difluoro-2-propenyl group, a 2,3-difluoro-2-propenyl group, a 2,3,3-trifluoro-2-propenyl group, a 4,4-difluoro-3-butenyl group, a 3,4,4-trifluoro-3-butenyl group, a 2,3-difluoro-2-butenyl group, a 2-fluoro-3-methyl-2-butenyl group, a 5,5-difluoro-4-pentenyl group, a 4,5,5-trifluoro-4-pentenyl group, a 4,4,4-trifluoro-3-(trifluoromethyl)-2-butenyl group, a 2,4,4,4-tetrafluoro-2-butenyl group, a 4,4,4-trifluoro-3-methyl-2-butenyl group, a 4,4,4-trifluoro-3-(trifluoromethyl)-2-butenyl group, a 3-chloro-2-propenyl group, a 2-chloro-2-propenyl group, a 3,3-dichloro-2-propenyl group, a 2,3-dichloro-2-propenyl group, a 2,3,3-trichloro-2-propenyl group, a 4-chloro-3-butenyl group, a 4,4-dichloro-3-butenyl group, a 3,4-dichloro-3-butenyl group, a 3-chloro-2-butenyl group, a 2-chloro-2-butenyl group, a 2,3-dichloro-2-butenyl group, a 2-chloro-3-methyl-2-butenyl group, a 5-chloro-4-pentenyl group, a 4-chloro-4-pentenyl group, a 4,5-dichloro-4-pentenyl group, a 3-bromo-2-propenyl group, a 2-bromo-2-propenyl group, a 3,3-dibromo-2-propenyl group, a 2,3-dibromo-2-propenyl group, a 4-bromo-3-butenyl group, a 4,4-dibromo-3-butenyl group, a 3,4-dibromo-3-butenyl group, a 3,4,4-tribromo-3-butenyl group, a 3-bromo-2-butenyl group, a 2-bromo-2-butenyl group, a 2,3-dibromo-2-butenyl group, a 2-bromo-3-methyl-2-butenyl group, a 4-bromo-4-pentenyl group, a 4,5-dibromo-4-pentenyl group, a 4,5,5-tribromo-4-pentenyl group, a 3-chloro-propynyl group, a 3-chloro-1-methyl-2-propynyl group, a 3-chloro-1,1-dimethyl-2-propynyl group, a 3-chloro-1-ethyl-2-propynyl group, a 3-chloro-1-propyl-2-propynyl group, a 3-chloro-1-(1-methylethyl)-2-propynyl group, a 4-chloro-3-butynyl group, a 4-chloro-1-methyl-3-butynyl group, a 4-chloro-1-ethyl-3-butynyl group, a 5-chloro-4-pentynyl group, a 6-chloro-5-hexynyl group, a 3-bromopropynyl group, a 3-bromo-1-methyl-2-propynyl group, a 3-bromo-1,1-dimethyl-2-propynyl group, a 3-bromo-1-ethyl-2-propynyl group, a 3-bromo-1-propyl-2-propynyl group, a 3-bromo-1-isopropyl-2-propynyl group, a 4-bromo-3-butynyl group, a 4-bromo-1-methyl-3-butynyl group, a 4-bromo-1-ethyl-3-butynyl group, a 5-bromo-4-pentynyl group, a 6-bromo-5-hexynyl group, a methoxymethyl group, a 2-methoxyethyl group, a 2-methoxy-1-methylethyl group, a 2-methoxy-2-methylethyl group, a 2-ethyl-2-methoxyethyl group, a 2-ethoxyethyl group, a 2-propoxyethyl group, a 2-(1-methylethyl)oxyethyl group, a 2-butoxyethyl group, a 2-isobutoxyethyl group, a 2-(sec-butoxy)ethyl group, a 2-(tert-butoxy)ethyl group, a 3-methoxypropyl group, a 3-methoxy-3-methylpropyl group, a 3-methoxy-3,3-dimethylpropyl group, a 3-ethoxypropyl group, a 3-propoxypropyl group, a 3-(1-methylethyl)oxypropyl group, a 3-butoxypropyl group, a 3-isobutoxypropyl group, a 3-(sec-butoxy)propyl group, a 3-(tert-butoxy)propyl group, a 3,3-diethoxypropyl group, a 2,2-diethoxyethyl group, any of groups represented by the following formulas:

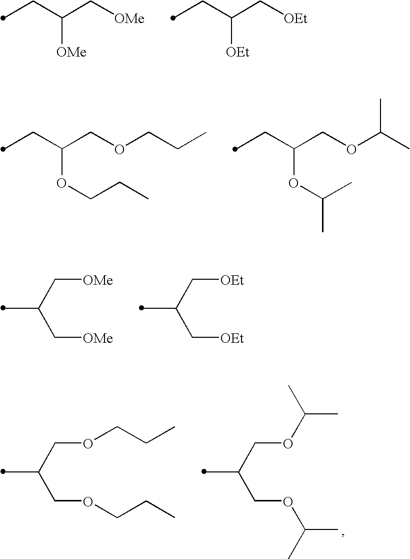

a methylthiomethyl group, a 2-methylthioethyl group, a 2-ethylthioethyl group, a 2-propylthioethyl group, a 2-isopropylthioethyl group, a 2-butylthioethyl group, a 2-isobutylthioethyl group, a 2-(sec-butylthio)ethyl group, a 2-(tert-butylthio)ethyl group, a 3-methylthiopropyl group, a 3-ethylthiopropyl group, a 3-propylthiopropyl group, a 3-butylthiobutyl group, a 3-(tert-butylthio)propyl group, a formylmethyl group, a 1-formylethyl group, a 2-formylethyl group, a 3-formylpropyl group, a 4-formylbutyl group, a 5-formylpentyl group, a cyanomethyl group, a 1-cyanoethyl group, a 2-cyanoethyl group, a 3-cyanopropyl group, a 4-cyanobutyl group, a 5-cyanopentyl group, a 1-hydroxyiminoethyl group, a 2-hydroxyiminoethyl group, a 3-hydroxyiminopropyl group, a 4-hydroxyiminobutyl group, a 5-(hydroxyimino)pentyl group, a 6-(hydroxyimino)hexyl group, a 2-(methoxyimino)ethyl group, a 2-(ethoxyimino)ethyl group, a 2-(propoxyimino)ethyl group, a 2-(isopropoxyimino)ethyl group, a 3-(methoxyimino)propyl group, a 3-(ethoxyimino)propyl group, a 3-(propoxyimino)propyl group, a 3-(isopropoxyimino)propyl group, a 4-(methoxyimino)butyl group, a 4-(ethoxyimino)butyl group, a 4-(propoxyimino)butyl group, a 4-(isopropoxyimino)butyl group, a 2-(methylamino)ethyl group, a 3-(methylamino)propyl group, a 4-(methylamino)butyl group, a 5-(methylamino)pentyl group, a 6-(methylamino)hexyl group, a 2-(dimethylamino)ethyl group, a 3-(dimethylamino)propyl group, a 4-(dimethylamino)butyl group, a 5-(dimethylamino)pentyl group, a 6-(dimethylamino)hexyl group, a 2-(methoxycarbonyl)ethyl group, a 2-(ethoxycarbonyl)ethyl group, a 3-(methoxycarbonyl)propyl group, a 3-(ethoxycarbonyl)propyl group, a 4-(methoxycarbonyl)butyl group, a 4-(ethoxycarbonyl)butyl group, a 5-(methoxycarbonyl)pentyl group, a 5-(ethoxycarbonyl)pentyl group, 1-hydroxyethyl group, a 2-hydroxyethyl group, a 3-hydroxyethyl group, a 4-hydroxybutyl group, a 5-hydroxypentyl group, a 6-hydroxyhexyl group, any of groups represented by the following formulas:

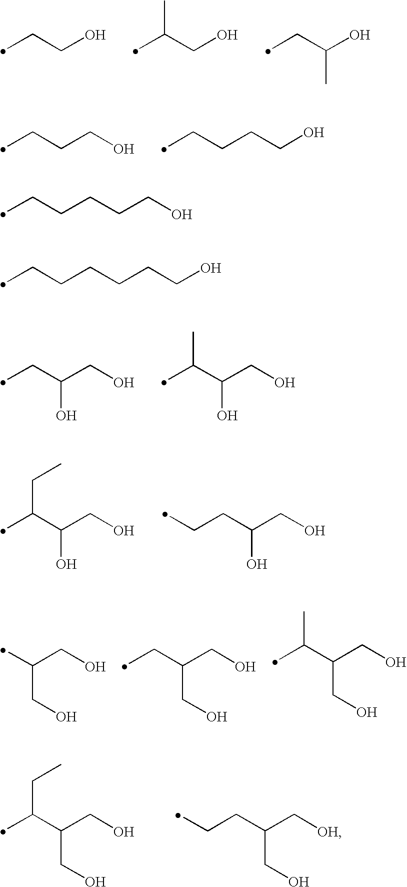

an acetylmethyl group, a propionylmethyl group, a butyrylmethyl group, a valerylmethyl group, a 2-acetylethyl group, a 2-propionylethyl group, a 2-butyrylethyl group, a 3-acetylpropyl group, a 3-propionylpropyl group, a 4-acetylbutyl group, a 2-(methoxymethoxy)ethyl group, any of groups represented by the following formulas:

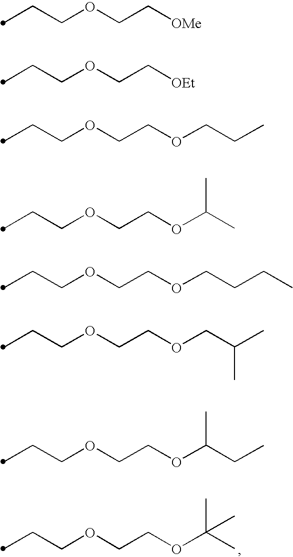

any of groups represented by the following formulas:

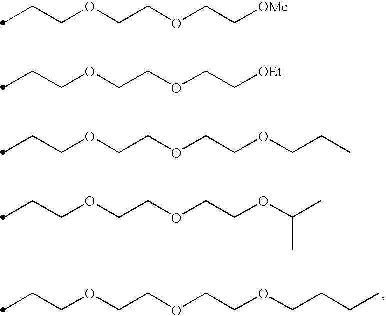

a 3-(2,2,2-ethoxy)propyl group, a 2-(2-fluoroethoxy)ethyl group, a 2-(2-chloroethoxy)ethyl group, a 2-(2-bromoethoxy)ethyl group, a 2-(2-iodoethoxy)ethyl group, a 2-(2,2,2-trifluoroethoxy)ethyl group, a 3-(2-chloroethoxy)propyl group, a 3-(2-bromoethoxy)propyl group, a 3-(2-iodoethoxy)propyl group, a 3-(2,2,2,-trifluoroethoxy)propyl group, any of groups represented by the following formulas:

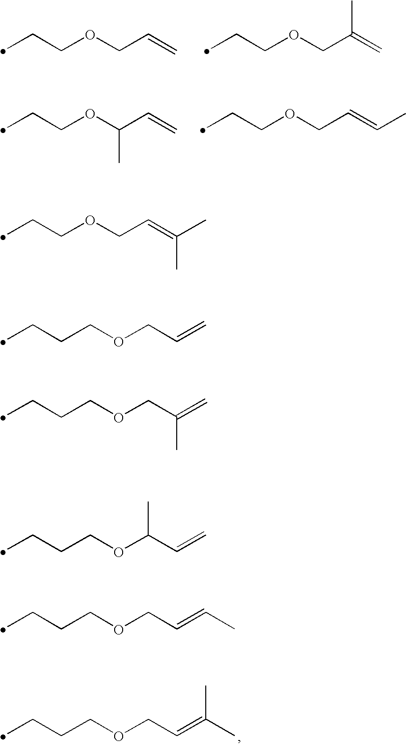

any of groups represented by the following formulas:

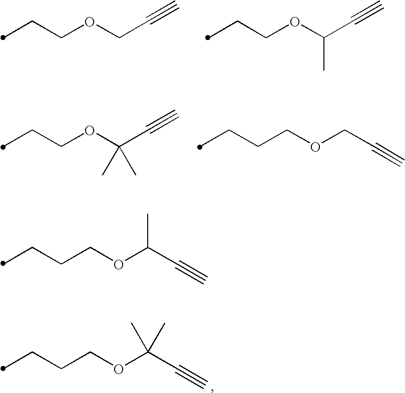

any of groups represented by the following formulas:

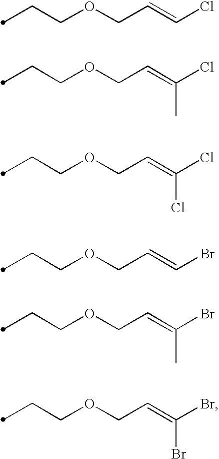

any of groups represented by the following formulas:

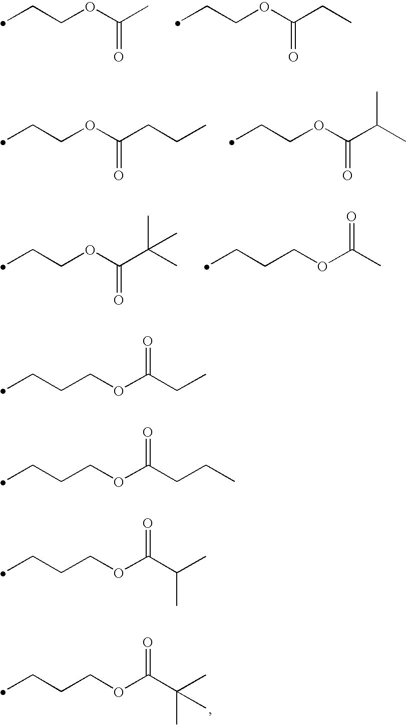

a propionyl group, a butyryl group, an isobutyryl group, a valeryl group, an isovaleryl group, a pivaloyl group, a cyclopropyl group, a cyclobutyl group, a cyclopentyl group, a cyclohexyl group, a cycloheptyl group, a cyclooctyl group, a 2-methylcyclohexyl group, a 3-methylcyclohexyl group, a 4-methylcyclohexyl group, a 1-vinylcyclohexyl group, a 1-allylcyclohexyl group, a 1-ethynylcyclohexyl group, a 2-chlorocyclohexyl group, a 4-chlorocyclohexyl group, a 2-fluorocyclohexyl group, a 2-methoxycyclobutyl group, a 2-methoxycyclopentyl group, a 3-methoxycyclopentyl group, a 2-methoxycyclohexyl group, a 3-methoxycyclohexyl group, a 4-methoxycyclohexyl group, a 2-methoxycycloheptyl group, a 2-methoxycyclooctyl group, any of groups represented by the following formulas:

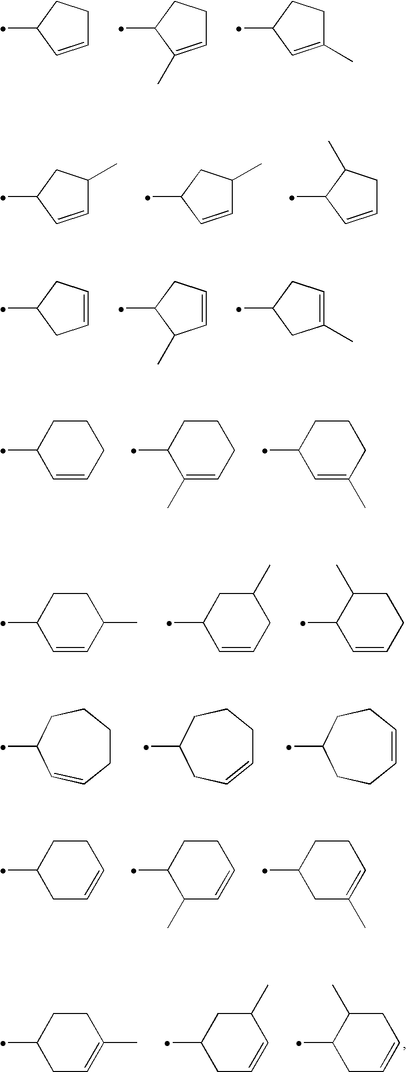

a phenyl group, a 2-fluorophenyl group, a 3-fluorophenyl group, a 4-fluorophenyl group, a 2,3-difluorophenyl group, a 2,4-difluorophenyl group, a 2,5-difluorophenyl group, a 2,6-difluorophenyl group, a 3,4-difluorophenyl group, a 3,5-difluorophenyl group, a 2-chlorophenyl group, a 3-chlorophenyl group, a 4-chlorophenyl group, a 2,3-dichlorophenyl group, a 2,4-dichlorophenyl group, a 2,5-dichlorophenyl group, a 2,6-dichlorophenyl group, a 3,4-dichlorophenyl group, a 3,5-dichlorophenyl group, a 2-bromophenyl group, a 3-bromophenyl group, a 4-bromophenyl group, a 2,3-dibromophenyl group, a 2,4-dibromophenyl group, a 2,5-dibromophenyl group, a 2,6-dibromophenyl group, a 3,4-dibromophenyl group, a 3,5-dibromophenyl group, a 2-iodophenyl group, a 3-iodophenyl group, a 4-iodophenyl group, a 2-methylphenyl group, a 3-methylphenyl group, a 4-methylphenyl group, a 2,3-dimethylphenyl group, a 2,4-dimethylphenyl group, a 2,5-dimethylphenyl group, a 2,6-dimethylphenyl group, a 3,4-dimethylphenyl group, a 3,5-dimethylphenyl group, a 2-methoxyphenyl group, a 3-methoxyphenyl group, a 4-methoxyphenyl group, a 2,3-dimethoxyphenyl group, a 2,4-dimethoxyphenyl group, a 2,5-dimethoxyphenyl group, a 2,6-dimethoxyphenyl group, a 3,4-dimethoxyphenyl group, a 3,5-dimethoxyphenyl group, a 2-ethylphenyl group, a 3-ethylphenyl group, a 4-ethylphenyl group, a 2-(trifluoromethyl)phenyl group, a 3-(trifluoromethyl)phenyl group, a 4-(trifluoromethyl)phenyl group, a 2-methylthiophenyl group, a 3-methylthiophenyl group, a 4-methylthiophenyl group, a 2-(trifluoromethoxy)phenyl group, a 3-(trifluoromethoxy)phenyl group, a 4-(trifluoromethoxy)phenyl group, a 2-nitrophenyl group, a 3-nitrophenyl group, a 4-nitrophenyl group, a 2-cyanophenyl group, a 3-cyanophenl group, a 4-cyanophenyl group, any of groups represented by the following formulas:

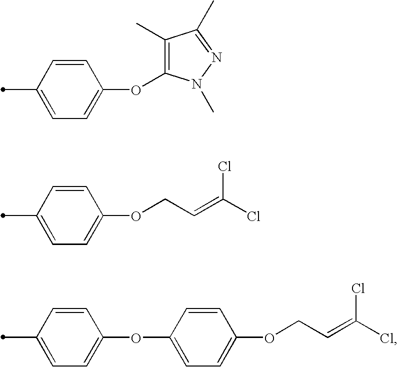

any of groups represented by the following formulas:

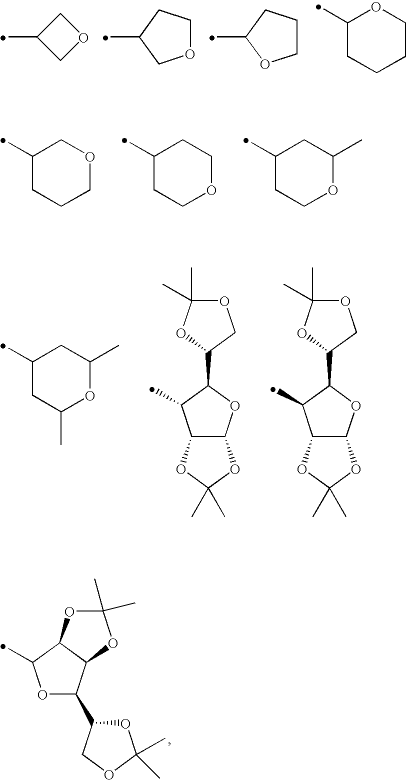

any of groups represented by the following formulas:

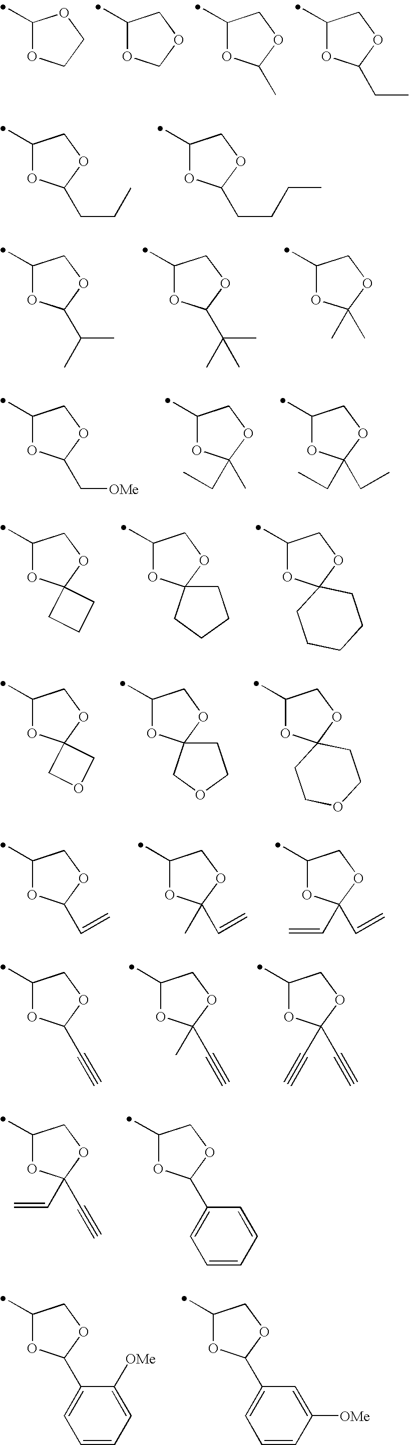

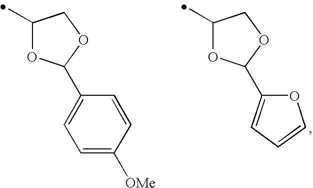

any of groups represented by the following formulas:

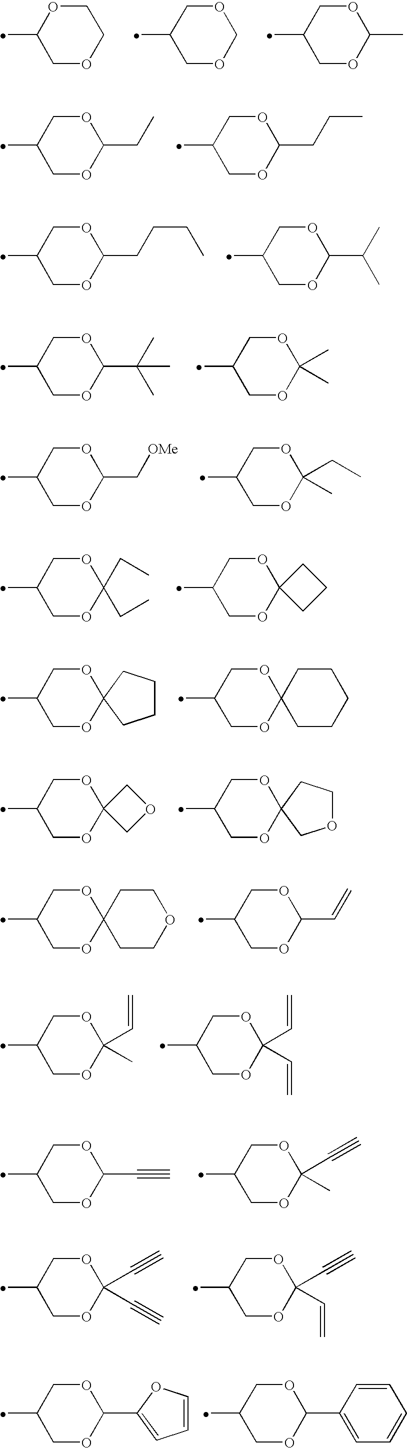

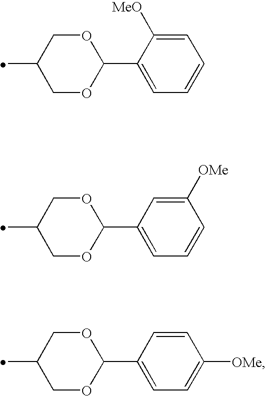

any of groups represented by the following formulas:

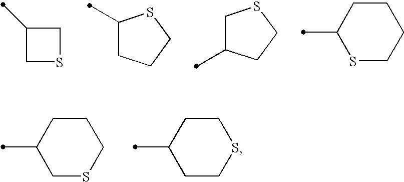

any of groups represented by the following formulas:

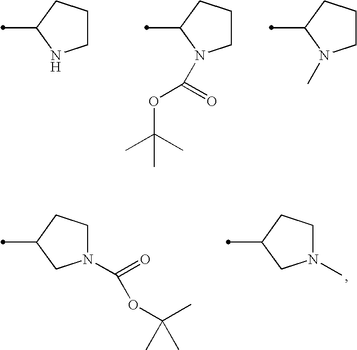

any of groups represented by the following formulas:

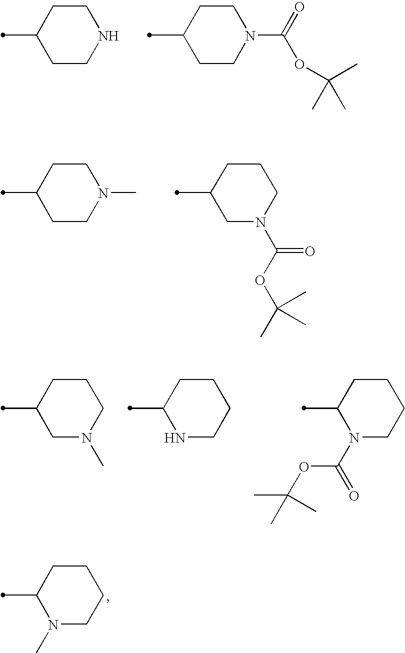

any of groups represented by the following formulas:

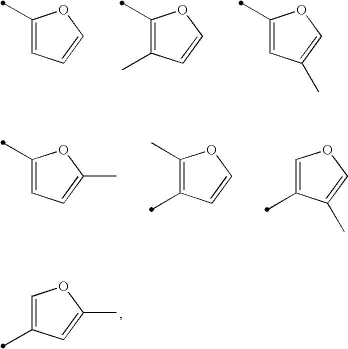

any of groups represented by the following formulas:

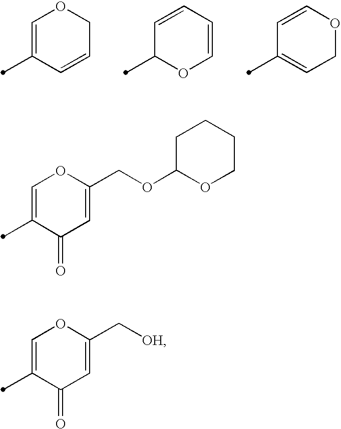

any of groups represented by the following formulas:

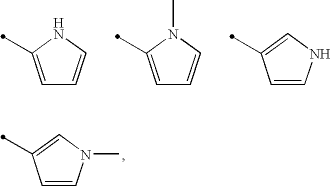

a 2-pyridyl group, a 3-pyridyl group, a 4-pyridyl group, a 2-pyrimidinyl group, a 4-pyrimidinyl group, a 5-pyrimidinyl group, a 2-pyrazinyl group, a 3-pyridazinyl group, a 4-pyridazinyl group, any of groups represented by the following formulas:

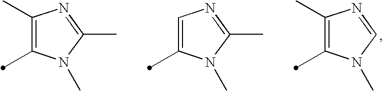

any of groups represented by the following formulas:

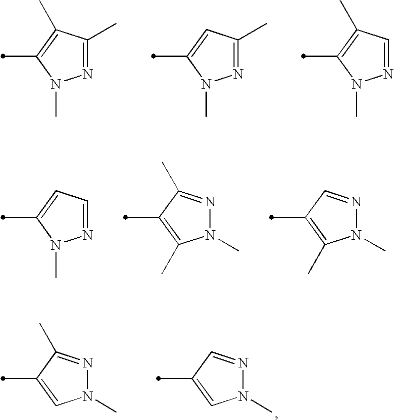

any of groups represented by the following formulas:

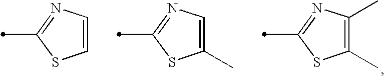

any of groups represented by the following formulas:

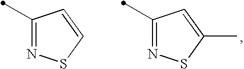

any of groups represented by the following formulas:

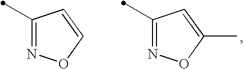

any of groups represented by the following formulas:

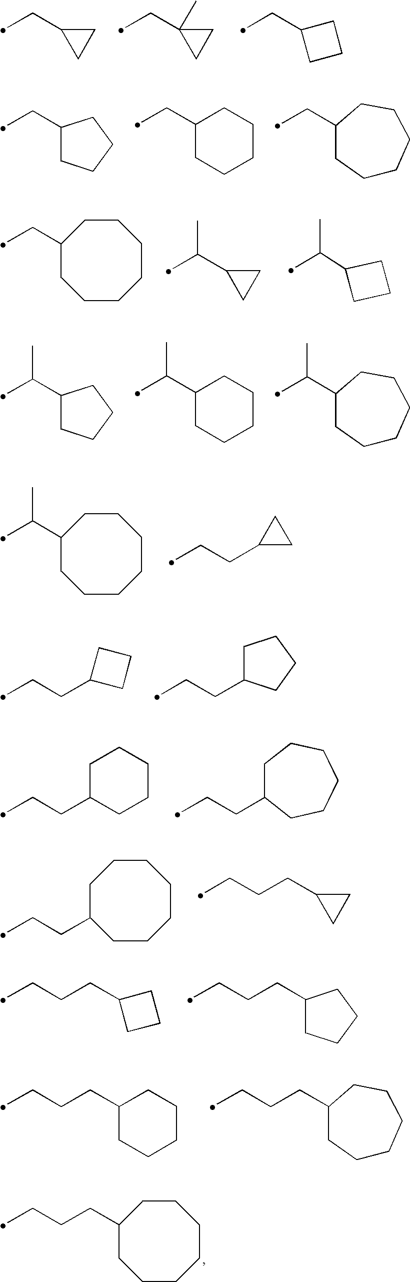

any of groups represented by the following formulas:

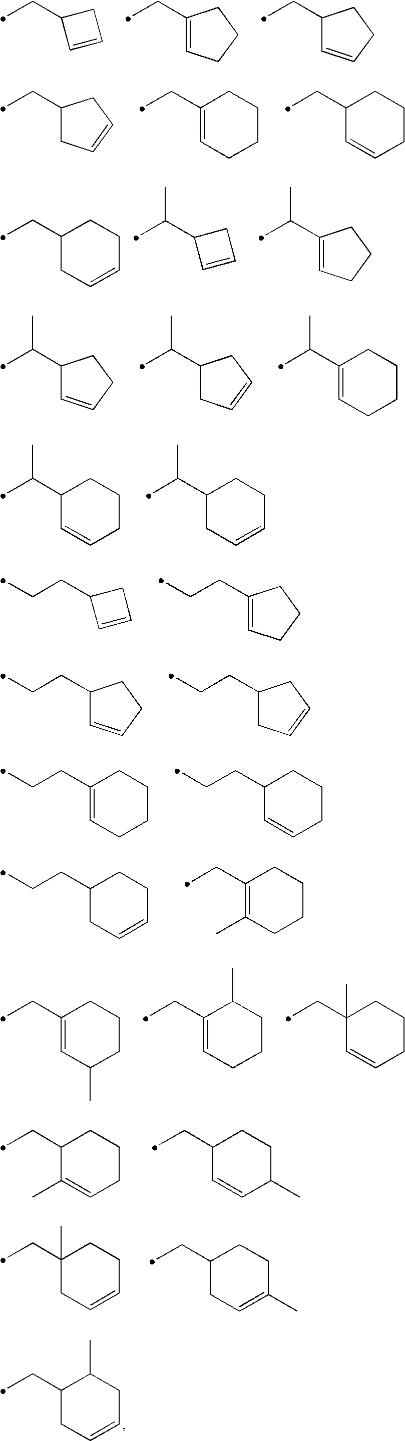

a benzyl group, a 2-fluorobenzyl group, a 3-fluorobenzyl group, a 4-fluorobenzyl group, a 2,3-difluorobenzyl group, a 2,4-difluorobenzyl group, a 2,5-difluorobenzyl group, a 2,6-difluorobenzyl group, a 3,4-difluorobenzyl group, a 3,5-difluorobenzyl group, a 2-chlorobenzyl group, a 3-chlorobenzyl group, a 4-chlorobenzyl group, a 2,3-dichlorobenzyl group, a 2,4-dichlorobenzyl group, a 2,5-dichlorobenzyl group, a 2,6-dichlorobenzyl group, a 3,4-dichlorobenzyl group, a 3,5-dichlorobenzyl group, a 2-bromobenzyl group, a 3-bromobenzyl group, a 4-bromobenzyl group, a 2,3-dibromobenzyl group, a 2,4-dibromobenzyl group, a 2,5-dibromobenzyl group, a 2,6-dibromobenzyl group, a 3,4-dibromobenzyl group, a 3,5-dibromobenzyl group, a 2-iodobenzyl group, a 3-iodobenzyl group, a 4-iodobenzyl group, a 2-methylbenzyl group, a 3-methylbenzyl group, a 4-methylbenzyl group, a 2-(trifluoromethyl)benzyl group, a 3-(trifluoromethyl)benzyl group, a 4-(trifluoromethyl)benzyl group, a 2-methoxybenzyl group, a 3-methoxybenzyl group, a 4-methoxybenzyl group, a 2,5-dimethoxybenzyl group, a 3,5-dimethoxybenzyl group, a 2-methylthiobenzyl group, a 3-methylthiobenzyl group, a 4-methylthiobenzyl group, a 2-(trifluoromethoxy)benzyl group, a 3-(trifluoromethoxy)benzyl group, a 4-(trifluoromethoxy)benzyl group, a 2-nitrobenzyl group, a 3-nitrobenzyl group, a 4-nitrobenzyl group, a 2-cyanobenzyl group, a 3-cyanobenzyl group, a 4-cyanobenzyl group, a 2-ethoxy-benzyl group, a 3-ethoxy-benzyl group, a 4-ethoxybenzyl group, a 4-isopropylbenzyl group, a 4-tert-butylbenzyl group, a 2-fluoro-4-(trifluoromethyl)benzyl group, a 2-fluoro-5-(trifluoromethyl)benzyl group, a 4-fluoro-3-(trifluoromethyl)benzyl group, a 2,4-bis(trifluoromethyl)benzyl group, a 5-fluoro-2-methylbenzyl group, a pentafluorobenzyl group, a phenethyl group, any of groups represented by the following formulas:

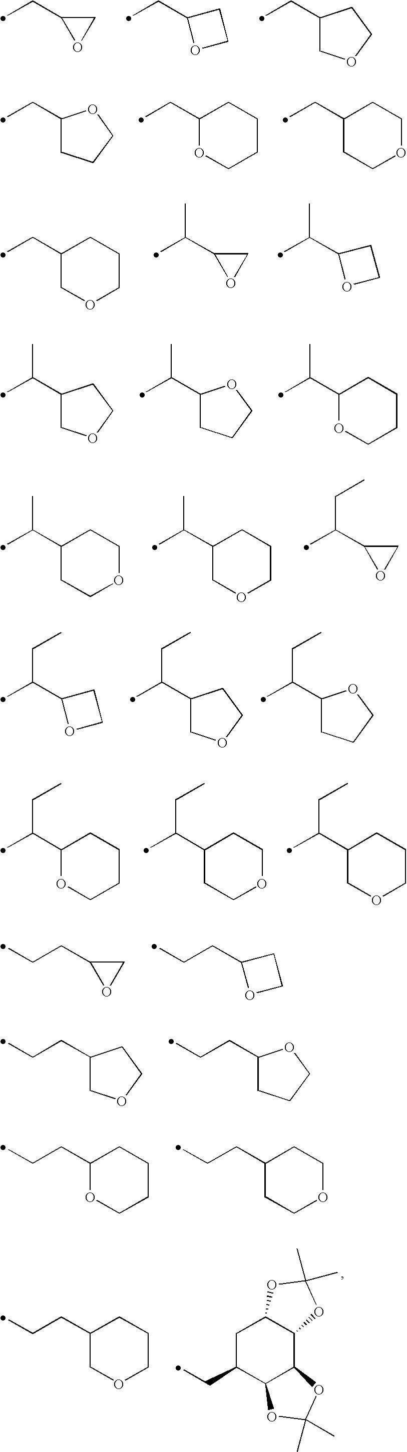

any of groups represented by the following formulas:

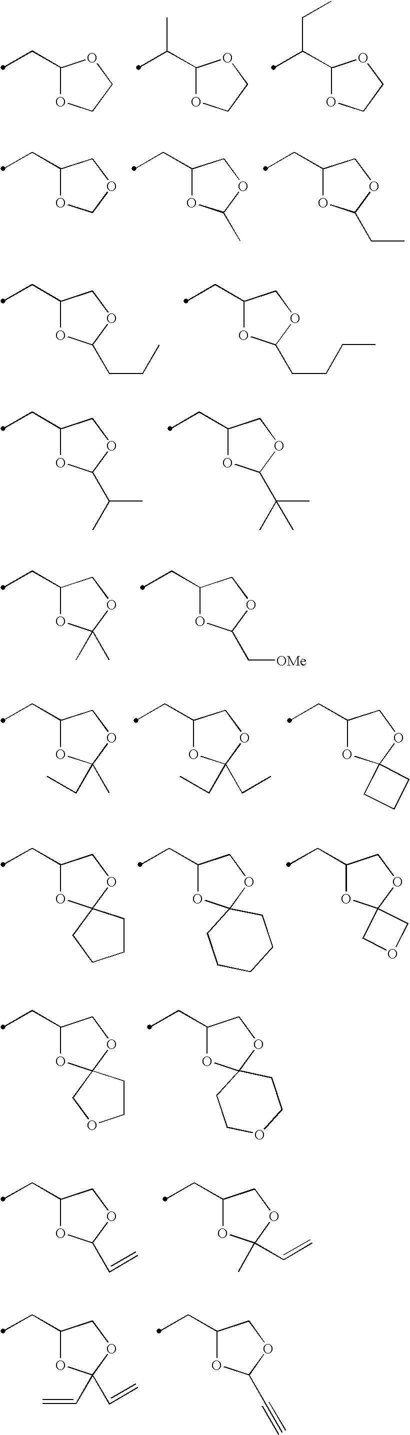

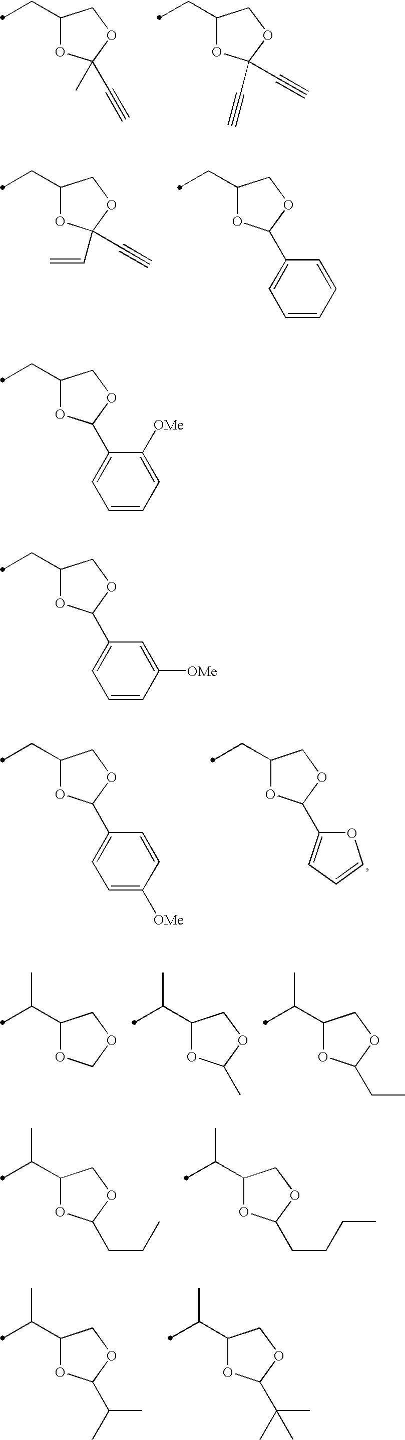

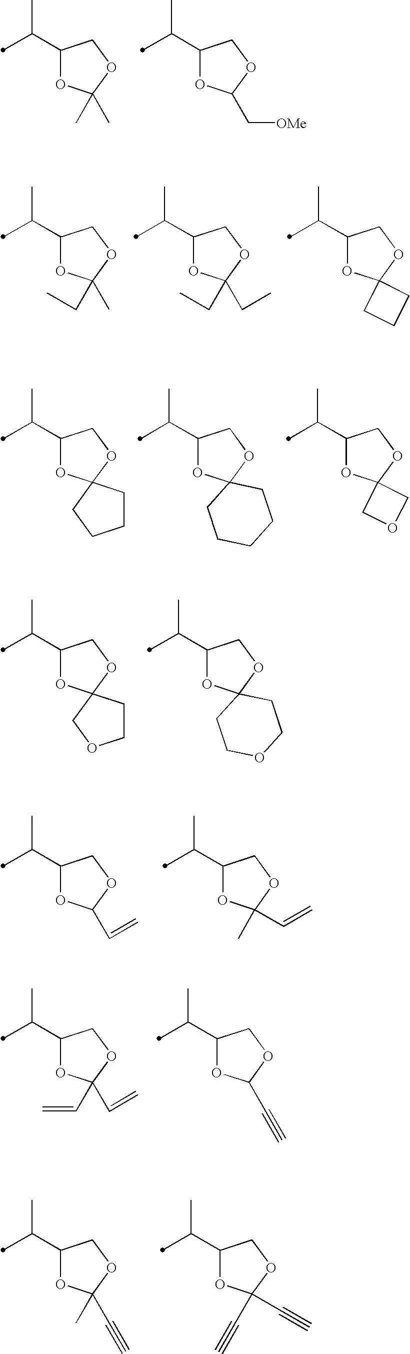

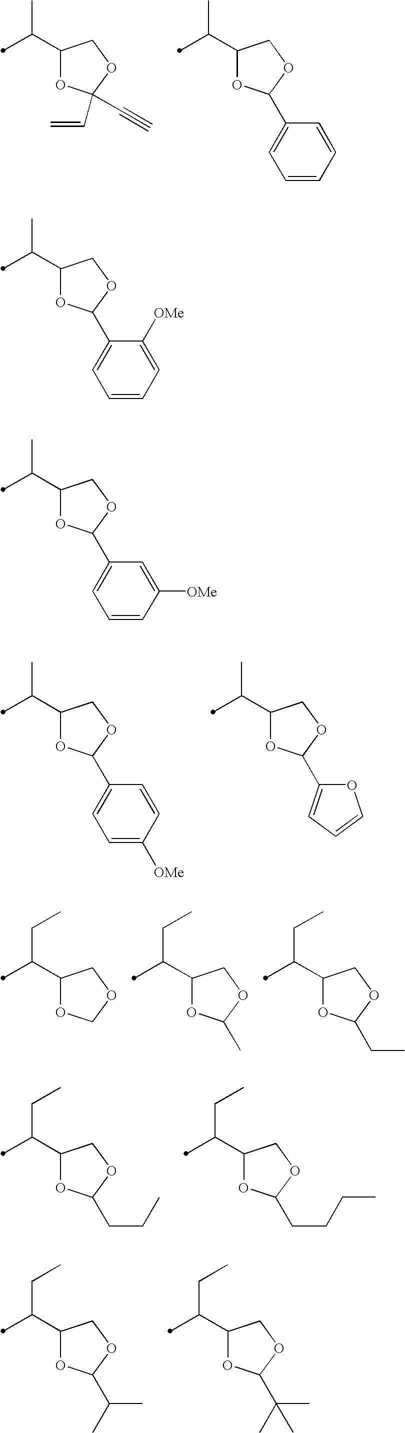

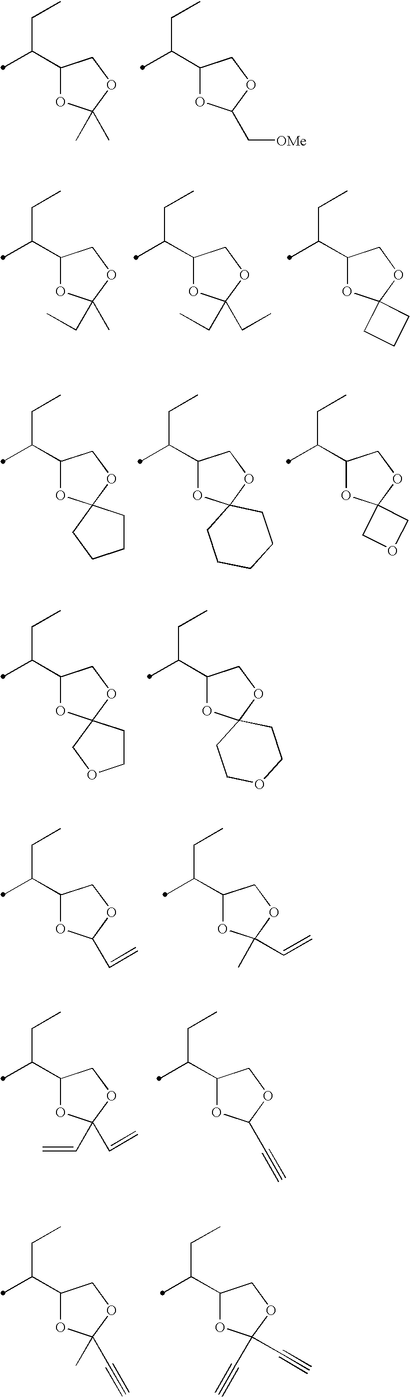

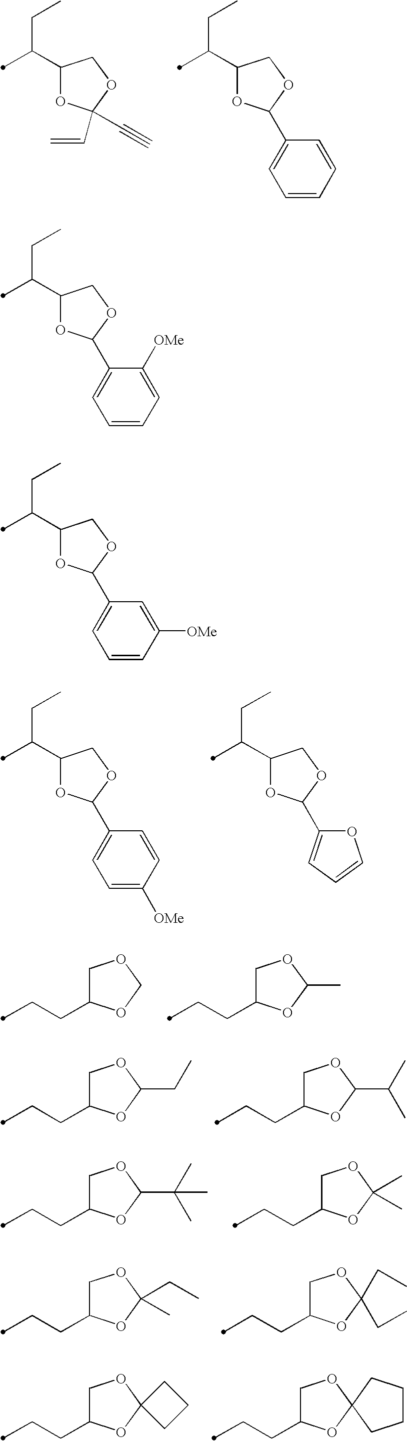

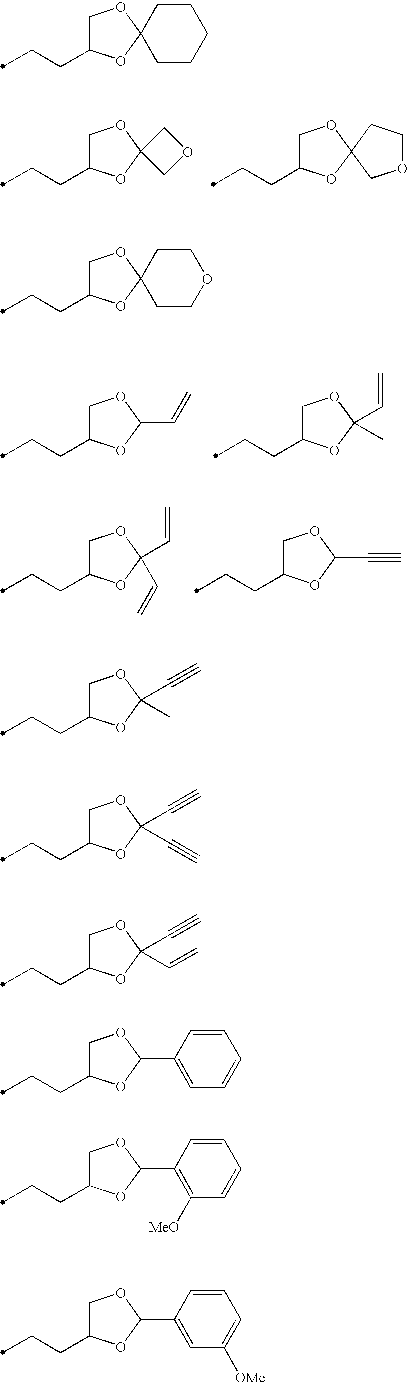

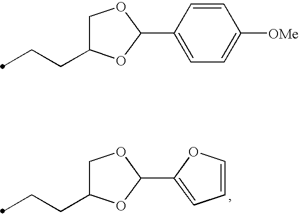

any of groups represented by the following formulas:

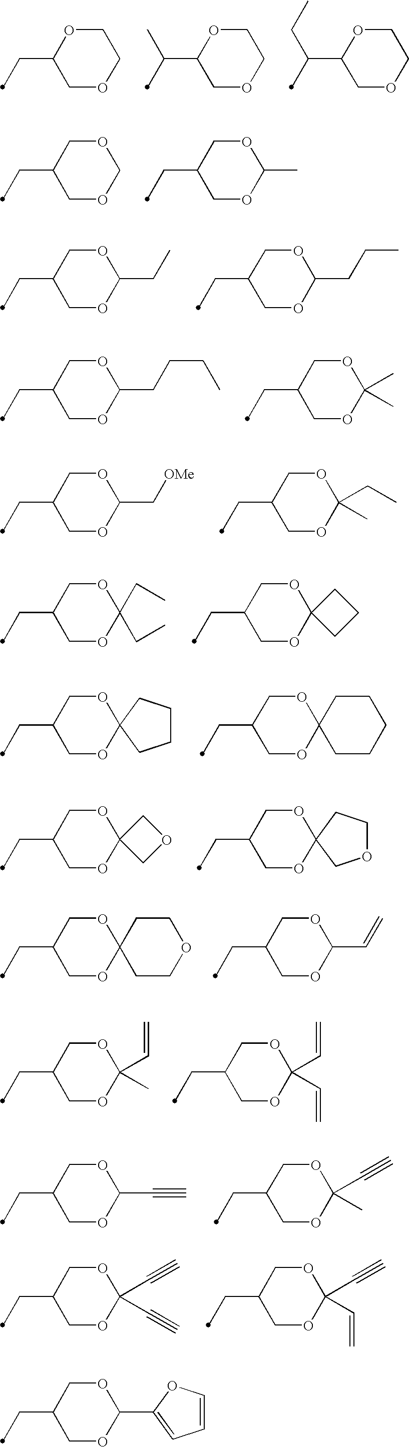

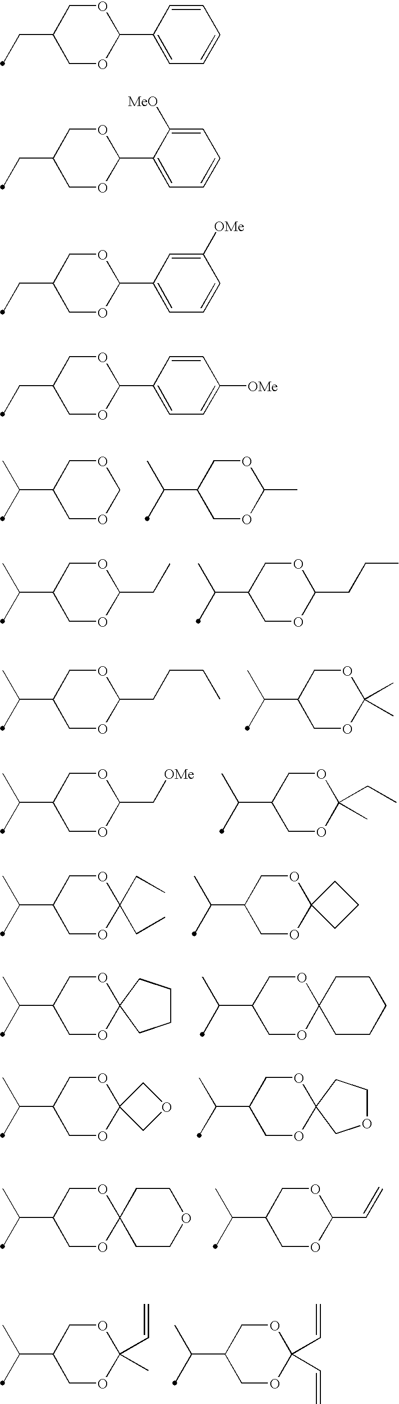

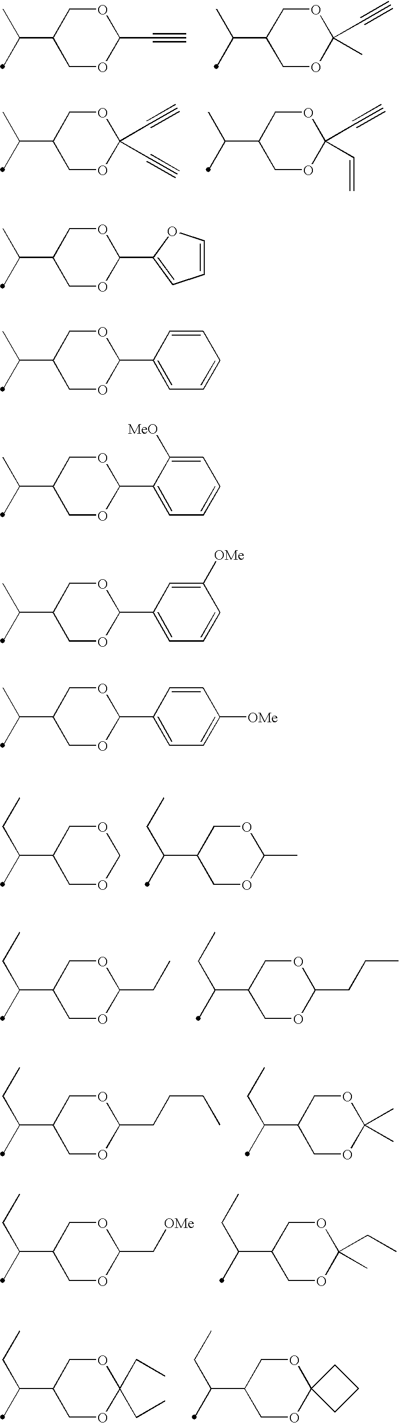

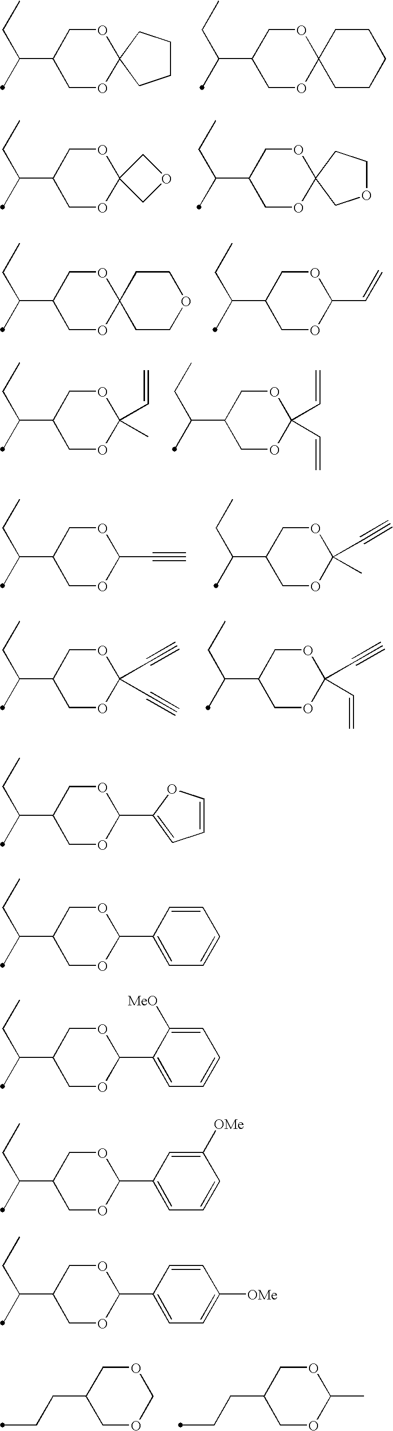

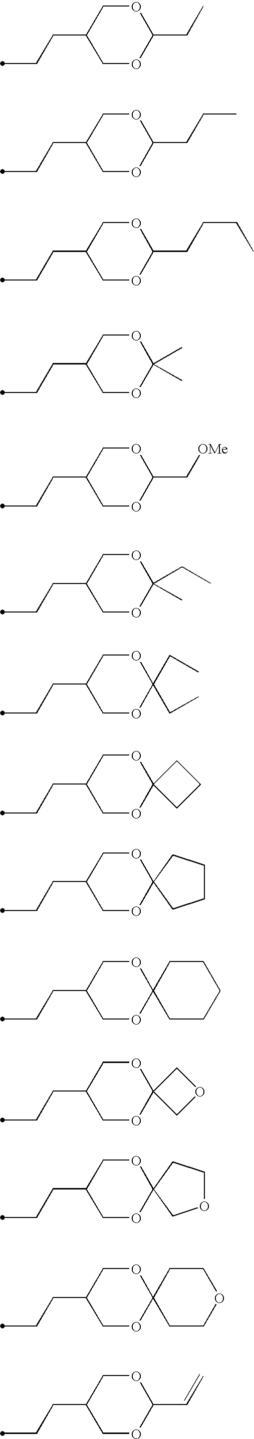

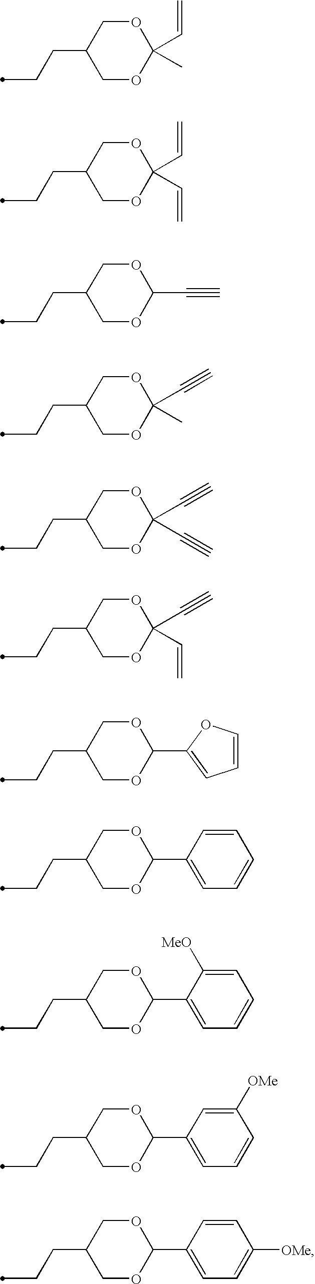

any of groups represented by the following formulas:

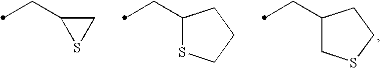

any of groups represented by the following formulas:

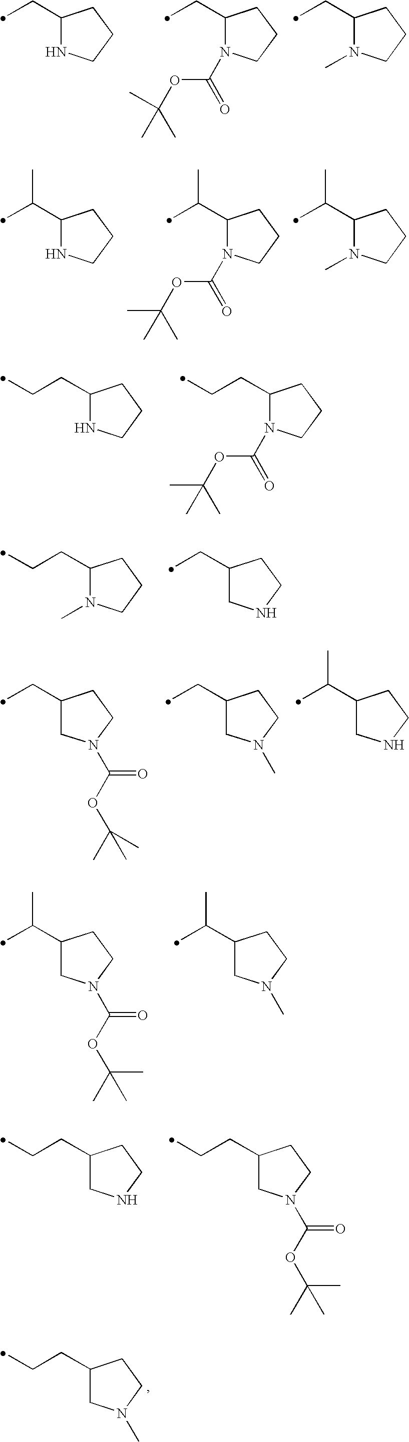

any of groups represented by the following formulas:

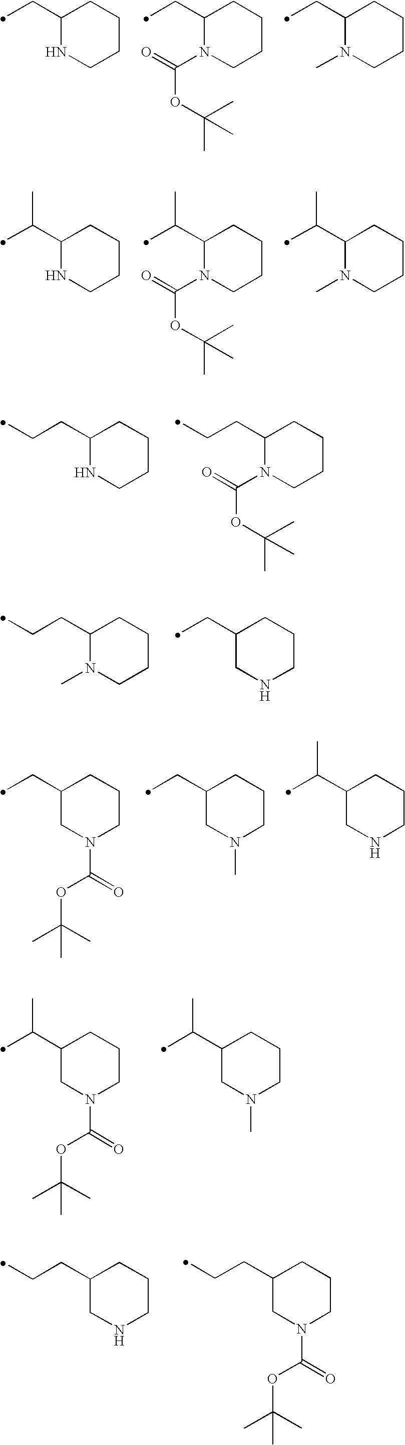

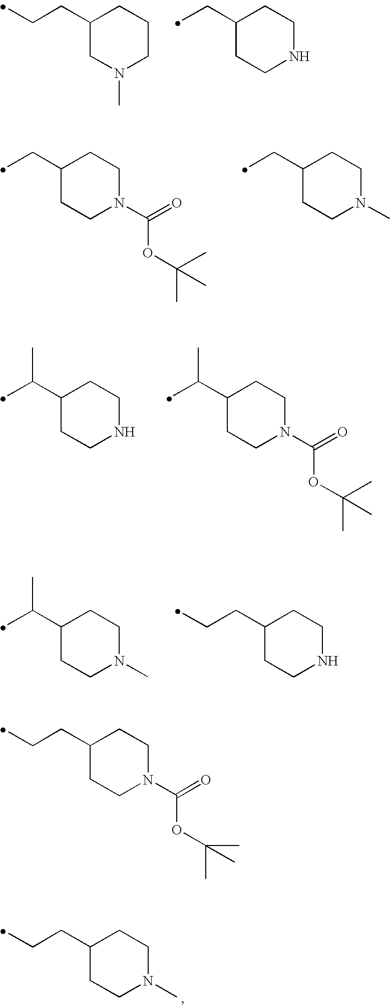

any of groups represented by the following formulas:

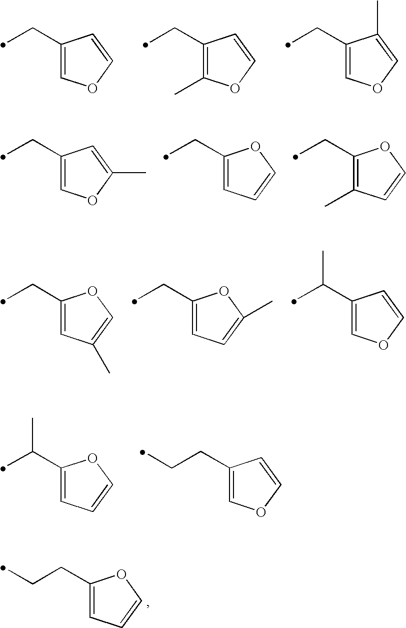

any of groups represented by the following formulas:

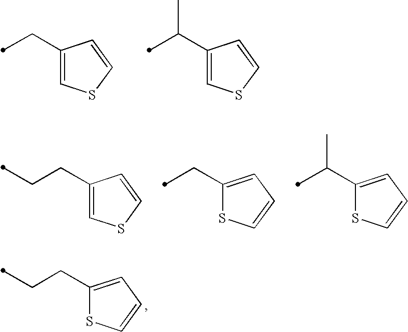

any of groups represented by the following formulas:

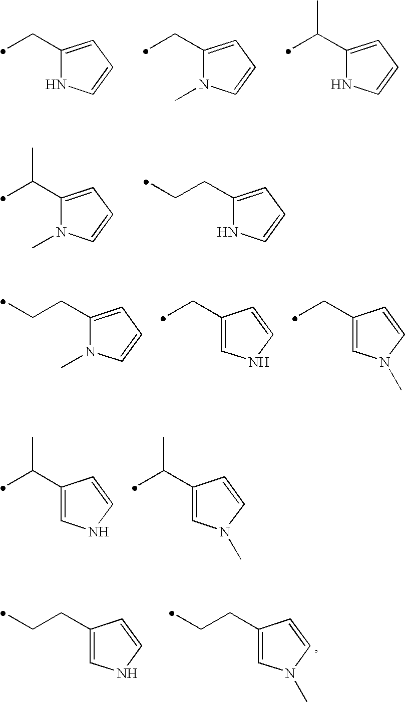

any of groups represented by the following formulas:

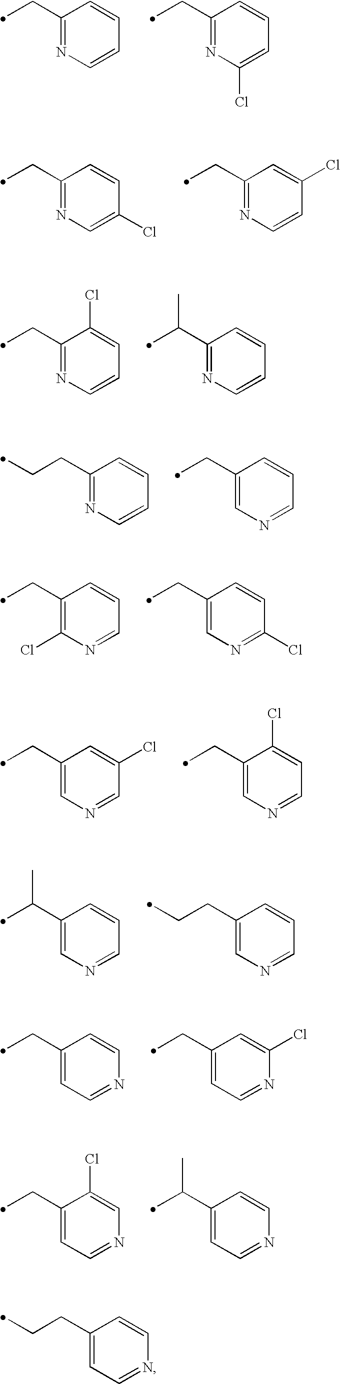

any of groups represented by the following formulas:

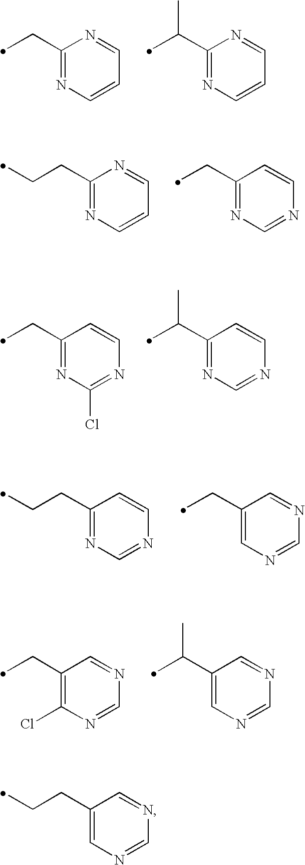

any of groups represented by the following formulas:

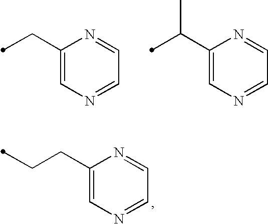

any of groups represented by the following formulas:

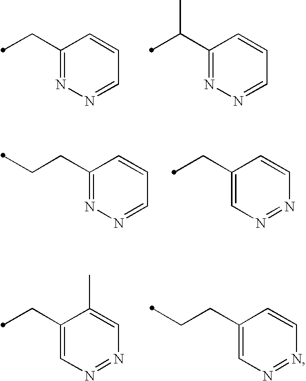

any of groups represented by the following formulas:

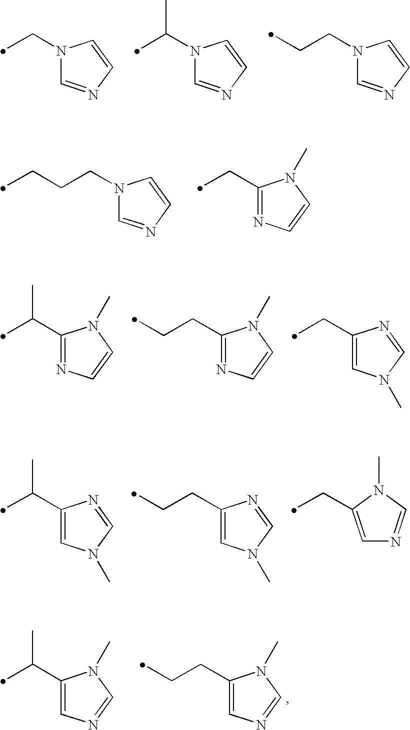

any of groups represented by the following formulas:

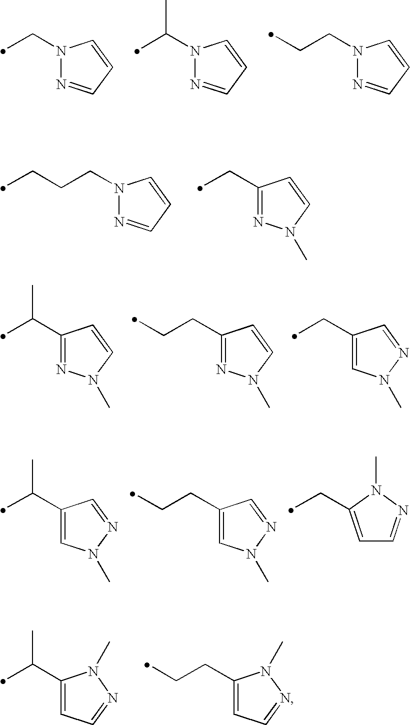

any of groups represented by the following formulas:

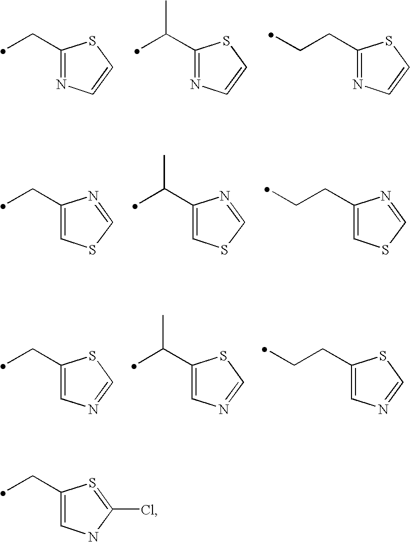

any of groups represented by the following formulas:

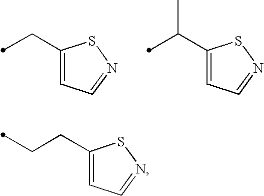

any of groups represented by the following formulas:

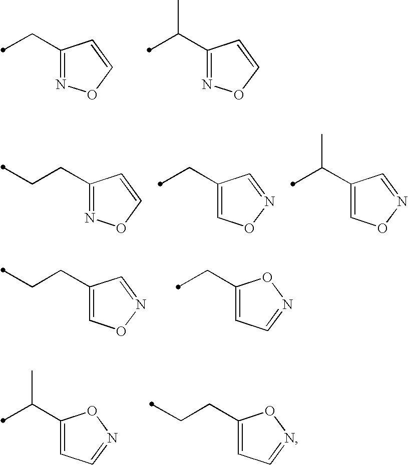

any of groups represented by the following formulas:

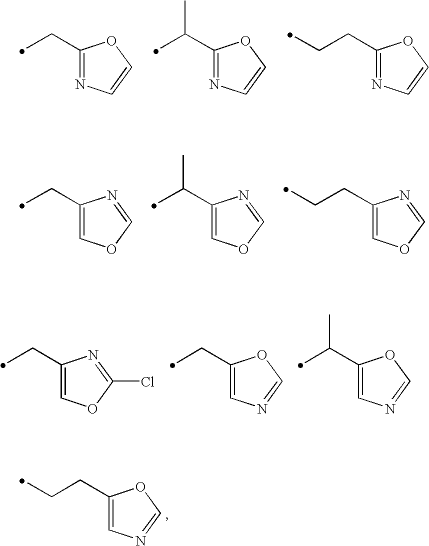

a 2-phenyloxyethyl group, a 2-(2-fluorophenyloxy)ethyl group, a 2-(3-fluorophenyloxy)ethyl group, a 2-(4-fluorophenyloxy)ethyl group, a 2-(2,3-difluorophenyloxy)ethyl group, a 2-(2,4-difluorophenyloxy)ethyl group, a 2-(2,5-difluorophenyloxy)ethyl group, a 2-(2,6-difluorophenyloxy)ethyl group, a 2-(3,4-difluorophenyloxy)ethyl group, a 2-(3,5-difluorophenyloxy)ethyl group, a 2-(2-chlorophenyloxy)ethyl group, a 2-(3-chlorophenyloxy)ethyl group, a 2-(4-chlorophenyloxy)ethyl group, a 2-(2,3-dichlorophenyloxy)ethyl group, a 2-(2,4-dichlorophenyloxy)ethyl group, a 2-(2,5-dichlorophenyloxy)ethyl group, a 2-(2,6-dichlorophenyloxy)ethyl group, a 2-(3,4-dichlorophenyloxy)ethyl group, a 2-(3,5-dichlorophenyloxy)ethyl group, a 2-(2-bromophenyloxy)ethyl group, a 2-(3-bromophenyloxy)ethyl group, a 2-(4-bromophenyloxy)ethyl group, a 2-(2,3-dibromophenyloxy)ethyl group, a 2-(2,4-dibromophenyloxy)ethyl group, a 2-(2,5-dibromophenyloxy)ethyl group, a 2-(2,6-dibromophenyloxy)ethyl group, a 2-(3,4-dibromophenyloxy)ethyl group, a 2-(3,5-dibromophenyloxy)ethyl group, a 2-(2-iodophenyloxy)ethyl group, a 2-(3-iodophenyloxy)ethyl group, a 2-(4-iodophenyloxy)ethyl group, a 2-(2-methylphenyloxy)ethyl group, a 2-(3-methylphenyloxy)ethyl group, a 2-(4-methylphenyloxy)ethyl group, a 2-(2,3-dimethylphenyloxy)ethyl group, a 2-(2,4-dimethylphenyloxy)ethyl group, a 2-(2,5-dimethylphenyloxy)ethyl group, a 2-(2,6-dimethylphenyloxy)ethyl group, a 2-(3,4-dimethylphenyloxy)ethyl group, a 2-(3,5-dimethylphenyloxy)ethyl group, a 2-(2-methoxyphenyloxy)ethyl group, a 2-(3-methoxyphenyloxy)ethyl group, a 2-(4-methoxyphenyloxy)ethyl group, a 2-(2,3-dimethoxyphenyloxy)ethyl group, a 2-(2,4-dimethoxyphenyloxy)ethyl group, a 2-(2,5-dimethoxyphenyl)ethyl group, a 2-(2,6-dimethoxyphenyloxy)ethyl group, a 2-(3,4-dimethoxyphenyloxy)ethyl group, a 2-(3,5-dimethoxyphenyloxy)ethyl group, a 2-(2-ethylphenyloxy)ethyl group, a 2-(3-ethylphenyloxy)ethyl group, a 2-(4-ethylphenyloxy)ethyl group, a 2-(2-(trifluoromethyl)phenyloxy)ethyl group, a 2-(3-(trifluoromethyl)phenyloxy)ethyl group, a 2-(4-(trifluoromethyl)phenyloxy)ethyl group, a 2-(2-methylthiophenyloxy)ethyl group, a 2-(3-methylthiophenyloxy)ethyl group, a 2-(4-methylthiophenyloxy)ethyl group, a 2-(2-(trifluoromethoxy)phenyloxy)ethyl group, a 2-(3-(trifluoromethoxy)phenyloxy)ethyl group, a 2-(4-(trifluoromethoxy)phenyloxy)ethyl group, a 2-(2-nitrophenyloxy)ethyl group, a 2-(3-nitrophenyloxy)ethyl group, a 2-(4-nitrophenyloxy)ethyl group, a 2-(2-cyanophenyloxy)ethyl group, a 2-(3-cyanophenyloxy)ethyl group, a 2-(4-cyanophenyloxy)ethyl group, a 3-phenyloxypropyl group, any of groups represented by the following formulas:

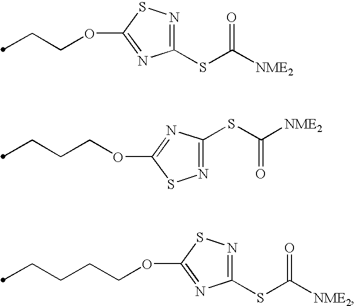

a 2-benzyloxyethyl group, a 2-(2-fluorobenzyloxy)ethyl group, a 2-(3-fluorobenzyloxy)ethyl group, a 2-(4-fluorobenzyloxy)ethyl group, a 2-(2,3-difluorobenzyloxy)ethyl group, a 2-(2,4-difluorobenzyloxy)ethyl group, a 2-(2,5-difluorobenzyloxy)ethyl group, a 2-(2,6-difluorobenzyloxy)ethyl group, a 2-(3,4-difluorobenzyloxy)ethyl group, a 2-(3,5-difluorobenzyloxy)ethyl group, a 2-(2-chlorobenzyloxy)ethyl group, a 2-(3-chlorobenzyloxy)ethyl group, a 2-(4-chlorobenzyloxy)ethyl group, a 2-(2,3-dichlorobenzyloxy)ethyl group, a 2-(2,4-dichlorobenzyloxy)ethyl group, a 2-(2,5-dichlorobenzyloxy)ethyl group, a 2-(2,6-dichlorobenzyloxy)ethyl group, a 2-(3,4-dichlorobenzyloxy)ethyl group, a 2-(3,5-dichlorobenzyloxy)ethyl group, a 2-(2-bromobenzyloxy)ethyl group, a 2-(3-bromobenzyloxy)ethyl group, a 2-(4-bromobenzyloxy)ethyl group, a 2-(2,3-dibromobenzyloxy)ethyl group, a 2-(2,4-dibromobenzyloxy)ethyl group, a 2-(2,5-dibromobenzyloxy)ethyl group, a 2-(2,6-dibromobenzyloxy)ethyl group, a 2-(3,4-dibromobenzyloxy)ethyl group, a 2-(3,5-dibromobenzyloxy)ethyl group, a 2-(2-iodobenzyloxy)ethyl group, a 2-(3-iodobenzyloxy)ethyl group, a 2-(4-iodobenzyloxy)ethyl group, a 2-(2-methylbenzyloxy)ethyl group, a 2-(3-methylbenzyloxy)ethyl group, a 2-(4-methylbenzyloxy)ethyl group, a 2-(2-(trifluoromethyl)benzyloxy)ethyl group, a 2-(3-(trifluoromethyl)benzyloxy)ethyl group, a 2-(4-(trifluoromethyl)benzyloxy)ethyl group, a 2-(2-methoxybenzyloxy)ethyl group, a 2-(3-methoxybenzyloxy)ethyl group, a 2-(4-methoxybenzyloxy)ethyl group, a 2-(2,5-dimethoxybenzyloxy)ethyl group, a 2-(3,5-dimethoxybenzyloxy)ethyl group, a 2-(2-methylthiobenzyloxy)ethyl group, a 2-(3-methylthiobenzyloxy)ethyl group, a 2-(4-methylthiobenzyloxy)ethyl group, a 2-(2-(trifluoromethoxy)benzyloxy)ethyl group, a 2-(3-(trifluoromethoxy)benzyloxy)ethyl group, a 2-(4-(trifluoromethoxy)benzyloxy)ethyl group, a 2-(2-nitrobenzyloxy)ethyl group, a 2-(3-nitrobenzyloxy)ethyl group, a 2-(4-nitrobenzyloxy)ethyl group, a 2-(2-cyanobenzyloxy)ethyl group, a 2-(3-cyanobenzyloxy)ethyl group, a 2-(4-cyanobenzyloxy)ethyl group, a 2-(2-ethoxybenzyloxy)ethyl group, a 2-(3-ethoxybenzyloxy)ethyl group, a 2-(4-ethoxybenzyloxy)ethyl group, a 2-(4-isopropylbenzyloxy)ethyl group, a 2-(4-tert-butylbenzyloxy)ethyl group, a 2-(2-fluoro-4-(trifluoromethyl)benzyloxy)ethyl group, a 2-(2-fluoro-5-(trifluoromethyl)benzyloxy)ethyl group, a 2-(4-fluoro-3-(trifluoromethyl)benzyloxy)ethyl group, a 2-(2,4-bis(trifluoromethyl)benzyloxy)ethyl group, a 2-(5-fluoro-2-methylbenzyloxy)ethyl group, a 2-(pentafluorobenzyloxy)ethyl group, or a 3-benzyloxypropyl group.

Hereinafter, Examples of the present compound will be shown.

### Example 1

In 2 ml of tetrahydrofuran was dissolved 224 mg of a compound represented by the formula (IIa-1):

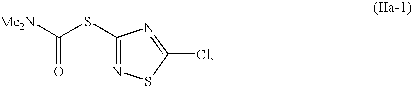

232 mg of a 28% methanol solution of sodium methoxide was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 163 mg of a compound represented by the formula (1):

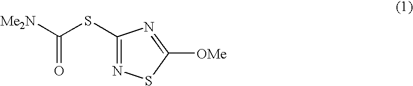

(hereinafter, referred to as present compound (1)).

¹H-NMR (CDCl₃, TMS) δ (ppm): 4.22 (3H, s), 3.04 (6H, br)

### Example 2

In 2 ml of tetrahydrofuran was dissolved 224 mg of the compound represented by the formula (IIa-1), 410 mg of a 20% ethanol solution of sodium ethoxide was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 183 mg of a compound represented by the formula (2):

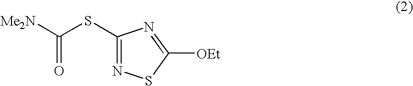

(hereinafter, referred to as present compound (2)).

¹H-NMR (CDCl₃, TMS) δ (ppm): 4.59 (2H, q), 3.04 (6H, br), 1.47 (3H, t)

### Example 3

In 4 ml of tetrahydrofuran were dissolved 450 mg of the compound represented by the formula (IIa-1), and 133 mg of 1-propanol, 90 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel column chromatography to obtain 370 mg of a compound represented by the formula (3):

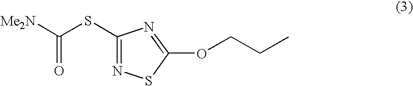

(hereinafter, referred to as present compound (3)).

¹H-NMR (CDCl₃, TMS) δ (ppm): 4.48 (2H, t), 3.04 (6H, br), 1.86 (2H, m), 1.03 (3H, t)

### Example 4

In 2 ml of tetrahydrofuran were dissolved 224 mg of the compound represented by the formula (IIa-1) and 72 mg of 2-propanol, 50 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 205 mg of a compound represented by the formula (4):

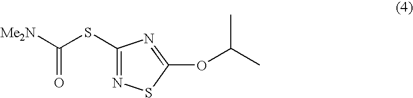

(hereinafter, referred to as present compound (4)).

¹H-NMR (CDCl₃, TMS) δ (ppm): 5.27 (1H, m), 3.04 (6H, br), 1.45 (6H, d)

### Example 5

According to the same manner as that of Example 4 except that 90 mg of 1-butanol was used in place of 2-propanol, 217 mg of a compound represented by the formula (5):

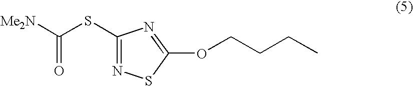

(hereinafter, referred to as present compound (5)) was obtained.

¹H-NMR (CDCl₃, TMS) δ (ppm): 4.52 (2H, t), 3.04 (6H, br), 1.81 (2H, m), 1.47 (2H, m), 0.97 (3H, t)

### Example 6

According to the same manner as that of Example 4 except that 86 mg of cyclopropylmethanol was used in place of 2-propanol, 230 mg of a compound represented by the formula (6):

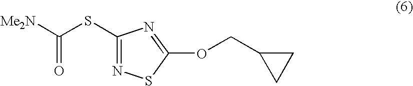

(hereinafter, referred to as present compound (6)) was obtained.

¹H-NMR (CDCl₃, TMS) δ (ppm): 4.36 (2H, d), 3.04 (6H, br), 1.34 (1H, m), 0.67 (2H, m), 0.40 (2H, m)

### Example 7

According to the same manner as that of Example 4 except that 67 mg of 2-propyne-1-ol was used in place of 2-propanol, 139 mg of a compound represented by the formula (7):

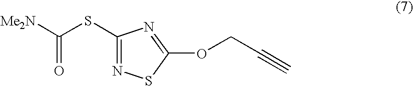

(hereinafter, referred to as present compound (7)) was obtained.

¹H-NMR (CDCl₃, TMS) δ (ppm): 5.15 (2H, d), 3.04 (6H, br), 2.66 (1H, t)

### Example 8

According to the same manner as that of Example 4 except that 84 mg of 2-butyne-1-ol was used in place of 2-propanol, 174 mg of a compound represented by the formula (8):

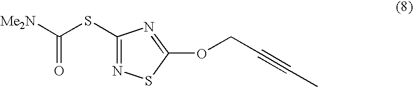

(hereinafter, referred to as present compound (8)) was obtained.

¹H-NMR (CDCl₃, TMS) δ (ppm): 5.10 (2H, q), 3.04 (6H, br), 1.90 (3H, t)

### Example 9

According to the same manner as that of Example 4 except that 101 mg of 2-pentyne-1-ol was used in place of 2-propanol, 220 mg of a compound represented by the formula (9):

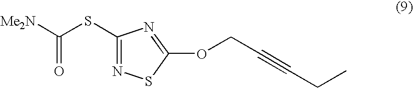

(hereinafter, referred to as present compound (9)) was obtained.

¹H-NMR (CDCl₃, TMS) δ (ppm): 5.12 (2H, t), 3.04 (6H, br), 2.27 (2H, tq), 1.16 (3H, t)

### Example 10

According to the same manner as that of Example 4 except that 91 mg of 2-methoxyethanol was used in place of 2-propanol, 165 mg of a compound represented by the formula (10):

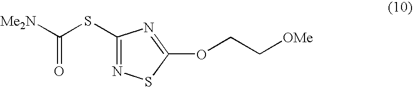

(hereinafter, referred to as present compound (10)) was obtained.

¹H-NMR (CDCl₃, TMS) δ (ppm): 4.69 (2H, m), 3.76 (2H, m), 3.42 (3H, s), 3.04 (6H, br)

### Example 11

According to the same manner as that of Example 4 except that 123 mg of tetrahydro-3-furanmethanol was used in place of 2-propanol, 136 mg of a compound represented by the formula (11):

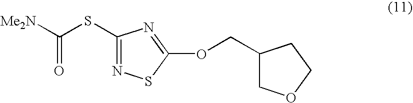

(hereinafter, referred to as present compound (11)) was obtained.

¹H-NMR (CDCl₃, TMS) δ (ppm): 4.48 (2H, m), 3.88 (2H, m), 3.77 (1H, m), 3.67 (1H, m), 3.04 (6H, br), 2.80 (1H, m), 2.11 (1H, m), 1.71 (1H, m)

### Example 12

According to the same manner as that of Example 4 except that 140 mg of tetrahydropyran-2-methanol was used in place of 2-propanol, 158 mg of a compound represented by the formula (12):

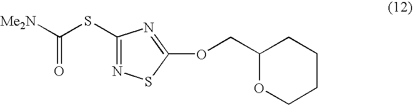

(hereinafter, referred to as present compound (12)) was obtained.

¹H-NMR (CDCl₃, TMS) δ (ppm): 4.54 (1H, dd), 4.45 (1H, dd), 4.04 (1H, m), 3.73 (1H, m), 3.49 (1H, m), 3.04 (6H, br), 1.90 (1H, m), 1.64-1.52 (3H, m), 1.40 (1H, m)

### Example 13

In 2 ml of tetrahydrofuran was dissolved 230 mg of 2,2-dimethyl-1,3-dioxolane-4 methanol, 70 mg of sodium hydride (60% oily) was added under ice-cooling, the mixture was stirred for 5 minutes, a solution in which 260 mg of the compound represented by the formula (IIa-1) had been dissolved in 2 ml of tetrahydrofuran was added, and the mixture was stirred at room temperature for 2 hours. Thereafter, an aqueous saturated ammonium chloride was added to the reaction mixture, followed by extraction with t-butyl methyl ether. The organic layer was dried with sodium sulfate, and concentrated under reduced pressure. The residue was subjected to silica gel preparative thin layer chromatography to obtain 140 mg of a compound represented by the formula (13):

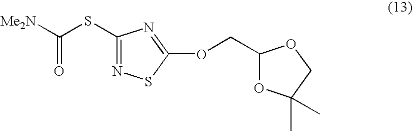

(hereinafter, referred to as present compound (13)).

¹H-NMR (CDCl₃, TMS) δ (ppm): 4.60 (1H, m), 4.52 (2H, m), 4.14 (1H, m), 3.83 (1H, m), 3.04 (6H, br), 1.45 (3H, s), 1.39 (3H, s)

### Example 14 and Example 15

In 2 ml of tetrahydrofuran were dissolved 224 mg of the compound represented by the formula (IIa-1) and 125 mg of glycerol formal, 50 mg of sodium hydride was added under ice-cooling, and the mixture was stirred at room temperature for 2 hours. Thereafter, an aqueous saturated ammonium chloride was added to the reaction mixture, followed by extraction with t-butyl methyl ether. The organic layer was dried with sodium sulfate, and concentrated under reduced pressure. The residue was subjected to medium pressure preparative liquid chromatography to obtain 67 mg of a compound represented by the formula (14):

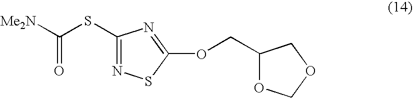

(hereinafter, referred to as present compound (14)) and 74 mg of a compound represented by the formula (15):

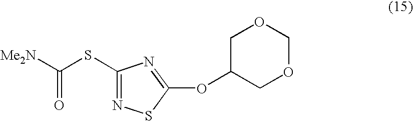

(hereinafter, referred to as present compound (15).

### Present Compound (14)

¹H-NMR (CDCl₃, TMS) δ (ppm): 5.07 (1H, s), 4.93 (1H, s), 4.59 (2H, m), 4.47 (1H, m), 4.04 (1H, dd), 3.79 (1H, dd), 3.04 (6H, br)

### Present Compound (15)

¹H-NMR (CDCl₃, TMS) δ (ppm): 5.06 (1H, m), 5.03 (1H, d), 4.83 (1H, d), 4.26 (2H, dd), 4.08 (2H, dd), 3.04 (6H, br)

### Example 16

According to the same manner as that of Example 4 except that 89mg of glycidol was used in place of 2-propanol, 44 mg of a compound represented by the formula (16):

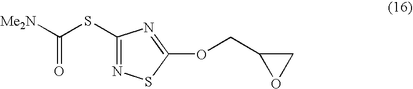

(hereinafter, referred to as present compound (16)) was obtained.

¹H-NMR (CDCl₃, TMS) δ (ppm): 4.87 (1H, dd), 4.38 (1H, dd), 3.41 (1H, m), 3.04 (6H, br), 2.92 (1H, dd), 2.73 (1H, dd)

### Example 17

According to the same manner as that of Example 4 except that 123 mg of tetrahydro-4-pyranol was used in place of 2-propanol, 156 mg of a compound represented by the formula (17):

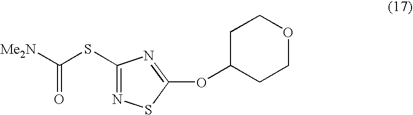

(hereinafter, referred to as present compound (17)) was obtained.

¹H-NMR (CDCl₃, TMS) δ (ppm): 5.26 (1H, m), 3.96 (2H, m), 3.59 (2H, m), 3.04 (6H, br), 2.15 (2H, m), 1.90 (2H, m)

### Example 18

According to the same manner as that of Example 4 except that 172 mg of 2-chloro-5-hydroxymethylpyridine was used in place of 2-propanol, 161 mg of a compound represented by the formula (18):

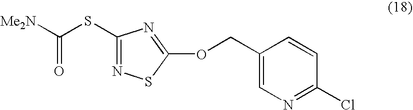

(hereinafter, referred to as present compound (18)) was obtained.

¹H-NMR (CDCl₃, TMS) δ (ppm): 8.50 (1H, d), 7.80 (1H, dd), 7.39 (1H, d), 5.56 (2H, s), 3.06 (6H, br)

### Example 19

According to the same manner as that of Example 4 except that 151 mg of 1H-pyrazole-1-propanol was used in place of 2-propanol, 151 mg of a compound represented by the formula (19):

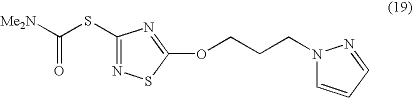

(hereinafter, referred to as present compound (19)) was obtained.

¹H-NMR (CDCl₃, TMS) δ (ppm): 7.52 (1H, d), 7.40 (1H, d), 6.24 (1H, t), 4.51 (2H, t), 4.31 (2H, t), 3.04 (6H, br), 2.41 (2H, m)

### Example 20

According to the same manner as that of Example 4 except that 180 mg of 2-chloro-5-(hydroxymethyl)thiazole was used in place of 2-propanol, 84 mg of a compound represented by the formula (20):

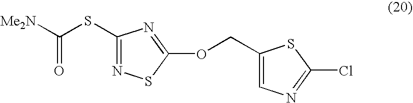

(hereinafter, referred to as present compound (20)) was obtained.

¹H-NMR (CDCl₃, TMS) δ (ppm): 7.67 (1H, s), 5.66 (2H, s), 3.07 (6H, br)

### Example 21

According to the same manner as that of Example 4 except that 120 mg of 2,2,2-trifluoroethanol was used in place of 2-propanol, 199 mg of a compound represented by the formula (21):

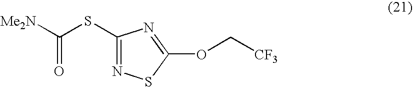

(hereinafter, referred to as present compound (21)) was obtained.

¹H-NMR (CDCl₃, TMS) δ (ppm): 4.91 (2H, q, J=8Hz), 3.05 (6H, br)

### Example 22

In 2 ml of tetrahydrofuran were dissolved 336 mg of the compound represented by the formula (IIa-1) and 120 mg of 3-butene-1-ol, 67 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel column chromatography to obtain 315 mg of a compound represented by the formula (22):

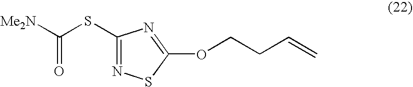

(hereinafter, referred to as present compound (22)).

¹H-NMR (CDCl₃, TMS) δ (ppm): 5.83 (1H, m), 5.20-5.11 (2H, m), 4.58 (2H, t), 3.04 (6H, br), 2.59 (2H, m)

### Example 23

In 2 ml of tetrahydrofuran were dissolved 336 mg of the compound represented by the formula (IIa-1) and 120 mg of 3-butyne-1-ol, 67 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The resulting solid was washed with toluene, and dried under reduced pressure to obtain 234 mg of a compound represented by the formula (23):

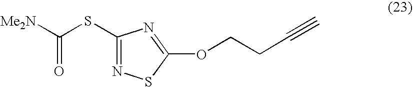

(hereinafter, referred to as present compound (23)).

¹H-NMR (CDCl₃, TMS) δ (ppm): 4.64 (2H, t), 3.04 (6H, br), 2.75 (2H, td), 2.05 (1H, t)

### Example 24

According to the same manner as that of Example 22 except that 150 mg of 3-methoxy-1-propanol was used in place of 3-butene-1-ol, 355 mg of a compound represented by the formula (24):

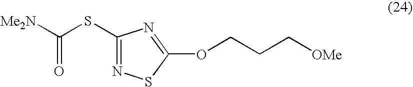

(hereinafter, referred to as present compound (24)) was obtained.

¹H-NMR (CDCl₃, TMS) δ (ppm): 4.61 (2H, t), 3.51 (2H, t), 3.34 (3H, s), 3.04 (6H, br), 2.09 (2H, m)

### Example 25

In 5 ml of tetrahydrofuran were dissolved 850 mg of the compound represented by the formula (IIa-1) and 607 mg of 5,5-dimethyl-1,3-dioxane-2-ethanol, 167 mg of sodium hydride (60% oily) was added under ice-cooling, and the mixture was stirred at room temperature for 2 hours. Thereafter, an aqueous saturated ammonium chloride was added to the reaction mixture, followed by extraction with t-butyl methyl ether. The organic layer was dried with sodium sulfate, and concentrated under reduced pressure. The residue was subjected to silica gel column chromatography to obtain 410mg of a compound represented by the formula (25):

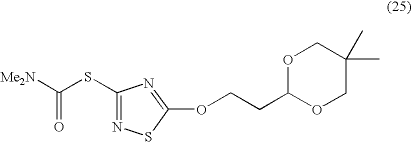

(hereinafter, referred to as present compound (25)).

¹H-NMR (CDCl₃, TMS) δ (ppm): 4.66-4.61 (3H, m), 3.60 (2H, d, J=11), 3.43 (2H, d, J=11 Hz), 3.04 (6H, br), 2.16 (2H, td), 1.18 (3H, s), 0.73 (3H, s)

### Example 26

In 2 ml of tetrahydrofuran were dissolved 340 mg of the compound represented by the formula (IIa-1) and 150 mg of 2-(methylthio)ethanol, 70 mg of sodium hydride (60% oily) and 0.5 ml of tetrahydrofuran were added under ice-cooling, and the mixture was stirred at room temperature for 4 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 66 mg of a compound represented by the formula (26):

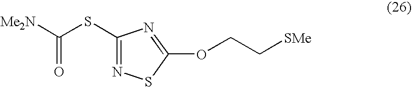

(hereinafter, referred to as present compound (26)).

¹H-NMR (CDCl₃, TMS) δ (ppm): 4.69 (2H, t), 3.05 (6H, br), 2.92 (2H, t), 2.19 (3H, s)

### Example 27

In 1 ml of tetrahydrofuran was dissolved 0.18 mg of 3-methyl-2-pentanol, 80 mg of sodium hydride (60% oily) was added, and the mixture was stirred at 30° C. for 1 hour. To the solution was added 2 ml of a tetrahydrofuran solution (0.5 M) of the compound represented by the formula (IIa-1), and the mixture was stirred at 30° C. for 2 hours. Thereafter, dilute hydrochloric acid was added to the reaction mixture, followed by extraction with ethyl acetate. The organic layer was washed with water, dried with magnesium sulfate, centrifuged and concentrated. The residue was subjected to medium pressure preparative liquid chromatography to obtain 167 mg of a compound represented by the formula (27):

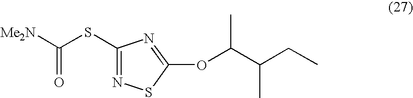

(hereinafter, referred to as present compound (27)).

¹H-NMR (CDCl₃, TMS) δ (ppm): 5.04 (1H, m), 3.04 (6H, br), 1.86-1.68 (1H, m), 1.58-1.47 (1H, m), 1.38 (3H, dd), 1.25-1.15 (1H, m), 0.99-0.91 (6H, m)

### Example 28

According to the same manner as that of Example 27 except that 0.18 g of 2-methyl-1-butanol was used in place of 3-methyl-2-pentanol, 63 mg of a compound represented by the formula (28):

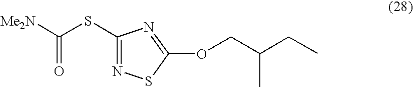

(hereinafter, referred to as present compound (28)) was obtained.

¹H-NMR (CDCl₃, TMS) δ (ppm): 4.34 (2H, ddd), 3.04 (6H, br), 1.92 (1H, m), 1.53 (1H, m), 1.27 (1H, m), 1.00 (3H, d), 0.94 (3H, t)

### Example 29

According to the same manner as that of Example 27 except that 0.18 g of 3-pentanol was used in place of 3-methyl-2-pentanol, 56 mg of a compound represented by the formula (29):

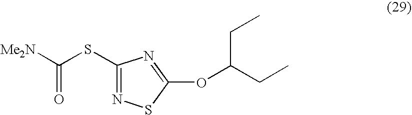

(hereinafter, referred to as present compound (29)) was obtained.

¹H-NMR (CDCl₃, TMS) δ (ppm): 4.98 (1H, quint), 3.04 (6H, br), 1.83-1.75 (4H, m), 0.96 (6H, t)

### Example 30

According to the same manner as that of Example 27 except that 0.18 g of 3-methyl-2-butanol was used in place of 3-methyl-2-pentanol, 125 mg of a compound represented by the formula (30):

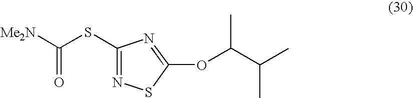

(hereinafter, referred to as present compound (30)) was obtained.

¹H-NMR (CDCl₃, TMS) δ (ppm): 4.96 (1H, m), 3.04 (6H, br), 2.01 (1H, m), 1.38 (3H, d), 0.99 (3H, d), 0.97 (3H, d)

### Example 31

According to the same manner as that of Example 27 except that 0.18 g of 2,2-dimethyl-1-propanol was used in place of 3-methyl-2-pentanol, 64 mg of a compound represented by the formula (31):

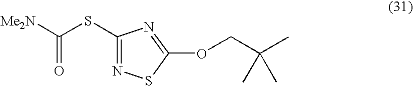

(hereinafter, referred to as present compound (31)) was obtained.

¹H-NMR (CDCl₃, TMS) δ (ppm): 4.18 (2H, s), 3.04 (6H, br), 1.03 (9H, s)

### Example 32

According to the same manner as that of Example 27 except that 0.23 g of 1-heptanol was used in place of 3-methyl-2-pentanol, 22 mg of a compound represented by the formula (32):

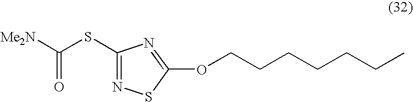

(hereinafter, referred to as present compound (32)) was obtained.

¹H-NMR (CDCl₃, TMS) δ (ppm): 4.50 (2H, t), 3.04 (6H, br), 1.86-1.79 (2H, m), 1.46-1.24 (8H, m), 0.89 (3H, t)

### Example 33

According to the same manner as that of Example 27 except that 0.20 g of 3,3-dimethyl-1-butanol was used in place of 3-methyl-2-pentanol, 44 mg of a compound represented by the formula (33):

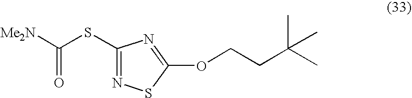

(hereinafter, referred to as present compound (33)) was obtained.

¹H-NMR (CDCl₃, TMS) δ (ppm): 4.57 (2H, t), 3.04 (6H, br), 1.77 (2H, t), 0.98 (9H, s)

### Example 34

According to the same manner as that of Example 27 except that 0.17 g of 4-pentene-1-ol was used in place of 3-methyl-2-pentanol, 54 mg of a compound represented by the formula (34):

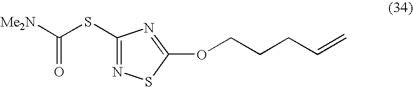

(hereinafter, referred to as present compound (34)) was obtained.

¹H-NMR (CDCl₃, TMS) δ (ppm): 5.81 (1H, m), 5.09-5.00 (2H, m), 4.53 (2H, t), 3.04 (6H, br), 2.21 (2H, m), 1.93 (2H, m)

### Example 35

According to the same manner as that of Example 27 except that 0.14 g of 3-butene-2-ol was used in place of 3-methyl-2-pentanol, 22 mg of a compound represented by the formula (35):

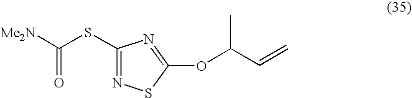

(hereinafter, referred to as present compound (35)) was obtained.

¹H-NMR (CDCl₃, TMS) δ (ppm): 5.96 (1H, m), 5.54 (1H, m), 5.40(1H, d, J=17 Hz), 5.27 (1H, d, J=10 Hz), 3.04 (6H, br), 1.53 (3H, d)

### Example 36

According to the same manner as that of Example 27 except that 0.14 g of 2-methyl-2-propene-1-ol was used in place of 3-methyl-2-pentanol, 53 mg of a compound represented by the formula (36):

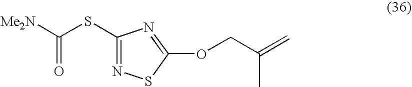

(hereinafter, referred to as present compound (36)) was obtained.

¹H-NMR (CDCl₃, TMS) δ (ppm): 5.13 (1H, br s), 5.06 (1H, br s), 4.93 (2H, s), 3.04 (6H, br), 1.84 (3H, s)

### Example 37

According to the same manner as that of Example 27 except that 0.17g of 1-pentyne-3-ol was used in place of 3-methyl-2-pentanol, 49 mg of a compound represented by the formula (37):

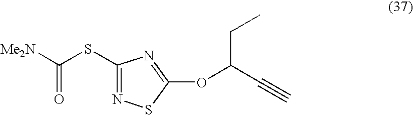

(hereinafter, referred to as present compound (37)) was obtained.

¹H-NMR (CDCl₃, TMS) δ (ppm): 5.55 (1H, td), 3.04 (6H, br), 2.61 (1H, d), 2.02 (2H, dq), 1.10 (3H, t)

### Example 38

According to the same manner as that of Example 27 except that 0.17 g of 4-pentyne-2-ol was used in place of 3-methyl-2-pentanol, 54 mg of a compound represented by the formula (38):

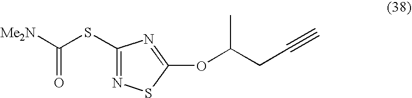

(hereinafter, referred to as present compound (38)) was obtained.

¹H-NMR (CDCl₃, TMS) δ (ppm): 5.28 (1H, m), 3.04 (6H, br), 2.70 (2H, dd), 2.06 (1H, t), 1.56 (3H, d)

### Example 39

According to the same manner as that of Example 27 except that 0.14 g of 3-butyne-2-ol was used in place of 3-methyl-2-pentanol, 34 mg of a compound represented by the formula (39):

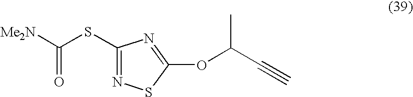

(hereinafter, referred to as present compound (39)) was obtained.

¹H-NMR (CDCl₃, TMS) δ (ppm): 5.70 (1H, dq), 3.04 (6H, br), 2.62 (1H, d), 1.72 (3H, d)

### Example 40

In 1 ml of tetrahydrofuran was dissolved 0.19 g of tetrahydro-3-furanol, 50mg of sodium hydride (60% oily) was added, and the mixture was stirred at 30° C. for 1 hour. To the solution was added 2 ml of a tetrahydrofuran solution (0.5 M) of the compound represented by the formula (IIa-1), and the mixture was stirred at 30° C. for 2 hours. Thereafter, dilute hydrochloric acid was added to the reaction mixture, followed by extraction with ethyl acetate. The organic layer was washed with water, dried with magnesium sulfate, centrifuged, and concentrated. The residue was subjected to medium pressure preparative liquid chromatography to obtain 90 mg of a compound represented by the formula (40):

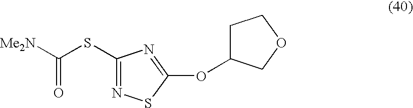

(hereinafter, referred to as present compound (40)).

¹H-NMR (CDCl₃, TMS) δ (ppm): 5.66-5.63 (1H, m), 4.10-3.88 (4H, m), 3.04 (6H, br), 2.32-2.27 (2H, m)

### Example 41

According to the same manner as that of Example 40 except that 0.20 g of tetrahydrofurfuryl alcohol was used in place of tetrahydro-3-furanol, 108 mg of a compound represented by the formula (41):

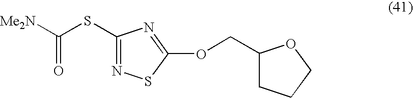

(hereinafter, referred to as present compound (41)) was obtained.

¹H-NMR (CDCl₃, TMS) δ (ppm): 4.60 (1H, dd), 4.45 (1H, dd), 4.30 (1H, m), 3.94-3.81 (2H, m), 3.04 (6H, br), 2.07 (1H, m), 1.94 (2H, m), 1.69 (1H, m)

### Example 42

According to the same manner as that of Example 40 except that 0.19 g of cyclopentanol alcohol was used in place of tetrahydro-3-furanol, 192 mg of a compound represented by the formula (42):

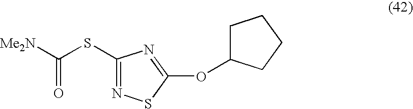

(hereinafter, referred to as present compound (42)) was obtained.

¹H-NMR (CDCl₃, TMS) δ (ppm): 5.40 (1H, m), 3.03 (6H, br), 1.97 (4H, m), 1.83-1.63 (4H, m)

### Example 43

According to the same manner as that of Example 40 except that 0.20 g of cyclohexanol alcohol was used in place of tetrahydro-3-furanol, 212 mg of a compound represented by the formula (43):

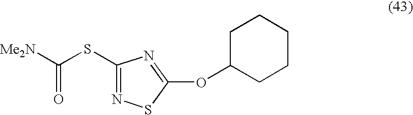

(hereinafter, referred to as present compound (43)) was obtained.

¹H-NMR (CDCl₃, TMS) δ (ppm): 5.03 (1H, m), 3.05 (6H, br), 2.08-1.24 (10H, m)

### Example 44

According to the same manner as that of Example 40 except that 0.19 g of 1-methylcyclopropanemethanol alcohol was used in place of tetrahydro-3-furanol, 130 mg of a compound represented by the formula (44):

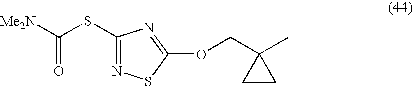

(hereinafter, referred to as present compound (44)) was obtained.

¹H-NMR (CDCl₃, TMS) δ (ppm): 4.30 (2H, s), 3.03 (6H, br), 1.22 (3H, s), 0.59 (2H, m), 0.47 (2H, m)

### Example 45

According to the same manner as that of Example 40 except that 0.19 g of cyclobutanemethanol alcohol was used in place of tetrahydro-3-furanol, 146 mg of a compound represented by the formula (45):

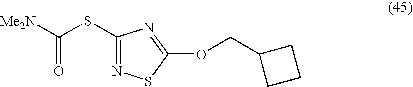

(hereinafter, referred to as present compound (45)) was obtained.

¹H-NMR (CDCl₃, TMS) δ (ppm): 4.49 (2H, d), 3.04 (6H, br), 2.81 (1H, m), 2.17-2.09 (2H, m), 2.01-1.81 (4H, m)

### Example 46

According to the same manner as that of Example 40 except that 0.23 g of 1-cyclopentylethanol alcohol was used in place of tetrahydro-3-furanol, 124 mg of a compound represented by the formula (46):

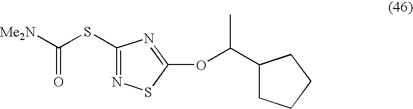

(hereinafter, referred to as present compound (46)) was obtained.

¹H-NMR (CDCl₃, TMS) δ (ppm): 5.00 (1H, m), 3.04 (6H, br), 2.18 (1H, m), 1.87-1.52 (6H, m), 1.43 (3H, d), 1.40-1.22 (2H, m)

### Example 47

According to the same manner as that of Example 40 except that 0.26 g of 1-cyclohexylethanol alcohol was used in place of tetrahydro-3-furanol, 154 mg of a compound represented by the formula (47):

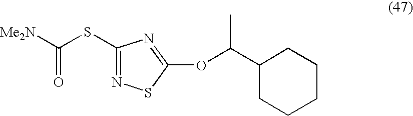

(hereinafter, referred to as present compound (47)) was obtained.

¹H-NMR (CDCl₃, TMS) δ (ppm): 4.94 (1H, m), 3.04 (6H, br), 1.86-1.61 (6H, m), 1.39 (3H, d), 1.31-1.00 (4H, m)

### Example 48

In 1 ml of tetrahydrofuran was dissolved 0.27 g of 2-chlorocyclohexanol, to the solution was added 2 ml of a tetrahydrofuran solution (0.5 M) of the compound represented by the formula (IIa-1), 50 mg of sodium hydride (60% oily) was added, and the mixture was stirred at 30° C. for 2 hours. Thereafter, dilute hydrochloric acid was added to the reaction mixture, followed by extraction with ethyl acetate. The organic layer was washed with water, dried with magnesium sulfate, centrifuged, and concentrated. The residue was subjected to medium pressure preparative liquid chromatography to obtain 240 mg of a compound represented by the formula (48):

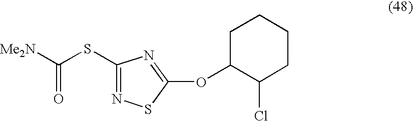

(hereinafter, referred to as present compound (48)).

¹H-NMR (CDCl₃, TMS) δ (ppm): 5.06 (1H, td), 4.06 (1H, m), 3.04 (6H, br), 2.41 (1H, m), 2.27 (1H, m), 1.82-1.72 (3H, m), 1.64-1.34 (3H, m)

### Example 49

According to the same manner as that of Example 48 except that 0.10 g of 3-chloro-1-propanol was used in place of 2-chlorocyclohexanol, 153 mg of a compound represented by the formula (49):

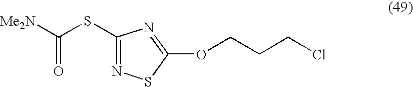

(hereinafter, referred to as present compound (49)) was obtained.

¹H-NMR (CDCl₃, TMS) δ (ppm): 4.69 (2H, t), 3.69 (2H, t), 3.04 (6H, br), 2.30 (2H ,m)

### Example 50

According to the same manner as that of Example 48 except that 0.11 g of 4-chloro-1-butanol was used in place of 2-chlorocyclohexanol, 78 mg of a compound represented by the formula (50):

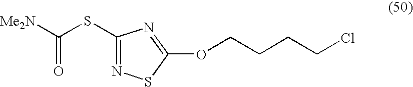

(hereinafter, referred to as present compound (50)) was obtained.

¹H-NMR (CDCl₃, TMS) δ (ppm): 4.57 (2H, t), 3.60 (2H, t), 3.04 (6H, br), 2.05-1.90 (4H, m)

### Example 51

According to the same manner as that of Example 48 except that 0.14 g of 6-chloro-1-hexanol was used in place of 2-chlorocyclohexanol, 167 mg of a compound represented by the formula (51):

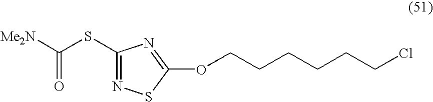

(hereinafter, referred to as present compound (51)) was obtained.

¹H-NMR (CDCl₃, TMS) δ (ppm): 4.52 (2H, t), 3.54 (2H, t), 3.04 (6H, br), 1.88-1.76 (4H, m), 1.53-1.44 (4H, m)

### Example 52

According to the same manner as that of Example 48 except that 0.11 g of 3-chloro-2,2-dimethyl-1-propanol was used in place of 2-chlorocyclohexanol, 186 mg of a compound represented by the formula (52):

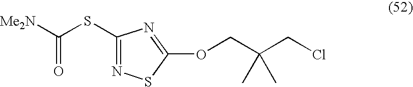

(hereinafter, referred to as present compound (52)) was obtained.

¹H-NMR (CDCl₃, TMS) δ (ppm): 4.36 (2H, s), 3.50 (2H, s), 3.04 (6H, br), 1.11 (6H, s)

### Example 53

According to the same manner as that of Example 48 except that 0.12 g of 2,2-dichloroethanol was used in place of 2-chlorocyclohexanol, 198 mg of a compound represented by the formula (53):

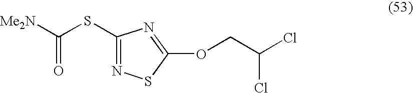

(hereinafter, referred to as present compound (53)) was obtained.

¹H-NMR (CDCl₃, TMS) δ (ppm): 6.05 (1H, t), 4.87 (2H, d), 3.05(6H, br)

### Example 54

According to the same manner as that of Example 48 except that 0.13 g of 2,3-dichloro-1-propanol was used in place of 2-chlorocyclohexanol, 203 mg of a compound represented by the formula (54):

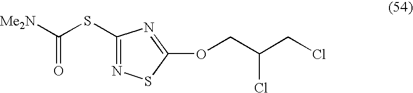

(hereinafter, referred to as present compound (54)) was obtained.

¹H-NMR (CDCl₃, TMS) δ (ppm): 4.85 (2H, m) , 4.42 (1H, m) 3.86 (2H, m), 3.05 (6H, br)

### Example 55

According to the same manner as that of Example 40 except that 0.10 g of 2-fluoroethanol was used in place of tetrahydro-3-furanol, 141 mg of a compound represented by the formula (55):

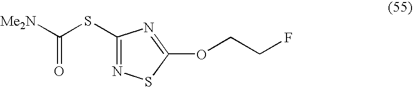

(hereinafter, referred to as present compound (55)) was obtained.

¹H-NMR (CDCl₃, TMS) δ (ppm): 4.82 (2H, s), 4.76-4.69 (2H, m), 3.04 (6H, br)

### Example 56

According to the same manner as that of Example 40 except that 0.10 g of 2,2-difluoroethanol was used in place of tetrahydro-3-furanol, 181 mg of a compound represented by the formula (56):

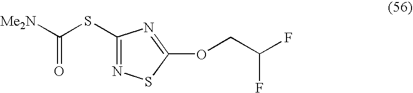

(hereinafter, referred to as present compound (56)) was obtained.

¹H-NMR (CDCl₃, TMS) δ (ppm): 6.14 (1H, tt, J=55 Hz, 4 Hz), 4.73 (2H, td, J=13 Hz, 4 Hz), 3.05 (6H, br)

### Example 57

According to the same manner as that of Example 40 except that 0.10 g of 1,3-difluoro-2-propanol was used in place of tetrahydro-3-furanol, 94 mg of a compound represented by the formula (57):

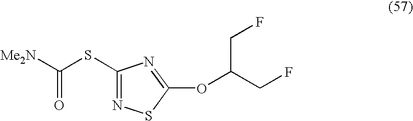

(hereinafter, referred to as present compound (57)) was obtained.

¹H-NMR (CDCl₃, TMS) δ (ppm): 5.50 (1H, m), 4.91-4.68 (4H, m), 3.05 (6H, br)

### Example 58

In 1 ml of tetrahydrofuran was dissolved 0.17 g of 1,1,1,3,3,3-hexafluoro-2-propanol, 50 mg of sodium hydride (60% oily) was added, and the mixture was stirred at 30° C. for 1 hour. After the solution was heated with a hot-air blower for a few minutes, 2 ml of a tetrahydrofuran solution (0.5 M) of the compound represented by the formula (IIa-1) was added, and the mixture was stirred at 30° C. for 2 hours. Thereafter, dilute hydrochloric acid was added to the reaction mixture, followed by extraction with magnesium sulfate. The organic layer was washed with water, dried with sodium sulfate, centrifuged, and concentrated. The residue was subjected to medium pressure preparative chromatography to obtain 49 mg of a compound represented by the formula (58):

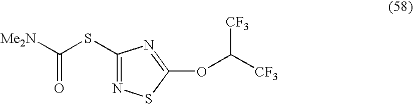

(hereinafter, referred to as present compound (58)).

¹H-NMR (CDCl₃, TMS) δ (ppm): 6.13 (1H, m), 3.06 (6H, br)

### Example 59

According to the same manner as that of Example 58 except that 0.15 g of 2,2,3,3,3-pentafluoro-1-propanol was used in place of 1,1,1,3,3,3-hexafluoro-2-propanol, 126 mg of a compound represented by the formula (59):

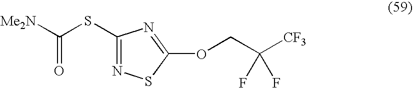

(hereinafter, referred to as present compound (59)) was obtained.

¹H-NMR (CDCl₃, TMS) δ (ppm): 4.98 (2H, t, J=12 Hz), 3.05 (6H, br)

### Example 60

According to the same manner as that of Example 40 except that 0.17 g of 2,2,3,4,4-pentafluoro-3-butene-1-ol was used in place of tetrahydro-3-furanol, 72 mg of a compound represented by the formula (60):

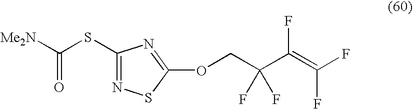

(hereinafter, referred to as present compound (60)) was obtained.

¹H-NMR (CDCl₃, TMS) δ (ppm): 4.93 (2H, td, J=12 Hz, 2 Hz), 3.05 (6H, br)

### Example 61

According to the same manner as that of Example 58 except that 0.17 g of 3,3,4,4,4-pentafluoro-2-butanol was used in place of 1,1,1,3,3,3-hexafluoro-2-propanol, 101 mg of a compound represented by the formula (61):

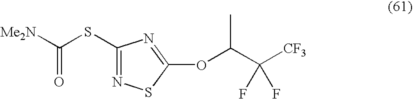

(hereinafter, referred to as present compound (61)) was obtained.

¹H-NMR (CDCl₃, TMS) δ (ppm): 5.70 (1H, m), 3.05 (6H, br), 1.65 (3H, d)

### Example 62

According to the same manner as that of Example 58 except that 0.19 g of 2,2,3,3,4,4,4-pentafluoro-1-butanol was used in place of 1,1,1,3,3,3-hexafluoro-2-propanol, 154 mg of a compound represented by the formula (62):

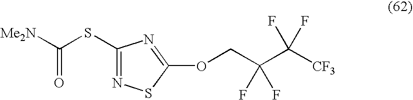

(hereinafter, referred to as present compound (62)) was obtained.

¹H-NMR (CDCl₃, TMS) δ (ppm): 5.02 (2H, t, J=13 Hz), 3.05 (6H, br)

### Example 63

According to the same manner as that of Example 40 except that 0.13 g of 1-ethynyl-1-hexanol was used in place of tetrahydro-3-furanol, 56 mg of a compound represented by the formula (63):

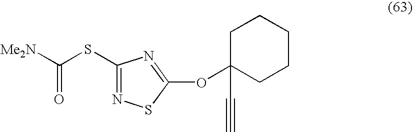

(hereinafter, referred to as present compound (63)) was obtained.

¹H-NMR (CDCl₃, TMS) δ (ppm): 3.04 (6H, br), 2.82 (1H, s), 2.28-2.21 (2H, m), 2.07-2.00 (2H, m), 1.81-1.72 (2H, m), 1.69-1.51 (3H, m), 1.44-1.32 (1H, m)

### Example 64

According to the same manner as that of Example 58 except that 0.12 g of 2,2-dimethyl-3-pentanol was used in place of 1,1,1,3,3,3-hexafluoro-2-propanol, 100 mg of a compound represented by the formula (64):

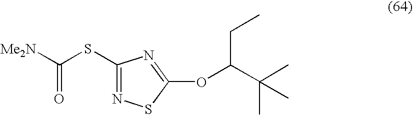

(hereinafter, referred to as present compound (64)) was obtained.

¹H-NMR (CDCl₃, TMS) δ (ppm): 4.87 (1H, dd), 3.04 (6H, br), 1.81-1.65 (2H, m), 1.00-0.96 (12H, m)

### Example 65

According to the same manner as that of Example 48 except that 0.15 g of 3-cyclohexyl-1-propanol was used in place of 2-chlorocyclohexanol, 260 mg of a compound represented by the formula (65):

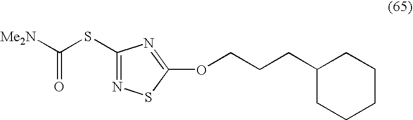

(hereinafter, referred to as present compound (65)) was obtained.

¹H-NMR (CDCl₃, TMS) δ (ppm): 4.49 (2H, t), 3.03 (6H, br), 1.86-1.77 (2H, m), 1.75-1.52 (3H, m), 1.34-1.08 (8H, m), 0.95-0.80 (2H, m)

### Example 66

According to the same manner as that of Example 48 except that 0.12 g of 2-cyclopentanethanol was used in place of 2-chlorocyclohexanol, 226 mg of a compound represented by the formula (66):

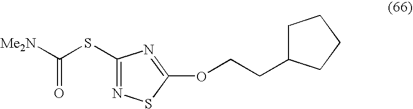

(hereinafter, referred to as present compound (66)) was obtained.

¹H-NMR (CDCl₃, TMS) δ (ppm): 4.53 (2H, t), 3.04 (6H, br), 1.96-1.78 (5H, m), 1.67-1.50 (4H, m), 1.19-1.09 (2H, m)

### Example 67

According to the same manner as that of Example 48 except that 0.10 g of 2-chloroethanol was used in place of 2-chlorocyclohexanol, 100 mg of a compound represented by the formula (67):

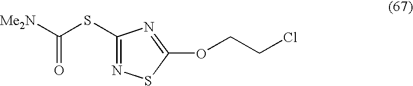

(hereinafter, referred to as present compound (67)) was obtained.

¹H-NMR (CDCl₃, TMS) δ (ppm): 4.78 (2H, t), 3.86 (2H, t), 3.04 (6H, br)

### Example 68

According to the same manner as that of Example 48 except that 0.10 g of 1-chloro-2-propanol was used in place of 2-chlorocyclohexanol, 173 mg of a compound represented by the formula (68):

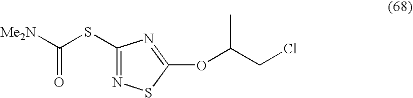

(hereinafter, referred to as present compound (68)) was obtained.

¹H-NMR (CDCl₃, TMS) δ (ppm): 5.39 (1H, m), 3.81 (1H, dd), 3.74 (1H, dd), 3.04 (6H, br), 1.55 (3H, d)

### Example 69

According to the same manner as that of Example 48 except that 0.10 g of 3-furanmethanol was used in place of 2-chlorocyclohexanol, 120 mg of a compound represented by the formula (69):

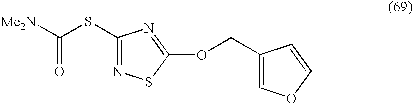

(hereinafter, referred to as present compound (69)) was obtained.

¹H-NMR (CDCl₃, TMS) δ (ppm): 7.61 (1H, d), 7.44 (1H, t), 6.53 (1H, d), 5.44 (2H, s), 3.06 (6H, br)

### Example 70

According to the same manner as that of Example 48 except that 0.12 g of 3-cyclohexene-methanol was used in place of 2-chlorocyclohexanol, 175 mg of a compound represented by the formula (70):

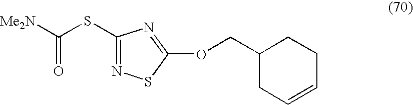

(hereinafter, referred to as present compound (70)) was obtained.

¹H-NMR (CDCl₃, TMS) δ (ppm): 5.69 (2H, m), 4.42 (2H, d), 3.05 (6H, br), 2.24-2.05 (4H, m), 1.91-1.76 (2H, m), 1.45-1.35 (1H, m)

### Example 71

According to the same manner as that of Example 48 except that 0.12 g of cyclohexylmethanol was used in place of 2-chlorocyclohexanol, 56 mg of a compound represented by the formula (71):

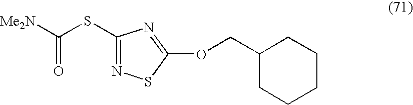

(hereinafter, referred to as present compound (71)) was obtained.

¹H-NMR (CDCl₃, TMS) δ (ppm): 4.31 (2H, d), 3.04 (6H, br), 1.90-1.65 (7H, m), 1.33-1.12 (4H, m)

### Example 72

In 1 ml of tetrahydrofuran was dissolved 0.10 g of furfuryl alcohol, to the solution was added 2 ml of a tetrahydrofuran solution (0.5 M) of the compound represented by the formula (IIa-1), 50 mg of sodium hydride (60% oily) was added, and the mixture was stirred at 30° C. for 2 hours. Thereafter, dilute hydrochloric acid was added to the reaction mixture, followed by extraction with ethyl acetate. The organic layer was washed with water, dried with magnesium sulfate, centrifuged, and concentrated to obtain 49 mg of the crude product of a compound represented by the formula (72):

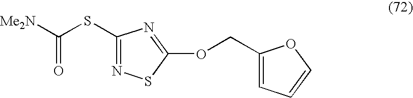

(hereinafter, referred to as present compound (72)).

¹H-NMR (CDCl₃, TMS) δ (ppm): 7.48 (1H, dd), 6.58 (1H, d), 6.41 (1H, dd), 5.51 (2H, s), 3.06 (6H, br)

### Example 73

In 1 ml of tetrahydrofuran was dissolved 0.12 g of 2-thiophenemethanol, to the solution was added 2 ml of a tetrahydrofuran solution (0.5M) of the compound represented by the formula (IIa-1), 50 mg of sodium hydride (60% oily) was added, and the mixture was stirred at 30° C. for 2 hours. Thereafter, dilute hydrochloric acid was added to the reaction mixture, followed by extraction with ethyl acetate. The organic layer was washed with water, dried with magnesium sulfate, centrifuged, and concentrated to obtain 164 mg of the crude product of a compound represented by the formula (73):

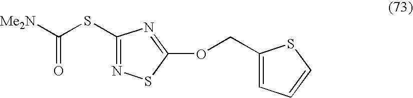

(hereinafter, referred to as present compound (73)).

¹H-NMR (CDCl₃, TMS) δ (ppm): 7.40 (1H, d), 7.23 (1H, m), 7.03 (1H, dd), 5.70 (2H, s), 3.06 (6H, br)

### Example 74

In 1 ml of tetrahydrofuran was dissolved 0.23 g of 3-thiophenemethanol, 50 mg of sodium hydride (60% oily) was added, and the mixture was stirred at 30° C. for 1 hour. To the solution was added 2 ml of a tetrahydrofuran solution (0.5 M) of the compound represented by the formula (IIa-1), and the mixture was stirred at 30° C. for 2 hours. Thereafter, dilute hydrochloric acid was added to the reaction mixture, followed by extraction with ethyl acetate. The organic layer was washed with water, dried with magnesium sulfate, centrifuged, and concentrated. The residue was subjected to medium pressure preparative liquid chromatography to obtain 67 mg of a compound represented by the formula (74):

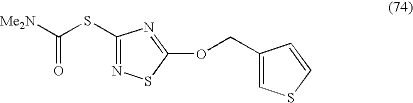

(hereinafter, referred to as present compound (74)).

¹H-NMR (CDCl₃, TMS) δ (ppm): 7.46 (1H, m), 7.36 (1H, dd), 7.18 (1H, dd), 5.55 (2H, s), 3.05 (6H, br)

### Example 75

According to the same manner as that of Example 58 except that 0.10 g of 2-methyl-3-butyne-2-ol was used in place of 1,1,1,3,3,3-hexafluoro-2-propanol, 87 mg of a compound represented by the formula (75):

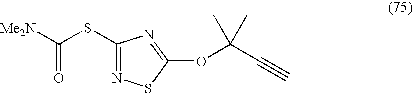

(hereinafter, referred to as present compound (75)) was obtained.

¹H-NMR (CDCl₃, TMS) δ (ppm): 3.04 (6H, br), 2.76 (1H, s), 1.86 (6H, s)

### Example 76

According to the same manner as that of Example 48 except that 0.12 g of cycloheptanol was used in place of 2-chlorocyclohexanol, 42 mg of a compound represented by the formula (76):

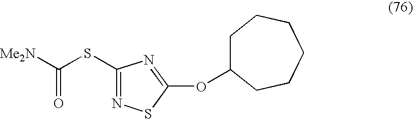

(hereinafter, referred to as present compound (76)) was obtained.

¹H-NMR (CDCl₃, TMS) δ (ppm): 5.19 (1H, m), 3.05 (6H, br), 2.15-2.07 (2H, m), 1.94-1.85 (2H, m), 1.76-1.42 (8H, m)

### Example 77

According to the same manner as that of Example 48 except that 0.13 g of cyclooctanol was used in place of 2-chlorocyclohexanol, 132 mg of a compound represented by the formula (77):

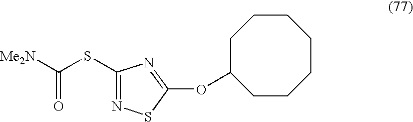

(hereinafter, referred to as present compound (77)) was obtained.

¹H-NMR (CDCl₃, TMS) δ (ppm): 5.15 (1H, m), 3.04 (6H, br), 2.07-1.93 (4H, m), 1.87-1.43 (10H, m)

### Example 78

After 2 ml of a tetrahydrofuran solution (0.5 M) of the compound represented by the formula (IIa-1) was added to 0.13 g of 2-fluorobenzyl alcohol, 50 mg of sodium hydride (60% oily) was added, and the mixture was stirred at 25° C. for 2 hours. Thereafter, a mixed solution of hexane-ethyl acetate was added to the reaction mixture, the resulting insolubles were filtered, and the filtrate was subjected to medium pressure preparative liquid chromatography to obtain 198 mg of a compound represented by the formula (78):

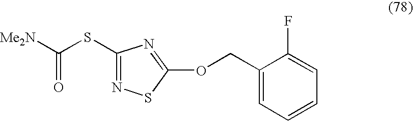

(hereinafter, referred to as present compound (78)).

¹H-NMR (CDCl₃, TMS) δ (ppm): 7.49 (1H, td), 7.39 (1H, m), 7.18 (1H, t), 7.12 (1H, t), 5.62 (2H, s), 3.06 (6H, br)

### Example 79

According to the same manner as that of Example 78 except that 0.13 g of 3-fluorobenzyl alcohol was used in place of 2-fluorobenzyl alcohol, 203 mg of a compound represented by the formula (79):

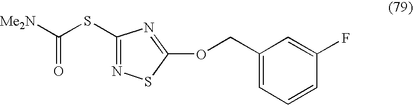

(hereinafter, referred to as present compound (79)) was obtained.

¹H-NMR (CDCl₃, TMS) δ (ppm): 7.38 (1H, td), 7.22 (1H, d), 7.16 (1H, dt), 7.08 (1H, td), 5.53 (2H, s), 3.06 (6H, br)

### Example 80

According to the same manner as that of Example 78 except that 0.13 g of 4-fluorobenzyl alcohol was used in place of 2-fluorobenzyl alcohol, 156 mg of a compound represented by the formula (80):

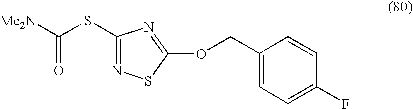

(hereinafter, referred to as present compound (80)) was obtained.

¹H-NMR (CDCl₃, TMS) δ (ppm): 7.47-7.42 (2H, m), 7.12-7.06 (2H, m), 5.51 (2H, s), 3.06 (6H, br)

### Example 81

According to the same manner as that of Example 78 except that 0.15 g of 2-chlorobenzyl alcohol was used in place of 2-fluorobenzyl alcohol, 214 mg of a compound represented by the formula (81):

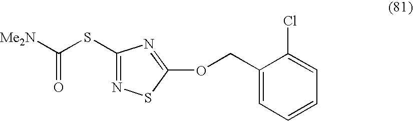

(hereinafter, referred to as present compound (81)) was obtained.

¹H-NMR (CDCl₃, TMS) δ (ppm): 7.53 (1H, m), 7.43 (1H, m), 7.36-7.29 (2H, m), 5.66 (2H, s), 3.06 (6H, br)

### Example 82

According to the same manner as that of Example 78 except that 0.15 g of 3-chlorobenzyl alcohol was used in place of 2-fluorobenzyl alcohol, 205 mg of a compound represented by the formula (82):

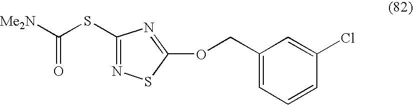

(hereinafter, referred to as present compound (82)) was obtained.

¹H-NMR (CDCl₃, TMS) δ (ppm): 7.45 (1H, br), 7.37-7.32 (3H, m), 5.52 (2H, s), 3.06. (6H, br)

### Example 83

According to the same manner as that of Example 78 except that 0.15 g of 4-chlorobenzyl alcohol was used in place of 2-fluorobenzyl alcohol, 198 mg of a compound represented by the formula (83):

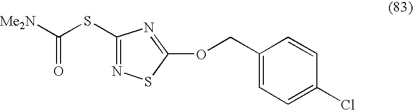

(hereinafter, referred to as present compound (83)) was obtained.

¹H-NMR (CDCl₃, TMS) δ (ppm): 7.38 (4H, br), 5.51 (2H, s), 3.05 (6H, br)

### Example 84

According to the same manner as that of Example 78 except that 0.19 g of 2-bromobenzyl alcohol was used in place of 2-fluorobenzyl alcohol, 208 mg of a compound represented by the formula (84):

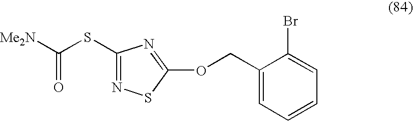

(hereinafter, referred to as present compound (84)) was obtained.

¹H-NMR (CDCl₃, TMS) δ (ppm): 7.62 (1H, d), 7.52 (1H, d), 7.36 (1H, t), 7.25 (1H, t), 5.64 (2H, s), 3.06 (6H, br)

### Example 85

According to the same manner as that of Example 78 except that 0.19 g of 3-bromobenzyl alcohol was used in place of 2-fluorobenzyl alcohol, 203 mg of a compound represented by the formula (85):

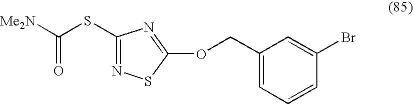

(hereinafter, referred to as present compound (85)) was obtained.

¹H-NMR (CDCl₃, TMS) δ (ppm): 7.60 (1H, s), 7.52 (1H, d), 7.37 (1H, d), 7.28 (1H, t), 5.51 (2H, s), 3.06 (6H, br)

### Example 86

According to the same manner as that of Example 78 except that 0.19 g of 4-bromobenzyl alcohol was used in place of 2-fluorobenzyl alcohol, 186 mg of a compound represented by the formula (86):

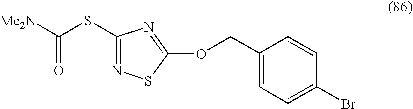

(hereinafter, referred to as present compound (86)) was obtained.

¹H-NMR (CDCl₃, TMS) δ (ppm): 7.56-7.52 (2H, m), 7.34-7.31 (2H, m), 5.50 (2H, s), 3.06 (6H, br)

### Example 87

According to the same manner as that of Example 78 except that 0.24 g of 2-iodobenzyl alcohol was used in place of 2-fluorobenzyl alcohol, 253 mg of a compound represented by the formula (87):

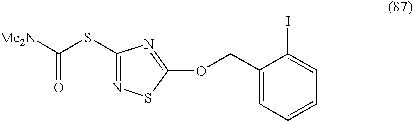

(hereinafter, referred to as present compound (87)) was obtained.

¹H-NMR (CDCl₃, TMS) δ (ppm): 7.81 (1H, m), 7.72 (1H, m), 7.41 (1H, d), 7.14 (1H, t), 5.48 (2H, s), 3.06 (6H, br)

### Example 88

According to the same manner as that of Example 78 except that 0.14 g of 4-ethylbenzyl alcohol was used in place of 2-fluorobenzyl alcohol, 185 mg of a compound represented by the formula (88):

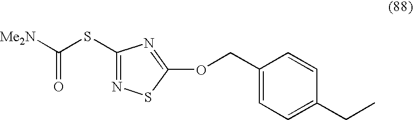

(hereinafter, referred to as present compound (88)) was obtained.

¹H-NMR (CDCl₃, TMS) δ (ppm): 7.37 (2H, d), 7.24 (2H, d), 5.50 (2H, s), 3.06 (6H, br), 2.67 (2H, q), 1.24 (3H, t)

### Example 89

According to the same manner as that of Example 78 except that 0.13 g of 3-methylbenzyl alcohol was used in place of 2-fluorobenzyl alcohol, 188 mg of a compound represented by the formula (89):

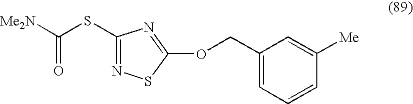

(hereinafter, referred to as present compound (89)) was obtained.

¹H-NMR (CDCl₃, TMS) δ (ppm): 7.32-7.19 (4H, m), 5.50 (2H, s), 3.06 (6H, br), 2.38(3H, s)

### Example 90

According to the same manner as that of Example 78 except that 0.13 g of 4-methylbenzyl alcohol was used in place of 2-fluorobenzyl alcohol, 199 mg of a compound represented by the formula (90):

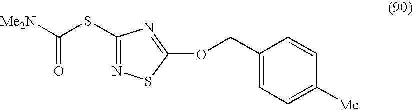

(hereinafter, referred to as present compound (90)) was obtained.

¹H-NMR (CDCl₃, TMS) δ (ppm): 7.34 (2H, d), 7.21 (2H, d), 5.49 (2H, s), 3.05 (6H, br), 2.37 (3H, s)

### Example 91

According to the same manner as that of Example 78 except that 0.14 g of 2-methoxybenzyl alcohol was used in place of 2-fluorobenzyl alcohol, 194 mg of a compound represented by the formula (91):

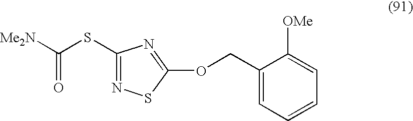

(hereinafter, referred to as present compound (91)) was obtained.

¹H-NMR (CDCl₃, TMS) δ (ppm): 7.43-7.35 (2H, m), 7.00-6.92 (2H, m), 5.58 (2H, s), 3.85 (3H, s), 3.06 (6H, br)

### Example 92

According to the same manner as that of Example 78 except that 0.14 g of 3-methoxybenzyl alcohol was used in place of 2-fluorobenzyl alcohol, 194 mg of a compound represented by the formula (92):

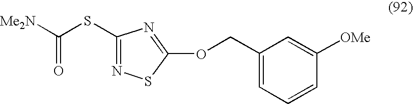

(hereinafter, referred to as present compound (92)) was obtained.

¹H-NMR (CDCl₃, TMS) δ (ppm): 7.32 (1H, t), 7.02 (1H, d), 6.99 (1H, s), 6.93 (1H, d), 5.51 (2H, s), 3.83 (3H, s), 3.05 (6H, br)

### Example 93

According to the same manner as that of Example 78 except that 0.14 g of 4-methoxybenzyl alcohol was used in place of 2-fluorobenzyl alcohol, 163 mg of a compound represented by the formula (93):

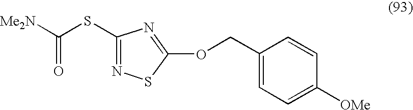

(hereinafter, referred to as present compound (93)) was obtained.

¹H-NMR (CDCl₃, TMS) δ (ppm): 7.39 (2H, d), 6.93 (2H, d), 5.47 (2H, s), 3.82 (3H, s), 3.05 (6H, br)

### Example 94

According to the same manner as that of Example 78 except that 0.16 g of 2-ethoxybenzyl alcohol was used in place of 2-fluorobenzyl alcohol, 73 mg of a compound represented by the formula (94):

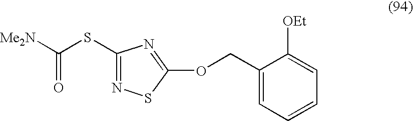

(hereinafter, referred to as present compound (94)) was obtained.

¹H-NMR (CDCl₃, TMS) δ (ppm): 7.40 (1H, d), 7.34 (1H, t), 6.96 (1H, t), 6.90 (1H, d), 5.60 (2H, s), 4.07 (2H, q), 3.05 (6H, br), 1.38 (3H, t)

### Example 95

According to the same manner as that of Example 78 except that 0.16 g of 4-ethoxybenzyl alcohol was used in place of 2-fluorobenzyl alcohol, 172 mg of a compound represented by the formula (95):

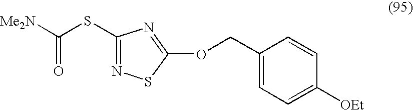

(hereinafter, referred to as present compound (95)) was obtained.

¹H-NMR (CDCl₃, TMS) δ (ppm): 7.37 (2H, d), 6.91 (2H, d), 5.46 (2H, s), 4.05 (2H, q), 3.06 (6H, br), 1.42 (3H, t)

### Example 96

According to the same manner as that of Example 78 except that 0.15 g of 4-isopropylbenzyl alcohol was used in place of 2-fluorobenzyl alcohol, 230 mg of a compound represented by the formula (96):

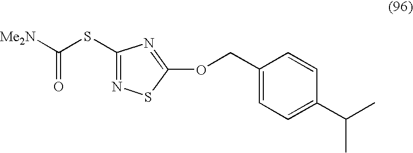

(hereinafter, referred to as present compound (96)) was obtained.

¹H-NMR (CDCl₃, TMS) δ (ppm): 7.38 (2H, d), 7.27 (2H, d), 5.50 (2H, s), 3.05 (6H, br), 2.93 (1H, m), 1.26 (6H, d)

### Example 97

According to the same manner as that of Example 78 except that 0.16 g of 4-(methylthio)benzyl alcohol was used in place of 2-fluorobenzyl alcohol, 164 mg of a compound represented by the formula (97):

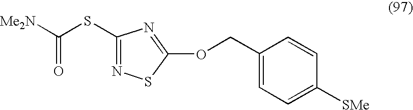

(hereinafter, referred to as present compound (97)) was obtained.

¹H-NMR (CDCl₃, TMS) δ (ppm): 7.37 (2H, d), 7.27 (2H, d), 5.49 (2H, s), 3.06 (6H, br), 2.49 (3H, s)

### Example 98

According to the same manner as that of Example 78 except that 0.17 g of 4-tert-butylbenzyl alcohol was used in place of 2-fluorobenzyl alcohol, 238 mg of a compound represented by the formula (98):

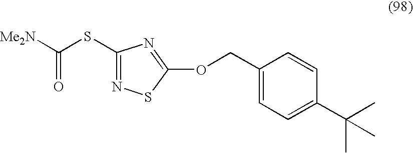

(hereinafter, referred to as present compound (98)) was obtained.

¹H-NMR (CDCl₃, TMS) δ (ppm): 7.45-7.38 (4H, m), 5.51 (2H, s), 3.05 (6H, br), 1.33 (9H, s)

### Example 99

According to the same manner as that of Example 78 except that 0.18 g of 2,3-dichlorobenzyl alcohol was used in place of 2-fluorobenzyl alcohol, 269 mg of a compound represented by the formula (99):

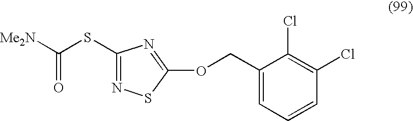

(hereinafter, referred to as present compound (99)) was obtained.

¹H-NMR (CDCl₃, TMS) δ (ppm): 7.51-7.44 (2H, m), 7.29-7.24 (1H, m), 5.67 (2H, s), 3.05 (6H, br)

### Example 100

According to the same manner as that of Example 78 except that 0.18 g of 2,4-dichlorobenzyl alcohol was used in place of 2-fluorobenzyl alcohol, 241 mg of a compound represented by the formula (100):

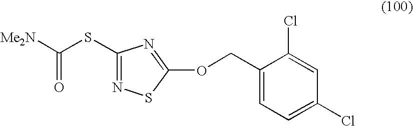

(hereinafter, referred to as present compound (100)) was obtained. ¹H-NMR (CDCl₃, TMS) δ (ppm): 7.49-7.43 (2H, m), 7.30 (1H, m), 5.62 (2H, s), 3.05 (6H, br)

### Example 101

According to the same manner as that of Example 78 except that 0.18 g of 2,5-dichlorobenzyl alcohol was used in place of 2-fluorobenzyl alcohol, 61 mg of a compound represented by the formula (101):

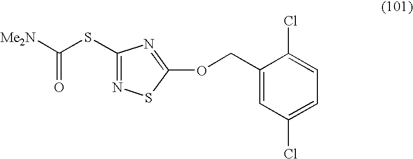

(hereinafter, referred to as present compound (101)) was obtained.

¹H-NMR (CDCl₃, TMS) δ (ppm): 7.54 (1H, d), 7.36 (1H, d), 7.30 (1H, dd), 5.62 (2H, s), 3.06 (6H, br)

### Example 102

According to the same manner as that of Example 78 except that 0.18 g of 2,6-dichlorobenzyl alcohol was used in place of 2-fluorobenzyl alcohol, 239 mg of a compound represented by the formula (102):

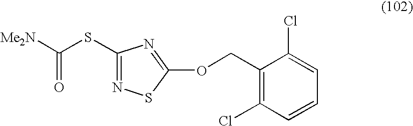

(hereinafter, referred to as present compound (102)) was obtained.

¹H-NMR (CDCl₃, TMS) δ (ppm): 7.40-7.36 (2H, m), 7.32-7.24 (1H, m), 5.83 (2H, s), 3.06 (6H, br)

### Example 103

According to the same manner as that of Example 78 except that 0.18 g of 3,4-dichlorobenzyl alcohol was used in place of 2-fluorobenzyl alcohol, 202 mg of a compound represented by the formula (103):

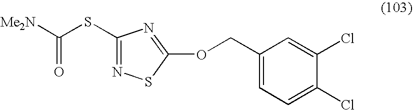

(hereinafter, referred to as present compound (103)) was obtained.

¹H-NMR (CDCl₃, TMS) δ (ppm): 7.56 (1H, m), 7.48 (1H, m), 7.29 (1H, m), 5.49 (2H, s), 3.05 (6H, br)

### Example 104

According to the same manner as that of Example 78 except that 0.18g of 3,5-dichlorobenzyl alcohol was used in place of 2-fluorobenzyl alcohol, 148 mg of a compound represented by the formula (104):

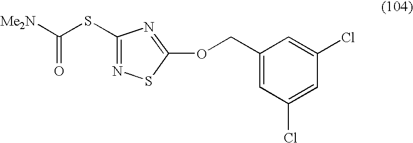

(hereinafter, referred to as present compound (104)) was obtained.

¹H-NMR (CDCl₃, TMS) δ (ppm): 7.37-7.33 (3H, m), 5.49 (2H, s), 3.05 (6H, br)

### Example 105

According to the same manner as that of Example 78 except that 0.15 g of 2,5-difluorobenzyl alcohol was used in place of 2-fluorobenzyl alcohol, 199 mg of a compound represented by the formula (105):

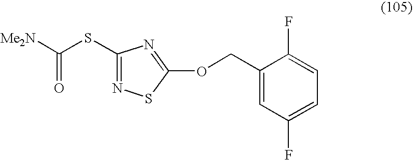

(hereinafter, referred to as present compound (105)) was obtained.

¹H-NMR (CDCl₃, TMS) δ (ppm): 7.23 (1H, m), 7.11-7.04 (2H, m), 5.59 (2H, s), 3.05 (6H, br)

### Example 106

According to the same manner as that of Example 78 except that 0.15 g of 2,6-difluorobenzyl alcohol was used in place of 2-fluorobenzyl alcohol, 206 mg of a compound represented by the formula (106):

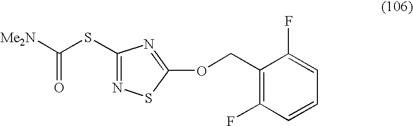

(hereinafter, referred to as present compound (106)) was obtained.

¹H-NMR (CDCl₃, TMS) δ (ppm): 7.39 (1H, m), 6.99-6.94 (2H, m), 5.64(2H, s), 3.05 (6H, br)

### Example 107

According to the same manner as that of Example 78 except that 0.15 g of 3,4-difluorobenzyl alcohol was used in place of 2-fluorobenzyl alcohol, 147 mg of a compound represented by the formula (107):

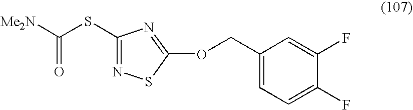

(hereinafter, referred to as present compound (107)) was obtained.

¹H-NMR (CDCl₃, TMS) δ (ppm): 7.30 (1H, m), 7.21-7.18 (2H, m), 5.49 (2H, s), 3.05 (6H, br)

### Example 108

According to the same manner as that of Example 78 except that 0.15 g of 3,5-difluorobenzyl alcohol was used in place of 2-fluorobenzyl alcohol, 246 mg of a compound represented by the formula (108):

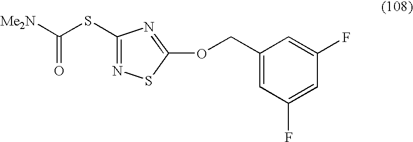

(hereinafter, referred to as present compound (108)) was obtained.

¹H-NMR (CDCl₃, TMS) δ (ppm): 6.98 (2H, m), 6.82 (1H, tt), 5.52 (2H, s), 3.06 (6H, br)

### Example 109

According to the same manner as that of Example 78 except that 0.20 g of 2-fluoro-4-(trifluoromethyl)benzyl alcohol was used in place of 2-fluorobenzyl alcohol, 280 mg of a compound represented by the formula (109):

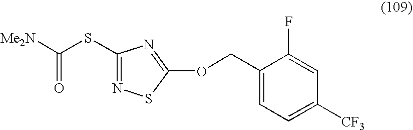

(hereinafter, referred to as present compound (109)) was obtained.

¹H-NMR (CDCl₃, TMS) δ (ppm): 7.66 (1H, m), 7.47 (1H, m), 7.39 (1H, m), 5.66 (2H, s), 3.05 (6H, br)

### Example 110

According to the same manner as that of Example 78 except that 0.20 g of 2-fluoro-5-(trifluoromethyl)benzyl alcohol was used in place of 2-fluorobenzyl alcohol, 119 mg of a compound represented by the formula (110):

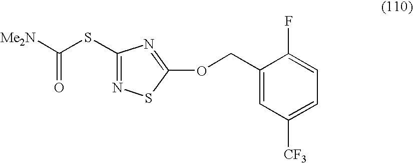

(hereinafter, referred to as present compound (110)) was obtained.

¹H-NMR (CDCl₃, TMS) δ (ppm): 7.83 (1H, m), 7.67 (1H, m), 7.24 (1H, t), 5.65 (2H, s), 3.06 (6H, br)

### Example 111

According to the same manner as that of Example 78 except that 0.20 g of 4-fluoro-3-(trifluoromethyl)benzyl alcohol was used in place of 2-fluorobenzyl alcohol, 233 mg of a compound represented by the formula (111):

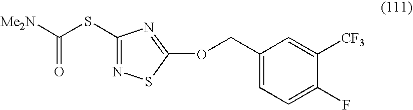

(hereinafter, referred to as present compound (111)) was obtained.

¹H-NMR (CDCl₃, TMS) δ (ppm): 7.72 (1H, m), 7.66 (1H, m), 7.25 (1H, m), 5.55 (2H, s), 3.06(6H, br)

### Example 112

According to the same manner as that of Example 78 except that 0.25 g of 2,4-bis(trifluoromethyl)benzyl alcohol was used in place of 2-fluorobenzyl alcohol, 243 mg of a compound represented by the formula (112):

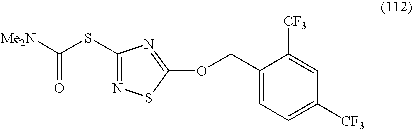

(hereinafter, referred to as present compound (112)) was obtained.

¹H-NMR (CDCl₃, TMS) δ (ppm): 7.97 (1H, s), 7.88 (2H, s), 5.80 (2H, s), 3.06 (6H, br)

### Example 113

According to the same manner as that of Example 78 except that 0.14 g of 2,4-dimethylbenzyl alcohol was used in place of 2-fluorobenzyl alcohol, 71 mg of a compound represented by the formula (113):

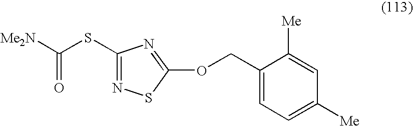

(hereinafter, referred to as present compound (113)) was obtained.

¹H-NMR (CDCl₃, TMS) δ (ppm): 7.28 (1H, d), 7.06-7.03 (2H, m), 5.52 (2H, s), 3.06 (6H, br), 2.36 (3H, s), 2.34 (3H, s)

### Example 114

According to the same manner as that of Example 78 except that 0.14 g of 3,4-dimethylbenzyl alcohol was used in place of 2-fluorobenzyl alcohol, 185 mg of a compound represented by the formula (114):

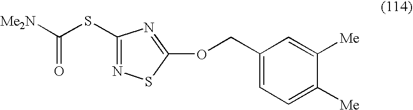

(hereinafter, referred to as present compound (114)) was obtained.

¹H-NMR (CDCl₃, TMS) δ (ppm): 7.22 (1H, s), 7.17 (2H, m), 5.47 (2H, s), 3.06 (6H, br), 2.28 (3H, s), 2.28(3H, s)

### Example 115

According to the same manner as that of Example 78 except that 0.17 g of 2,5-dimethoxybenzyl alcohol was used in place of 2-fluorobenzyl alcohol, 96 mg of a compound represented by the formula (115):

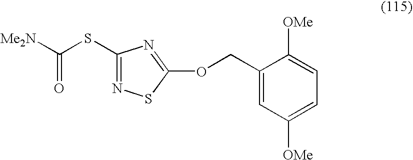

(hereinafter, referred to as present compound (115)) was obtained.

¹H-NMR (CDCl₃, TMS) δ (ppm): 7.00 (1H, s), 6.92-6.80 (2H, m), 5.56 (2H, s), 3.81 (3H, s), 3.78 (3H, s), 3.05 (6H, br)

### Example 116

According to the same manner as that of Example 78 except that 0.17 g of 3,5-dimethoxybenzyl alcohol was used in place of 2-fluorobenzyl alcohol, 134 mg of a compound represented by the formula (116):

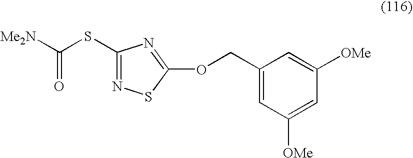

(hereinafter, referred to as present compound (116)) was obtained.

¹H-NMR (CDCl₃, TMS) δ (ppm): 6.59-6.39 (3H, m), 5.47 (2H, s), 3.80 (6H, s), 3.05 (6H, br)

### Example 117

According to the same manner as that of Example 78 except that 0.14 g of 5-fluoro-2-methylbenzyl alcohol was used in place of 2-fluorobenzyl alcohol, 177 mg of a compound represented by the formula (117):

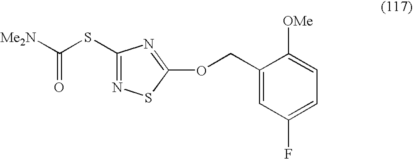

(hereinafter, referred to as present compound (117)) was obtained.

¹H-NMR (CDCl₃, TMS) δ (ppm): 7.20-7.13 (2H, m), 6.99 (1H, td), 5.52 (2H, s), 3.06 (6H, br), 2.34 (3H, s)

### Example 118

According to the same manner as that of Example 78 except that 0.20 g of pentafluorobenzyl alcohol was used in place of 2-fluorobenzyl alcohol, 123 mg of a compound represented by the formula (118):

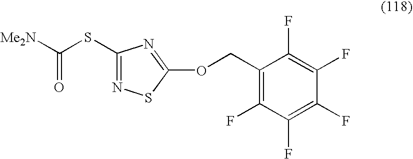

(hereinafter, referred to as present compound (118)) was obtained.

¹H-NMR (CDCl₃, TMS) δ (ppm): 5.64 (2H, s), 3.05 (6H, br)

### Example 119

In 1 ml of tetrahydrofuran was dissolved 90 mg of 2,2-dimethyl-1,3-dioxolane-4-methanol, 32 mg of sodium hydride (60% oily) was added under ice-cooling, the mixture was stirred for 5 minutes, a solution in which 180 mg of a compound represented by the formula (IIa-1):

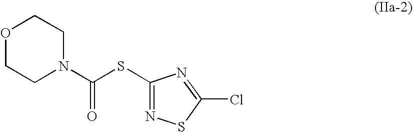

had been dissolved in 2 ml of tetrahydrofuran was added, and the mixture was stirred at room temperature for 2 hours. Thereafter, an aqueous saturated ammonium chloride solution was added to the reaction mixture, followed by extraction with ethyl acetate. The organic layer was dried with sodium sulfate, and concentrated under reduced pressure. The residue was subjected to silica gel preparative thin layer chromatography to obtain 73mg of a compound represented by the formula (119):

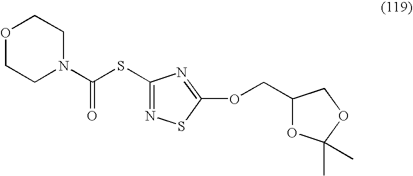

(hereinafter, referred to as present compound (119)).

¹H-NMR (CDCl₃, TMS) δ (ppm): 4.61-4.51 (3H, m), 4.14 (1H, m), 3.83 (1H, m), 3.72 (4H, t), 3.56 (4H, br), 1.45 (3H, s), 1.38 (3H, s)

### Example 120

In 1 ml of tetrahydrofuran was dissolved 81 mg of 1-propanol, 54 mg of sodium hydride (60% oily) was added under ice-cooling, the mixture was stirred for 5 minutes, a solution in which 300 mg of a compound represented by the formula (IIa-2) had been dissolved in 2 ml of tetrahydrofuran was added, and the mixture was stirred at room temperature for 2 hours. Thereafter, an aqueous saturated ammonium chloride solution was added to the reaction solution, followed by extraction with ethyl acetate. The organic layer was dried with sodium sulfate, and concentrated under reduced pressure. The residue was subjected to silica gel preparative thin layer chromatography to obtain 230 mg of a compound represented by the formula (120):

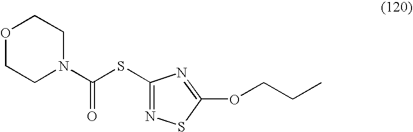

(hereinafter, referred to as present compound (120)).

¹H-NMR (CDCl₃, TMS) δ (ppm): 4.48 (2H, t), 3.72 (4H, t), 3.57 (4H, br), 1.87 (2H, m), 1.03 (3H, t)

### Example 121

In 2 ml of tetrahydrofuran was dissolved 266 mg of a compound represented by the formula (IIa-2), 230 mg of a 28% methanol solution of sodium methoxide was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 126mg of a compound represented by the formula (121):

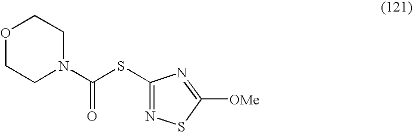

(hereinafter, referred to as present compound (121)).

¹H-NMR (CDCl₃, TMS) δ (ppm): 4.22 (3H, s), 3.73 (4H, t), 3.57 (4H, br)

### Example 122

In 2 ml of tetrahydrofuran was dissolved 266mg of a compound represented by the formula (IIa-2), 410 mg of a 20% methanol solution of sodium methoxide was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 182 mg of a compound represented by the formula (122):

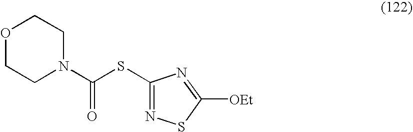

(hereinafter, referred to as present compound (122)).

¹H-NMR (CDCl₃, TMS) δ (ppm): 4.59 (2H, q), 3.72 (4H, t), 3.57 (4H, br), 1.48 (3H, t)

### Example 123

In 2 ml of tetrahydrofuran were dissolved 266 mg of a compound represented by the formula (IIa-2) and 90 mg of 1-butanol, 50 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 166 mg of a compound represented by the formula (123):

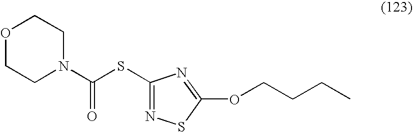

(hereinafter, referred to as present compound (123)).

¹H-NMR (CDCl₃, TMS) δ (ppm): 4.52 (2H, t), 3.72 (4H, t), 3.57 (4H, br), 1.82 (2H, m), 1.47 (2H, m), 0.97 (3H, t)

### Example 124

According to the same manner as that of Example 123 except that 67 mg of 2-propyne-1-ol was used in place of 1-butanol, 162 mg of a compound represented by the formula (124):

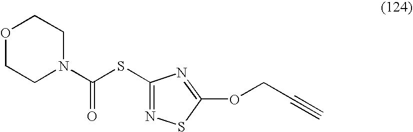

(hereinafter, referred to as present compound (124)) was obtained.

¹H-NMR (CDCl₃, TMS) δ (ppm): 5.15 (2H, d), 3.73 (4H, t), 3.57 (4H, br), 2.66 (1H, t)

### Example 125

According to the same manner as that of Example 123 except that 84 mg of 2-butyne-1-ol was used in place of 1-butanol, 192 mg of a compound represented by the formula (125):

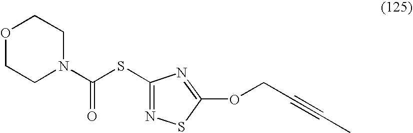

(hereinafter, referred to as present compound (125)) was obtained.

¹H-NMR (CDCl₃, TMS) δ (ppm): 5.10 (2H, q), 3.72 (4H, t), 3.57 (4H, br), 1.90 (3H, t)

### Example 126

According to the same manner as that of Example 123 except that 101 mg of 2-pentyne-1-ol was used in place of 1-butanol, 147 mg of a compound represented by the formula (126):

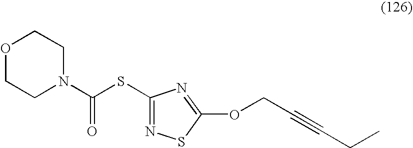

(hereinafter, referred to as present compound (126)) was obtained.

¹H-NMR (CDCl₃, TMS) δ (ppm): 5.12 (2H, t), 3.72 (4H, t), 3.57 (4H, br), 2.27 (2H, tq), 1.16 (3H, t)

### Example 127

According to the same manner as that of Example 123 except that 123 mg of tetrahydro-3-furanmethanol was used in place of 1-butanol, 170 mg of a compound represented by the formula (127):

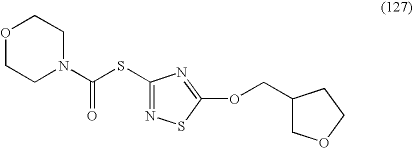

(hereinafter, referred to as present compound (127)) was obtained.

¹H-NMR (CDCl₃, TMS) δ (ppm): 4.49 (2H, m), 3.92-3.49 (12H, m), 2.80 (1H, m), 2.11 (1H, m), 1.71 (1H, m)

### Example 128

According to the same manner as that of Example 123 except that 139 mg of tetrahydropyran-2-methanol was used in place of 1-butanol, 120 mg of a compound represented by the formula (128):

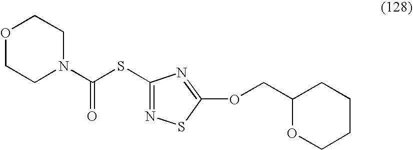

(hereinafter, referred to as present compound (128)) was obtained.

¹H-NMR (CDCl₃, TMS) δ (ppm): 4.55-4.44 (2H, m), 4.04 (1H, m), 3.77-3.45 (10H, m), 1.91 (1H, m), 1.65-1.33 (5H, m)

### Example 129 and Example 130

In 2 ml of tetrahydrofuran were dissolved 532 mg of a compound represented by the formula (IIa-2) and 230 mg of glycerol formal, 100 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 210 mg of a compound represented by the formula (129):

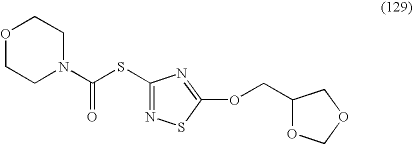

(hereinafter, referred to as present compound (129)) and 204 mg a compound represented by the formula (130):

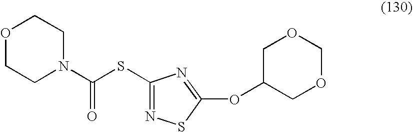

(hereinafter, referred to as present compound (130)).

### Present Compound (129)

¹H-NMR (CDCl₃, TMS) δ (ppm): 5.07 (1H, s), 4.93 (1H, s), 4.59 (2H, m), 4.47 (1H, m), 4.04 (1H, dd), 3.80 (1H, dd), 3.72 (4H, t), 3.57 (4H, s)

### Present Compound (130)

¹H-NMR (CDCl₃, TMS) δ (ppm): 5.06 (1H, m), 5.03 (1H, d), 4.83 (1H, d), 4.25 (2H, dd), 4.08 (2H, dd), 3.72 (4H, t), 3.56 (4H, s)

### Example 131

In 2 ml of tetrahydrofuran was dissolved 160 mg of 2,2-dimethyl-1,3-dioxolane-4-methanol, 50 mg of sodium hydride (60% oily) was added under ice-cooling, the mixture was stirred for 5 minutes, 350 mg of a compound represented by the formula (IIa-3):

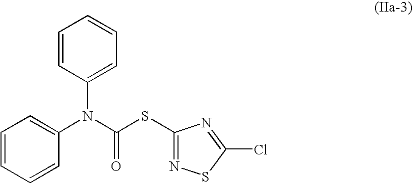

was added, and the mixture was stirred at room temperature for 2 hours. Thereafter, an aqueous saturated ammonium chloride solution was added to the reaction mixture, followed by extraction with ethyl acetate. The organic layer was dried with sodium sulfate, and concentrated under reduced pressure. The residue was subjected to silica gel preparative thin layer chromatography to obtain 287 mg of a compound represented by the formula (131):

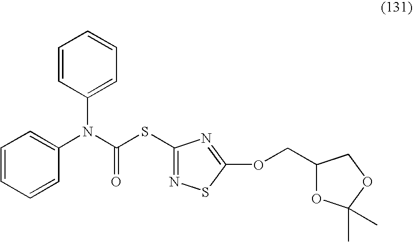

(hereinafter, referred to as compound (131)).

¹H-NMR (CDCl₃, TMS) δ (ppm): 7.44-7.28 (10H, m), 4.60 (1H, m), 4.51 (2H, m), 4.15 (1H, m), 3.83 (1H, m), 1.45 (3H, s), 1.38 (3H, s)

### Example 132

In 2 ml of tetrahydrofuran were dissolved 286 mg of a compound represented by the formula (IIa-4):

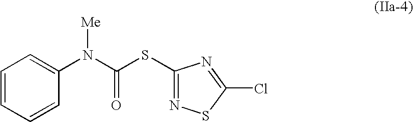

and 145 mg of 2,2-dimethyl-1,3-dioxolane-4-methanol, 50 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 339 mg of a compound represented by the formula (132):

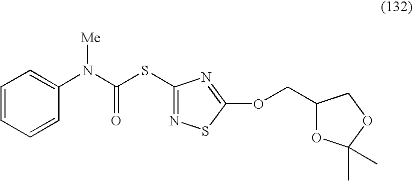

(hereinafter, referred to as compound (132)).

¹H-NMR (CDCl₃, TMS) δ (ppm): 7.48-7.36 (5H, m), 4.59 (1H, m), 4.51 (2H, m), 4.14 (1H, m), 3.83 (1H, m), 3.34 (3H, s), 1.45 (3H, s), 1.38 (3H, s)

### Example 133

In 2 ml of tetrahydrofuran were dissolved 264 mg of a compound represented by the formula (IIa-5):

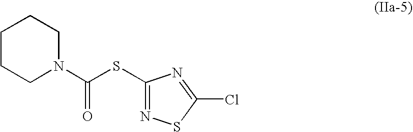

and 139 mg of 2,2-dimethyl-1,3-dioxolane-4-methanol, 44 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 318 mg of a compound represented by the formula (133):

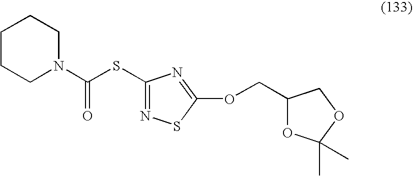

(hereinafter, referred to as compound (133)).

¹H-NMR (CDCl₃, TMS) δ (ppm): 4.61 (1H, m), 4.52 (2H, m), 4.14 (1H, m), 3.83 (1H, m), 3.49 (4H, br), 1.65 (6H, br), 1.45 (3H, s), 1.39 (3H, s)

### Example 134

In 2 ml of tetrahydrofuran were dissolved 250 mg of a compound represented by the formula (IIa-6):

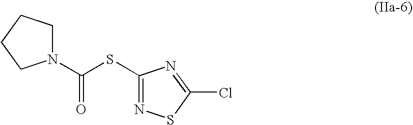

and 160 mg of 2,2-dimethyl-1,3-dioxolane-4-methanol, 50 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 209 mg of a compound represented by the formula (134):

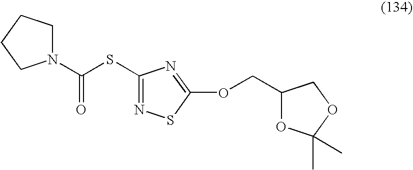

(hereinafter, referred to as compound (134)).

¹H-NMR (CDCl₃, TMS) δ (ppm): 4.61 (1H, m), 4.52 (2H, m), 4.14 (1H, dd), 3.83 (1H, dd), 3.53 (2H, t), 3.45 (2H, t), 2.03-1.87 (4H, m), 1.45 (3H, s), 1.39 (3H, s)

### Example 135

In 2 ml of tetrahydrofuran was dissolved 250 mg of a compound represented by the formula (IIa-6), 210 mg of a 28% methanol solution of sodium methoxide was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The resulting solid was washed with a mixed solution of toluene-hexane to obtain 160 mg of a compound represented by the formula (135):

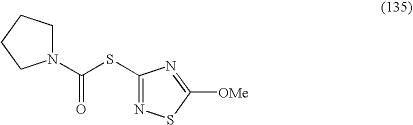

(hereinafter, referred to as compound (135)).

¹H-NMR (CDCl₃, TMS) δ (ppm): 4.22 (3H, s), 3.54 (2H, t), 3.45 (2H, t), 2.03-1.87 (4H, m)

### Example 136

In 2 ml of tetrahydrofuran was dissolved 250 mg of a compound represented by the formula (IIa-6), 374 mg of a 20% ethanol solution of sodium ethoxide was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel column chromatography to obtain 215 mg of a compound represented by the formula (136):

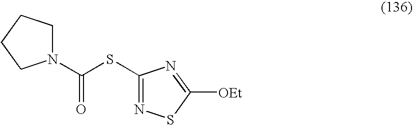

(hereinafter, referred to as compound (136)).

¹H-NMR (CDCl₃, TMS) δ (ppm): 4.59 (2H, q), 3.54 (2H, t), 3.45 (2H, t), 2.03-1.87 (4H, m), 1.47 (3H, t)

### Example 137

In 2 ml of tetrahydrofuran were dissolved 250 mg of a compound represented by the formula (IIa-6) and 112 mg of tetrahydro-4-pyranol, 44 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The resulting solid was washed with a mixed solution of toluene-hexane to obtain 269 mg of a compound represented by the formula (137):

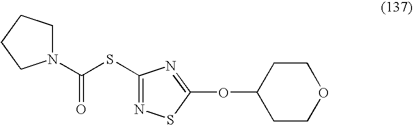

(hereinafter, referred to as compound (137)).

¹H-NMR (CDCl₃, TMS) δ (ppm): 5.27 (1H, m), 3.96 (2H, m), 3.59 (2H, m), 3.53 (2H, t), 3.45 (2H, t), 2.15 (2H, m), 2.03-1.86 (6H, m)

### Example 138

In 2 ml of tetrahydrofuran were dissolved 252 mg of a compound represented by the formula (IIa-7):

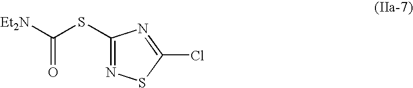

and 139 mg of 2,2-dimethyl-1,3-dioxolane-4-methanol, 42 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 321 mg of a compound represented by the formula (138):

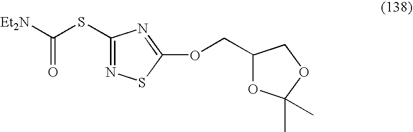

(hereinafter, referred to as compound (138)).

¹H-NMR (CDCl₃, TMS) δ (ppm): 4.62 (1H, m), 4.52 (2H, m), 4.14 (1H, m), 3.83 (1H, m), 3.40 (4H, br), 1.45 (3H, s), 1.39 (3H, s), 1.27 (3H, br), 1.17 (3H, br)

### Example 139

In 2 ml of tetrahydrofuran was dissolved 252 mg of a compound represented by the formula (IIa-7), 263 mg of a 28% methanol solution of sodium methoxide under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 219 mg of a compound represented by the formula (139):

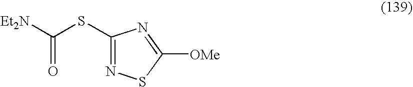

(hereinafter, referred to as compound (139)).

¹H-NMR (CDCl₃, TMS) δ (ppm): 4.22 (3H, s), 3.40 (4H, br), 1.28 (3H, br), 1.17 (3H, br)

### Example 140

In 2 ml of tetrahydrofuran was dissolved 252 mg of a compound represented by the formula (IIa-7), 340 mg of a 20% ethanol solution of sodium ethoxide was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel column chromatography to obtain 250 mg of a compound represented by the formula (140):

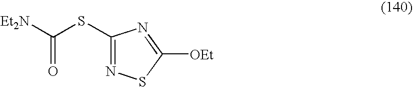

(hereinafter, referred to as compound (140)).

¹H-NMR (CDCl₃, TMS) δ (ppm): 4.59 (2H, q), 3.40 (4H, br), 1.47 (3H, t), 1.28 (3H, br), 1.17 (3H, br)

### Example 141

In 2 ml of tetrahydrofuran were dissolved 252 mg of a compound represented by the formula (IIa-7) and 107 mg of tetrahydro-4-pyranol, 42 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer thin layer chromatography to obtain 292 mg of a compound represented by the formula (141):

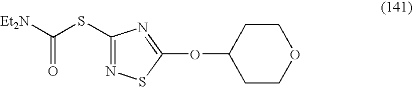

(hereinafter, referred to as compound (141)).

¹H-NMR (CDCl₃, TMS) δ (ppm): 5.26 (1H, m), 3.95 (2H, m), 3.60 (2H, m), 3.40 (4H, br), 2.15 (2H, m), 1.90 (2H, m), 1.28 (3H, br), 1.17 (3H, br)

### Example 142

In 150 ml of ethyl acetate were suspended 13.8 g of a compound represented by the formula (IXa-1):

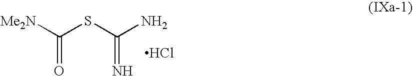

and 31.5 g of sodium bicarbonate, 18.2 g of perchloromethylmercaptan was added dropwise to the reaction mixture, and the mixture was stirred at room temperature for a whole day and night. Thereafter, water was added to the reaction mixture, followed by extraction with ethyl acetate. The organic layer was washed sequentially with an aqueous saturated sodium bicarbonate solution, and an aqueous saturated sodium chloride solution, dried with sodium sulfate, and concentrated under reduced pressure. The residue was subjected to silica gel column chromatography to obtain 10.4 g of a compound represented by the formula (IIa-1) (hereinafter, referred to as present compound (142)).

¹H-NMR (CDCl₃, TMS) δ (ppm): 3.08 (3H, brs), 3.05 (3H, brs)

### Example 143

In 55 ml of ethyl acetate were suspended 5.0 g of a compound represented by the formula (IXa-2):

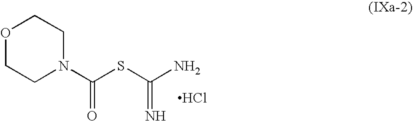

and 9.3 g of sodium bicarbonate, 5.4 g of perchloromethylmercaptan was added dropwise to the reaction mixture, and the mixture was stirred at room temperature for a whole day and night. Thereafter, water was added to the reaction mixture, followed by extraction with ethyl acetate. The organic layer was washed sequentially with an aqueous saturated sodium bicarbonate solution, and an aqueous saturated sodium chloride solution, dried with sodium sulfate, and concentrated under reduced pressure. The residue was subjected to silica gel column chromatography to obtain 3.7 g of a compound represented by the formula (ii-1) (hereinafter, referred to as present compound (143)).

¹H-NMR (CDCl₃, TMS) δ (ppm): 3.73 (4H, t), 3.58 (4H, brs)

### Example 144

In 20 ml of ethyl acetate were suspended 3.08 g of a compound represented by the formula (IXa-3):

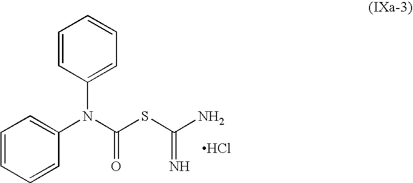

and 4.20 g of sodium bicarbonate, 2.42 g of perchloromethylmercaptan was added dropwise to the reaction mixture, and the mixture was stirred at room temperature for a whole day and night. Thereafter, water was added to the reaction mixture, followed by extraction with ethyl acetate. The organic layer was washed sequentially with an aqueous saturated sodium bicarbonate solution, and an aqueous saturated sodium chloride solution, dried with sodium sulfate, and concentrated under reduced pressure. The resulting crystal was washed with methanol to obtain 1.22 g of a compound represented by the formula (IIa-3) (hereinafter, referred to as present compound (144)).

¹H-NMR (CDCl₃, TMS) δ (ppm): 7.41 (10H, br)

### Example 145

In 5 ml of ethyl acetate were suspended 1.23 g of a compound represented by the formula (IXa-4):

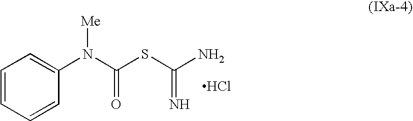

and 2.10 g of sodium bicarbonate, 1.21 g of perchloromethylmercaptan was added dropwise to the reaction mixture, and the mixture was stirred at room temperature for a whole day and night. Thereafter, water was added to the reaction mixture, followed by extraction with ethyl acetate. The organic layer was washed sequentially with an aqueous saturated sodium bicarbonate solution, and an aqueous saturated sodium chloride solution, dried with sodium sulfate, and concentrated under reduced pressure. The resulting crystal was washed with ethyl acetate to obtain 0.94 g of a compound represented by the formula (IIa-4) (hereinafter, referred to as present compound (145)).

¹H-NMR (CDCl₃, TMS) δ (ppm): 7.50-7.44 (3H, m), 7.40-7.37 (2H, m), 3.35 (3H, s)

### Example 146

In 5 ml of ethyl acetate were suspended 1.12 g of a compound represented by the formula (IXa-5):

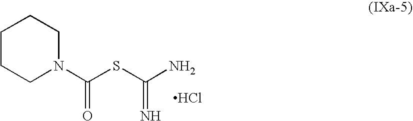

and 2.52 g of sodium bicarbonate, 1.39 g of perchloromethylmercaptan was added dropwise to the reaction mixture, and the mixture was stirred at room temperature for a whole day and night. Thereafter, water was added to the reaction mixture, followed by extraction with ethyl acetate. The organic layer was washed sequentially with an aqueous saturated sodium bicarbonate solution, and an aqueous saturated sodium chloride solution, dried with sodium sulfate, and concentrated under reduced pressure. The residue was subjected to silica gel column chromatography to obtain 0.92 g of a compound represented by the formula (IIa-5) (hereinafter, referred to as present compound (146)).

¹H-NMR (CDCl₃, TMS) δ (ppm): 3.51 (4H, br), 1.66 (6H, br)

### Example 147

In 30 ml of ethyl acetate were suspended 5.3 g of a compound represented by the formula (IXa-6):

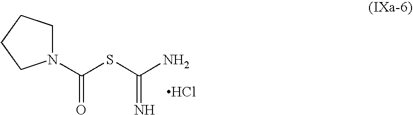

and 10.6 g of sodium bicarbonate, 6.1 g of perchloromethylmercaptan was added dropwise to the reaction mixture, and the mixture was stirred at room temperature for a whole day and night. Thereafter, water was added to the reaction mixture, followed by extraction with ethyl acetate. The organic layer was washed sequentially with an aqueous saturated sodium bicarbonate solution, and an aqueous saturated sodium chloride solution, dried with sodium sulfate, and concentrated under reduced pressure. The residue was subjected to silica gel column chromatography to obtain 4.21 g of a compound represented by the formula (IIa-6) (hereinafter, referred to as present compound (147)).

¹H-NMR (CDCl₃, TMS) δ (ppm): 3.55 (2H, t), 3.48 (2H, t), 2.05-4.89 (4H, m)

### Example 148

In 20 ml of ethyl acetate were suspended 2.12 g of a compound represented by the formula (IXa-7):

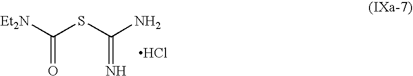

and 4.20 g of sodium bicarbonate, 2.41 g of perchloromethylmercaptan was added dropwise to the reaction mixture, and the mixture was stirred at room temperature for a whole day and night. Thereafter, water was added to the reaction mixture, followed by extraction with ethyl acetate. The organic layer was washed sequentially with an aqueous saturated sodium bicarbonate solution, and an aqueous saturated sodium chloride solution, dried with sodium sulfate, and concentrated under reduced pressure. The residue was subjected to silica gel column chromatography to obtain 1.67 g of a compound represented by the formula (IIa-7) (hereinafter, referred to as present compound (148)).

¹H-NMR (CDCl₃, TMS) δ (ppm): 3.41 (4H, br), 1.30 (3H, br), 1.18 (3H, br)

### Example 201

In 6 ml of tetrahydrofuran were dissolved 670 mg of the compound represented by the formula (IIa-1) and 470 mg of 3,3-diethoxy-1-propanol, 130 mg of sodium hydride (60% oily) was added, and the mixture was stirred for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 450 mg of a compound represented by the formula (201):

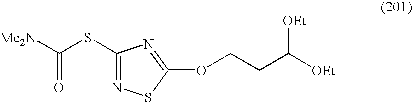

(hereinafter, referred to as compound (201)).

¹H-NMR (CDCl₃, TMS) δ (ppm): 4.69 (1H, t), 4.61 (2H, t), 3.71-3.64 (2H, m), 3.55-3.48 (2H, m), 3.04 (6H, br), 2.14 (2H, dt, J=6 Hz, 6 Hz), 1.21 (6H, t)

### Example 202

In 5 ml of tetrahydrofuran were dissolved 340 mg of the compound represented by the formula (IIa-1) and 330 mg of 4-((3,3-dichloro-2-propen-1-yl)oxy)phenol, 230 mg of potassium carbonate was added, and the mixture was stirred at room temperature for 10 hours. Thereafter, an aqueous saturated ammonium chloride solution was added to the reaction mixture, followed by extraction with t-butyl methyl ether. The organic layer was dried with sodium sulfate, and concentrated under reduced pressure. The resulting solid was washed with toluene to obtain 500 mg of a compound represented by the formula (202):

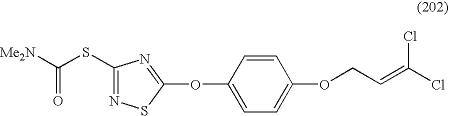

(hereinafter, referred to as compound (202)).

¹H-NMR (CDCl₃, TMS) δ (ppm): 7.30-7.24 (2H, m), 6.97-6.92 (2H, m), 6.17 (1H, t), 4.67 (2H, d), 3.04 (6H, br).

### Example 203 and Example 204

In 2 ml of tetrahydrofuran were dissolved 340 mg of the compound represented by the formula (IIa-1) and 110 mg of 1,3-propanediol, 60 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 110 mg of a compound represented by the formula (203):

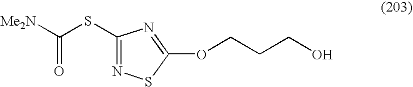

(hereinafter, referred to as compound (203)) and 60 mg of a compound represented by the formula (204):

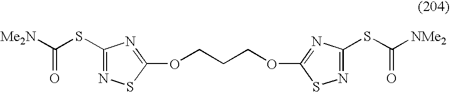

(hereinafter, referred to as compound (203)).

### Present Compound (203)

¹H-NMR (CDCl₃, TMS) δ (ppm): 4.75 (2H, t, J=6 Hz), 3.75 (2H, dt, J=5 Hz, 6 Hz), 3.04 (6H, br), 2.38(1H, t, J=5 Hz), 2.07-2.02 (2H, m)

### Present Compound (204)

¹H-NMR (CDCl₃, TMS) δ (ppm): 4.70 (4H, t), 3.04 (12H, br), 2.40-2.34 (2H, m)

### Example 205 and Example 206

In 2 ml of tetrahydrofuran were dissolved 340 mg of the compound represented by the formula (IIa-1) and 140 mg of 1,4-butanediol, 60 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 160 mg of a compound represented by the formula (205):

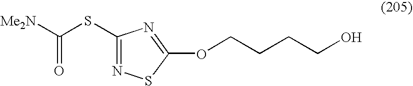

(hereinafter, referred to as compound (205)) and 90 mg of the crude product of a compound represented by the formula (206):

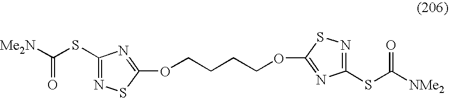

(hereinafter, referred to as compound (206)).

### Present Compound (205)

¹H-NMR (CDCl₃, TMS) δ (ppm): 4.58 (2H, t) , 3.73-3.69 (2H, m), 3.04 (6H, br), 2.91 (1H, brs), 1.98-1.91 (2H, m), 1.74-1.67 (2H, m)

### Present Compound (206)

¹H-NMR (CDCl₃, TMS) δ (ppm): 4.60-4.58 (4H, m), 3.04 (12H, br), 2.01-1.99 (4H, m)

### Example 207

In 2 ml of tetrahydrofuran were dissolved 340 mg of the compound represented by the formula (IIa-1) and 140 mg of 2-hydroxyacetic acid methyl ester, 60 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 110 mg of a compound represented by the formula (207):

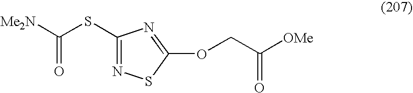

(hereinafter, referred to as compound (207)).

¹H-NMR (CDCl₃, TMS) δ (ppm): 5.10 (2H, s), 3.82 (3H, s), 3.04 (6H, br)

### Example 208

In 2 ml of tetrahydrofuran was dissolved 340 mg of the compound represented by the formula (IIa-1), 110 mg of a methanethiol sodium salt was added, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 180 mg of a compound represented by the formula (208):

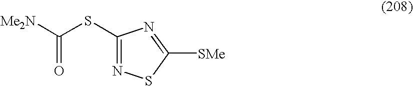

(hereinafter, referred to as compound (208)).

¹H-NMR (CDCl₃, TMS) δ (ppm): 3.06 (6H, br), 2.75 (3H, s)

### Example 209

In 2 ml of tetrahydrofuran was dissolved 340 mg of the compound represented by the formula (IIa-1), 130 mg of an ethanethiol sodium salt was added, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 130 mg of a compound represented by the formula (209):

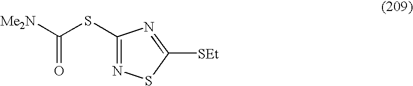

(hereinafter, referred to as compound (209)).

¹H-NMR (CDCl₃, TMS) δ (ppm): 3.29 (2H, q), 3.06 (6H, br), 1.49 (3H, t)

### Example 210

In 2 ml of tetrahydrofuran were dissolved 340 mg of the compound represented by the formula (IIa-1) and 160 mg of phenol, 230 mg of potassium carbonate was added, and the mixture was stirred at room temperature for 10 hours. Thereafter, an aqueous saturated ammonium chloride solution was added to the reaction mixture, followed by extraction with t-butyl methyl ether. The organic layer was dried with sodium sulfate, and concentrated under reduced pressure. The resulting solid was washed with toluene and dried under reduced pressure to obtain 210 mg of a compound represented by the formula (210):

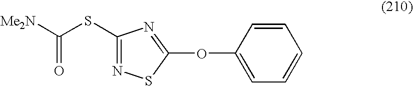

(hereinafter, referred to as compound (210)).

¹H-NMR (CDCl₃, TMS) δ (ppm): 7.50-7.46 (2H, m), 7.37-7.33 (3H, m), 3.05 (6H, br)

### Example 211

In 2 ml of tetrahydrofuran were dissolved 340 mg of the compound represented by the formula (IIa-1) and 180 mg of benzyl alcohol, 70 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 320 mg of a compound represented by the formula (211):

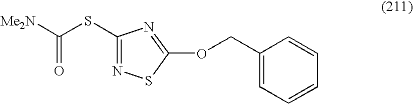

(hereinafter, referred to as compound (211)).

¹H-NMR (CDCl₃, TMS) δ (ppm): 7.46-7.39 (5H, m), 5.54 (2H, s), 3.06 (6H, br)

### Example 212

In 2 ml of tetrahydrofuran were dissolved 340 mg of the compound represented by the formula (IIa-1) and 160 mg of 2-pyridinol, 230 mg of potassium carbonate was added, and the mixture was stirred at room temperature for 10 hours. Thereafter, an aqueous saturated ammonium chloride solution was added to the reaction mixture, followed by extraction with chloroform. The organic layer was washed sequentially with an aqueous saturated sodium bicarbonate solution and water, dried with sodium sulfate, and concentrated under reduced pressure. The resulting solid was recrystallized from toluene to obtain 180 mg of a compound represented by the formula (212):

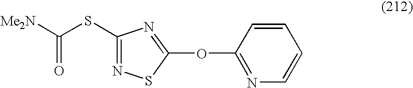

(hereinafter, referred to as compound (212)).

¹H-NMR (CDCl₃, TMS) δ (ppm): 8.72-8.70 (1H, m), 7.59-7.55 (1H, m), 6.87 (1H, d), 6.54-6.51 (1H, m), 3.12 (3H, brs), 3.05 (3H, brs)

### Example 213

In 2 ml of tetrahydrofuran were dissolved 340 mg of the compound represented by the formula (IIa-1) and 160 mg of 3-pyridinol, 230 mg of potassium carbonate was added, and the mixture was stirred at room temperature for 10 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 180 mg of a compound represented by the formula (213):

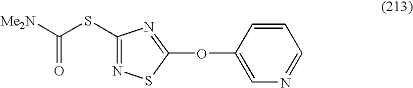

(hereinafter, referred to as compound (213)).

¹H-NMR (CDCl₃, TMS) δ (ppm): 8.70 (1H, d), 8.58 (1H, dd), 7.80 (1H, ddd), 7.42 (1H, ddd), 3.04 (6H, br)

### Example 214

In 2 ml of tetrahydrofuran were dissolved 340 mg of the compound represented by the formula (IIa-1) and 160 mg of 4-pyridinol, 230 mg of potassium carbonate was added, and the mixture was stirred at room temperature for 10 hours. Thereafter, an aqueous saturated ammonium chloride solution was added to the reaction mixture, followed by extraction with chloroform. The organic layer was washed sequentially with an aqueous saturated sodium bicarbonate solution and water, dried with sodium sulfate, and concentrated under reduced pressure. The resulting solid was washed with toluene to obtain 300 mg of a compound represented by the formula (214):

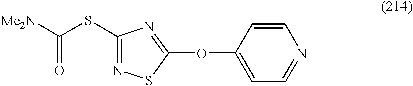

(hereinafter, referred to as compound (214)).

¹H-NMR (CDCl₃, TMS) δ (ppm): 8.00 (2H, d), 6.48 (2H, d), 3.10 (3H, brs), 3.06 (3H, brs).

### Example 215

In 2 ml of tetrahydrofuran were dissolved 340 mg of the compound represented by the formula (IIa-1) and 180 mg of 2-pyridinemethanol, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 240 mg of a compound represented by the formula (215):

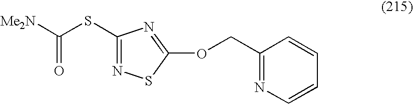

(hereinafter, referred to as compound (215)).

¹H-NMR (CDCl₃, TMS) δ (ppm): 8.65-8.63 (1H, m), 7.78-7.73 (1H, m), 7.49-7.47 (1H, m), 7.31-7.28 (1H, m), 5.66 (2H, s), 3.05 (6H, br)

### Example 216

In 2 ml of tetrahydrofuran were suspended 340 mg of the compound represented by the formula (IIa-1) and 510 mg of 4-(4-((3,3-dichloro-2-propenyl)oxy)phenoxy)phenol, 250 mg of potassium carbonate was added, and the mixture was stirred at room temperature for 10 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 630 mg of a compound represented by the formula (216):

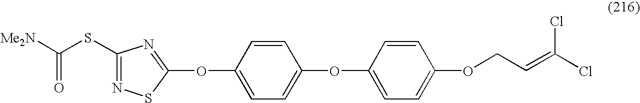

(hereinafter, referred to as compound (216)).

¹H-NMR (CDCl₃, TMS) δ (ppm): 7.29-7.25 (2H, m), 7.04-6.97 (4H, m), 6.93-6.88 (2H, m), 6.17 (1H, t), 4.66 (2H, d), 3.05 (6H, br)

### Example 217

In 2 ml of tetrahydrofuran were dissolved 340 mg of the compound represented by the formula (IIa-1) and 360 mg of 4-((1,3,4-trimethyl-1H-pyrazol-5-yl)oxy)phenol, 250 mg of potassium carbonate was added, and the mixture was stirred at room temperature for 10 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 540 mg of a compound represented by the formula (217):

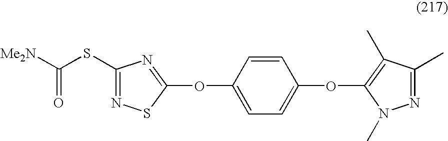

(hereinafter, referred to as compound (217)).

¹H-NMR (CDCl₃, TMS) δ (ppm): 7.33-7.28 (2H, m), 6.98-6.94 (2H, m), 3.59 (3H, s), 3.04 (6H, br), 2.19 (3H, s), 1.77 (3H, s)

### Example 218

In 2 ml of tetrahydrofuran were dissolved 340 mg of the compound represented by the formula (IIa-1) and 210 mg of 1,3,4-trimethyl-1H-pyrazol-5-ol, 250 mg of potassium carbonate was added, and the mixture was stirred at room temperature for 10 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 200 mg of a compound represented by the formula (218):

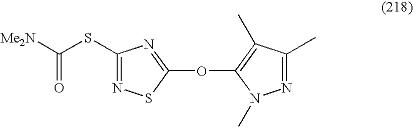

(hereinafter, referred to as compound (218)).

¹H-NMR (CDCl₃, TMS) δ (ppm): 3.67 (3H, s), 3.05 (6H, br), 2.18 (3H, s), 1.88 (3H, s)

### Example 219

In 2 ml of tetrahydrofuran were dissolved 340 mg of the compound represented by the formula (IIa-1) and 370 mg of 5-hydroxy-2-(((tetrahydro-2H-pyran-2-yl)oxy)methyl)-4H-pyran-4-one, 70 mg of sodium hydride (60% oily) and 2 ml of tetrahydrofuran were added, and the mixture was stirred at room temperature for 10 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 570 mg of a compound represented by the formula (219):

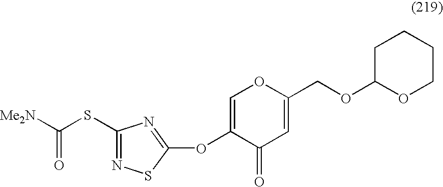

(hereinafter, referred to as compound (219)).

¹H-NMR (CDCl₃, TMS) δ (ppm): 8.29 (1H, s), 6.63 (1H, s), 4.75 (1H, t), 4.57 (1H, d), 4. 37 (1H, d), 3.86-3.80 (1H, m), 3.60-3.55 (1H, m), 3.02 (6H, br), 1.86-1.51 (6H, m)

### Example 220

In 2 ml of tetrahydrofuran were dissolved 340 mg of the compound represented by the formula (IIa-1) and 36b mg of N-(tert-butoxycarbonyl)-4-piperidinemeathanol, 70 mg of sodium hydride (60% oily) was added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 330 mg of a compound represented by the formula (220):

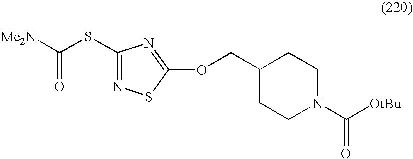

(hereinafter, referred to as compound (220)).

¹H-NMR (CDCl₃, TMS) δ (ppm): 4.38 (2H, d), 4.15 (1H, br), 3.04 (6H, br), 2.73 (2H, br), 2.07-1.97 (1H, m), 1.78-1.75 (2H, br), 1.31-1.22 (2H, m)

### Example 221

In 2 ml of tetrahydrofuran were dissolved 340 mg of the compound represented by the formula (IIa-1) and 150 mg of 2-ethoxyethanol, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 330 mg of a compound represented by the formula (221):

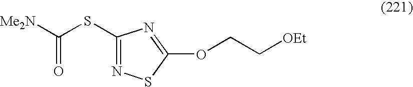

(hereinafter, referred to as compound (221)).

¹H-NMR (CDCl₃, TMS) δ (ppm): 4.69-4.67 (2H, m), 3.81-3.79 (2H, m), 3.57 (2H, q), 3.04 (6H, br), 1.23 (3H, t)

### Example 222

In 2 ml of tetrahydrofuran were dissolved 340 mg of the compound represented by the formula (IIa-1) and 200 mg of 2-(tert-butoxy)ethanol, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 400mg of a compound represented by the formula (222):

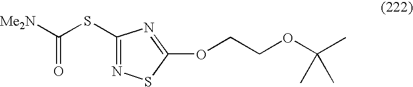

(hereinafter, referred to as present compound (222)).

¹H-NMR (CDCl₃, TMS) δ (ppm): 4.64-4.61 (2H, m), 3.74-3.72 (2H, m), 3.04 (6H, br), 1.21 (9H, s)

### Example 223

In 2 ml of tetrahydrofuran were dissolved 340 mg of the compound represented by the formula (IIa-1) and 210 mg of 2-(2-chloroethoxy)ethanol, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 400 mg of a compound represented by the formula (223):

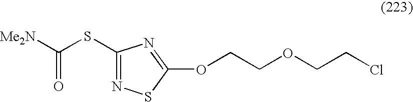

(hereinafter, referred to as present compound (223)).

¹H-NMR (CDCl₃, TMS) δ (ppm): 4.72-4.69 (2H, m), 3.91-3.89 (2H, m), 3.79 (2H, t), 3.64 (2H, t), 3.04 (6H, br)

### Example 224

In 1 ml of tetrahydrofuran was dissolved 190 mg of 1-methyl-4-piperidinol, 70 mg of sodium hydride (60% oily) was added under ice-cooling, and the mixture was stirred at room temperature for 30 minutes. Thereafter, 1.5 ml of a solution of 340 mg of the compound represented by the formula (IIa-1) in tetrahydrofuran was added dropwise, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 210 mg of a compound represented by the formula (224):

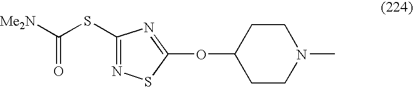

(hereinafter, referred to as present compound (224)).

¹H-NMR (CDCl₃, TMS) δ (ppm): 5.13-5.07 (1H, m), 3.04 (6H, br), 2.64 (2H, br), 2.35 (2H, br), 2.31 (3H, s), 2.15-2.08 (2H, m), 2.01-1.93 (2H, m)

### Example 225 and Example 226

In 4 ml of tetrahydrofuran were dissolved 670 mg of the compound represented by the formula (IIa-1) and 150 mg of ethylene glycol, 120 mg of sodium hydride (60% oily) and 1 ml of tetrahydrofuran were added under ice-cooling, and the mixture was stirred at room temperature for 6 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 240 mg of a compound represented by the formula (225):

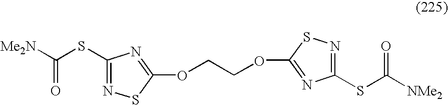

(hereinafter, referred to as present compound (225)) and 200 mg of a compound represented by the formula (226):

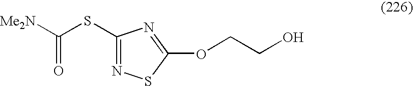

(hereinafter, referred to as present compound (226)), respectively.

### Present Compound (225)

¹H-NMR (CDCl₃, TMS) δ (ppm): 4.91 (4H, s), 3.05 (12H, br)

### Present Compound (226)

¹H-NMR (CDCl₃, TMS) δ (ppm): 4.69-4.67 (2H, m), 4.03-3.99 (2H, m), 3.04 (6H, br), 2.51 (1H, t)

### Example 227

In 2 ml of tetrahydrofuran were dissolved 340 mg of the compound represented by the formula (IIa-1) and 170 mg of 2-propoxyethanol, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 290 mg of a compound represented by the formula (227):

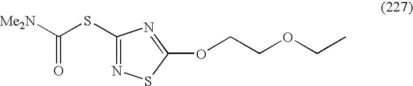

(hereinafter, referred to as present compound (227)).

¹H-NMR (CDCl₃, TMS) δ (ppm): 4.69-4.67 (2H, m), 3.81-3.78 (2H, m), 3.46 (2H, t), 3.04 (6H, br), 1.66-1.57 (2H, m), 0.92 (3H, t)

### Example 228

According to the same manner as that of Example 227 except that 2-isopropoxyethanol was used in place of 2-propoxyethanol, 330 mg of a compound represented by the formula (228):

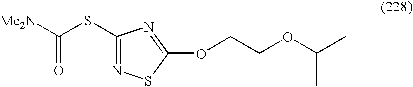

(hereinafter, referred to as present compound (228)) was obtained.

¹H-NMR (CDCl₃, TMS) δ (ppm): 4.67-4.65 (2H, m), 3.80-3.77 (2H, m), 3.70-3.60 (1H, m), 3.04 (6H, br), 1.18 (6H, d)

### Example 229

According to the same manner as that of Example 227 except that 170 mg of 2-allyloxyethanol was used in place of 2-propoxyethanol, 340 mg of a compound represented by the formula (229):

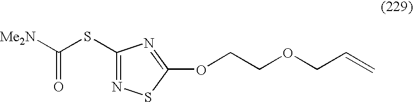

(hereinafter, referred to as present compound (229)) was obtained.

¹H-NMR (CDCl₃, TMS) δ (ppm): 5.96-5.86 (1H, m), 5.33-5.27 (1H, m), 5.24-5.20 (1H, m), 4.71-4.68 (2H, m), 4.07-4.05 (2H, m), 3.82-3.80 (2H, m), 3.04 (6H, br)

### Example 230

According to the same manner as that of Example 227 except that 220 mg of 2-phenoxyethanol was used in place of 2-propoxyethanol, 410 mg of a compound represented by the formula (230):

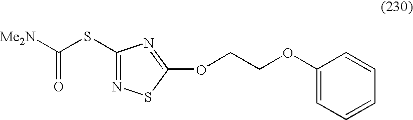

(hereinafter, referred to as present compound (230)) was obtained.

¹H-NMR (CDCl₃, TMS) δ (ppm): 7.32-7.26 (2H, m), 7.00-6.96 (1H, m), 6.94-6.91 (2H, m), 4.90-4.88 (2H, m), 4.35-4.33 (2H, m), 3.05 (6H, br)

### Example 231

According to the same manner as that of Example 227 except that 240 mg of 2-benzyloxyethanol was used in place of 2-propoxyethanol, 460 mg of a compound represented by the formula (231):

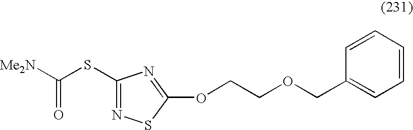

(hereinafter, referred to as present compound (231)) was obtained.

¹H-NMR (CDCl₃, TMS) δ (ppm): 7.36-7.30 (5H, m), 4.72-4.70 (2H, m), 4.57 (2H, s), 3.85-3.83 (2H, m), 3.04(6H, br)

### Example 232

According to the same manner as that of Example 227 except that 230 mg of 2-(2,2,2,-trifluoroethoxy)ethanol was used in place of 2-propoxyethanol, 400 mg of a compound represented by the formula (232):

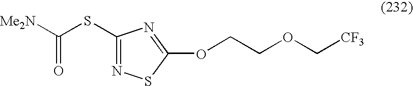

(hereinafter, referred to as present compound (232)) was obtained.

¹H-NMR (CDCl₃, TMS) δ (ppm): 4.72-4.70 (2H, m), 4.02-3.99 (2H, m), 3.92 (2H, q, J=9 Hz), 3.04 (6H, br)

### Example 233

In 2 ml of tetrahydrofuran were dissolved 340 mg of the compound represented by the formula (IIa-1) and 190 mg of 2-isobutoxyethanol, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 260 mg of a compound represented by the formula (233):

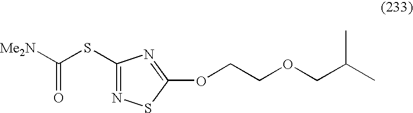

(hereinafter, referred to as present compound (233)).

¹H-NMR (CDCl₃, TMS) δ (ppm): 4.69-4.66 (2H, m), 3.80-3.78 (2H, m), 3.26 (2H, d), 3.04 (6H, br), 1.93-1.82 (1H, m), 0.90 (6H, d)

### Example 234

In 2 ml of tetrahydrofuran were dissolved 190 mg of the present compound (226):

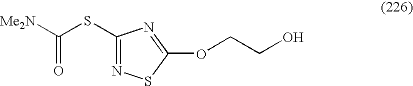

and 90 mg of triethylamine, 70 mg of acetyl chloride was added, and the solution was stirred at room temperature for 2 hours. Thereafter, 20 mg of acetyl chloride and 20 mg of triethylamine were further added, and the mixture was stirred for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 190 mg of a compound represented by the formula (234):

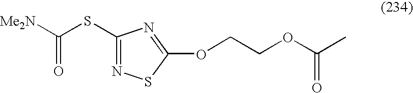

(hereinafter, referred to as present compound (234)).

¹H-NMR (CDCl₃, TMS) δ (ppm): 4.75-4.73 (2H, m), 4.45-4.43 (2H, m), 3.04 (6H, br), 2.11 (3H, s)

### Example 235

In 2 ml of tetrahydrofuran were dissolved 340 mg of the compound represented by the formula (IIa-1) and 190 mg of 1-methyl-3-piperidinol, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 60 mg of the crude product of a compound represented by the formula (235):

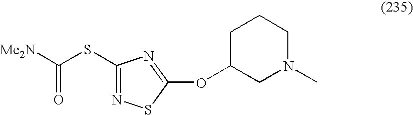

(hereinafter, referred to as present compound (235)).

¹H-NMR (CDCl₃, TMS) δ (ppm): 5.22 (1H, br), 3.04 (6H, br), 2.72-2.51 (3H, br), 2.30 (4H, m), 1.86 (3H, br), 1.63 (1H, br)

### Example 236

According to the same manner as that of Example 227 except that 150 mg of 1-methoxy-2-propanol was used in place of 2-propoxyethanol, 220 mg of a compound represented by the formula (236):

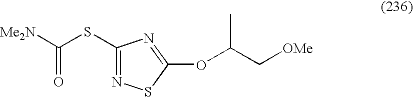

(hereinafter, referred to as present compound (236)) was obtained.

¹H-NMR (CDCl₃, TMS) δ (ppm): 5.35-5.28 (1H, m), 3.64-3.57 (2H, m), 3.40 (3H, s), 3.04 (6H, br), 1.45 (3H, d)

### Example 237

In 2 ml of tetrahydrofuran were dissolved 340 mg of the compound represented by the formula (IIa-1) and 200 mg of diethylene glycol monomethyl ether, 70 mg of sodium hydride (60% oily) and 0.5 ml of tetrahydrofuran were added under ice-cooling, and the mixture was stirred at room temperature for 6 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 400 mg of a compound represented by the formula (237):

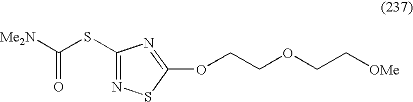

(hereinafter, referred to as present compound (237)).

¹H-NMR (CDCl₃, TMS) δ (ppm): 4.71-4.69 (2H, m), 3.88-3.86 (2H, m), 3.69-3.67 (2H, m), 3.58-3.55 (2H, m), 3.39 (3H, s), 3.04 (6H, br)

### Example 238

In 3 ml of tetrahydrofuran were dissolved 340 mg of the compound represented by the formula (IIa-1) and 430 mg of 1,2,:3,4-di-O-isoproplidene-D-galactopyranose, 70 mg of sodium hydride (60% oily) and 0.5 ml of tetrahydrofuran were added under ice-cooling, and the mixture was stirred at room temperature for 6 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 670 mg of a compound represented by the formula (238):

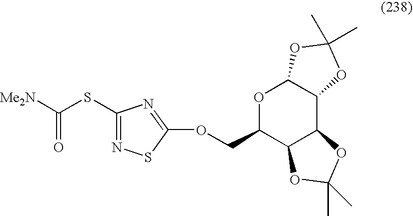

(hereinafter, referred to as present compound (238)).

¹H-NMR (CDCl₃, TMS) δ (ppm): 5.56 (1H, d), 4.73 (1H, dd), 4.66-4.60 (2H, m), 4.35 (1H, dd), 4.29 (1H, dd), 4.26-4.22 (1H, m), 3.04 (6H, br), 1.51 (3H, s), 1.46 (3H, s), 1.34 (6H, s)

### Example 239

In 2 ml of tetrahydrofuran were dissolved 220 mg of the compound represented by the formula (IIa-1) and 130 mg of tetrahydro-2H-thiopyran-4-ol, 40 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 270 mg of the crude product of a compound represented by the formula (239):

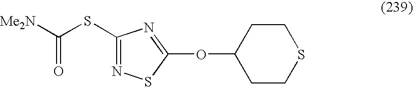

(hereinafter, referred to as present compound (239)).

¹H-NMR (CDCl₃, TMS) δ (ppm): 5.19-5.13 (1H, m), 3.04 (6H, br), 2.90-2.84 (2H, m), 2.66-2.57 (2H, m), 2.34-2.27 (2H, m), 2.17-2.11 (2H, m)

### Example 240

In 2.5 ml of chloroform were dissolved 250 mg of the present compound (226):

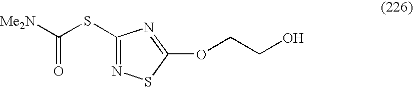

and 390 mg of disopropylethylamine, a solution of 200 mg of chloromethyl methyl ether in 0.5 ml of chloroform was added dropwise under ice-cooling, and the mixture was stirred at room temperature for 5 hours. Thereafter, 130 mg of diisopropylethylamine and 100 mg of chloromethyl methyl ether were further added, and the mixture was heated at refluxing for 30 minutes. Thereafter, the reaction mixture was cooled to room temperature, and water was added, followed by extraction with chloroform. The organic layer was dried with sodium sulfate, and concentrated under reduced pressure. The residue was subjected to silica gel preparative thin layer chromatography to obtain 270 mg of the crude product of a compound represented by the formula (240):

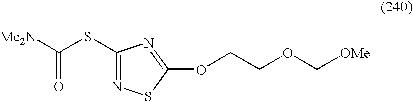

(hereinafter, referred to as present compound (240)).

¹H-NMR (CDCl₃, TMS) δ (ppm): 4.72-4.70 (2H, m), 4.68 (2H, s), 3.92-3.90 (2H, m), 3.38 (3H, s), 3.04 (6H, br)

### Example 241

According to the same manner as that of Example 227 except that 170 mg of 3-ethoxy-1-propanol was used in place of 2-propoxyethanol, 280 mg of a compound represented by the formula (241):

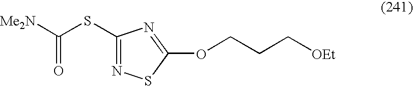

(hereinafter, referred to as present compound (241)) was obtained.

¹H-NMR (CDCl₃, TMS) δ (ppm): 4.62 (2H, t), 3.55 (2H, t), 3.48 (2H, q), 3.04 (6H, br), 2.13-2.06 (2H, m), 1.19 (3H, t)

### Example 242

According to the same manner as that of Example 227 except that 250 mg of 1,3-diethoxy-2-propanol was used in place of 2-propoxyethanol, 330 mg of a compound represented by the formula (242):

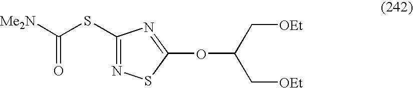

(hereinafter, referred to as present compound (242)) was obtained.

¹H-NMR (CDCl₃, TMS) δ (ppm): 5.31-5.27 (1H, m), 3.82-3.73 (4H, m), 3.58-3.50 (4H, m), 3.04 (6H, br), 1.18 (6H, t)

### Example 243

In 2 ml of tetrahydrofuran were dissolved 250 mg of the compound represented by the formula (IIa-6) and 80 mg of 2-methoxyethanol, 70 mg of sodium hydride (60% oily) and 0.5 ml of tetrahydrofuran were added under ice-cooling, and the mixture was stirred at room temperature for 1 hour. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 260 mg of a compound represented by the formula (243):

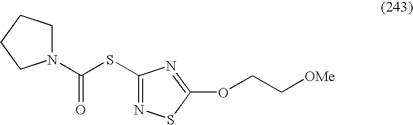

(hereinafter, referred to as present compound (243)).

¹H-NMR (CDCl₃, TMS) δ (ppm): 4.70-4.68 (2H, m), 3.77-3.75 (2H, m), 3.54 (2H, t), 3.45 (2H, t), 3.43 (3H, s), 2.03-1.96 (2H, m), 1.94-1.87 (2H, m)

### Example 244

According to the same manner as that of Example 243 except that 2-ethoxyethanol was used in place of 2-methoxyethanol, 280 mg of a compound represented by the formula (244):

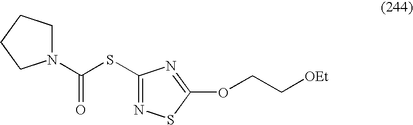

(hereinafter, referred to as present compound (244)) was obtained.

¹H-NMR (CDCl₃, TMS) δ (ppm): 4.70-4.67 (2H, m), 3.81-3.79 (2H, m), 3.57 (2H, q), 3.54 (2H, t), 3.45 (2H, t), 2.03-1.96 (2H, t), 1.94-1.87 (2H, m), 1.23 (3H, m)

### Example 245

In 2 ml of tetrahydrofuran were dissolved 340 mg of the compound represented by the formula (IIa-1) and 220 mg of trans-2-methoxycyclohexanol, 70 mg of sodium hydride (60% oily) and 0.5 ml of tetrahydrofuran were added under ice-cooling, and the mixture was stirred at room temperature for 4 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 250 mg of a compound represented by the formula (245):

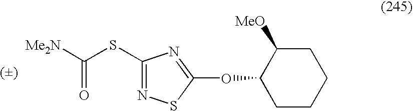

(hereinafter, referred to as present compound (245)).

¹H-NMR (CDCl₃, TMS) δ (ppm): 4.89-4.83 (1H, m), 3.39 (3H, s), 3.38-3.32 (1H, m), 3.04 (6H, br), 2.31-2.24 (1H, m), 2.11-2.06 (1H, m), 1.75-1.70 (2H, m), 1.60-1.50 (1H, m), 1.43-1.22 (3H, m)

### Example 246

According to the same manner as that of Example 237 except that 220 mg of diethylene glycol monoethyl ether was used in place of diethylene glycol monomethyl ether, 440 mg of a compound represented by the formula (246):

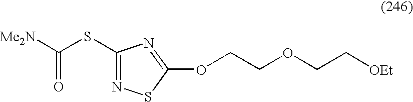

(hereinafter, referred to as present compound (246)) was obtained.

¹H-NMR (CDCl₃, TMS) δ (ppm): 4.70-4.68 (2H, m), 3.89-3.87 (2H, m), 3.70-3.67 (2H, m), 3.61-3.59 (2H, m), 3.53 (2H, q), 3.04 (6H, br), 1.21 (3H, t)

### Example 247

According to the same manner as that of Example 237 except that 260 mg of triethylene glycol monomethyl ether was used in place of diethylene glycol monomethyl ether, 440 mg of a compound represented by the formula (247):

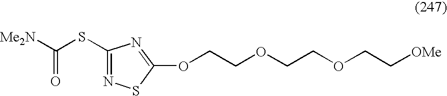

(hereinafter, referred to as present compound (247)) was obtained.

¹H-NMR (CDCl₃, TMS) δ (ppm): 4.70-4.68 (2H, m), 3.88-3.86 (2H, m), 3.72-3.64 (6H, m), 3.56-3.54 (2H, m), 3.38 (3H, s), 3.04 (6H, br)

### Example 248

In 2 ml of 1,1-diethoxyethane was dissolved 320 mg of the present compound (13):

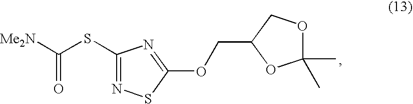

30 mg of p-toluenesulfonic acid monohydrate was added, and the mixture was stirred at room temperature for 5 hours, and allowed to stand for a whole day and night. Thereafter, the reaction mixture was concentrated, and an aqueous saturated sodium bicarbonate solution was added to the residue, followed by extraction with chloroform. The organic layer was dried with sodium sulfate, and concentrated under reduced pressure. The residue was subjected to medium pressure preparative liquid chromatography to obtain 220 mg of a compound represented by the formula (248):

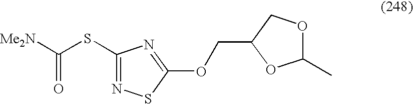

(hereinafter, referred to as present compound (248)).

¹H-NMR (CDCl₃, TMS) δ (ppm): 5.16 (0.5H, q), 5.07 (0.5H, q), 4.62-4.43 (3H, m), 4.23 (0.5H, dd), 3.98 (0.5H, dd), 3.89 (0.5H, dd), 3.67 (0.5H, dd), 3.04 (6H, br), 1.41 (1.5H, d), 1.39 (1.5H, d)

### Example 249

In 2 ml of methyl ethyl ketone was dissolved 320 mg of the present compound (13), 20 mg of p-toluenesulfonic acid monohydrate was added, and the mixture was stirred at room temperature for 8 hours, and allowed to stand for a whole day and night. Thereafter, the reaction mixture was concentrated, and an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography and medium pressure preparative liquid chromatography to obtain 270 mg of a compound represented by the formula (249):

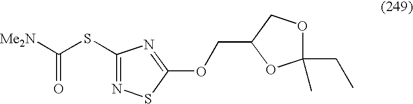

(hereinafter, referred to as present compound (249)).

¹H-NMR (CDCl₃, TMS) δ (ppm): 4.64-4.59 (1H, m), 4.56-4.44 (2H, m), 4.17-4.12 (1H, m), 3.84-3.77 (1H, m), 3.04 (6H, br), 1.74-1.64 (2H, m), 1.38 (1.5H, s), 1.33 (1.5H, s), 0.96-0.92 (3H, m)

### Example 250

In 2 ml of tetrahydrofuran were dissolved 340 mg of the compound represented by the formula (IIa-1) and 190 mg of trans-2-methoxycyclopentane, 70 mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 280 mg of a compound represented by the formula (250):

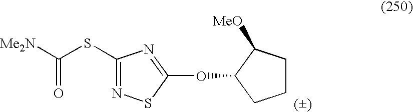

(hereinafter, referred to as present compound (250)).

¹H-NMR (CDCl₃, TMS) δ (ppm): 5.26-5.23 (1H, m), 3.93-3.90 (1H, m), 3.39 (3H, s), 3.04 (6H, br), 2.24-2.15 (1H, m), 2.06-1.98 (1H, m), 1.94-1.65 (4H, m)

### Example 251

In 2 ml of cyclopentanone was dissolved 320 mg of the present compound (13), 20 mg of p-toluenesulfonic acid monohydrate was added, and the mixture was stirred at room temperature for 6 hours. Thereafter, the reaction mixture was slightly concentrated under reduced pressure, the mixture was stirred for 3 hours and then allowed to stand for a whole day and night. Thereafter, the reaction mixture was concentrated, an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 290 mg of a compound represented by the formula (251):

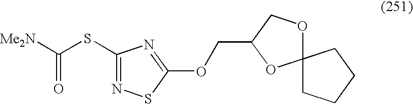

(hereinafter, referred to as present compound (251)).

¹H-NMR (CDCl₃, TMS) δ (ppm): 4.60 (1H, dd), 4.54-4.43 (2H, m), 4.06 (1H, dd), 3.79 (1H, dd), 3.04 (6H, br), 1.90-1.66 (8H, m)

### Example 252

In 2 ml of tetrahydrofuran were dissolved 140 mg of the compound represented by the formula (IIa-1) and 100 mg of the compound represented by the formula (XX-1):

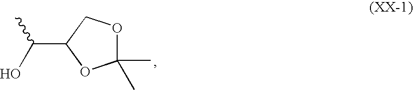

70 mg of sodium hydride (60% oily) and 0.5 ml of tetrahydrofuran were added under ice-cooling, and the mixture was stirred at room temperature for 4 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 270 mg of a compound represented by the formula (252):

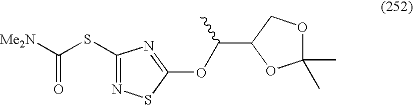

(hereinafter, referred to as present compound (252)).

¹H-NMR (CDCl₃, TMS) δ (ppm): 5.23-5.15 (1H, m), 4.33-4.27 (1H, m), 4.12 (0.7H, dd), 4.08 (0.3H, dd), 3.87 (0.7H, dd), 3.80 (0.3H, dd), 1.48-1.37 (9H, m)

### Example 253

In 3 ml of diethyl ketone was dissolved 320 mg of the present compound (13), 20 mg of p-toluenesulfonic acid monohydrate was added, and the mixture was stirred at room temperature for 5 hours, and allowed to stand for a whole day and night. Thereafter, the reaction mixture was concentrated under reduced pressure, 3 ml of diethyl ketone and 20 mg of p-toluenesulfonic acid monohydrate were added to the residue, and the mixture was further stirred for 5 hours. Thereafter, the reaction mixture was concentrated under reduced pressure, and an aqueous saturated sodium chloride solution was added to the residue, followed by extraction with chloroform. The organic layer was dried with sodium sulfate, and concentrated under reduced pressure. The residue was subjected to medium pressure preparative liquid chromatography to obtain 190 mg of a compound represented by the formula (253):

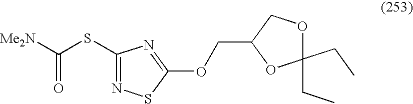

(hereinafter, referred to as present compound (253)).

¹H-NMR (CDCl₃, TMS) δ (ppm): 4.64-4.60 (1H, m), 4.56-4.47 (2H, m), 4.15 (1H, dd), 3.77 (1H, dd), 3.04(6H, br), 1.71-1.62 (4H, m), 0.93-0.89 (6H, m)

### Example 254

In 2.5 ml of tetrahydrofuran were dissolved 390 mg of the compound represented by the formula (IIa-1) and 250 mg of 1,3-dimethoxy-2-propanol, 70 mg of sodium hydride (60% oily) and 0.5 ml of tetrahydrofuran were added under ice-cooling, and the mixture was stirred at room temperature for 5 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 390 mg of a compound represented by the formula (254):

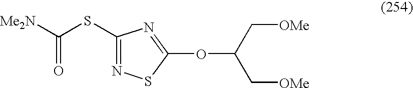

(hereinafter, referred to as present compound (254)).

¹H-NMR (CDCl₃, TMS) δ (ppm): 5.36-5.31 (1H, m), 3.77-3.70 (4H, m), 3.39 (6H, s), 3.04 (6H, br)

### Example 255

In 2 ml of tetrahydrofuran was dissolved 340 mg of a compound represented by the formula (IIa-1) and 100 mg of allyl alcohol, 70mg of sodium hydride (60% oily) and 0.5 ml of tetrahydrofuran were added under ice-cooling, and the mixture was stirred at room temperature for 2 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 310 mg of a compound represented by the formula (255):

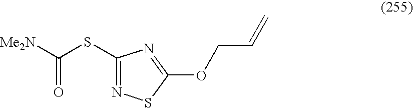

(hereinafter, referred to as present compound (255)).

¹H-NMR (CDCl₃, TMS) δ (ppm): 6.11-6.01 (1H, m), 5.49-5.44 (1H, m), 5.40-5.36 (1H, m), 5.03-5.01 (2H, m), 3.05 (6H, br)

### Example 256

In 2.5 ml of toluene were dissolved 320mg of the present compound (13) and 200 mg of tetrahydro-4H-pyran-4-one, 40 mg of p-toluenesulfonic acid monohydrate was added, and the mixture was stirred at room temperature for 5 hours. The reaction mixture was concentrated under reduced pressure, and an aqueous saturated sodium chloride solution was added to the residue, followed by extraction with chloroform. The organic layer was dried with sodium sulfate, and concentrated under reduced pressure. The residue was subjected to silica gel preparative thin layer chromatography to obtain 330 mg of a compound represented by the formula (256):

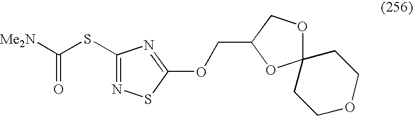

(hereinafter, referred to as present compound (256)).

¹H-NMR (CDCl₃, TMS) δ (ppm): 4.64-4.51 (3H, m), 4.19-4.14 (1H, m), 3.90-3.86 (1H, m), 3.81-3.73 (4H, m), 3.04 (6H, br), 1.82-1.73 (4H, m)

### Example 257

In 2 ml of propionaldehyde was dissolved 320 mg of the present compound (13), 30 mg of p-toluenesulfonic acid monohydrate was added, and the mixture was stirred at room temperature for 5 hours. Thereafter, the reaction mixture was slightly concentrated under reduced pressure, stirred for 3 hours, and allowed to stand overnight. Thereafter, the reaction mixture was concentrated under reduced pressure, and an aqueous saturated sodium chloride solution was added to the residue, followed by extraction with chloroform. The organic layer was dried with sodium sulfate, and concentrated under reduced pressure. The residue was subjected to silica gel preparative thin layer chromatography to obtain 210 mg of a compound represented by the formula (257):

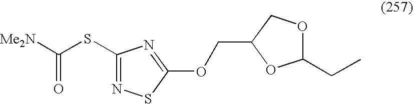

(hereinafter, referred to as present compound (257)).

¹H-NMR (CDCl₃, TMS) δ (ppm): 4.99 (0.5H, t), 4.91 (0.5H, t), 4.60-4.45 (3H, m), 4.21 (0.5H, dd), 3.99 (0.5H, dd), 3.88 (0.5H, dd), 3.69 (0.5H, dd), 3.04 (6H, br), 1.73-1.69 (2H, m), 0.99-0.95 (3H, m)

### Example 258

In 2 ml of 1,1-diethoxyethane were dissolved 300 mg of the present compound (134):

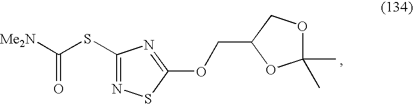

30 mg of p-toluenesulfonic acid monohydrate was added, and the mixture was stirred at room temperature for 8 hours, and allowed to stand overnight. Thereafter, the reaction mixture was concentrated under reduced pressure, an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 250 mg of a compound represented by the formula (258):

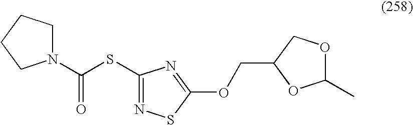

(hereinafter, referred to as present compound (258)).

¹H-NMR (CDCl₃, TMS) δ (ppm): 5.16 (0.4H, q), 5.07 (0.6H, q), 4.62-4.44 (3H, m), 4.23 (0.4H, dd), 3.98 (0.6H, dd), 3.89 (0.6H, dd),3.67 (0.4H, dd), 3.54 (2H, t), 3.45 (2H, t), 2.03-1.96 (2H, m), 1.94-1.88 (2H, m)

### Example 259

In 2 ml of tetrahydrofuran were dissolved 220 mg of the compound represented by the formula (IIa-1) and 145 mg of (R)-(−)-2,2-dimethyl-1,3-dioxolane-4-methanol, 40 mg of sodium hydride (60% oily) and 0.5 ml of tetrahydrofuran were added under ice-cooling, and the mixture was stirred at room temperature for 7 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 250 mg of a compound represented by the formula (259):

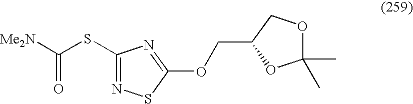

(hereinafter, referred to as present compound (259)).

¹H-NMR (CDCl₃, TMS) δ (ppm): 4.64-4.58 (1H, m), 4.54-4.48 (2H, m), 4.16-4.12 (1H, m), 3.85-3.81 (1H, m), 3.04 (6H, br), 1.45 (3H, s), 1.39 (3H, s)

### Example 260

According to the same manner as that of Example 259 except that (S)-(+)-2,2-dimethyl-1,3-dioxolane-4-methanol was used in place of (R)-(−)-2,2-dimethyl-1,3-dioxolane-4-methanol, 250 mg of a compound represented by the formula (260):

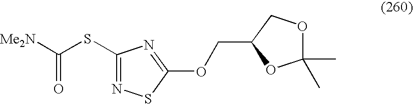

(hereinafter, referred to as present compound (260)) was obtained.

¹H-NMR (CDCl₃, TMS) δ (ppm): 4.64-4.58 (1H, m), 4.54-4.48 (2H, m), 4.16-4.12 (1H, m), 3.85-3.81 (1H, m), 3.04 (6H, br), 1.45 (3H, s), 1.39 (3H, s)

### Example 261 and Example 262

In 4 ml of tetrahydrofuran were dissolved 500 mg of the compound represented by the formula (IIa-6) and 320 g of a compound represented by the formula (XX-2):

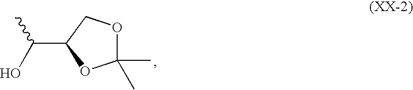

90 mg of sodium hydride (60% oily) and 0.5 ml of tetrahydrofuran were added under ice-cooling, and the mixture was stirred at room temperature for 5 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to medium pressure preparative liquid chromatography to obtain 450 mg of a compound represented by the formula (261):

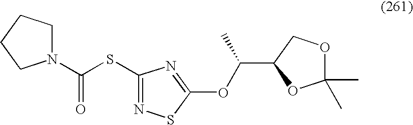

(hereinafter, referred to as present compound (261)) and 130 mg of a compound represented by the formula (262):

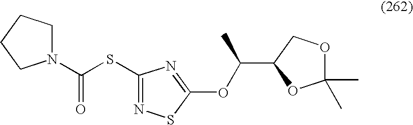

(hereinafter, referred to as present compound (262).

### Present Compound (261)

¹H-NMR (CDCl₃, TMS) δ (ppm): 5.21-5.15 (1H, m), 4.33-4.29 (1H, m), 4.12 (1H, dd), 3.87 (1H, dd), 3.54 (2H, t), 3.45 (2H, t), 2.03-1.96 (2H, m), 1.94-1.87 (2H, m), 1.47 (3H, d), 1.43 (3H, s), 1.37 (3H, s)

### Present Compound (262)

¹H-NMR (CDCl₃, TMS) δ (ppm): 5.24-5.17 (1H, m), 4.32-4.27 (1H, m), 4.08 (1H, dd), 3.79 (1H, dd), 3.54 (2H, t), 3.45 (2H, t), 2.03-1.96 (2H, m), 1.94-1.87 (2H, m), 1.44-1.42 (6H, m), 1.38(3H, s)

### Example 263

In 3 ml of tetrahydrofuran were dissolved 320 mg of the compound represented by the formula (IIa-1) and 230 mg of a compound represented by the formula (XX-2):

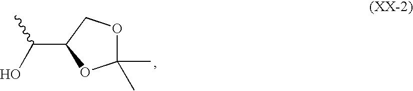

60 mg of sodium hydride (60% oily) and 0.5 ml of tetrahydrofuran were added under ice-cooling, and the mixture was stirred at room temperature for 4 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 380 mg of a compound represented by the formula (263):

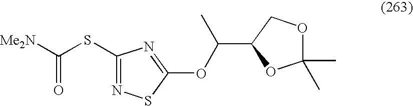

(hereinafter, referred to as present compound (263)). H-NMR (CDCl₃, TMS) δ (ppm): 5.23-5.15 (1H, m), 4.33-4.27 (1H, m), 4.12 (0.7H, dd), 4.08 (0.3H, dd), 3.87 (0.7H, dd), 3.80 (0.3H, dd), 1.48-1.37 (9H, m)

### Example 264 and Example 265

In 2 ml of 1,1-diethoxyethane was dissolved 160 mg of the present compound (263), 20 mg of p-toluenesulfonic acid monohydrate was added, and the mixture was stirred at room temperature for 8 hours, and allowed to stand for a whole day and night. Thereafter, the reaction mixture was concentrated under reduced pressure, and an aqueous saturated sodium chloride solution was added to the residue, followed by extraction with chloroform. The organic layer was dried with sodium sulfate, and concentrated under reduced pressure. The residue was subjected to medium pressure preparative liquid chromatography to obtain 30 mg and 45 mg of two compounds represented by the following formula:

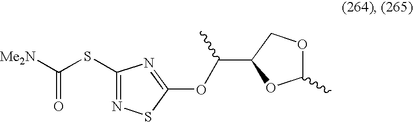

(hereinafter, referred to as present compound (264) and present compound (265)), respectively.

Stereochemistry of present compounds (264) and (265) is unknown, but each compound is a single isomer, and has a relationship of diastereomer.

### Present Compound (264)

¹H-NMR (CDCl₃, TMS) δ (ppm): 5.26-5.20 (1H, m) , 5.13 (1H, q) , 4.27-4.20 (2H, m) , 3.81 (1H, dd) , 3.04 (6H, br) , 1.48 (3H, d), 1.36 (3H, d)

### Present Compound (265)

¹H-NMR (CDCl₃, TMS) δ (ppm): 5.20-5.15 (1H, m), 5.05 (1H, q), 4.32-4.28 (1H, m), 3.97-3.90 (2H, m), 3.04 (6H, br), 1.47 (3H, d), 1.39 (3H, d)

### Example 266

In 2 ml of 1,1-diethoxyethane was dissolved 290 mg of the present compound (261), 30 mg of p-toluenesulfonic acid monohydrate was added, and the mixture was stirred at room temperature for 8 hours, and allowed to stand for a whole day and night. Thereafter, the reaction mixture was concentrated under reduced pressure, and an aqueous saturated sodium chloride solution was added to the residue, followed by extraction with chloroform. The organic layer was dried with sodium sulfate, and concentrated under reduced pressure. The residue was subjected to silica gel preparative thin layer chromatography to obtain 210 mg of a compound represented by the formula (266):

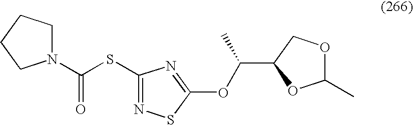

(hereinafter, referred to as present compound (266)).

¹H-NMR (CDCl₃, TMS) δ (ppm): 5.27-5.16 (1H, m), 5.13 (0.4H, q), 5.05 (0.6H, q), 4.32-4.20 (1.4H, m), 3.97-3.90 (0.6H, m), 3.83-3.81 (0.4H, m), 3.54(2H, t), 3.45(2H, t), 2.03-1.96 (2H, m) , 1.94-1.87 (2H, m), 1.49-1.46 (3H, m), 1.39 (1.8H, d), 1.36 (1.2H ,d)

### Example 267

In 1 ml of 1,1-diethoxyethane was dissolved 74 mg of the present compound (262), 10 mg of p-toluenesulfonic acid monohydrate was added, and the mixture was stirred at room temperature for 8 hours, and allowed to stand for a whole day and night. Thereafter, the reaction mixture was concentrated under reduced pressure, and an aqueous saturated sodium chloride solution was added to the residue, followed by extraction with chloroform. The organic layer was dried with sodium sulfate, and concentrated under reduced pressure. The residue was subjected to silica gel preparative thin layer chromatography to obtain 48 mg of a compound represented by the formula (267):

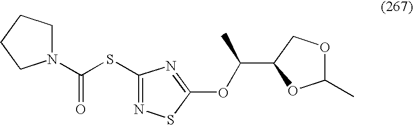

(hereinafter, referred to as present compound (267)).

¹H-NMR (CDCl₃, TMS) δ (ppm): 5.27-5.21 (1H, m), 5.15 (0.3H, q), 5.06 (0.7H, q), 4.29-4.15 (1H, m), 4.19-4.15 (0.3H, m), 3.96-3.88 (1.4H, m), 3.64 (0.3H, dd), 3.54 (2H, t), 3.45 (2H, t), 2.03-1.96 (2H, m), 1.94-1.87 (2H, m),1.45-1.43 (3H, m), 1.40 (2.1H, d), 1.37 (0.9H, d)

### Example 268

In 2 ml of tetrahydrofuran and 0.5 ml of water was dissolved 320 mg of the present compound (13), 20 mg of p-toluenesulfonic acid monohydrate was added, and the mixture was stirred at room temperature for 3 hours, and further stirred at about 50° C. for 4 hours. Thereafter, the reaction mixture was cooled to room temperature, and concentrated under reduced pressure, and an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 130 mg of a compound represented by the formula (268):

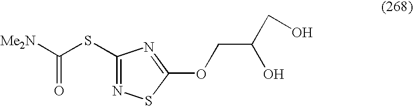

(hereinafter, referred to as present compound (268)).

¹H-NMR (CDCl₃, TMS) δ (ppm): 4.71 (1H, dd), 4.64 (1H, dd), 4.12-4.06 (1H, m), 3.78-3.65 (2H, m), 3.09-3.03 (7H, m), 2.62 (1H, t)

### Example 269

In 2 ml of toluene was dissolved 300 mg of the present compound (134) and 150 mg of propionaldehyde, 30 mg of p-toluenesulfonic acid monohydrate was added, and the mixture was stirred at room temperature for 2 hours, and allowed to stand overnight. Thereafter, the reaction mixture was concentrated under reduced pressure, and an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 240 mg of a compound represented by the formula (269):

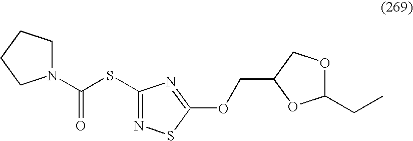

(hereinafter, referred to as present compound (269)).

¹H-NMR (CDCl₃, TMS) δ (ppm): 4.99 (0.5H, t), 4.91 (0.5H, t), 4.60-4.45 (2H, m), 4.21 (0.5H, dd), 3.99 (0.5H, dd), 3.88 (0.5H, dd), 3.68 (0.5H, dd), 3.54 (2H, t), 3.45 (2H, t), 2.03-1.96 (2H, m), 1.94-1.87 (2H, m), 1.75-1.66 (2H, m), 0.99-0.94 (3H, m)

### Example 270

In 3 ml of tetrahydrofuran were dissolved 420 mg of a compound represented by the formula (IIa-1) and 270 mg of 2,2-dimethyl-1,3-dioxane-5-ol, 70 mg of sodium hydride (60% oily) and 0.5 ml of tetrahydrofuran were added under ice-cooling, and the mixture was stirred at room temperature for 3 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 550 mg of a compound represented by the formula (270):

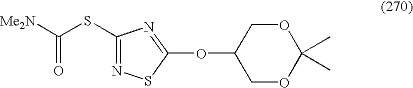

(hereinafter, referred to as present compound (270)).

¹H-NMR (CDCl₃, TMS) δ (ppm): 5.03 (1H, quint), 4.21 (2H, dd), 4.09 (2H, dd), 3.04 (6H, br), 1.47 (3H, s), 1.46 (3H, s)

### Example 271

In 2 ml of toluene were dissolved 320 mg of the present compound (13) and 300 mg of 1,1-diethoxy-2-methoxyethane, 20 mg of p-toluenesulfonic acid monohydrate was added, and the mixture was stirred at about 80° C. for 3 hours. Thereafter, the reaction mixture was cooled to room temperature, 300 mg of 1,1-diethoxy-2-methoxyethane and 20 mg of p-toluenesulfonic acid monohydrate were added, and the mixture was further stirred at about 80° C. for 5 hours. Thereafter, the reaction mixture was cooled to room temperature, and concentrated under reduced pressure, and an aqueous saturated sodium bicarbonate solution was added to the residue, followed by extraction with chloroform. The organic layer was dried with sodium sulfate, and concentrated under reduced pressure. The residue was subjected to medium pressure preparative liquid chromatography to obtain 130 mg of a compound represented by the formula (271):

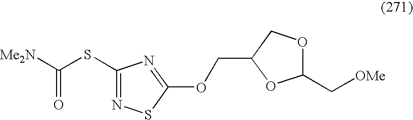

(hereinafter, referred to as present compound (271)).

¹H-NMR (CDCl₃, TMS) δ (ppm): 5.21 (0.2H, t), 5.10 (0.8H, t), 4.64-4.49 (3H, m), 4.23 (0.2H, d), 4.04 (0.8H, d), 3.93 (0.7H, d), 3.53-3.51 (1.6H, m), 3.50-3.49 (0.4H, m), 3.43 (0.6H, s), 3.42 (2.4H, s)

### Example 272

In 2 ml of toluene were dissolved 250 mg of the present compound (13) and 90 mg of butanal, 20 mg of p-toluenesulfonic acid monohydrate was added, and the mixture was allowed to stand for a whole day and night, and stirred at room temperature for 7 hours. Thereafter, the reaction mixture was concentrated under reduced pressure, and an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 210 mg of a compound represented by the formula (272):

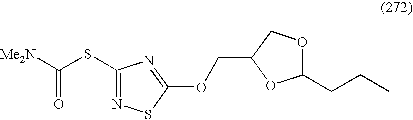

(hereinafter, referred to as present compound (272)).

¹H-NMR (CDCl₃, TMS) δ (ppm): 5.02 (0.4H, t), 4.94 (0.6H, t), 4.59-4.44 (3H, m), 4.21 (0.4H, dd), 3.97 (0.6H, dd), 3.88 (0.6H, dd), 3.67 (0.4H, dd), 3.04 (6H, br), 1.70-1.62 (2H, m), 1.50-1.39 (2H, m), 0.95 (3H, t)

### Example 273

In 2 ml of toluene were dissolved 320 mg of the present compound (270) and 180 mg of 1,1-diethoxyethane, 40 mg of p-toluenesulfonic acid monohydrate was added, and the mixture was stirred at room temperature for 10 hours, and allowed to stand for a whole day and night. Thereafter, the reaction mixture was slightly concentrated under reduced pressure. After 90 mg of 1,1-diethoxyethane was added and stirred at room temperature for another 10 hours, then allowed to stand for a whole day and night. Thereafter, the reaction mixture was concentrated under reduced pressure, and an aqueous saturated sodium bicarbonate solution was added to the residue, followed by extraction with chloroform. The organic layer was dried with sodium sulfate, and concentrated under reduced pressure. The residue was subjected to medium pressure preparative liquid chromatography to obtain 80 mg of a compound represented by the formula (273):

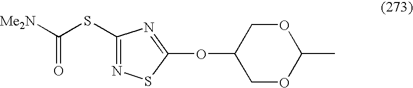

(hereinafter, referred to as present compound (273)).

¹H-NMR (CDCl₃, TMS) δ (ppm): 4.99-4.98 (1H, m), 4.79 (1H, q), 4.39 (2H, dd), 4.03 (2H, dd), 3.03 (6H, br), 1.39 (3H, d)

### Example 274

In 2 ml of toluene were dissolved 280 mg of the present compound (13) and 110 mg of heptanal, 30 mg of p-toluenesulfonic acid monohydrate was added, allowed to stand for a whole day and night, then the mixture was stirred at room temperature for 7 hours. Thereafter, the reaction mixture was concentrated under reduced pressure, llOmg of heptanal, 30 mg of p-toluenesulfonic acid monohydrate and 3 ml of toluene were added, and the mixture was stirred at room temperature for 7 hours. Thereafter, the reaction mixture was concentrated under reduced pressure, and an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 73 mg of a compound represented by the formula (274):

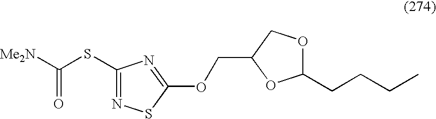

(hereinafter, referred to as present compound (274)).

¹H-NMR (CDCl₃, TMS) δ (ppm): 5.01 (0.3H, t), 4.93 (0.7H, t), 4.61-4.43 (3H, m), 4.21 (0.3H, dd), 3.98 (0.7H, dd), 3.88 (0.7H, dd), 3.67 (0.3H, dd), 3.04 (6H, br), 1.71-1.65 (2H, m), 1.45-1.30 (4H, m), 0.91 (3H, t)

### Example 275

In 3 ml of tetrahydrofuran were dissolved 320 mg of the present compound (13) and 110 mg of isobutylaldehyde, 40 mg of p-toluenesulfonic acid monohydrate was added, and the mixture was stirred at room temperature for 10 hours, and allowed to stand for a whole day and night. Thereafter, the reaction mixture was concentrated under reduced pressure, 50 mg of isobutylaldehyde, and 2 ml of tetrahydrofuran were added to the residue, and the mixture was further stirred at room temperature for 7 hours. Thereafter, the reaction mixture was concentrated under reduced pressure, and an aqueous saturated sodium bicarbonate solution was added to the residue, followed by extraction with chloroform. The organic layer was dried with sodium sulfate, and concentrated under reduced pressure. The residue was subjected to medium pressure preparative liquid chromatography to obtain 170 mg of a compound represented by the formula (275):

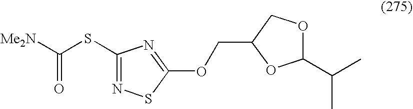

(hereinafter, referred to as present compound (275)).

¹H-NMR (CDCl₃, TMS) δ (ppm): 4.78 (0.3H, t), 4.70 (0.7H, t), 4.59-4.43 (3H, t), 4.19 (0.3H, dd), 3.98 (0.7H, dd), 3.87 (0.7H, dd), 3.69 (0.3H, dd), 3.04 (6H, br), 1.90-1.80 (1H, m), 0.97-0.94 (6H, m)

### Example 276

In 3 ml of tetrahydrofuran were dissolved 320 mg of the present compound (13) and 130 mg of pivalaldehyde, 40 mg of p-toluenesulfonic acid monohydrate was added, and the mixture was stirred at room temperature for 10 hours, and allowed to stand for a whole day and night. Thereafter, the reaction mixture was concentrated under reduced pressure, 50 mg of pivalaldehyde, and 2 ml of tetrahydrofuran were added to the residue, and stirred at room temperature for 7 hours, and allowed to stand for a whole day and night. Thereafter, 200 mg of pivalic aldehyde, and 20 mg of p-toluenesulfonic acid monohydrate were added to the residue, the mixture was further stirred at about 50° C. for 10 hours. Thereafter, the reaction mixture was cooled to room temperature, and concentrated under reduced pressure, and an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 160 mg of a compound represented by the formula (276):

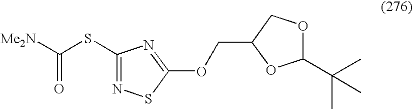

(hereinafter, referred to as present compound (276)).

¹H-NMR (CDCl₃, TMS) δ (ppm): 4.66 (0.3H, s), 4.60-4.53 (2.7H, m), 4.50-4.43 (1H, m), 4.18 (0.3H, dd), 3.99 (0.7H, dd), 3.86 (0.3H, dd), 3.70 (0.3H, dd), 3.04 (6H, br), 0.93 (6.3H, s), 0.92 (2.7H, s)

### Example 277

In 3 ml of tetrahydrofuran were dissolved 410 mg of a compound represented by the formula (IIa-1) and 240 mg of 2,3-dimethoxypropanol, 80 mg of sodium hydride (60% oily) and 0.5 ml of tetrahydrofuran were added under ice-cooling, and the mixture was stirred at room temperature for 3 hours. Thereafter, 10 mg of sodium hydride was added, and the mixture was further stirred at room temperature for 1 hour. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 510 mg of a compound represented by the formula (277):

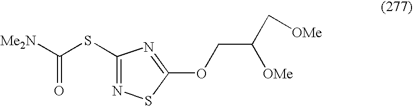

(hereinafter, referred to as present compound (277)).

¹H-NMR (CDCl₃, TMS) δ (ppm): 4.69 (1H, dd), 4.58 (1H, dd), 3.75-3.70 (1H, m), 3.55-3.53 (2H, m), 3.49 (3H, s), 3.38 (3H, s), 3.04 (6H, br)

### Example 278

In 2 ml of tetrahydrofuran were dissolved 340 mg of a compound represented by the formula (IIa-1) and 240 mg of 2,2-dimethyl-1,3-dioxolane-4-ethanol, 70 mg of sodium hydride (60% oily) and 0.5 ml of tetrahydrofuran were added under ice-cooling, and the mixture was stirred at room temperature for 3 hours. Thereafter, 10 mg of sodium hydride was added, and the mixture was further stirred at room temperature for 1 hour. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 410 mg of the crude product of a compound represented by the formula (278):

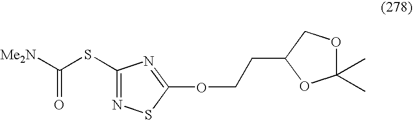

(hereinafter, referred to as present compound (278)).

¹H-NMR (CDCl₃, TMS) δ (ppm): 4.71-4.60 (2H, m), 4.29-4.22 (1H, m), 4.12-4.08 (1H, m), 3.63-3.58 (1H, m), 3.04 (6H, br), 2.13-2.00 (2H, m), 1.41 (3H, s), 1.35 (3H, s)

### Example 279

In 3 ml of tetrahydrofuran were dissolved 330 mg of a compound represented by the formula (IIa-1) and 240 mg of 3-diethoxypropanol, 70 mg of sodium hydride (60% oily) and 0.5 ml of tetrahydrofuran were added under ice-cooling, and the mixture was stirred at room temperature for 4 hours. Thereafter, an aqueous saturated ammonium chloride solution was added to the reaction mixture, followed by extraction with t-butyl methyl ether. The organic layer was dried with sodium sulfate, and concentrated under reduced pressure. The residue was subjected to silica gel preparative thin layer chromatography to obtain 460 mg of a compound represented by the formula (279):

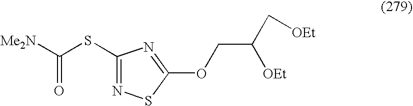

(hereinafter, referred to as present compound (279)).

¹H-NMR (CDCl₃, TMS) δ (ppm): 4.67 (1H, dd), 4.57 (1H, dd), 3.84-3.79 (1H, m), 3.66 (2H, q), 3.59-3.49 (4H, m), 3.04 (6H, br), 1.23-1.17 (6H, m)

### Example 280

In 3 ml of tetrahydrofuran were dissolved 320 mg of the present compound (13) and 160 mg of benzaldehyde, 60 mg of p-toluenesulfonic acid monohydrate was added, and the mixture was stirred at room temperature for 7 hours, and allowed to stand for a whole day and night. Thereafter, the mixture was heated at refluxing for 10 hours, and cooled to room temperature. Thereafter, the reaction mixture was concentrated under reduced pressure, 30 mg of p-toluenesulfonic acid monohydrate and 3 ml of tetrahydrofuran were added, and the mixture was further heated at refluxing for 4 hours. Thereafter, the reaction mixture was cooled to room temperature, and concentrated under reduced pressure, and an aqueous saturated sodium bicarbonate solution was added to the residue, followed by extraction with chloroform. The organic layer was dried with sodium sulfate, and concentrated under reduced pressure. The residue was subjected to silica gel preparative thin layer chromatography to obtain 62 mg of a compound represented by the formula (280):

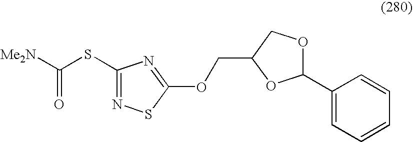

(hereinafter, referred to as present compound (280)).

¹H-NMR (CDCl₃, TMS) δ (ppm): 7.50-7.46 (2H, m), 7.41-7.37 (3H, m), 5.86 (1H, s), 4.72-4.60 (3H, m), 4.21-4.17 (1H, m), 4.09-4.06 (1H, m), 3.04 (6H, br)

Then, production of intermediates for producing the present compound will be shown by Production

### Examples.

### Production Example 1

In 100 ml of tetrahydrofuran were suspended 10.0 g of N,N-dimethylcarbamoyl chloride and 5.9 g of thiourea, and the suspension was heated at refluxing for 10 hours. To the mixed solution was added 1.0 g of N,N-dimethylcarbamoyl chloride, and the mixture was heated at refluxing for 2 hours. Thereafter, the reaction mixture was cooled to room temperature, and the resulting crystal was filtered off and washed sequentially with tetrahydrofuran and hexane to obtain 13.8 g of a compound represented by the formula (IXa-1).

¹H-NMR (DMSO-d₆, TMS) δ (ppm): 9.71 (4H, br), 3.01 (3H, s), 2.99 (3H, s)

### Production Example 2

In 40ml of tetrahydrofuran were suspended 5.0 g of 4-morpholinecarbonyl chloride and 2.3 g of thiourea, and the suspension was heated at refluxing for 5 hours. Thereafter, the reaction mixture was cooled to room temperature, and the resulting crystal was filtered off and washed sequentially with tetrahydrofuran and hexane to obtain 6.6 g of a compound represented by the formula (IXa-2).

¹H-NMR(DMSO-d₆, TMS) δ (ppm): 9.82 (4H, brs), 3.64 (4H, t), 3.50 (4H, br)

### Production Example 3

In 40ml of tetrahydrofuran were suspended 5.0 g of N,N-diphenylcarbamoyl chloride and 1.5 g of thiourea, and the suspension was heated at refluxing for 5 hours. Thereafter, the reaction mixture was cooled to room temperature, and the resulting crystal was filtered off and washed sequentially with tetrahydrofuran and hexane to obtain 4.86 g of a compound represented by the formula (IXa-3).

¹H-NMR(DMSO-d₆, TMS) δ (ppm): 9.72 (2H, brs), 9.66 (2H, brs), 7.51 (10H, s)

### Production Example 4

In 30 ml of tetrahydrofuran were suspended 5.6 g of N-methyl-N-phenylcarbamoyl chloride and 2.3 g of thiourea, and the suspension was heated at refluxing for 10 hours. Thereafter, the reaction mixture was cooled to room temperature, and the resulting crystal was filtered off and washed sequentially with ethyl acetate and hexane to obtain 7.25 g of a compound represented by the formula (IXa-4).

¹H-NMR (DMSO-d6, TMS) δ (ppm): 9.65 (4H, brs), 7.58-7.50 (5H, m), 3.31 (3H, s)

### Production Example 5

In 30 ml of tetrahydrofuran were suspended 4.0 g of 1-piperidinecarbonyl chloride and 1.88 g of thiourea, and the suspension was heated at refluxing for 10 hours. Thereafter, the reaction mixture was cooled to room temperature, and the resulting crystal was filtered off and washed sequentially with ethyl acetate and hexane to obtain 4.28 g of a compound represented by the formula (IXa-5).

¹H-NMR (DMSO-d6, TMS) δ (ppm): 9.81 (4H, br), 3.52 (2H, br), 3.39 (2H, br), 1.57-1.52 (6H, brm)

### Production Example 6

In 40 ml of tetrahydrofuran were suspended 5.0 g of 1-pyrrolidinecarbonyl chloride and 2.6 g of thiourea, and the suspension was heated at refluxing for 10 hours. Thereafter, to the reaction mixture was added 1.3 g of 1-pyrrolidinecarbonyl chloride, and the mixture was heated at refluxing for 2 hours. Thereafter, the reaction mixture was cooled to room temperature, and the resulting crystal was filtered off and washed sequentially with tetrahydrofuran and hexane to obtain 6.67 g of a compound represented by the formula (IXa-6).

¹H-NMR (DMSO-d6, TMS) δ (ppm): 9.77 (4H, brs), 3.46 (2H, t), 3.39 (2H, t), 1.97-1.82 (4H, m)

### Production Example 7

In 60 ml of tetrahydrofuran were suspended 8.1 g of N,N-diethylcarbamoyl chloride and 3.8 g of thiourea, and the suspension was stirred at 50° C. for 8 hours. To the mixed solution was added 1.0 g of 1-pyrrolidinecarbonyl chloride, and the mixture was further stirred at 50° C. for 2 hours. Thereafter, the reaction mixture was cooled to room temperature, and the resulting crystal was filtered off and washed sequentially with tetrahydrofuran and hexane to obtain 8.23 g of a compound represented by the formula (IXa-7).

¹H-NMR(DMSO-d₆, TMS) δ (ppm): 9.73 (4H, brs), 3.37 (4H, br), 1.19 (3H, br), 1.11 (3H, br)

### Production Example 8

In 2 ml of diethyl ether was dissolved 130 mg of (±)-2,2-dimethyl-1,3-dioxolane-4-carboxyaldehyde, 0.5 ml of a solution (ca. 3 M) of methylmagnesium bromide in diethyl ether was added dropwise under ice-cooling, and the mixture was stirred at room temperature for 2 hours. Thereafter, to the reaction mixture was added an aqueous saturated ammonium chloride solution, followed by extraction with t-butyl methyl ether. The organic layer was dried with sodium sulfate, and concentrated under reduced pressure to obtain 100 mg of a compound represented by the formula (XX-1):

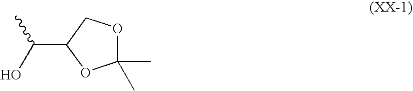

as the crude product.

¹H-NMR (CDCl₃, TMS) δ (ppm): 4.04-3.89 (3H, m), 3.71-3.67 (1H, m), 1.44 (3H, s), 1.37 (3H, s), 1.17-1.15 (3H, m)

### Production Example 9

In 10 ml of diethyl ether was dissolved 1.07 g of (R)-(+)-2,2-dimethyl-1,3-dioxolane-4-carboxyaldehyde, 3.8 ml of a solution (ca. 3 M) of methylmagnesium bromide in diethyl ether was added dropwise under ice-cooling, and the mixture was stirred at room temperature for 2 hours. Thereafter, to the reaction mixture was added an aqueous saturated ammonium chloride solution, followed by extraction with t-butyl methyl ether. The organic layer was dried with sodium sulfate, and concentrated under reduced pressure to obtain 550 mg of a compound represented by the formula (XX-2):

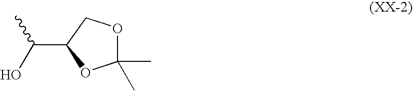

as the crude product.

¹H-NMR (CDCl₃, TMS) δ (ppm): 4.04-3.89 (3H, m), 3.71-3.67 (1H, m), 1.44 (3H, s), 1.37 (3H, s), 1.17-1.15 (3H, m)

Then, Preparation Examples will be shown. All the parts are by weight.

### Preparation Example 1

In a mixture of 35 parts of xylene and 35 parts of N,N-dimethylformamide is dissolved 10 parts of each of present compounds (1) to (148) and (201) to (277), 14 parts of polyoxyethylene styryl phenyl ether and 6 parts of calcium dodecylbenzenesulfonate are added, and the mixture is thoroughly stirred to obtain a 10% emulsion of each compound.

### Preparation Example 2

To a mixture of 4 parts of sodium laurylsulfate, 2 parts of calcium ligninsulfonate, 20 parts of a synthetic hydrated silicon dioxide fine powder and 54 parts of diatomaceous earth is added 20 parts of each of present compounds (1) to (148) and (201) to (277), and the mixture is thoroughly stirred to obtain a 20% wettable of each compound.

### Preparation Example 3

To 2 parts of each of present compounds (1) to (148) and (201) to (277) are added 1 part of a synthetic hydrated silicon dioxide fine powder, 2 parts of calcium ligninsulfonate, 30 parts of bentonite and 65 parts of kaolin clay, and the mixture is thoroughly stirred. Then, to the mixture is added a suitable amount of water, and the mixture is further stirred, granulated with a granulator, and circulation-dried to obtain 2% granules of each compound.

### Preparation Example 4

In a suitable amount of acetone is dissolved 1 part of each of present compounds (1) to (148) and (201) to (277), 5 parts of a synthetic hydrated silicon dioxide fine powder, 0.3 part of PAP, and 93.7 parts of fubasami clay, substances are mixed well , and acetone is removed by evaporation to obtain a 1% flowable of each compound.

### Preparation Example 5

Each 10 parts of each of present compounds (1) to (148) and (201) to (277); 35 parts of white carbon containing 50 parts of polyoxyethylene alkyl ether sulfate ammonium salt; and 55 parts of water are mixed, and finely-divided by a wet grinding method to obtain a 10% flowable of each compound.

### Preparation Example 6

In 5 parts of xylene and 5 parts of trichloroethane is dissolved 0.1 part of each of present compounds (1) to (148) and (201) to (277), and the solution is mixed with 89.9 parts of debrominated kerosene to obtain a 0.1% oily preparation of each compound.

### Preparation Example 7

In 0.5 ml of acetone is dissolved 10 mg of each of present compounds (1) to (148) and (201) to (277), the solution is added to 5 g of an animal solid feed powder (CLEA Rodent Diet CE-2 for rearing and breeding, trade name of Clea Japan, Inc.), and the mixture is uniformly mixed. Then, acetone is evaporated to obtain poison bait of each compound.

Then, the noxious arthropod controlling efficacy of the present compound will be shown by Test Examples.

### Test Example 1

Each of preparations of present compounds (1) to (15), (17) to (19), (21) to (31), (33), (34), (36) to (45), (48) to (57), (59), (61), (62), (64), (67) to (70), (74), (77) to (83), (85) to (89), (91) to (93), (95) to (97), (99), (102), (103), (105) to (112), (117) to (130), (132) to (143), (146) to (148), (201), (204), (206) to (209), (211), (213) to (215), (218) to (223), (225), (227) to (267), and (269) to (277) obtained in Preparation Example 5 was diluted with water so that the active ingredient concentration became 500 ppm to prepare a test spray solution.

On the other hand, a cucumber was planted in a polyethylene cup, and grown until a first true leaf was developed, and 30 cotton aphids were parasitized thereon. One day after, the test spray solution was sprayed to the cucumber at a ratio of 20 ml/cup. Six days after spraying, the number of cotton aphids was investigated, and a controlling value was calculated according to the following equation.

Controlling value (%)={1−(Cb×Tai)/(Cai×Tb)}×100

wherein:

Cb is the number of worms before treatment of a non-treated group.

Cai is the number of worms at observation of a non-treated group.

Tb is the number of worms before treatment of a treated group.

Tai is the number of worms at observation of a treated group.

As a result, the groups treated with the test spray solutions of present compounds (1) to (15), (17) to (19), (21) to (31), (33), (34), (36) to (45), (48) to (57), (59), (61), (62), (64), (67) to (70), (74), (77) to (83), (85) to (89), (91) to (93), (95) to (97), (99), (102), (103), (105) to (112), (117) to (130), (132) to (143), (146) to (148), (201), (204), (206) to (209), (211), (213) to (215), (218) to (223), (225), (227) to (267), and (269) to (277) showed a controlling value of not lower than 90%, respectively.

### Test Example 2

Each of present compounds (1) to (15), (17), (19), (21) to (26), (28) to (30), (34), (36) to (41), (49), (50), (53) to (57), (59), (61), (62), (67) to (69), (79), (119) to (123), (125), (127), (129), (130), (133) to (142), (147), (201), (207) to (210), (213) to (215), (218), (221) to (223), (227) to (252), (254) to (267), (269) to (275), and (277) was formulated into a preparation according to Preparation Example 5. This preparation was diluted with water so that the active ingredient concentration became 500 ppm to prepare a test diluted solution.

Into a beer cup were poured 5 ml of the test diluted solution and 40 ml of water, where a cucumber planted in a polyethylene cup and grown to develop a first true leaf was accommodated, and the soil part was subjected to immersion-treatment. The plant was retained in a greenhouse at 25° C. for 7 days, 30 cotton aphids (all stages) were inoculated on a cucumber leaf surface, the plant was further retained in a greenhouse at 25° C. for 6 days, the number of surviving worms of cotton aphids parasitized on the leaf surface was investigated, and a controlling value was calculated according to the following equation.

Controlling value (%)={1−(Cb×Tai)/(Cai×Tb)}×100

wherein:

Cb is the number of worms before treatment of a non-treated group.

Cai is the number of worms at observation of a non-treated group.

Tb is the number of worms before treatment of a treated group.

Tai is the number of worms at observation of a treated group.

As a result, the groups treated by test the test diluted sections of present compounds (1) to (15), (17), (19), (21) to (26), (28) to (30), (34), (36) to (41), (49), (50), (53) to (57), (59), (61), (62), (67) to (69), (79), (119) to (123), (125), (127), (129), (130), (133) to (142), (147), (201), (207) to (210), (213) to (215), (218), (221) to (223), (227) to (252), (254) to (267), (269) to (275), and (277) showed a controlling value of not lower than 90%, respectively.

### Test Example 3

Each of present compounds (1) to (3), (5), (7), (8), (10) to (15), (17), (21) to (25), (31), (34), (38), (39), (49), (52), (55) to (57), (59), (61), (67), (111), (112), (119), (120), (122), (125) to (130), (133) to (141), (201), (206), (211), (215), (220) to (223), (226) to (239), (241) to (244), (246) to (267), and (269) to (277), and a comparative compound (A) described later was formulated into a preparation according to Preparation Example 5. This preparation was diluted with water so that the active ingredient concentration became 500 ppm to prepare a test diluted solution.

Meanwhile, a cabbage was planted in a polyethylene cup, grown until a first true leaf was developed, other leaves other than a first true leaf were removed, and tobacco whitefly imagoes were released therein, and made to lay eggs for about 24 hours. The cabbage was retained in a greenhouse for 8 days and, in the sate where larvae were hatched from produced eggs, the test diluted solution was sprayed at a ratio of 20 ml/cup. Seven days after spraying, the number of surviving worms on the cabbage leaf was investigated, and a controlling value was calculated according to the following equation.

Controlling value (%)={1−(Cb×Tai)/(Cai×Tb)}×100

wherein:

Cb is the number of worms before treatment of a non-treated group.

Cai is the number of worms at observation of a non-treated group.

Tb is the number of worms before treatment of a treated group.

Tai is the number of worms at observation of a treated group.

As a result, the groups treated with the test diluted solutions of present compounds (1) to (3), (5), (7), (8), (10) to (15), (17), (21) to (25), (31), (34), (38), (39), (49), (52), (55) to (57), (59), (61), (67), (111), (112), (119), (120), (122), (125) to (130), (133) to (141), (201), (206), (211), (215), (220) to (223), (226) to (239), (241) to (244), (246) to (267), and (269) to (277) showed a controlling value of not lower than 90%, respectively, but a controlling value was 0% in the group treated with the test diluted solution of the comparative compound (A).

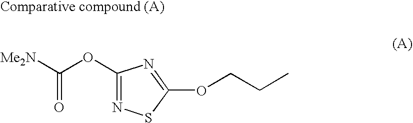

### Compound of J. Heterocyclic Chem.,16,961-971 (1979), Compound No. 21c

### Test Example 4

Each of present compounds (1) to (3), (5), (17), (39), (55) to (57), (120) to (124), (129), (130), (135), (136), (139) to (141), (232), (236), (243), (255), (262), and the comparative compound (A) was formulated into a preparation according to Preparation Example 5. This preparation was diluted with water so that the concentration of the present compound or the comparative compound (A) became 500 ppm.

Meanwhile, about 60 Tetranychus urticae female imagoes were released on a vineless common bean plantlet (7 days after seeding, first leaf development phase) planted in a plastic cup, which was allowed to stand for 1 day. To this plantlet was sprayed 20 ml of each of the above diluted solutions.

Eight days after spraying, the number of surviving mites on a leaf of the vineless common bean was investigated, and a controlling rate was calculated according to the following equation.

Controlling rate (%)=100×{1−(number of surviving mites of treated group)/(number of surviving mites of non-treated group)}

As a result, in all groups treated with each of present compounds (1) to (3), (5), (17), (39), (55) to (57), (120) to (124), (129), (130), (135), (136), (139) to (141), (232), (236), (243), (255), and (262), a controlling rate was not lower than 90%, but a controlling rate was 0% in the group treated with the comparative compound (A).

### INDUSTRIAL APPLICABILITY

Since the thiadiazole compound represented by the formula (I) of the present invention has the excellent controlling efficacy on a noxious arthropod, it is useful as an active ingredient of a noxious arthropod controlling agent.

1. A thiadiazole compound represented by the formula (I): 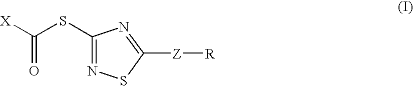
wherein
R is a hydrogen atom, (1) a C1-C7 chain hydrocarbon group optionally substituted with one or more substituents selected from the following A group, (2) a C3-C6 alkanoyl group, (3) a -Q group, (4) a -T-Q group, (5) a -T-O-Q group, or (6) a -T-O-T-Q group;
X is a —NR²R³ group or a group represented by the formula: 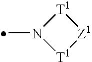
wherein Z is an oxygen atom or a sulfur atom;
Q is (1) a 3- to 10-membered carbocyclic group optionally substituted with one or more substituents selected from the following B group, or optionally substituted with one or more substituents selected from the following C group at the same position or adjacent positions, or (2) a 3- to 10-membered heterocyclic group optionally substituted with one or more substituents selected from the following B group, or optionally substituted with one or more substituents selected from the following C group at the same position or adjacent positions;
T is a C1-C4 alkanediyl group;
R² and R³ each independently are a hydrogen atom, a C1-C4 alkyl group, a C3-C4 alkenyl group, a C1-C4 alkoxy group, a benzyl group, or a phenyl group, or R² and R³ bind to each other at the ends thereof to form a C2-C7 alkanediyl group;
T¹ is a C2-C7 alkanediyl group; and
Z¹ is an oxygen atom, a sulfur atom, a —NH— group, or a —N(C1-C6 alkyl)- group;
A group: a monovalent substituent selected from the group consisting of a halogen atom, a cyano group, a nitro group, a —Z²-(T-Z²)r-R¹⁰ group, a -(Z²)p-C(═O)—(Z³)q-R¹⁰ group, and a —C(═NO—R¹⁰)—R¹¹ group;
B group: a monovalent substituent selected from the group consisting of a halogen atom, a cyano group, a nitro group, a —R¹² group, a —Z²-(T-Z²)r-R¹⁰ group, a -(T-Z²)s-R¹⁰ group, a —(Z²)p-C(═O)-(Z³)q-R¹⁰ group, a —C(═NO—R¹⁰)—R¹¹ group, a -Q¹ group, a -Z²-Q¹ group, a -T-Q¹ group, a -Z²-T-Q¹ group, and a -T-Z²-Q¹ group;
C group: a divalent substituent selected from the group consisting of an oxygen atom, a sulfur atom, a -T- group, a —Z⁴-T-Z⁵— group, and a -T-Z⁴-T- group;
wherein r is 0, 1 or 2, p and q each independently are 0 or 1, s is 1 or 2,
Z² and Z³ each independently are an oxygen atom, a sulfur atom, a —NH— group, or —N(C1-C6 alkyl)- group,
Z⁴ and Z⁵ each independently are an oxygen atom or a sulfur atom,
R¹⁰ and R¹¹ each independently are (1) a C1-C7 chain hydrocarbon group optionally substituted with a halogen atom, or (2) a hydrogen atom,
R¹² is a C1-C7 chain hydrocarbon group optionally substituted with a halogen atom, and
Q¹ is (1) a 3- to 10-membered carbocyclic group optionally substituted with one or more substituents selected from the above A group, or optionally substituted with one or more substituents selected from the above C group at the same position or adjacent positions, or (2) a 3- to 10-membered heterocyclic group optionally substituted with one or more substituents selected from the above A group, or optionally substituted with one or more substituents selected from the above C group at the same position or adjacent positions.

2. The thiadiazole compound according to claim 1, wherein X is a —NR²R³ group or a morpholino group, and R² and R³ each independently are a hydrogen atom, a C1-C4 alkyl group, a C3-C4 alkenyl group, a C1-C4 alkoxy group, a benzyl group, or a phenyl group, or a R² and R³ bind to each other at the ends thereof to form a C2-C7 alkanediyl group in the formula (I).

3. The thiadiazole compound according to claim 1, wherein X is a —NR²R³ group or a morpholino group, and R² and R³ each independently are a C1-C4 alkyl group or a phenyl group, or R² and R³ bind to each other at the ends thereof to form a C2-C7 alkanediyl group in the formula (I).

4. The thiadiazole compound according to claim 1, wherein R¹ is a C1-C7 chain hydrocarbon group optionally substituted with one or more substituents selected from the above A group, a -Q group, a -T-Q group, a -T-O-Q group, or a -T-O-T-Q group,
Q is (1) a 3- to 10-membered carbocyclic group optionally substituted with one or more substituents selected from the above B group, or optionally substituted with one or more substituents selected from the above C group at the same position or adjacent positions, or (2) a 3- to 10-membered heterocyclic group optionally substituted with one or more substituents selected from the above B group, or optionally substituted with one or more substituents selected from the above C group at the same position or adjacent positions, and
T is a C1-C4 alkanediyl group in the formula (I).

5. The thiadiazole compound according to claim 1, wherein R is a C1-C7 chain hydrocarbon group optionally substituted with one or more substituents selected from the following D group, a -Q² group, a -T-Q² group, a -T-O-Q² group, or a -T-O-T-Q² group,
wherein Q² is (1) a 3- to 10-membered carbocyclic group optionally substituted with one or more substituents selected from the following E group, or optionally substituted with one or more substituents selected from the following F group at the same position or adjacent positions, or (2) a 3- to 10-membered heterocyclic group optionally substituted with one or more substituents selected from the following E group, or optionally substituted with one or more substituents selected from the above F group at the same position or adjacent positions, and
T is a C1-C4 alkanediyl group in the formula (I);
D group: a monovalent substituent selected from the group consisting of a halogen atom, a —Z²-(T-Z²)r-R¹⁰ group, and a —(Z²)p-C(═O)-(Z³)q-R¹⁰ group;
E group: a monovalent substituent selected from the group consisting of a halogen atom, a —R¹² group, a —Z²-(T-Z²)r-R¹⁰ group, a -(T-Z²)s-R¹⁰ group, a —(Z²)p-C(═O)-(Z³)q-R¹⁰ group, a -Q³ group, a —Z²-Q³ group, a -T-Q³ group, a —Z²-T-Q³ group, and a -T-Z²-Q group;
F group: a divalent substituent selected from the group consisting of an oxygen atom, a -T- group, and a —Z⁴-T-Z⁵— group;
wherein Q³ is a 3- to 10-membered carbocyclic group or a 3- to 10-membered heterocyclic group and r, p, q, s, Z², Z³, Z⁴, Z⁵,R¹⁰ and R¹² are as defined above.

6. The thiadiazole compound according to claim 1, wherein R is (1) a C1-C7 chain hydrocarbon group optionally substituted with one or more substituents selected from the above D group, (2) a -Q⁴ group, (3) a -T-Q⁴ group, (4) a T-O-Q⁴ group, or (5) a -T-O-T-Q⁴ group,
Q⁴ is (1) a 3- to 6-membered carbocyclic group optionally substituted with one or more substituents selected from the above B group, or optionally substituted with one or more substituents selected from the above C group at the same position or adjacent positions, or (2) a 3- to 6-membered saturated heterocyclic group optionally substituted with one or more substituents selected from the above B group, or optionally substituted with one or more substituents selected from the above C group at the same position or adjacent positions in the formula (I).

7. The thiadiazole compound according to claim 1, wherein R is (1) a C1-C7 chain hydrocarbon group optionally substituted with one or more substituents selected from the above D group, (2) a -Q⁶ group, (3) -T-Q⁶ group, (4) a -T-O-Q⁶ group, or (5) a -T-O-T-Q⁶ group,
Q⁶ is a 3- to 6-membered carbocyclic group optionally substituted with one or more substituents selected from the above E group, or optionally substituted with one or more substituents selected from the above F group at the same position or adjacent positions, or (2) a 3- to 6-membered saturated heterocyclic group optionally substituted with one or more substituents selected from the above E group, or optionally substituted with one or more substituents selected from the above F group at the same position or adjacent positions, and
T is a C1-C4 alkanediyl group in the formula (I).

8. The thiadiazole compound according to claim 1, wherein R is (1) a C1-C7 chain hydrocarbon group optionally substituted with one or more substituents selected from the above D group, (2) a -Q⁷ group or (3) a -T-Q⁷ group, Q⁷ is (1) a C3-C8 cycloalkyl group optionally substituted with one or more substituents selected from the above E group, or optionally substituted with one or more substituents selected from the above F group at the same position or adjacent positions, or (2) a group represented by the formula: 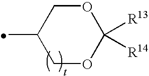
wherein t is 0 or 1, and
R¹³ and R¹⁴ each independently are a hydrogen atom, a C1-C4 alkyl group, a C2-C7 alkenyl group, a C2-C4 alkynyl group, a C1-C4 alkoxyalkyl group, or a -Q⁸ group, or R¹³ and R¹⁴ bind to each other at the ends thereof to form a C2-C7 alkanediyl group, or a —Z⁴-T-Z⁵— group,
Q⁸ is (1) a 3- to 10-membered carbocyclic group optionally substituted with one or more substituents selected from the above D group, or optionally substituted with one or more substituents selected from the above F group at the same position or adjacent positions, or (2) a 3- to 10-membered heterocyclic group optionally substituted with one or more substituents selected from the above D group, or optionally substituted with one or more substituents selected from the above F group at the same position or adjacent positions,
Z⁴ and Z⁵ each independently are an oxygen atom or a sulfur atom, and
T is a C1-C4 alkanediyl group in the formula [I].

9. A thiadiazole compound represented by the formula (I′): 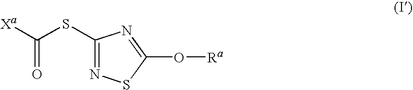
wherein
R^{a} is (1) a hydrogen atom, (2) a C1-C7 alkyl group, (3) a C1-C6 haloalkyl group, (4) a C3-C6 alkenyl group, (5) a C3-C6 haloalkenyl group, (6) a C3-C6 alkynyl group, (7) a C3-C6 haloalkynyl group, (8) a C2-C7 alkoxyalkyl group, (9) a C2-C6 alkylthioalkyl group, (10) a C3-C8 cycloalkyl group optionally substituted with one or more substituents selected from the following H group, (11) a C1-C4 alkyl group substituted with a C3-C8 cycloalkyl group optionally substituted with one or more substituents selected from the following H group, (12) a C5-C8 cycloalkenyl group optionally substituted with one or more substituents selected from the following H group, (13) a C1-C4 alkyl group substituted with a C5-C8 cycloalkenyl group optionally substituted with one or more substituents selected from the following H group, (14) a heterocyclic group optionally substituted with one or more substituents selected from the I group, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-membered heterocyclic group containing only one sulfur atom as the heteroatom thereof, (d) a 6-membered heterocyclic group containing only one or two sulfur atoms as the heteroatoms thereof, (e) a 5-membered heterocyclic group containing only one or two nitrogen atoms as the heteroatoms thereof, (f) a 5-membered heterocyclic group containing only a sulfur atom and a nitrogen atom as the heteroatoms thereof, (g) a 5-member heterocyclic group containing only a oxygen atom and a nitrogen atom as the heteroatoms thereof, and (h) a 6-membered heterocyclic group containing only one or two nitrogen atoms as the heteroatoms thereof, (15) a C1-C4 alkyl group substituted with a heterocyclic group optionally substituted with one or more substituents selected from the following I group, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-memberaed heterocyclic group containing only one sulfur atom as the heteroatom thereof, (d) a 6-membered heterocyclic group containing only one or two sulfur atoms as the heteroatoms thereof, (e) a 5-membered heterocyclic group containing only one or two nitrogen atoms as the heteroatoms thereof, (f) a 5-membered heterocyclic group containing only a sulfur atom and a nitrogen atom as the heteroatoms thereof, (g) a 5-member heterocyclic group containing only an oxygen atom and a nitrogen atom as the heteroatoms thereof, and (h) a 6-membered heterocyclic group containing only one or two nitrogen atoms as the heteroatoms thereof, (16) a phenyl group optionally substituted with one or more substituents selected from the following I group, (17) a C1-C4 alkyl group substituted with a phenyl group optionally substituted with one or more substituents selected from the following I group, (18) a C2-C6 formylalkyl group, (19) a C2-C6 cyanoalkyl group, (20) a C2-C6 hydroxyiminoalkyl group, (21) a C3-C7 alkoxyiminoalkyl group, (22) a C2-C8 alkylaminoalkyl group, (23) a C2-C6 alkoxycarbonylalkyl group, (24) a C2-C6 hydroxyalkyl group, or (25) a C3-C6 alkanoyl group; and
X^{a} is a morpholino group, or a —NR²R³ group, wherein R² and R³ each independently represent a hydrogen atom, a C1-C4 alkyl group, a C3-C4 alkenyl group, a C1-C4 alkoxy group, or a phenyl group, or R² and R³ bind to each other at the ends thereof to form a C2-C7 alkanediyl group;
H group: a monovalent substituent selected from the group consisting of a C1-C4 alkyl group optionally substituted with a halogen atom, a C2-C4 alkenyl group optionally substituted with a halogen atom, a C2-C4 alkynyl group optionally substituted with a halogen atom, and a halogen atom;
I group: a monovalent substituent selected from the group consisting of a C1-C4 alkyl group optionally substituted with a halogen atom, a C1-C4 alkoxy group optionally substituted with a halogen atom, a C1-C4 alkylthio group, a halogen atom, a cyano group, a nitro group, and a formyl group.

10. The thiadiazole compound according to claim 9, wherein R^{a} is (1) a C1-C7 alkyl group, (2) a C1-C6 haloalkyl group, (3) a C3-C6 alkenyl group, (4) a C3-C6 haloalkenyl group, (5) a C3-C6 alkynyl group, (6) a C2-C7 alkoxyalkyl group, (7) a C2-C6 alkylthioalkyl group, (8) a C3-C8 cycloalkyl group optionally substituted with one or more substituents selected from the following J group, (9) a C1-C4 alkyl group substituted with a C3-C8 cycloalkyl group optionally substituted with one or more substituents selected from the following J group, (10) a C1-C4 alkyl group substituted with a C5-C8 cycloalkenyl group optionally substituted with one or more substituents selected from the following J group, (11) a heterocyclic group optionally substituted with one or more substituents selected from the following K group, said heterocyclic group being selected from the group consisting of (a) a 5-membered heterocyclic group containing only one or two oxygen atoms as the heteroatoms thereof, and (b) a 6-membered heterocyclic group containing only one or two oxygen atoms as the heteroatoms thereof, (12) a C1-C4 alkyl group substituted with a heterocyclic group optionally substituted with one or more substituents selected from the following K group, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-membered heterocyclic group containing only one or two nitrogen atoms as the heteroatoms thereof, (d) a 5-membered heterocyclic group containing only a sulfur atom and a nitrogen atom as the heteroatoms thereof, and (e) a 6-membered heterocyclic group containing only one or two nitrogen atoms as the heteroatom thereof, or (13) a C1-C4 alkyl group substituted with a phenyl group optionally substituted with one or more substituent selected from the following L group in the formula (I′);
J group: a monovalent substituent selected from the group consisting of a C1-C4 alkyl group optionally substituted with a halogen atom, a C2-C4 alkynyl group, and a halogen atom;
K group: a monovalent substituent selected from the group consisting of a C1-C4 alkyl group, and a halogen atom;
L group: a monovalent substituent selected from the group consisting of a C1-C4 alkyl group optionally substituted with a halogen atom, a C1-C4 alkoxy group optionally substituted with a halogen atom, an alkylthio group, and a halogen atom.

11. The thiadiazole compound according to claim 9, wherein R^{a} is (1) a C1-C7 alkyl group, (2) a C1-C6 haloalkyl group, (3) a C3-C6 alkenyl group, (4) a C3-C6 haloalkenyl group, (5) a C3-C6 alkynyl group, (6) a C2-C7 alkoxyalkyl group, (7) a heterocyclic group optionally substituted with one or more C1-C4 alkyl groups, said heterocyclic group being selected from the group consisting of (a) a 5-membered heterocyclic group containing only one or two oxygen atoms as the heteroatom thereof, and (b) a 6-membered heterocyclic group containing only one or two oxygen atoms as the heteroatoms thereof, or (8) a C1-C4 alkyl group substituted with a heterocyclic group optionally substituted with one or more C1-C4 alkyl groups, said heterocyclic group being selected from the group consisting of (a) a 5-membered heterocyclic group containing only one or two oxygen atoms as the heteroatoms thereof, (b) a 6-membered heterocyclic group containing only one or two oxygen atoms as the heteroatoms thereof, (c) a 5-membered heterocyclic group containing only one or two nitrogen atoms as the heteroatoms thereof, and (d) and a 6-membered heterocyclic group containing one or two nitrogen atoms as the heteroatoms thereof in the formula (I′).

12. The thiadiazole compound according to claim 9, wherein X^{a} is a morpholino group, or a —NR²R³ group, wherein R² and R³ each independently are a C1-C4 alkyl group or a phenyl group, or R² and R bind to each other at the ends thereof to form a C2-C7 alkanediyl group in the formula (I′).

13. A thiadiazole compound represented by the formula (II): 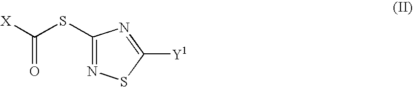
wherein
Y¹ is a halogen atom,
X is a —NR²R³ group or a group represented by the formula: 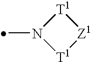
R² and R³ each independently are a hydrogen atom, a C1-C4 alkyl group, a C3-C4 alkenyl group, a C1-C4 alkoxy group, a benzyl group or a phenyl group, or R² and R³ bind to each other at the ends thereof to form a C2-C7 alkanediyl group,
T¹ is a C2-C7 alkanediyl group, and
Z¹ is an oxygen atom, a sulfur atom, a —NH— group or a —N(C1-C6 alkyl)- group.

14. The thiadiazole compound according to claim 13, wherein X is a —NR²R³ group or a morpholino group, and R² and R³ each independently are a C1-C4 alkyl group, or a phenyl group, or R² and R³ bind at an end to be C2-C7 alkanediyl group in the formula (II).

15. An agent for controlling a noxious arthropod comprising a compound according to claim 1 as an active ingredient.

16. Use of a compound according to claim 1 for controlling a noxious arthropod.

17. A method for controlling a noxious arthropod comprising applying a compound according to claim 1 to a noxious arthropod or a place where a noxious arthropod inhabits.
